(19) United States
(12) Patent Application Publication
(10) Pub. No.: US 2025/0161307 A1
(43) Pub. Date: May 22, 2025

(54) PYRAZOLOPYRIMIDINES AS MODULATORS OF SPERMINE OXIDASE

(71) Applicant: Janssen Pharmaceutica NV, Beerse (BE)

(72) Inventors: Lichao Fang, Shanghai (CN); Josep Llaveria Cros, Madrid (ES); Carlos Manuel Martinez Viturro, Toledo (ES); Carolina Martinez Lamenca, Beerse (BE); Christophe Buyck, Beerse (BE)

(21) Appl. No.: 18/833,141

(22) PCT Filed: Jan. 27, 2022
(86) PCT No.: PCT/CN2022/074253

§ 371 Date: Jul. 25, 2024

(51) Int. Cl. A61K 31/519; A61P 35/00; C07D 487/04

(52) U.S. Cl. CPC A61K 31/519; A61P 35/00; C07D 487/04

(57) ABSTRACT

The present invention comprises compounds of Formula I.

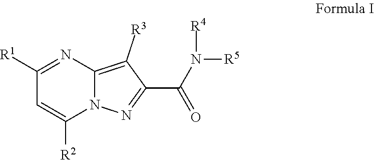

wherein:
R¹, R², R³, R⁴ and R⁵ are defined in the specification, and methods of treating or ameliorating a syndrome, disorder or disease described herein by administration of a therapeutically effective amount of at least one compound of Formula I.

### FIELD OF THE INVENTION

The invention is directed to substituted pyrazolopyrimidines compounds, which are inhibitors of spermine oxidase, pharmaceutical compositions, and methods for use thereof. More particularly, the spermine oxidase modulators are useful for preventing, treating or ameliorating a spermine oxidase mediated syndrome, disorder or disease.

### BACKGROUND OF THE INVENTION

Spermidine and spermine are polyamine members involved in many fundamental processes of cell growth and survival. Spermine oxidase (SMOX) oxidizes spermine to produce spermidine. The products of SMOX-mediated reactions are H₂O₂ and highly toxic aldehydes, which may also cause oxidative damage.

SMOX levels increase during chronic inflammation due to the presence of two cytokines, TNF-α and IL-6. Consequently, more SMOX is available to facilitate the polyamine catabolic reactions.

In chronic inflammation, as the intracellular concentrations of polyamine and SMOX increase, H₂O₂, 3-aminopropanal, and acrolein generate oxidative DNA damage, leading to modification of the immune microenvironment and carcinogenesis. (Stewart et al., J Biol Chem. 30:18736-18745 (2018)). In addition to contributing to oxidative damage of DNA, it is possible that SMOX contributes to tumorigenesis by silencing important tumor suppressor genes (Murray-Stewart et al., Biochemical Journal 473:2937-2953 (2016); Chaturvedi, et al., Oncogene 34:3429-3440 (2015); Tingting Hu et al., Hepatology Research 48:967-977 (2018)).

Targeting SMOX is a potential treatment for inhibiting cancer development. The relationship between inflammation, high levels of SMOX, DNA damage, and cancer formation has been tested in several cancer models. In a mouse model of colon tumorigenesis induced by Bacteroides fragilis infection, it has been found that a high level of polyamine oxidase induced upon infection, was responsible for DNA damage (Goodwin et al., Proc Natl Acad Sci USA. 13:15354-15359, 2011). In other studies, increased SMOX expression and activity has been found in the early stages of colorectal cancer (CRC) in an analysis of 50 human samples stages I-IV, supporting the possible use of SMOX inhibition as a target for CRC interception strategy (Snezhkina et al., Oxid Med Cell Longev. 2016:2353560 (2016)).

The suggestion that hSMOX could serve as a target in chemoprevention mainly stems from works in which hSMOX is inhibited by the polyamine oxidase inhibitor MDL72527 (Seiler, N., Duranton, B. & Raul, F. Prog Drug Res. 1-40 (2002). Chaturvedi, R. et al. J. Biol. Chem. 279, 40161-40173 (2004). Chaturvedi, R. et al. Oncogene. 2015 June; 34(26):3429-40. Bianchi, M. et al. FEBS J. 273, 1115-1123 (2006)) and more recently through 3,5-diamino-1,2,4-triazole analogues and N1-nonyl-1,4-diaminobutane (Holshouser, S. et al. Medchemcomm 10, 778-790 (2019), Masuko, T. et al. Neurosci. Lett. 672, 118-122 (2018)). MDL 72527 (N¹,N⁴-di-2,3-butadienyl-1,4-butanediamine dihydrochloride) is a non-specific inhibitor of polyamine oxidases that inhibits both SMOX and peroxisomal oxidase. In animal cancer models, MDL 72527 decreased inflammation, allowing tissue repair and significantly reduced cancer incidence (Goodwin et al., Proc Natl Acad Sci USA. 13:15354-15359, 2011).

SMOX is also a potential target for treatment of other diseases. For example, SMOX and deregulation of the polyamine metabolic pathway is implicated in various neurodegenerative diseases, renal failure and diabetes (Seiler et al., Neurochem. Res. 25:471-490 (2000); Casero et al., Biochem. J. 421:323-338 (2009); Zahedi, et al., J. Neurotrauma 27:515-525 (2010); Narayanan et al. Pharmacological Research 147:104299 (2019)). Studies have demonstrated that polyamine oxidation is involved in mediating neuronal and vascular damage in the retina (Narayanan, et al., Cell Death Dis. 5:e1075 (2014); Patel, et al., Biochim. Biophys. Acta 1862:1628-1639 (2016); Pichavaram, et al., Front. Neurosci. 12:956 (2018)). Increased expression of SMOX was observed in response to hyperoxia-induced neuronal damage in the retina as well as excitotoxicity-induced retinal neurodegeneration. Treatment with SMOX inhibitor showed significant reduction of neuronal death and retinal degeneration in significant models. For example, MDL72527 reduced neuronal death and retinal degeneration in associated disease models, predicting that blocking hSMOX could also be effective in preventing or delaying vision loss in diabetic patients (Wu, D. et al. Investig. Ophthalmol. Vis. Sci. 61, (2020). Liu, F. et al. J. Clin. Med. 9, 340 (2020), Narayanan, S. P., Shosha, E. & Palani, C. D. Pharmacol. Res. 147, (2019)).

Thus there remains a need for small molecule SMOX inhibitors to be investigated for the treatment of colorectal cancer, the prevention of cancers generally, and the delay or prevention of vision loss in diabetic patients.

### SUMMARY OF THE INVENTION

The invention comprises a method of inhibiting spermine oxidase in a cell comprising contacting said cell with a compound of Formula I

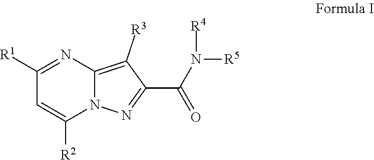

wherein:
R¹ is phenyl,

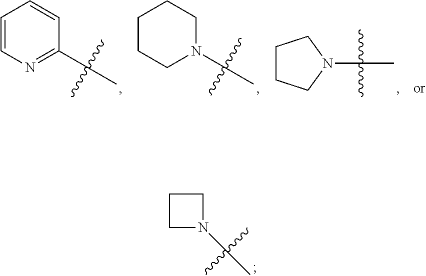 [SMILES: *c1ccccn1 ; *N1CCCCC1 ; *N1CCCC1 ; *N1CCC1]

herein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl,

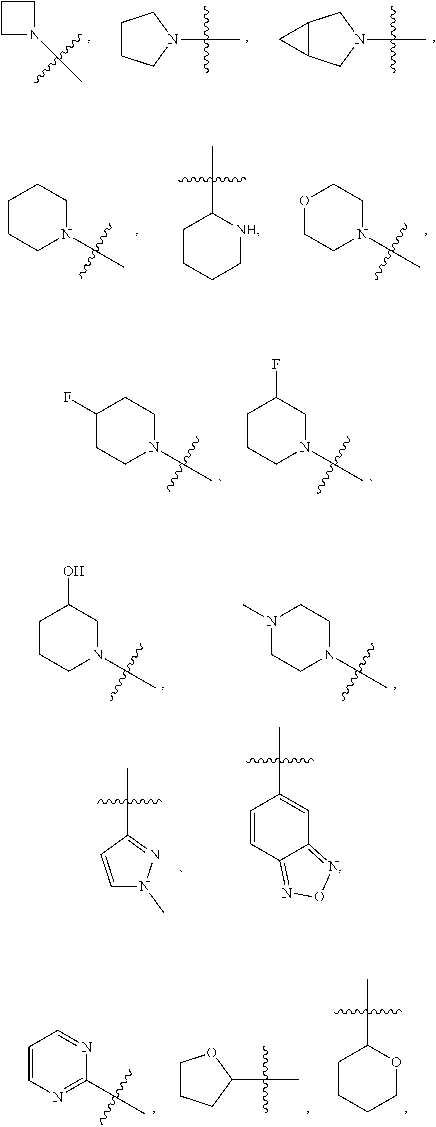

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H —I, —Cl, —F, —CN, or —CH₃;
R⁴ is —H₂, or —CH₃;
R⁵ is —C₍₂₋₃₎alkyl-R^{a}, cyclobutyl,

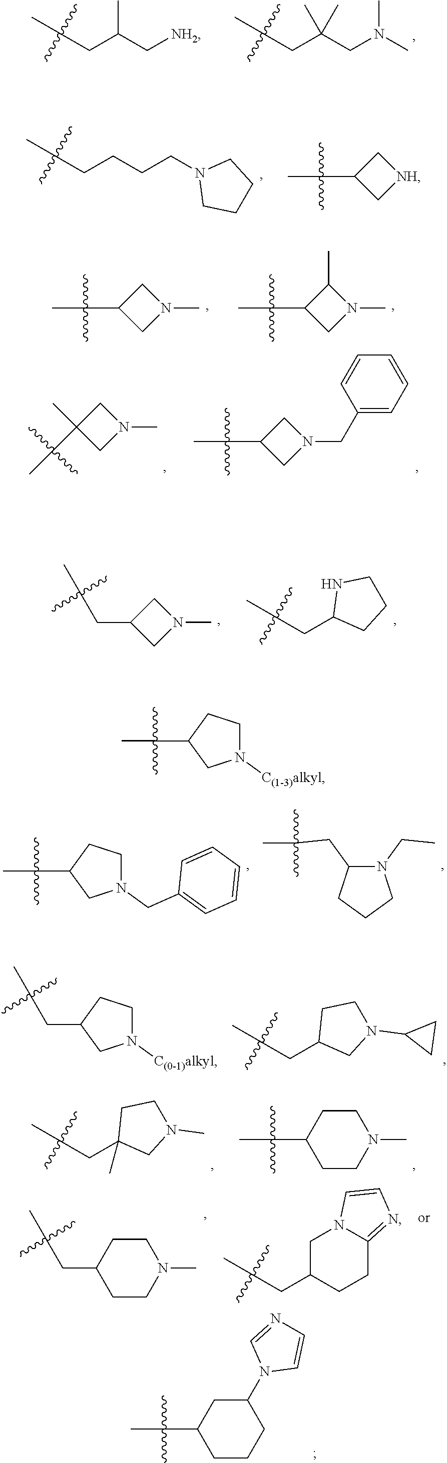

R^{a} is phenyl, imidazolyl, pyridinyl, azetidinyl, morpholinyl, —NH₂, —NHCH₃, —N(CH₃)₂, —NHpyridinyl, or —N(CH₃)pyridinyl; wherein said imidazolyl is optionally substituted with —CH₃;
and pharmaceutically acceptable salts thereof.

Another embodiment of the invention is a method for treating or ameliorating a SMOX mediated syndrome, disorder or disease comprising administering to a subject in need thereof an effective amount of a compound of Formula I

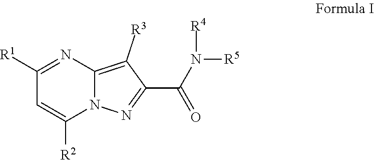

wherein:
R¹ is phenyl,

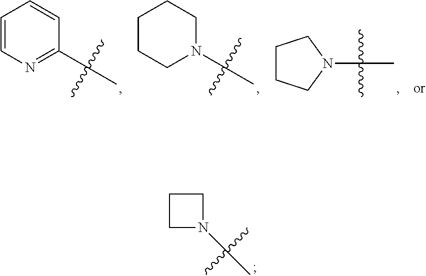 [SMILES: *c1ccccn1 ; *N1CCCCC1 ; *N1CCCC1 ; *N1CCC1]

wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl,

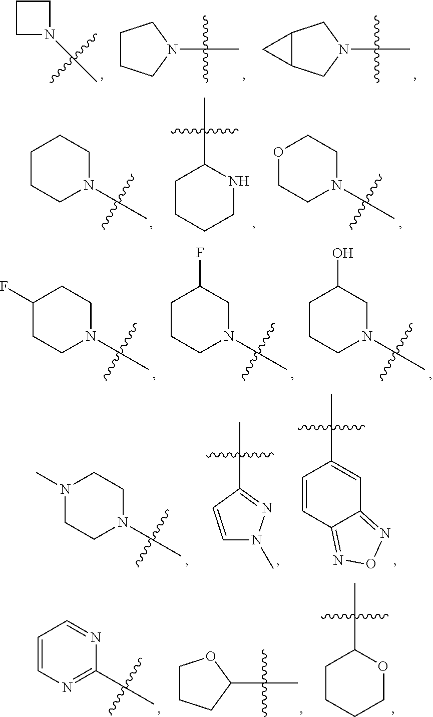

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H —I, —Cl, —F, —CN, or —CH₃;
R⁴ is —H, or —CH₃;
R⁵ is —C₍₂₋₃₎alkyl-R^{a}, cyclobutyl,

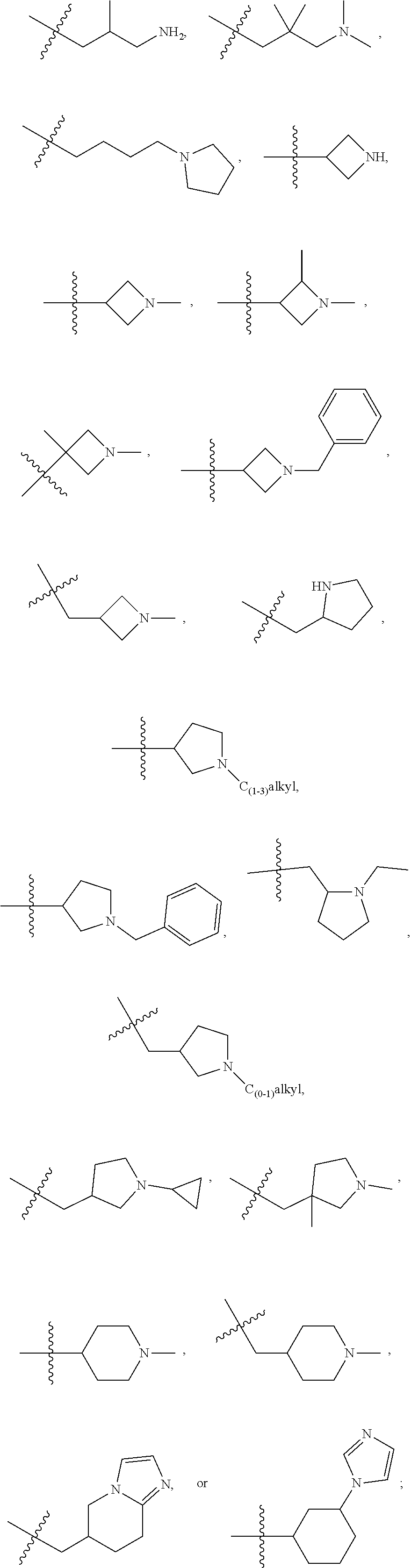

R^{a} is phenyl, imidazolyl, pyridinyl, azetidinyl, morpholinyl, —NH₂, —NHCH₃, —N(CH₃)₂, —NHpyridinyl, or —N(CH₃)pyridinyl; wherein said imidazolyl is optionally substituted with —CH₃;
and pharmaceutically acceptable salts thereof.

Another embodiment of the invention is a compound of Formula I

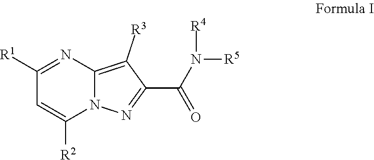

wherein:
R¹ is phenyl,

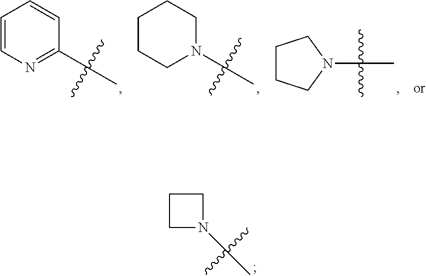 [SMILES: *c1ccccn1 ; *N1CCCCC1 ; *N1CCCC1 ; *N1CCC1]

wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl,

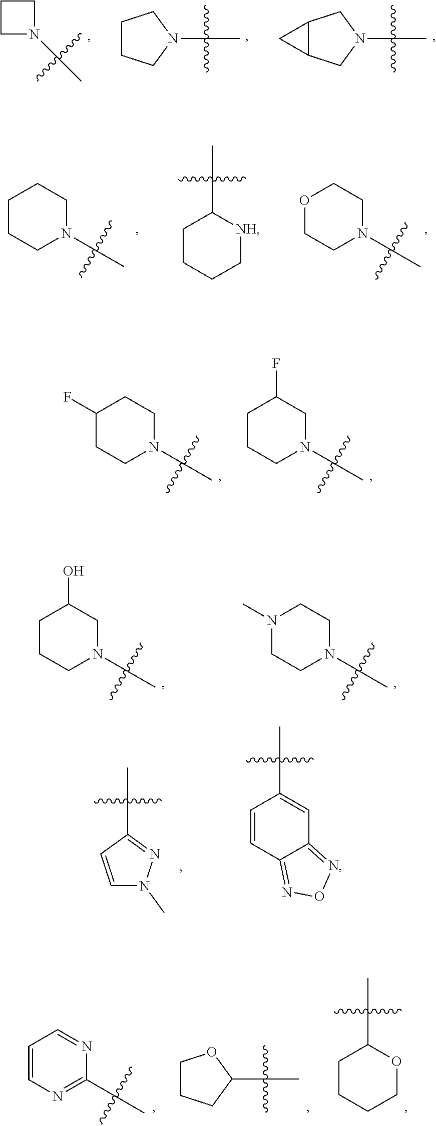

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H —I, —Cl, —F, —CN, or —CH₃;
R⁴ is —H, or —CH₃;
R⁵ is —C₍₂₋₃₎alkyl-R^{a}, cyclobutyl,

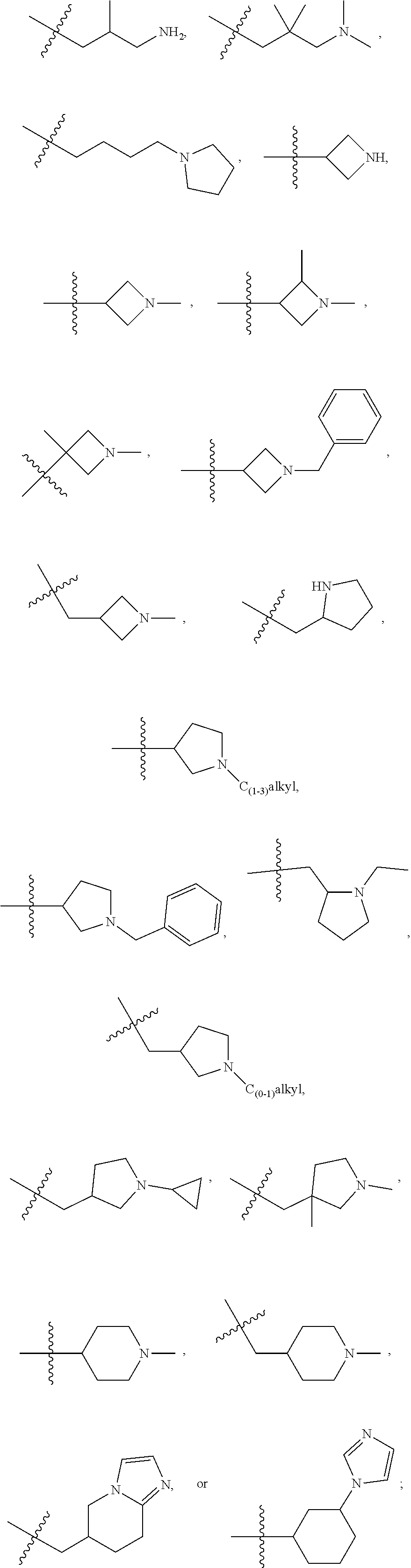

R^{a} is phenyl, imidazolyl, pyridinyl, azetidinyl, morpholinyl, —NH₂, —NHCH₃, —N(CH₃)₂, —NHpyridinyl, or —N(CH₃)pyridinyl; wherein said imidazolyl is optionally substituted with —CH₃;
and pharmaceutically acceptable salts thereof.

### DETAILED DESCRIPTION OF THE INVENTION

The invention comprises a method of inhibiting spermine oxidase in a cell comprising contacting said cell with a compound of Formula I

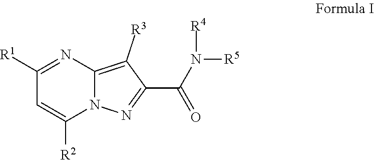

wherein:
R¹ is phenyl,

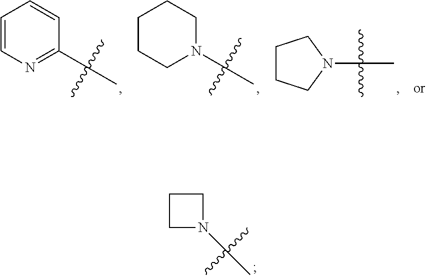 [SMILES: *c1ccccn1 ; *N1CCCCC1 ; *N1CCCC1 ; *N1CCC1]

wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl,

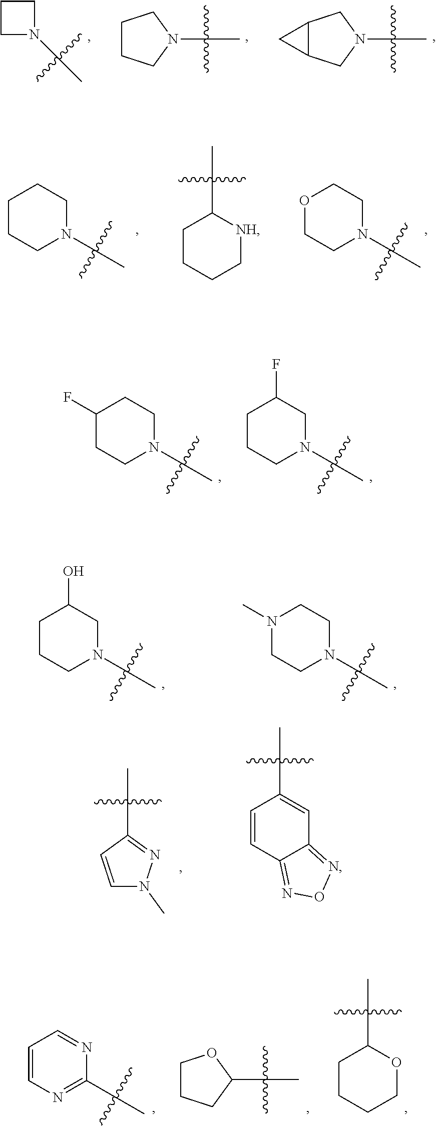

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H, —I, —Cl, —F, —CN, or —CH₃;
R⁴ is —H, or —CH₃;
R⁵ is —C₍₂₋₃₎alkyl-R^{a}, cyclobutyl,

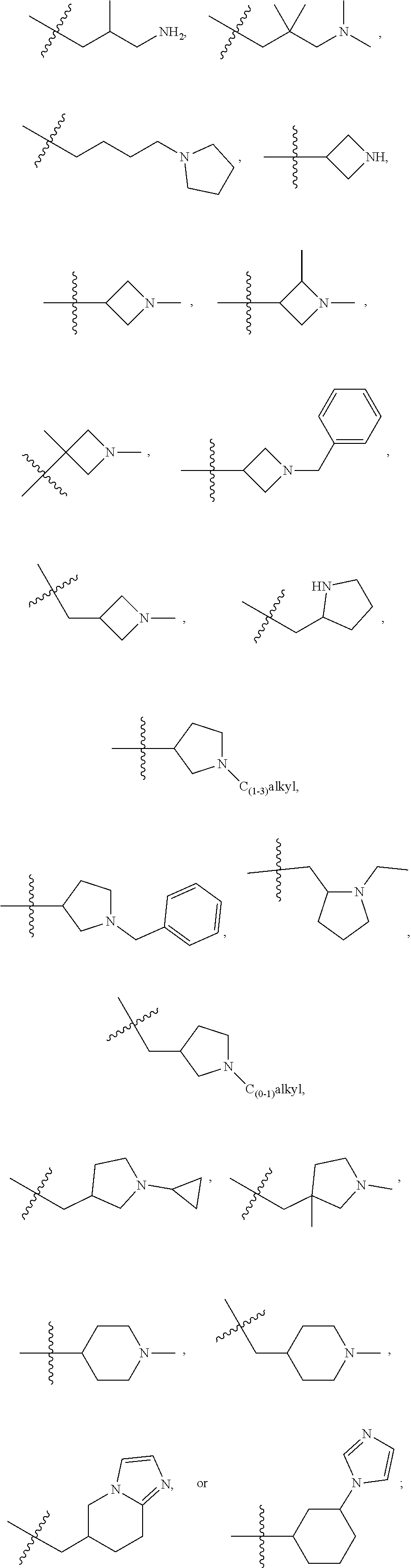

R^{a} is phenyl, imidazolyl, pyridinyl, azetidinyl, morpholinyl, —NH₂, —NHCH₃, —N(CH₃)₂, —NHpyridinyl, or —N(CH₃)pyridinyl; wherein said imidazolyl is optionally substituted with —CH₃;
and pharmaceutically acceptable salts thereof.

Another embodiment of the invention is a method for treating or ameliorating a SMOX mediated syndrome, disorder or disease comprising administering to a subject in need thereof an effective amount of a compound of Formula I

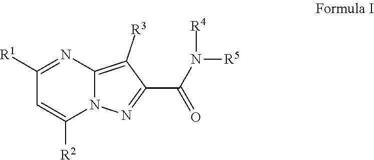

wherein:
R¹ is phenyl,

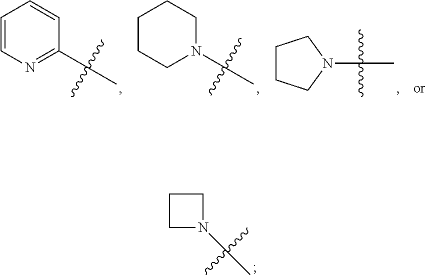 [SMILES: *c1ccccn1 ; *N1CCCCC1 ; *N1CCCC1 ; *N1CCC1]

wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl,

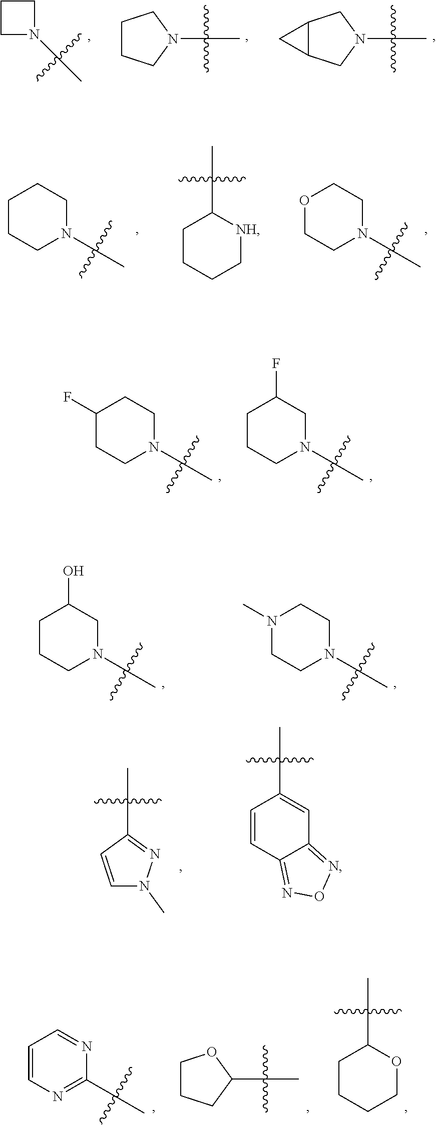

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H —I, —Cl, —F, —CN, or —CH₃;
R⁴ is —H, or —CH₃;
R⁵ is —C₍₂₋₃₎alkyl-R^{a}, cyclobutyl,

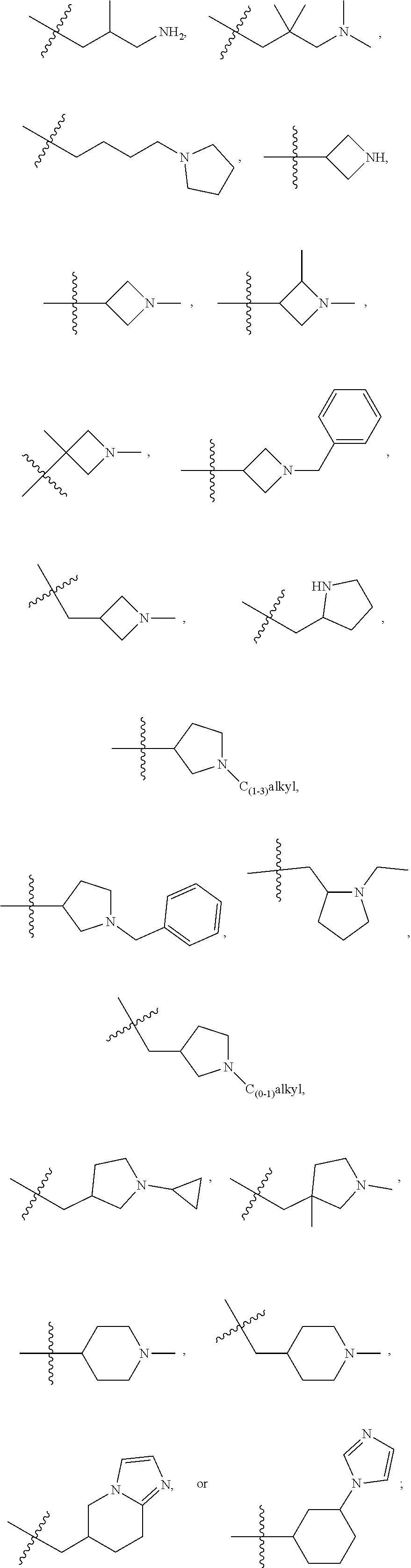

R^{a} is phenyl, imidazolyl, pyridinyl, azetidinyl, morpholinyl, —NH₂, —NHCH₃, —N(CH₃)₂, —NHpyridinyl, or —N(CH₃)pyridinyl; wherein said imidazolyl is optionally substituted with —CH₃;
and pharmaceutically acceptable salts thereof.

In another embodiment of the invention:
R¹ is phenyl;
R² is phenyl, pyridinyl, —CH₃, —CH₂CH(CH₃)₂, —C₍₃₋₆₎cycloalkyl, —NHC₍₄₋₅₎cycloalkyl,

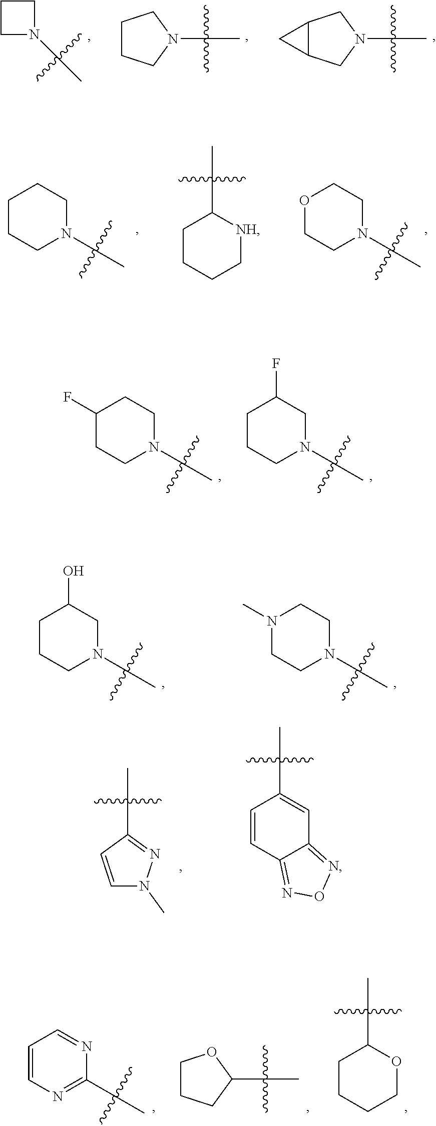

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H —I, —Cl, —F, —CN, or —CH₃;
R⁴ is —H, or —CH₃;
R⁵ is —C₍₂₋₃₎alkyl-R^{a}, cyclobutyl,

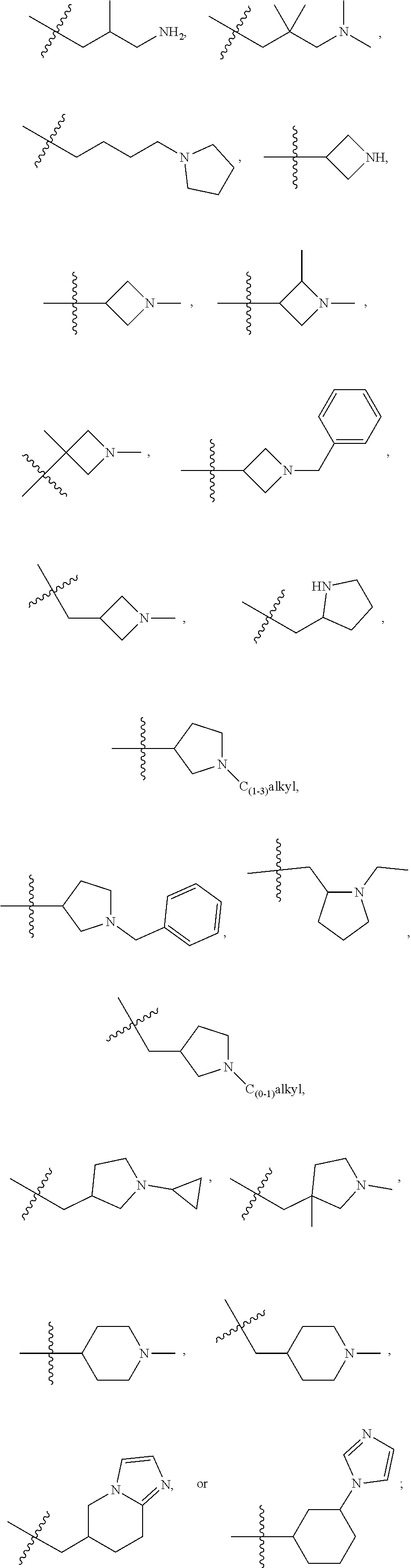

R^{a} is phenyl, imidazolyl, pyridinyl, azetidinyl, morpholinyl, —NH₂, —NHCH₃, —N(CH₃)₂, —NHpyridinyl, or —N(CH₃)pyridinyl; wherein said imidazolyl is optionally substituted with —CH₃;
and pharmaceutically acceptable salts thereof.

In another embodiment of the invention:
R¹ is phenyl,

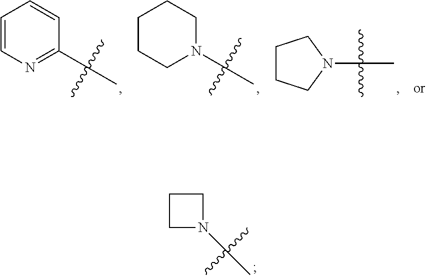 [SMILES: *c1ccccn1 ; *N1CCCCC1 ; *N1CCCC1 ; *N1CCC1]

wherein said phenyl is optionally substituted with —F, or —CF₃;
R² is phenyl;
R³ is —H —I, —Cl, —F, —CN, or —CH₃;
R⁴ is —H, or —CH₃;
R⁵ is —C₍₂₋₃₎alkyl-R^{a}, cyclobutyl,

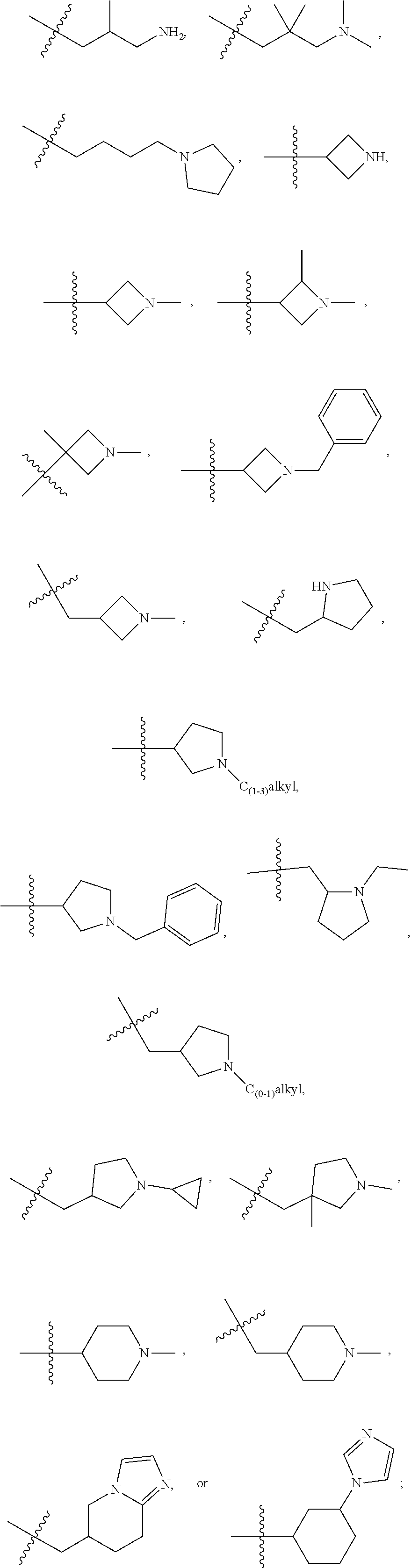

R^{a} is phenyl, imidazolyl, pyridinyl, azetidinyl, morpholinyl, —NH₂, —NHCH₃, —N(CH₃)₂, —NHpyridinyl, or —N(CH₃)pyridinyl; wherein said imidazolyl is optionally substituted with —CH₃;
and pharmaceutically acceptable salts thereof.

In another embodiment of the invention:
R¹ is phenyl,

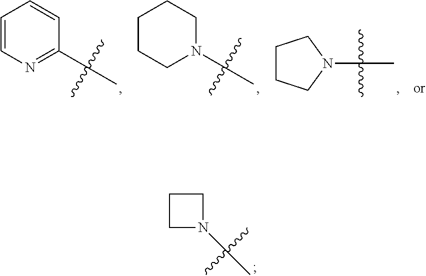 [SMILES: *c1ccccn1 ; *N1CCCCC1 ; *N1CCCC1 ; *N1CCC1]

wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl,

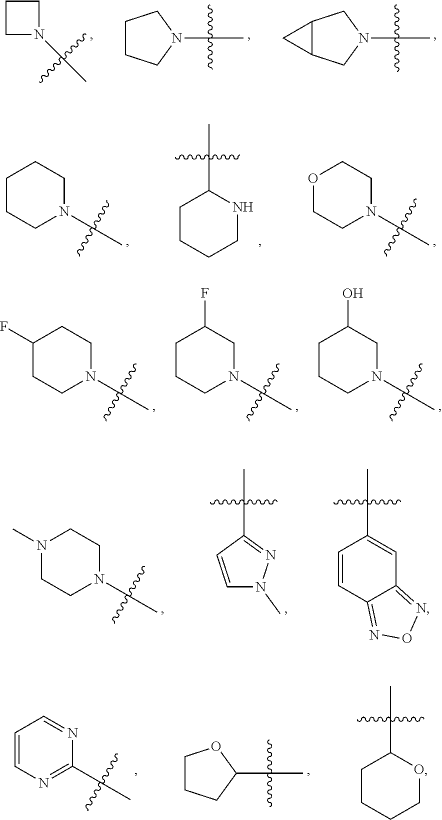

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H;
R⁴ is —H, or —CH₃;
R⁵ is —C₍₂₋₃₎alkyl-R^{a}, cyclobutyl,

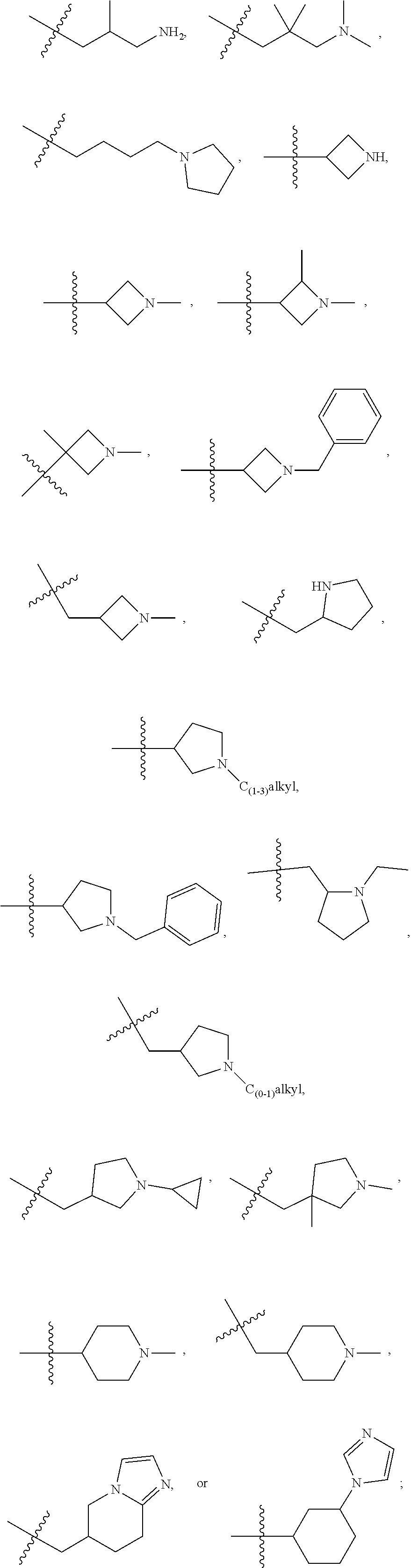

R^{a} is imidazolyl, pyridinyl, azetidinyl, morpholinyl, —NH₂, —NHCH₃, —N(CH₃)₂, —NHpyridinyl, or —N(CH₃)pyridinyl; wherein said imidazolyl is optionally substituted with —CH₃;
and pharmaceutically acceptable salts thereof.

In another embodiment of the invention:
R¹ is phenyl,

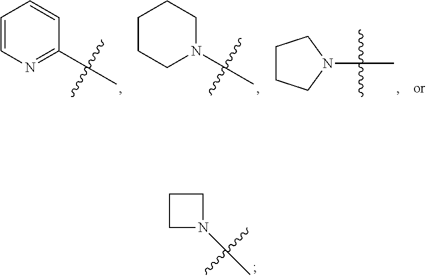 [SMILES: *c1ccccn1 ; *N1CCCCC1 ; *N1CCCC1 ; *N1CCC1]

wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl,

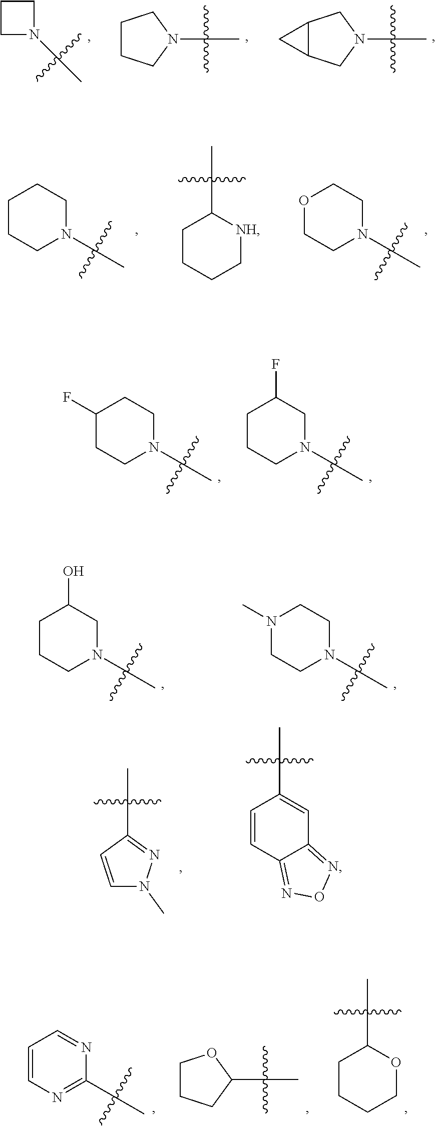

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H —I, —Cl, —F, —CN, or —CH₃;
R⁴ is —H;
R⁵ is —C₍₂₋₃₎alkyl-R^{a}, cyclobutyl,

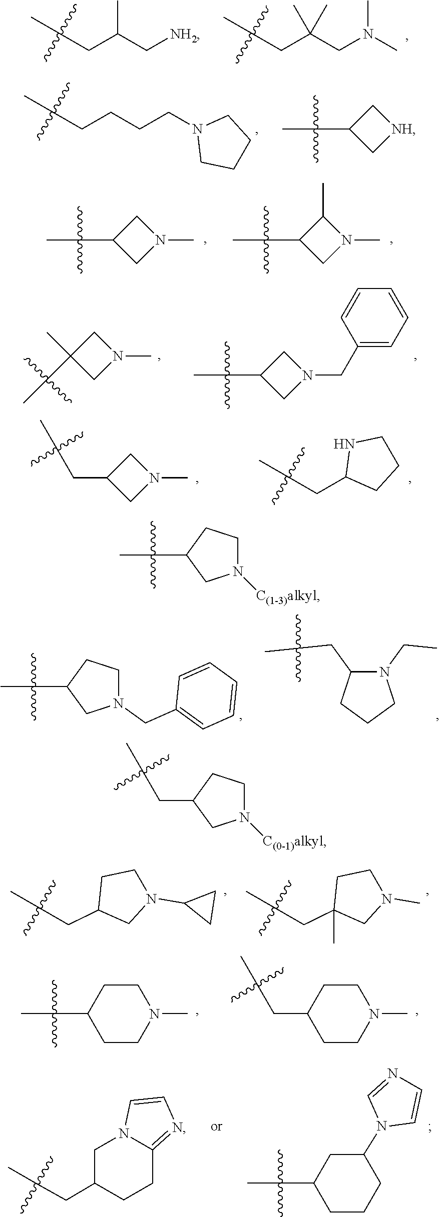

R^{a} is imidazolyl, pyridinyl, azetidinyl, morpholinyl, —NH₂, —NHCH₃, —N(CH₃)₂, —NHpyridinyl, or —N(CH₃)pyridinyl; wherein said imidazolyl is optionally substituted with —CH₃;
and pharmaceutically acceptable salts thereof.

Another embodiment of the invention is a method of inhibiting spermine oxidase in a cell comprising contacting said cell with a compound selected from the group consisting of:

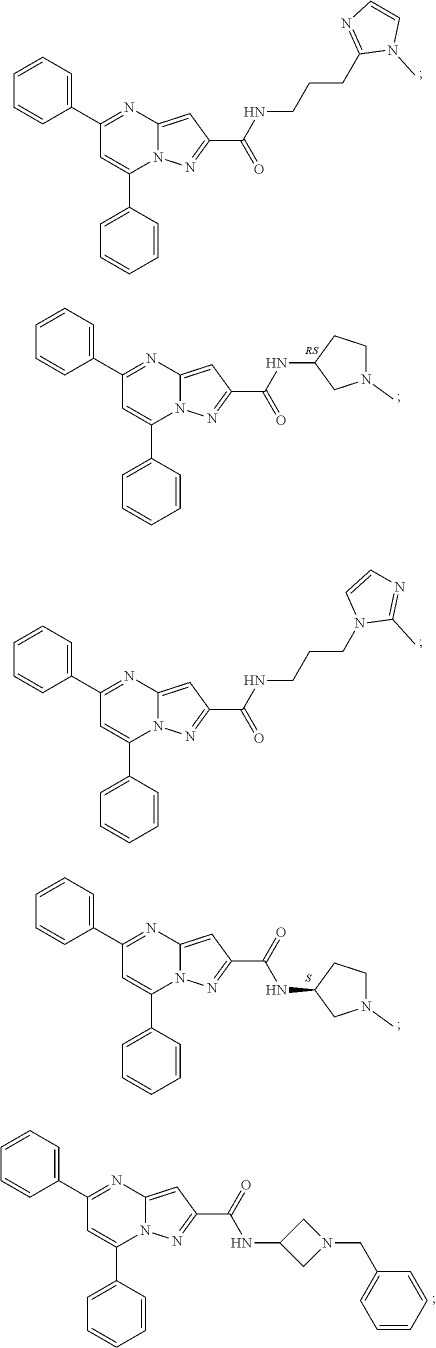

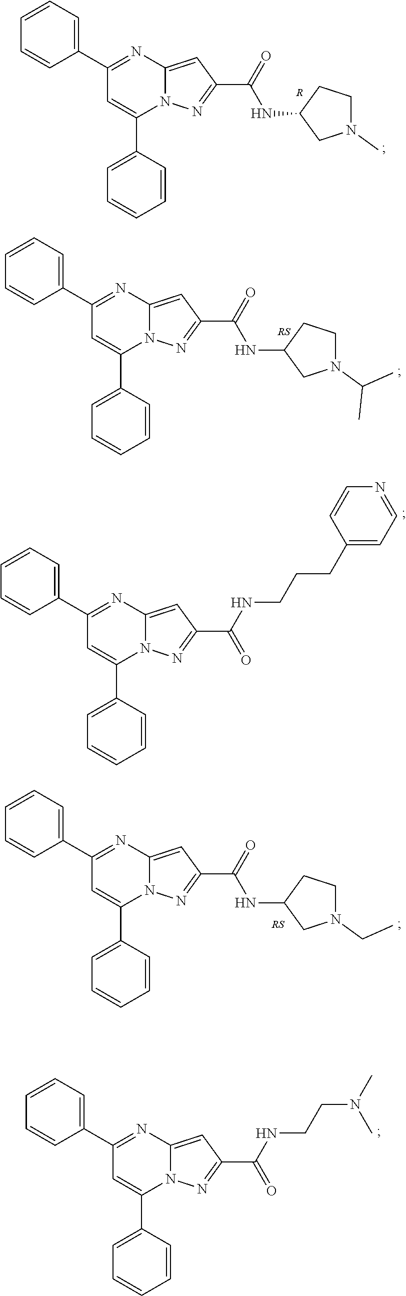

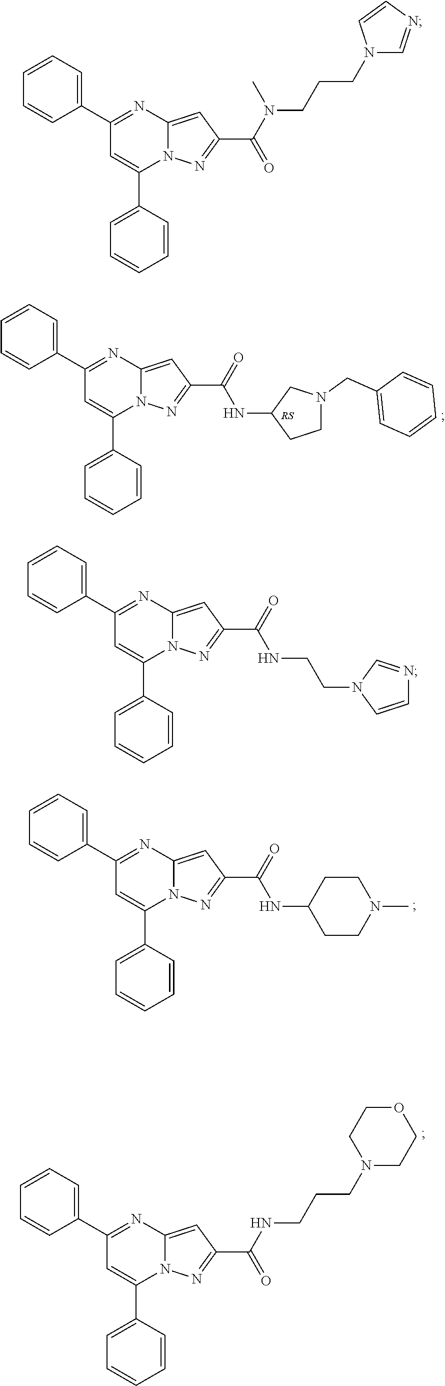

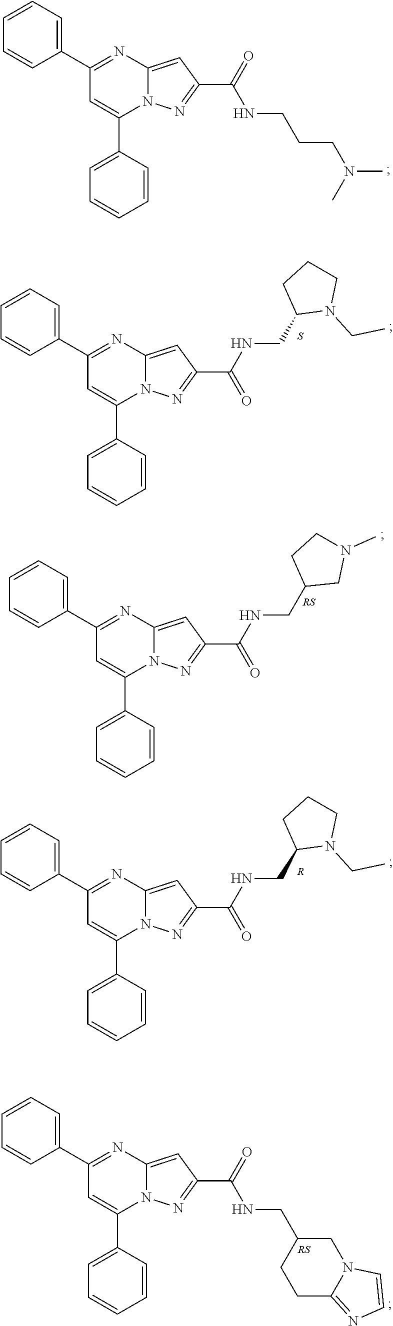

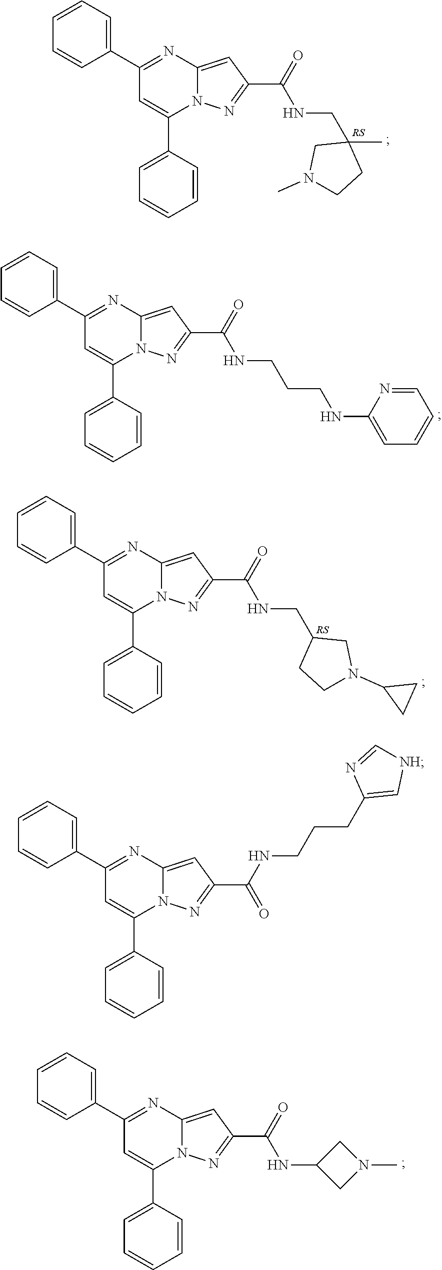

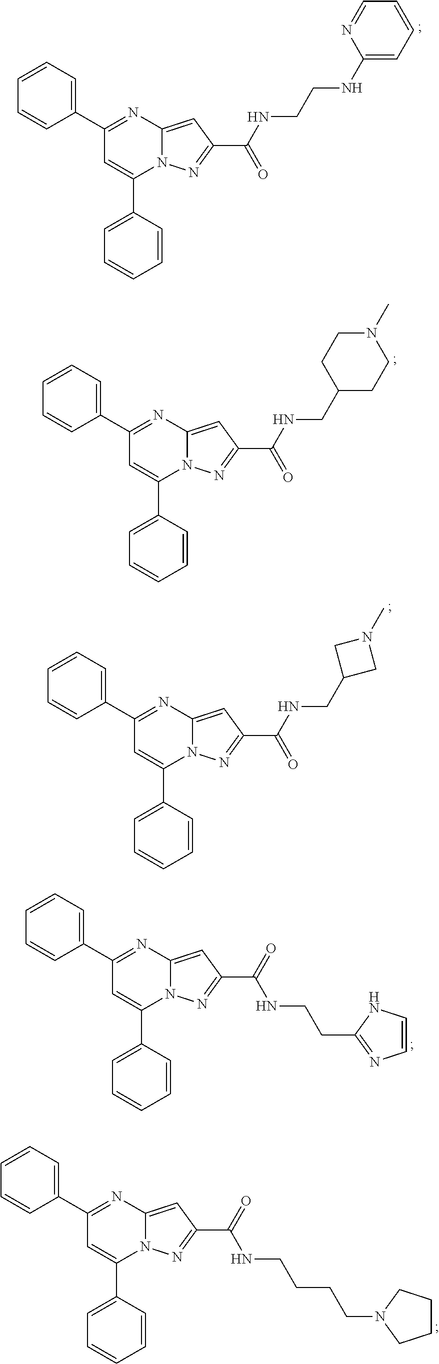 [SMILES: O=C(NCCNc1ccccn1)c1cc2nc(-c3ccccc3)cc(-c3ccccc3)n2n1 ; CN1CCC(CNC(=O)c2cc3nc(-c4ccccc4)cc(-c4ccccc4)n3n2)CC1 ; CN1CC(CNC(=O)c2cc3nc(-c4ccccc4)cc(-c4ccccc4)n3n2)C1 ; O=C(NCCc1ncc[nH]1)c1cc2nc(-c3ccccc3)cc(-c3ccccc3)n2n1 ; O=C(NCCCCN1CCCC1)c1cc2nc(-c3ccccc3)cc(-c3ccccc3)n2n1]

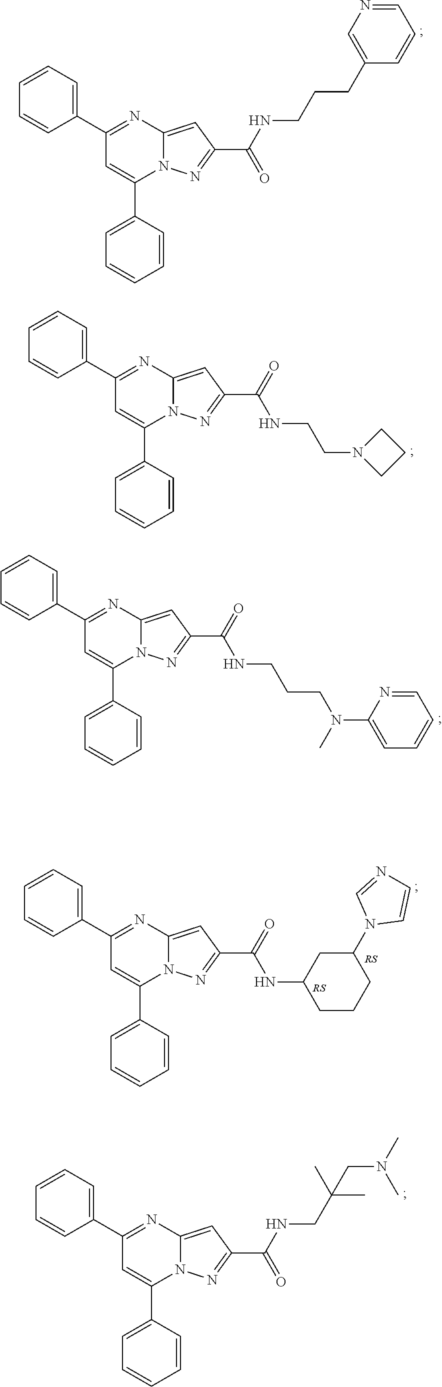 [SMILES: O=C(NCCCc1cccnc1)c1cc2nc(-c3ccccc3)cc(-c3ccccc3)n2n1 ; O=C(NCCN1CCC1)c1cc2nc(-c3ccccc3)cc(-c3ccccc3)n2n1 ; CN(CCCNC(=O)c1cc2nc(-c3ccccc3)cc(-c3ccccc3)n2n1)c1ccccn1 ; O=C(NC1CCCC(n2ccnc2)C1)c1cc2nc(-c3ccccc3)cc(-c3ccccc3)n2n1 ; CN(C)CC(C)(C)CNC(=O)c1cc2nc(-c3ccccc3)cc(-c3ccccc3)n2n1]

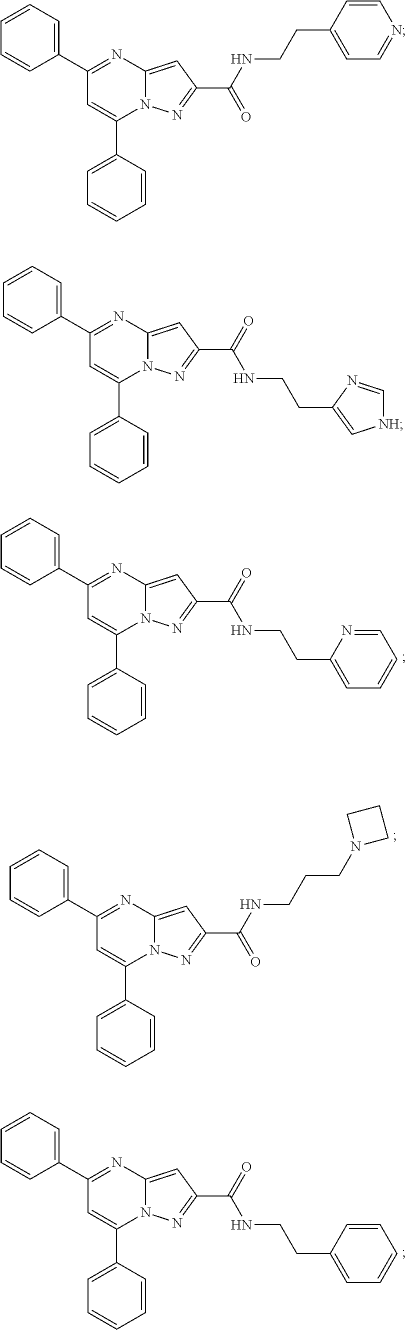 [SMILES: O=C(NCCc1ccncc1)c1cc2nc(-c3ccccc3)cc(-c3ccccc3)n2n1 ; O=C(NCCc1c[nH]cn1)c1cc2nc(-c3ccccc3)cc(-c3ccccc3)n2n1 ; O=C(NCCc1ccccn1)c1cc2nc(-c3ccccc3)cc(-c3ccccc3)n2n1 ; O=C(NCCCN1CCC1)c1cc2nc(-c3ccccc3)cc(-c3ccccc3)n2n1 ; O=C(NCCc1ccccc1)c1cc2nc(-c3ccccc3)cc(-c3ccccc3)n2n1]

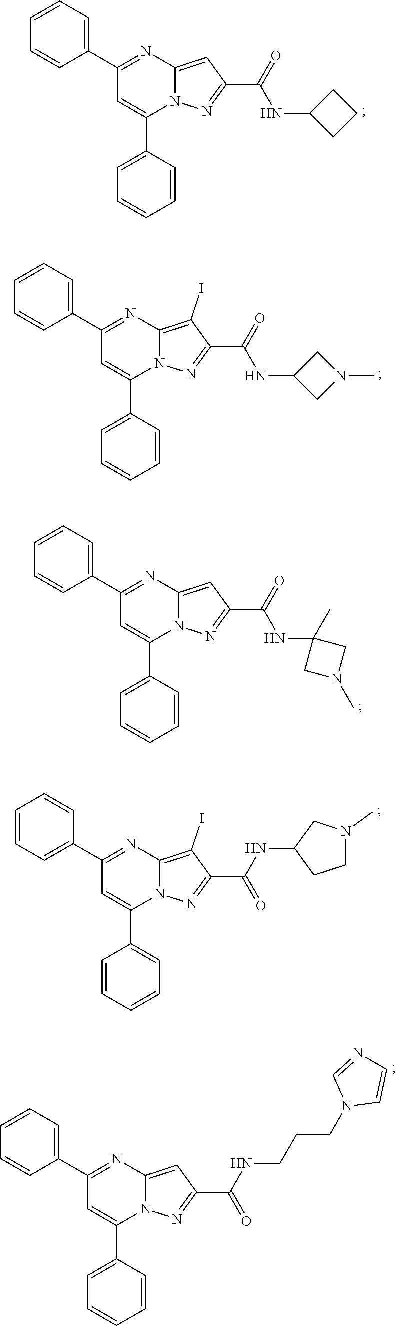 [SMILES: O=C(NC1CCC1)c1cc2nc(-c3ccccc3)cc(-c3ccccc3)n2n1 ; CN1CC(NC(=O)c2nn3c(-c4ccccc4)cc(-c4ccccc4)nc3c2I)C1 ; CN1CC(C)(NC(=O)c2cc3nc(-c4ccccc4)cc(-c4ccccc4)n3n2)C1 ; CN1CCC(NC(=O)c2nn3c(-c4ccccc4)cc(-c4ccccc4)nc3c2I)C1 ; O=C(NCCCn1ccnc1)c1cc2nc(-c3ccccc3)cc(-c3ccccc3)n2n1]

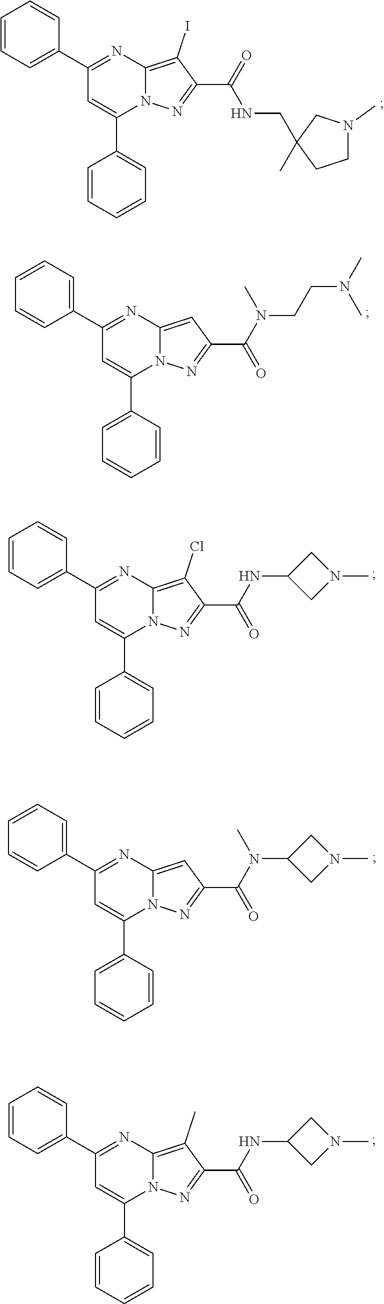 [SMILES: CN1CCC(C)(CNC(=O)c2nn3c(-c4ccccc4)cc(-c4ccccc4)nc3c2I)C1 ; CN(C)CCN(C)C(=O)c1cc2nc(-c3ccccc3)cc(-c3ccccc3)n2n1 ; CN1CC(NC(=O)c2nn3c(-c4ccccc4)cc(-c4ccccc4)nc3c2Cl)C1 ; CN1CC(N(C)C(=O)c2cc3nc(-c4ccccc4)cc(-c4ccccc4)n3n2)C1 ; Cc1c(C(=O)NC2CN(C)C2)nn2c(-c3ccccc3)cc(-c3ccccc3)nc12]

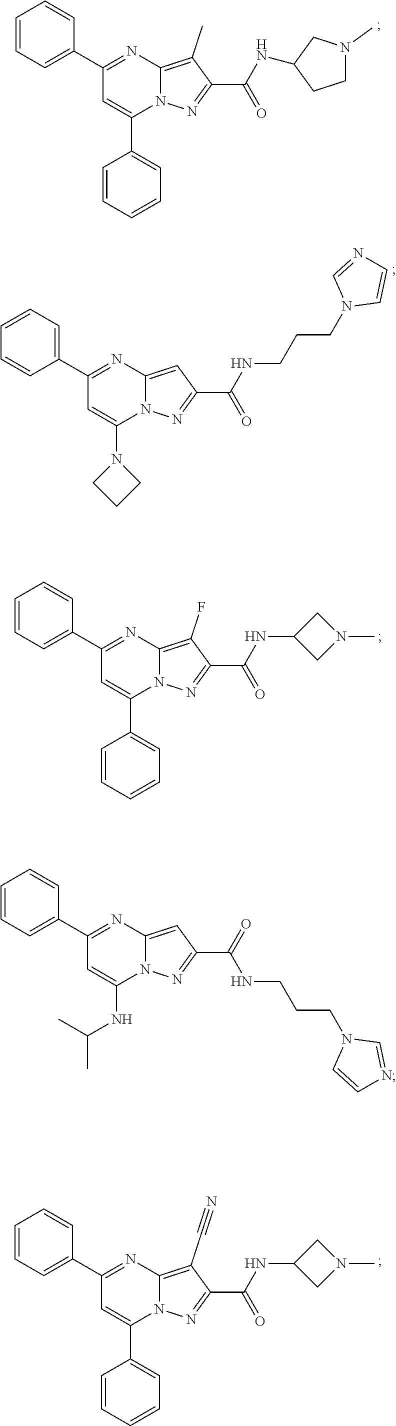 [SMILES: Cc1c(C(=O)NC2CCN(C)C2)nn2c(-c3ccccc3)cc(-c3ccccc3)nc12 ; O=C(NCCCn1ccnc1)c1cc2nc(-c3ccccc3)cc(N3CCC3)n2n1 ; CN1CC(NC(=O)c2nn3c(-c4ccccc4)cc(-c4ccccc4)nc3c2F)C1 ; CC(C)Nc1cc(-c2ccccc2)nc2cc(C(=O)NCCCn3ccnc3)nn12 ; CN1CC(NC(=O)c2nn3c(-c4ccccc4)cc(-c4ccccc4)nc3c2C#N)C1]

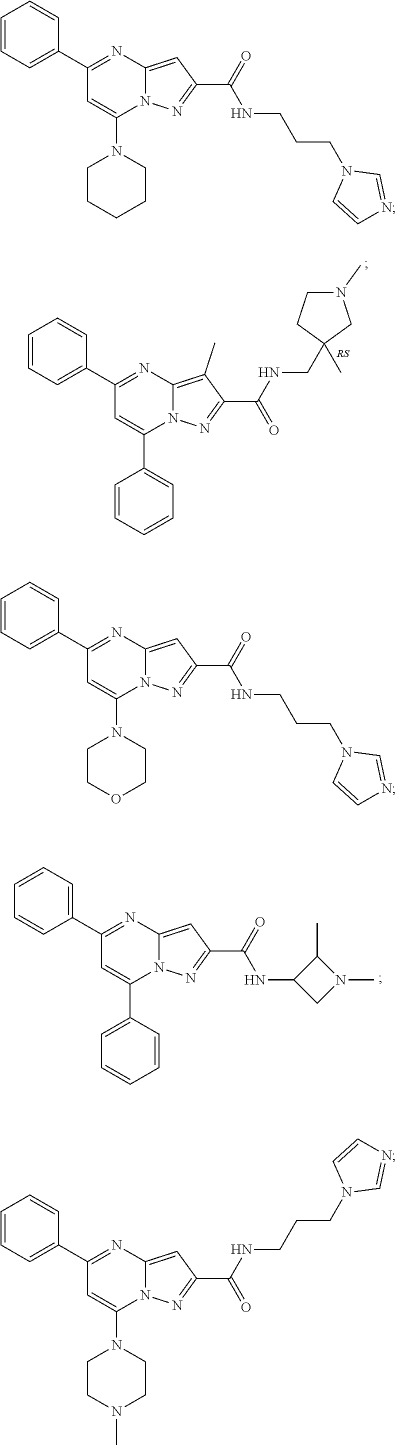

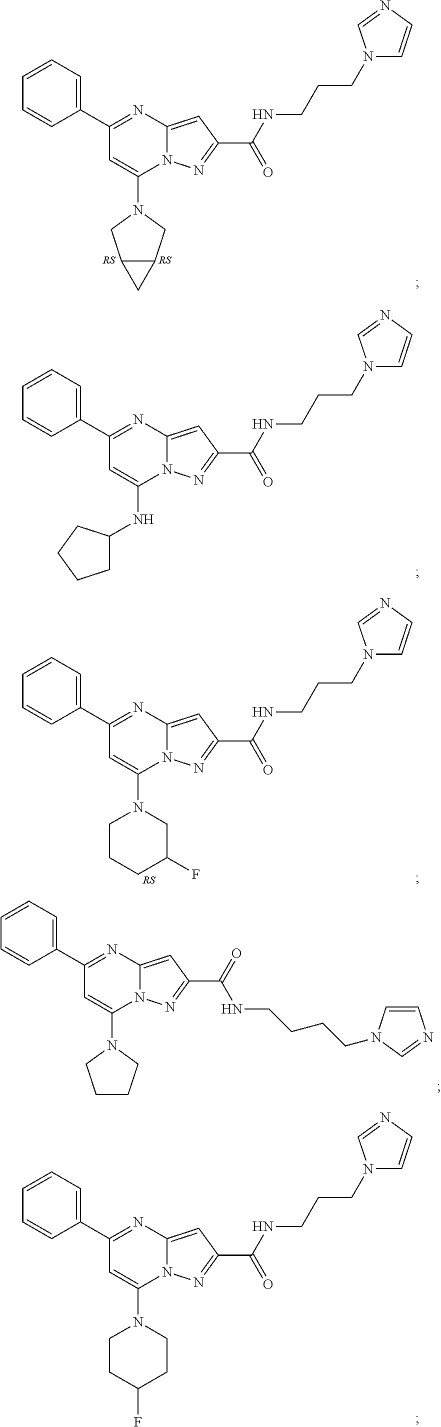 [SMILES: O=C(NCCCn1ccnc1)c1cc2nc(-c3ccccc3)cc(N3CC4CC4C3)n2n1 ; O=C(NCCCn1ccnc1)c1cc2nc(-c3ccccc3)cc(NC3CCCC3)n2n1 ; O=C(NCCCn1ccnc1)c1cc2nc(-c3ccccc3)cc(N3CCCC(F)C3)n2n1 ; O=C(NCCCCn1ccnc1)c1cc2nc(-c3ccccc3)cc(N3CCCC3)n2n1 ; O=C(NCCCn1ccnc1)c1cc2nc(-c3ccccc3)cc(N3CCC(F)CC3)n2n1]

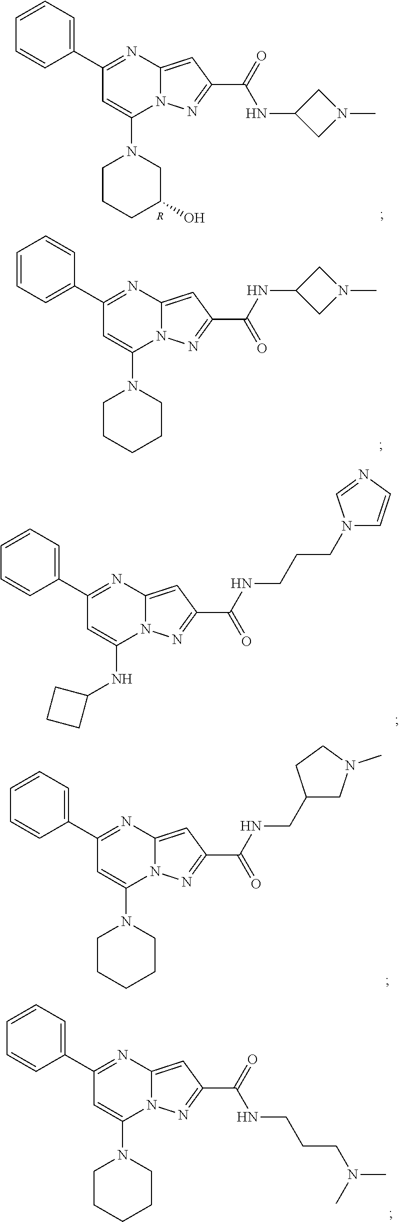

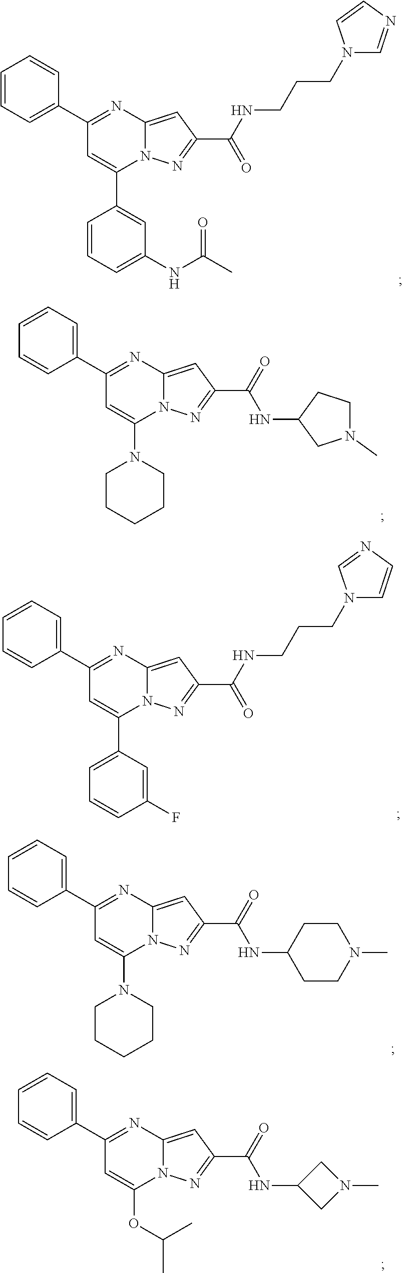 [SMILES: CC(=O)Nc1cccc(-c2cc(-c3ccccc3)nc3cc(C(=O)NCCCn4ccnc4)nn23)c1 ; CN1CCC(NC(=O)c2cc3nc(-c4ccccc4)cc(N4CCCCC4)n3n2)C1 ; O=C(NCCCn1ccnc1)c1cc2nc(-c3ccccc3)cc(-c3cccc(F)c3)n2n1 ; CN1CCC(NC(=O)c2cc3nc(-c4ccccc4)cc(N4CCCCC4)n3n2)CC1 ; CC(C)Oc1cc(-c2ccccc2)nc2cc(C(=O)NC3CN(C)C3)nn12]

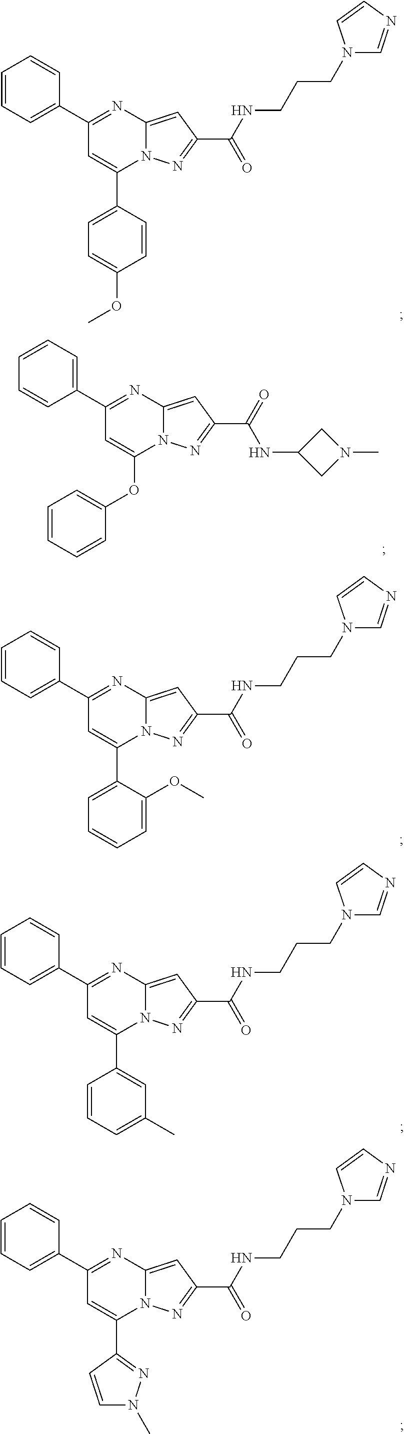

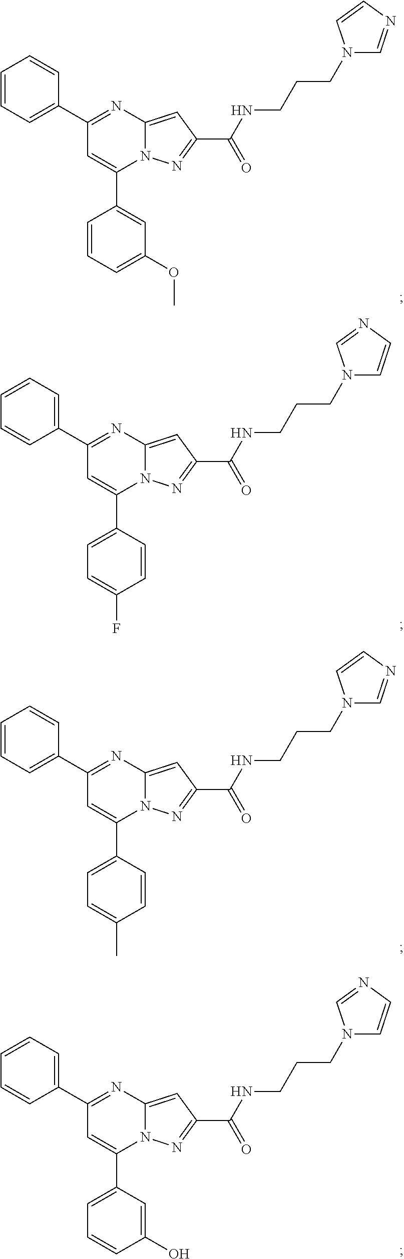 [SMILES: COc1cccc(-c2cc(-c3ccccc3)nc3cc(C(=O)NCCCn4ccnc4)nn23)c1 ; O=C(NCCCn1ccnc1)c1cc2nc(-c3ccccc3)cc(-c3ccc(F)cc3)n2n1 ; Cc1ccc(-c2cc(-c3ccccc3)nc3cc(C(=O)NCCCn4ccnc4)nn23)cc1 ; O=C(NCCCn1ccnc1)c1cc2nc(-c3ccccc3)cc(-c3cccc(O)c3)n2n1]

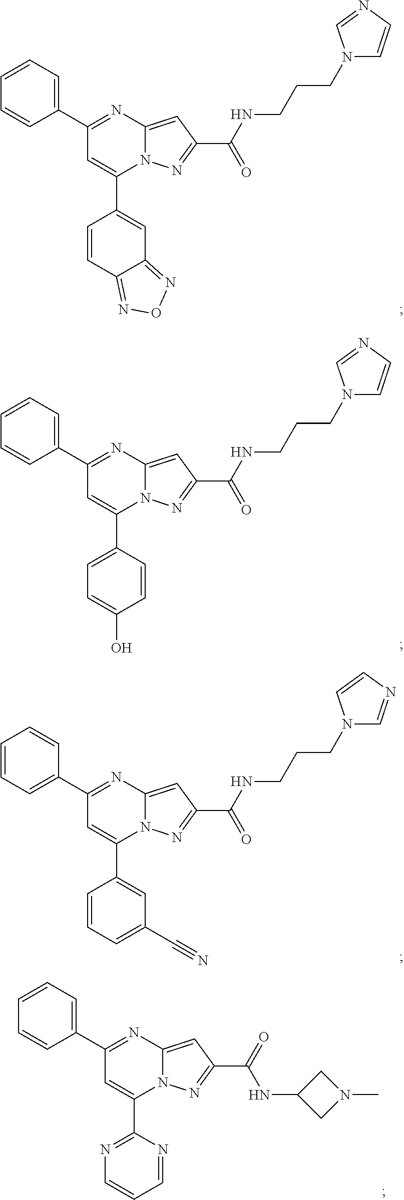 [SMILES: O=C(NCCCn1ccnc1)c1cc2nc(-c3ccccc3)cc(-c3ccc4nonc4c3)n2n1 ; O=C(NCCCn1ccnc1)c1cc2nc(-c3ccccc3)cc(-c3ccc(O)cc3)n2n1 ; N#Cc1cccc(-c2cc(-c3ccccc3)nc3cc(C(=O)NCCCn4ccnc4)nn23)c1 ; CN1CC(NC(=O)c2cc3nc(-c4ccccc4)cc(-c4ncccn4)n3n2)C1]

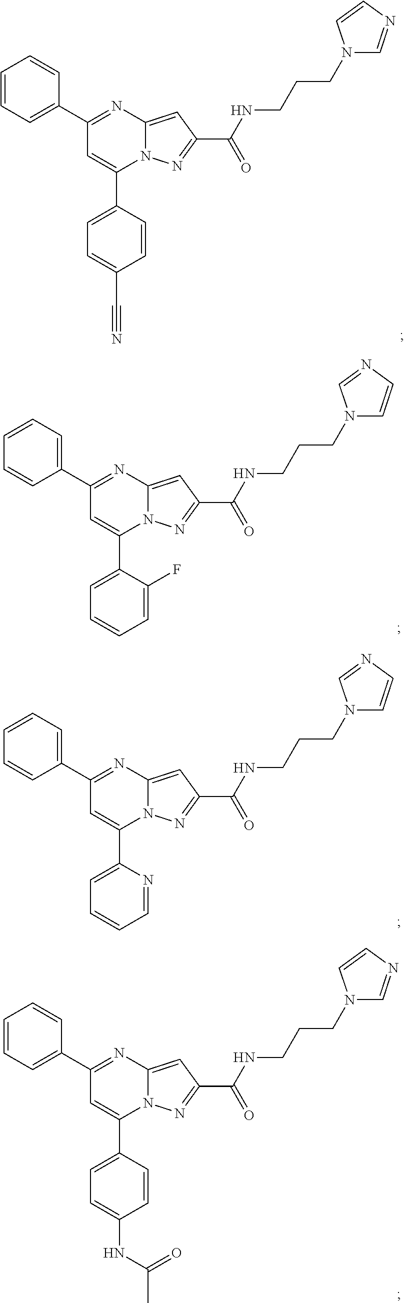 [SMILES: N#Cc1ccc(-c2cc(-c3ccccc3)nc3cc(C(=O)NCCCn4ccnc4)nn23)cc1 ; O=C(NCCCn1ccnc1)c1cc2nc(-c3ccccc3)cc(-c3ccccc3F)n2n1 ; O=C(NCCCn1ccnc1)c1cc2nc(-c3ccccc3)cc(-c3ccccn3)n2n1 ; CC(=O)Nc1ccc(-c2cc(-c3ccccc3)nc3cc(C(=O)NCCCn4ccnc4)nn23)cc1]

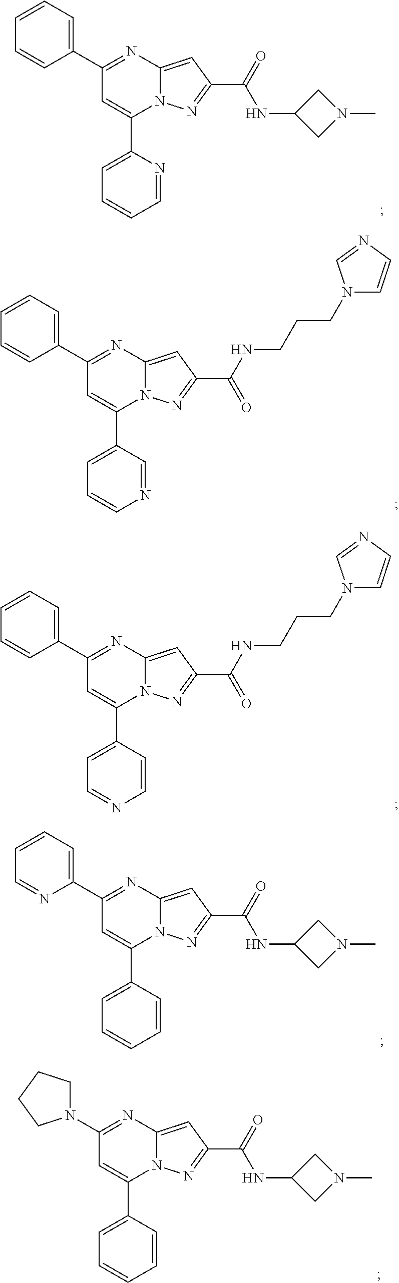 [SMILES: CN1CC(NC(=O)c2cc3nc(-c4ccccc4)cc(-c4ccccn4)n3n2)C1 ; O=C(NCCCn1ccnc1)c1cc2nc(-c3ccccc3)cc(-c3cccnc3)n2n1 ; O=C(NCCCn1ccnc1)c1cc2nc(-c3ccccc3)cc(-c3ccncc3)n2n1 ; CN1CC(NC(=O)c2cc3nc(-c4ccccn4)cc(-c4ccccc4)n3n2)C1 ; CN1CC(NC(=O)c2cc3nc(N4CCCC4)cc(-c4ccccc4)n3n2)C1]

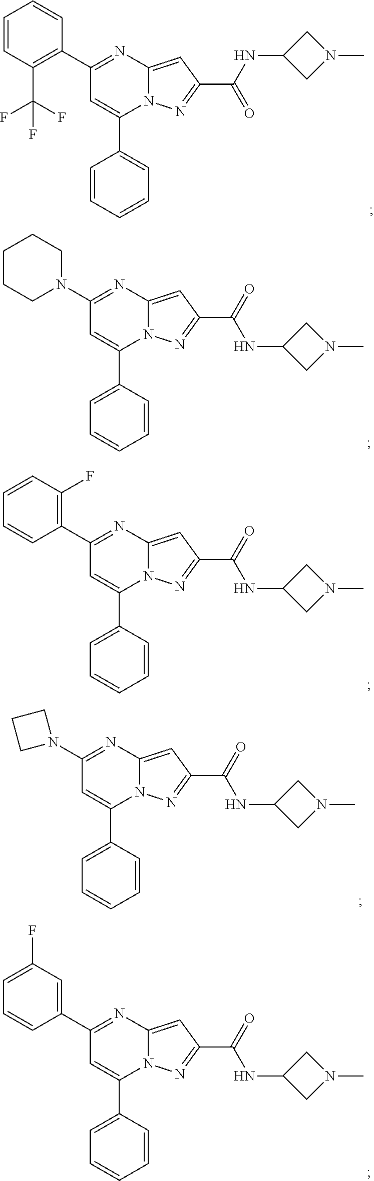

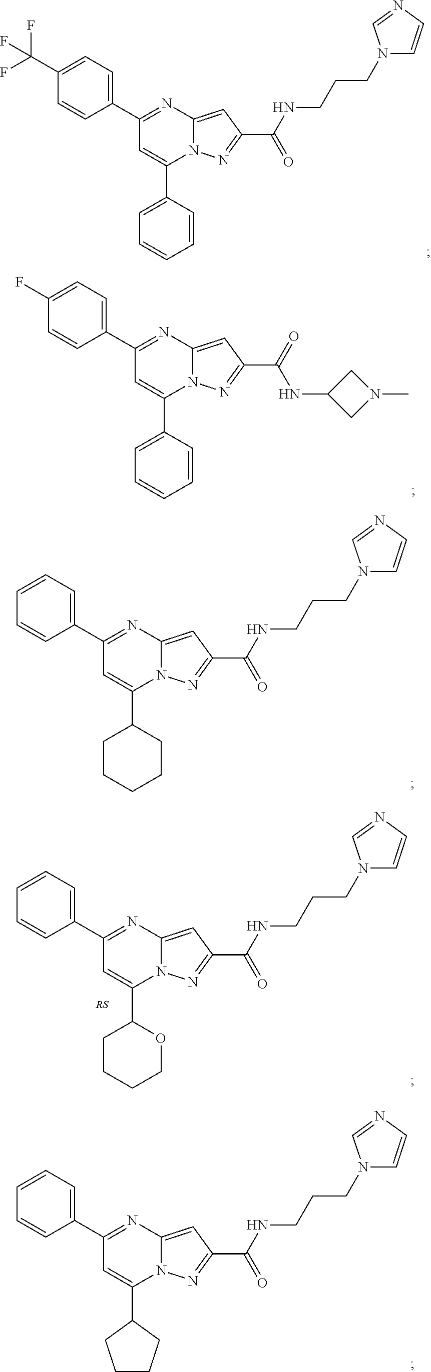 [SMILES: O=C(NCCCn1ccnc1)c1cc2nc(-c3ccc(C(F)(F)F)cc3)cc(-c3ccccc3)n2n1 ; CN1CC(NC(=O)c2cc3nc(-c4ccc(F)cc4)cc(-c4ccccc4)n3n2)C1 ; O=C(NCCCn1ccnc1)c1cc2nc(-c3ccccc3)cc(C3CCCCC3)n2n1 ; O=C(NCCCn1ccnc1)c1cc2nc(-c3ccccc3)cc(C3CCCCO3)n2n1 ; O=C(NCCCn1ccnc1)c1cc2nc(-c3ccccc3)cc(C3CCCC3)n2n1]

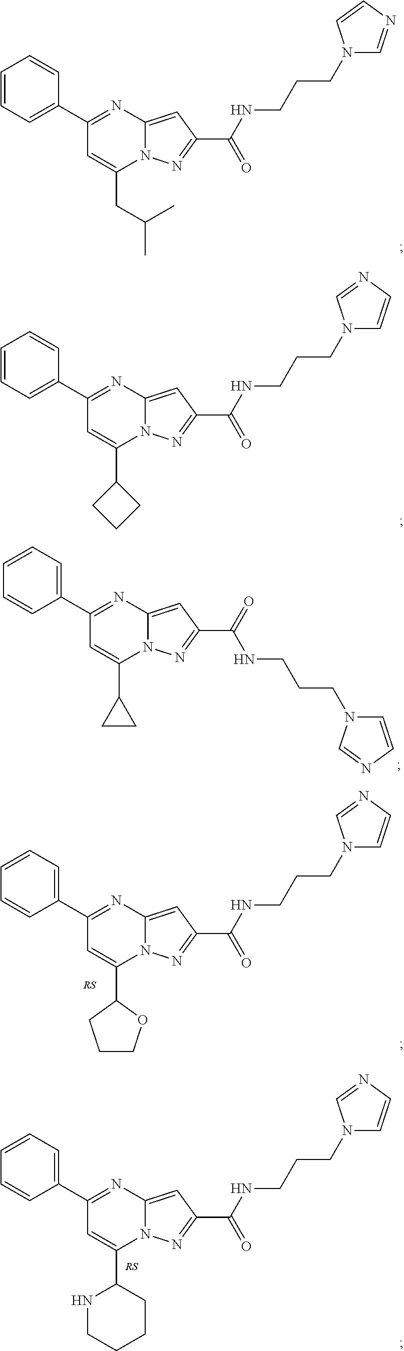

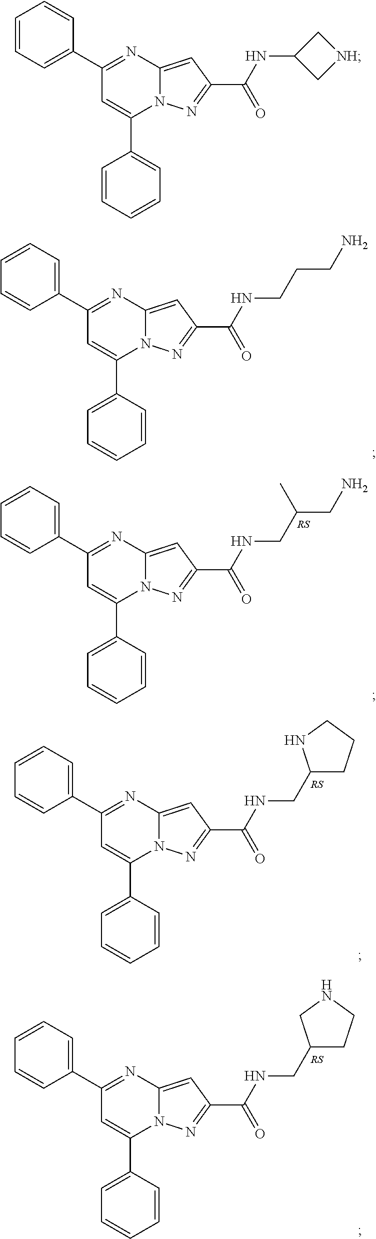 [SMILES: O=C(NC1CNC1)c1cc2nc(-c3ccccc3)cc(-c3ccccc3)n2n1 ; NCCCNC(=O)c1cc2nc(-c3ccccc3)cc(-c3ccccc3)n2n1 ; CC(CN)CNC(=O)c1cc2nc(-c3ccccc3)cc(-c3ccccc3)n2n1 ; O=C(NCC1CCCN1)c1cc2nc(-c3ccccc3)cc(-c3ccccc3)n2n1 ; O=C(NCC1CCNC1)c1cc2nc(-c3ccccc3)cc(-c3ccccc3)n2n1]

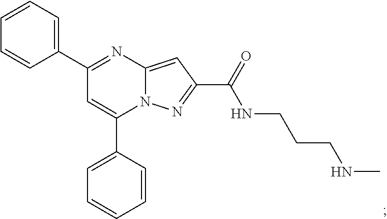 [SMILES: CNCCCNC(=O)c1cc2nc(-c3ccccc3)cc(-c3ccccc3)n2n1]

and pharmaceutically acceptable salts thereof.

Another embodiment of the invention is a method for treating or ameliorating a SMOX mediated syndrome, disorder or disease comprising administering to a subject in need thereof an effective amount of a compound selected from the group consisting of

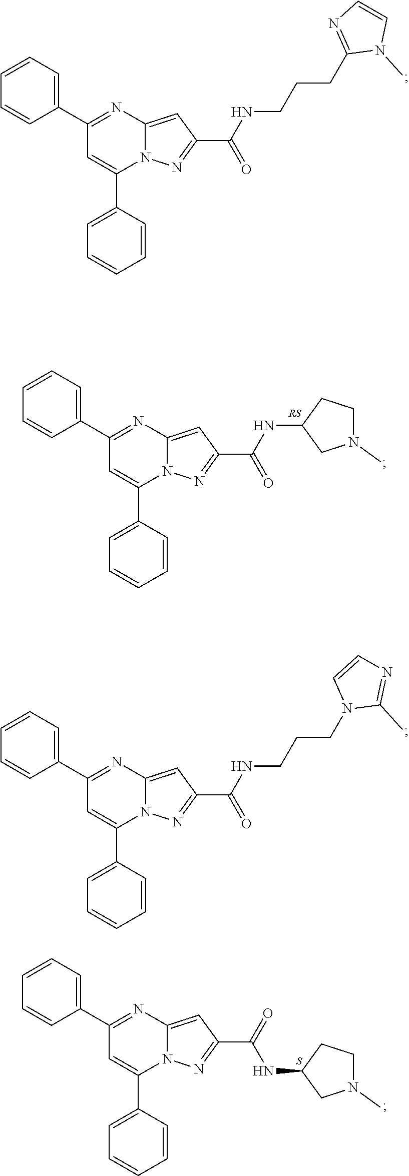

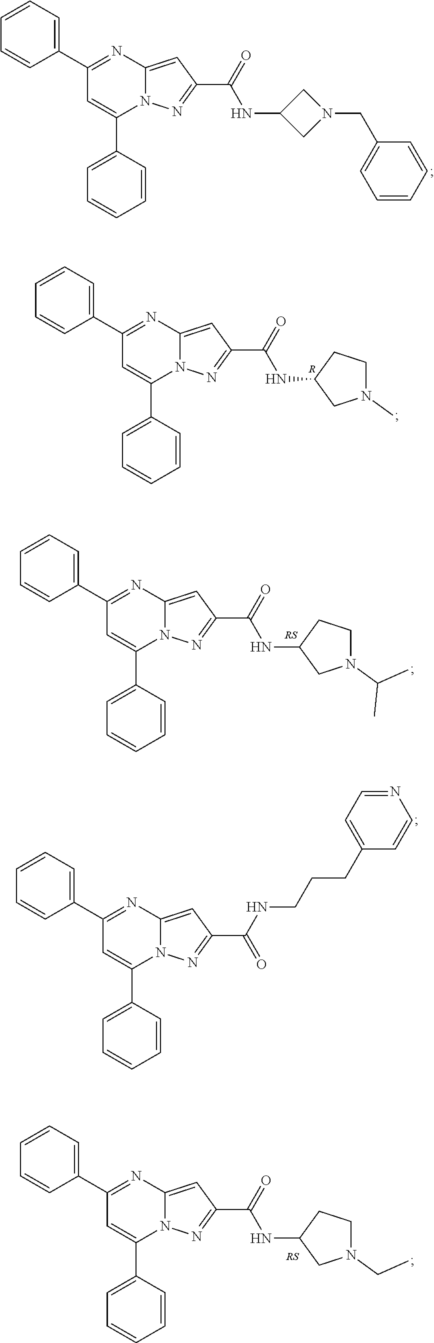

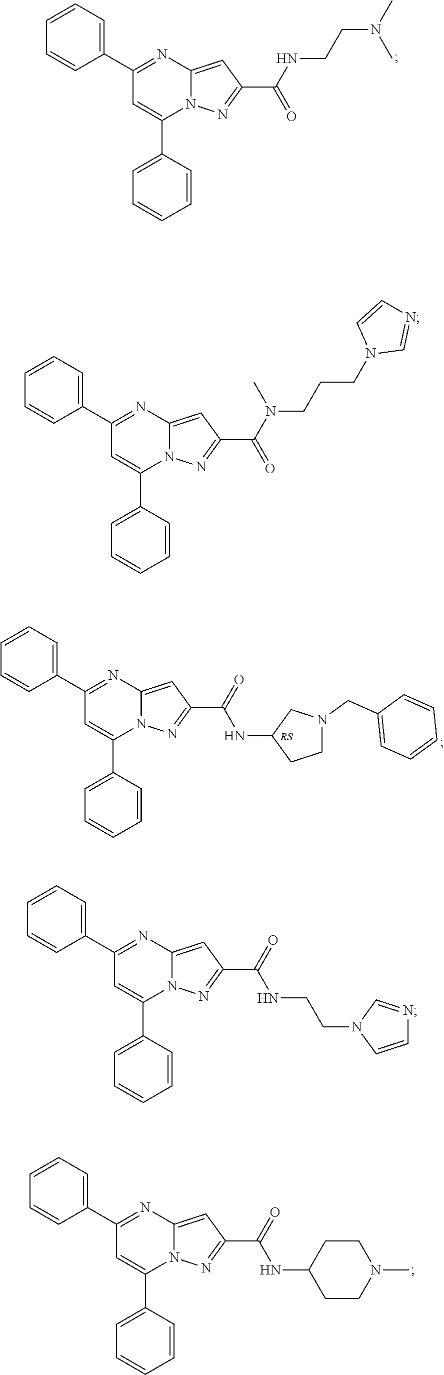 [SMILES: CN(C)CCNC(=O)c1cc2nc(-c3ccccc3)cc(-c3ccccc3)n2n1 ; CN(CCCn1ccnc1)C(=O)c1cc2nc(-c3ccccc3)cc(-c3ccccc3)n2n1 ; O=C(NC1CCN(Cc2ccccc2)C1)c1cc2nc(-c3ccccc3)cc(-c3ccccc3)n2n1 ; O=C(NCCn1ccnc1)c1cc2nc(-c3ccccc3)cc(-c3ccccc3)n2n1 ; CN1CCC(NC(=O)c2cc3nc(-c4ccccc4)cc(-c4ccccc4)n3n2)CC1]

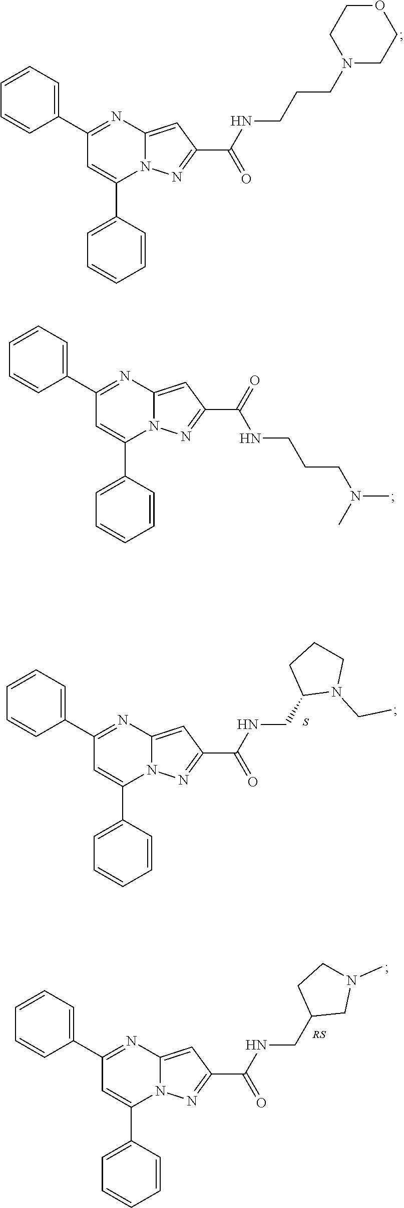

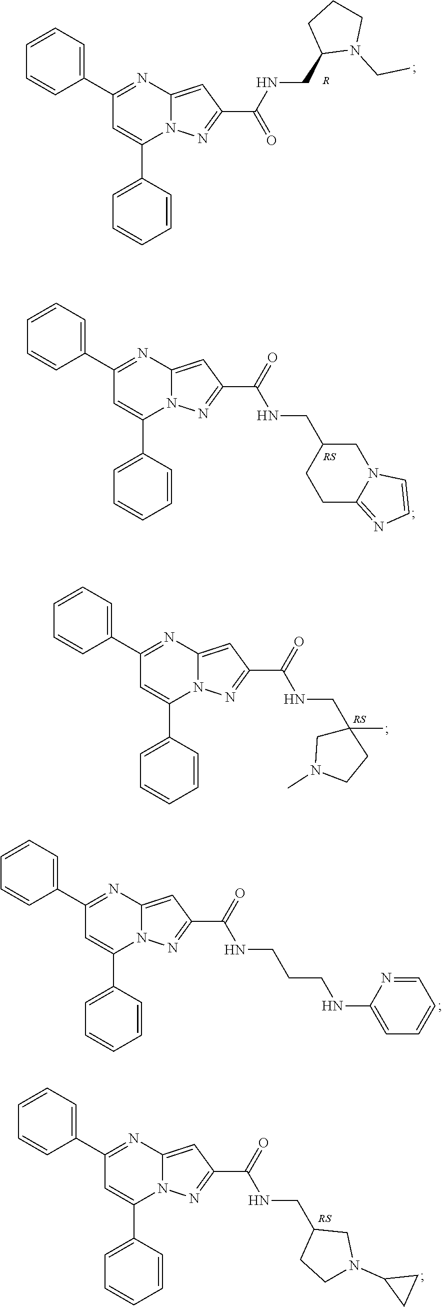

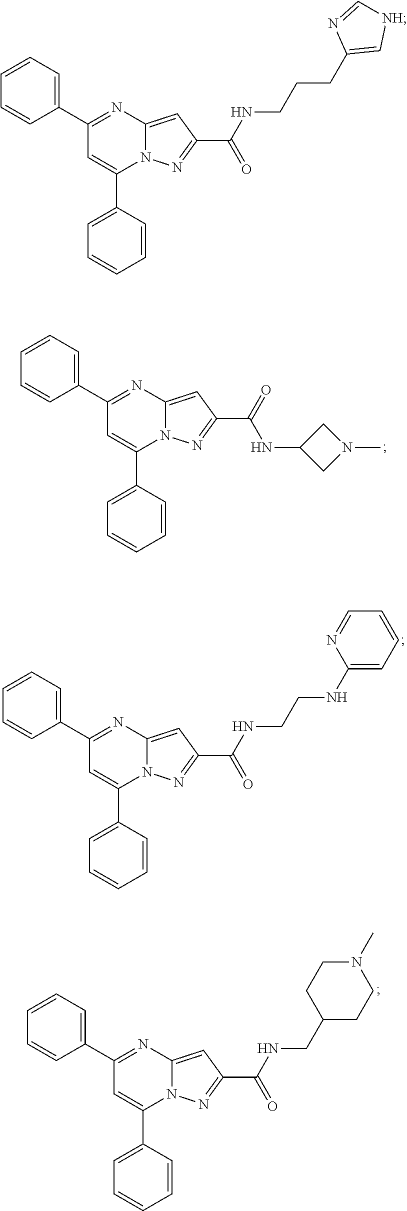 [SMILES: O=C(NCCCc1c[nH]cn1)c1cc2nc(-c3ccccc3)cc(-c3ccccc3)n2n1 ; CN1CC(NC(=O)c2cc3nc(-c4ccccc4)cc(-c4ccccc4)n3n2)C1 ; O=C(NCCNc1ccccn1)c1cc2nc(-c3ccccc3)cc(-c3ccccc3)n2n1 ; CN1CCC(CNC(=O)c2cc3nc(-c4ccccc4)cc(-c4ccccc4)n3n2)CC1]

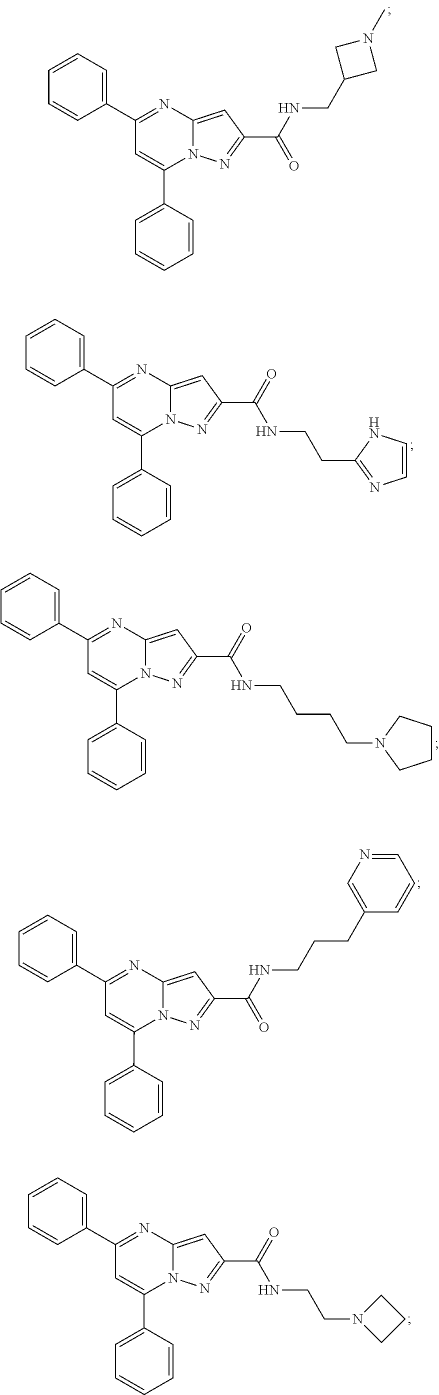 [SMILES: CN1CC(CNC(=O)c2cc3nc(-c4ccccc4)cc(-c4ccccc4)n3n2)C1 ; O=C(NCCc1ncc[nH]1)c1cc2nc(-c3ccccc3)cc(-c3ccccc3)n2n1 ; O=C(NCCCCN1CCCC1)c1cc2nc(-c3ccccc3)cc(-c3ccccc3)n2n1 ; O=C(NCCCc1cccnc1)c1cc2nc(-c3ccccc3)cc(-c3ccccc3)n2n1 ; O=C(NCCN1CCC1)c1cc2nc(-c3ccccc3)cc(-c3ccccc3)n2n1]

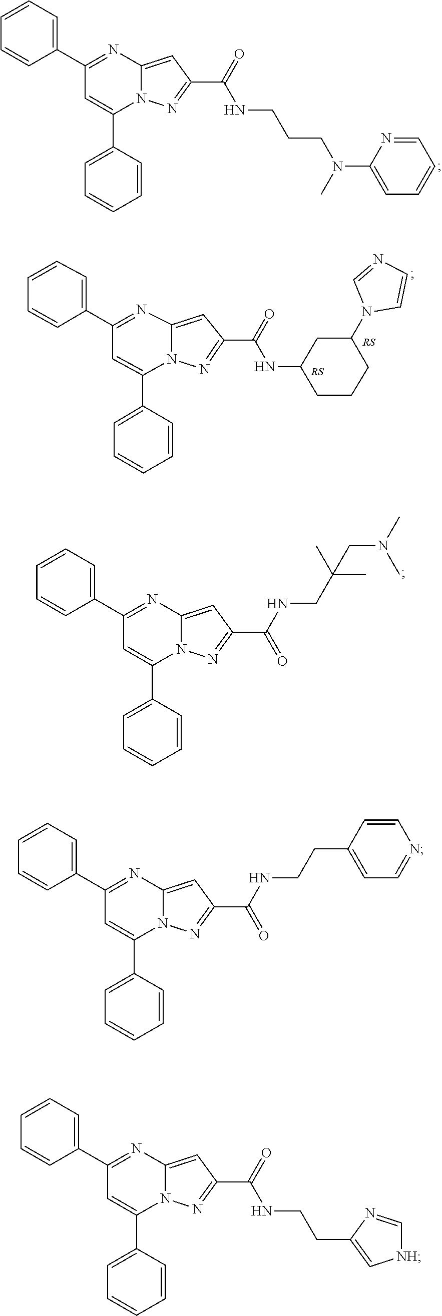 [SMILES: CN(CCCNC(=O)c1cc2nc(-c3ccccc3)cc(-c3ccccc3)n2n1)c1ccccn1 ; O=C(NC1CCCC(n2ccnc2)C1)c1cc2nc(-c3ccccc3)cc(-c3ccccc3)n2n1 ; CN(C)CC(C)(C)CNC(=O)c1cc2nc(-c3ccccc3)cc(-c3ccccc3)n2n1 ; O=C(NCCc1ccncc1)c1cc2nc(-c3ccccc3)cc(-c3ccccc3)n2n1 ; O=C(NCCc1c[nH]cn1)c1cc2nc(-c3ccccc3)cc(-c3ccccc3)n2n1]

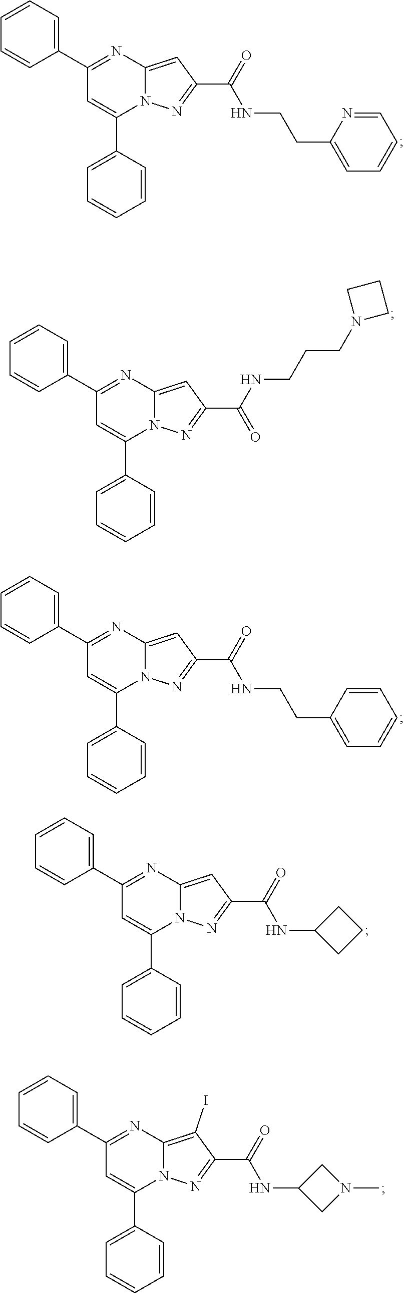 [SMILES: O=C(NCCc1ccccn1)c1cc2nc(-c3ccccc3)cc(-c3ccccc3)n2n1 ; O=C(NCCCN1CCC1)c1cc2nc(-c3ccccc3)cc(-c3ccccc3)n2n1 ; O=C(NCCc1ccccc1)c1cc2nc(-c3ccccc3)cc(-c3ccccc3)n2n1 ; O=C(NC1CCC1)c1cc2nc(-c3ccccc3)cc(-c3ccccc3)n2n1 ; CN1CC(NC(=O)c2nn3c(-c4ccccc4)cc(-c4ccccc4)nc3c2I)C1]

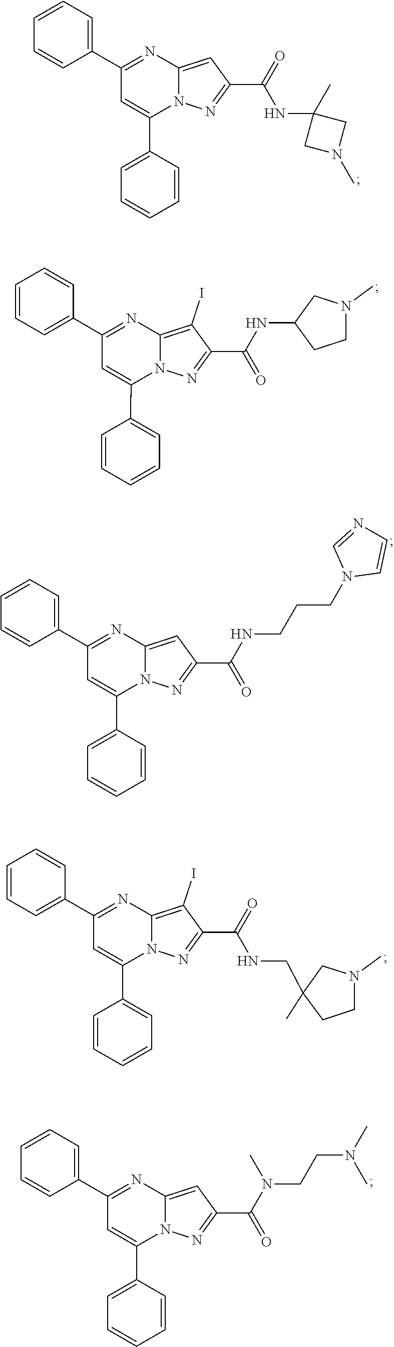 [SMILES: CN1CC(C)(NC(=O)c2cc3nc(-c4ccccc4)cc(-c4ccccc4)n3n2)C1 ; CN1CCC(NC(=O)c2nn3c(-c4ccccc4)cc(-c4ccccc4)nc3c2I)C1 ; O=C(NCCCn1ccnc1)c1cc2nc(-c3ccccc3)cc(-c3ccccc3)n2n1 ; CN1CCC(C)(CNC(=O)c2nn3c(-c4ccccc4)cc(-c4ccccc4)nc3c2I)C1 ; CN(C)CCN(C)C(=O)c1cc2nc(-c3ccccc3)cc(-c3ccccc3)n2n1]

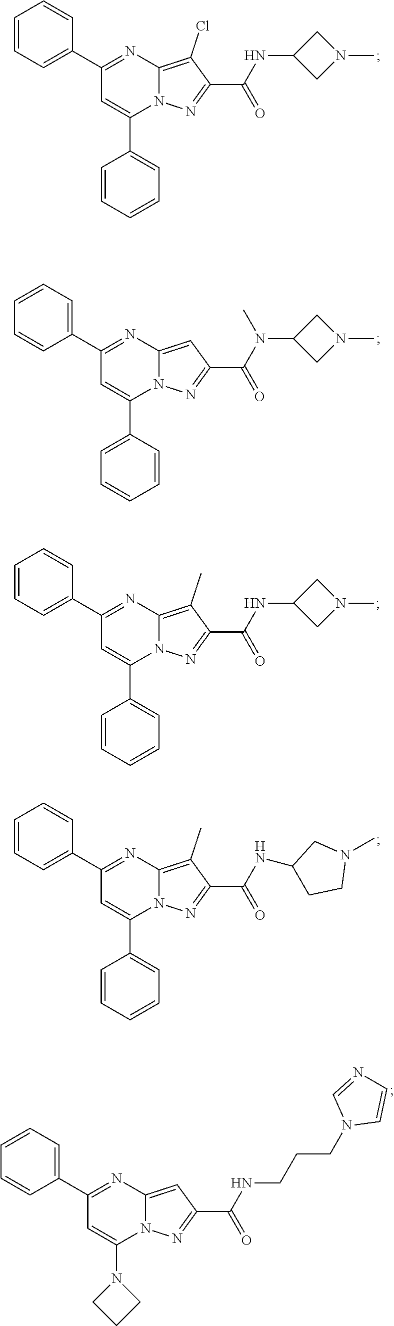 [SMILES: CN1CC(NC(=O)c2nn3c(-c4ccccc4)cc(-c4ccccc4)nc3c2Cl)C1 ; CN1CC(N(C)C(=O)c2cc3nc(-c4ccccc4)cc(-c4ccccc4)n3n2)C1 ; Cc1c(C(=O)NC2CN(C)C2)nn2c(-c3ccccc3)cc(-c3ccccc3)nc12 ; Cc1c(C(=O)NC2CCN(C)C2)nn2c(-c3ccccc3)cc(-c3ccccc3)nc12 ; O=C(NCCCn1ccnc1)c1cc2nc(-c3ccccc3)cc(N3CCC3)n2n1]

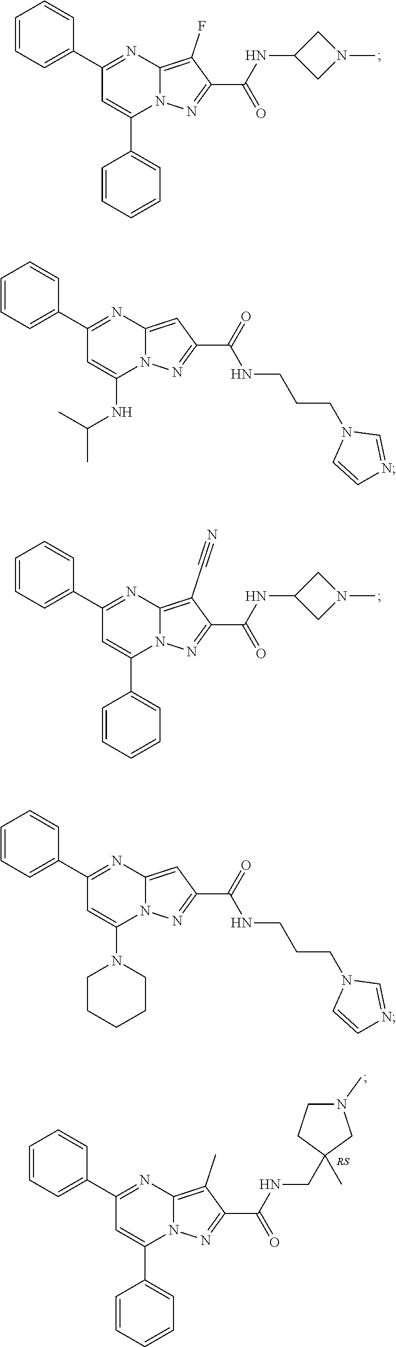

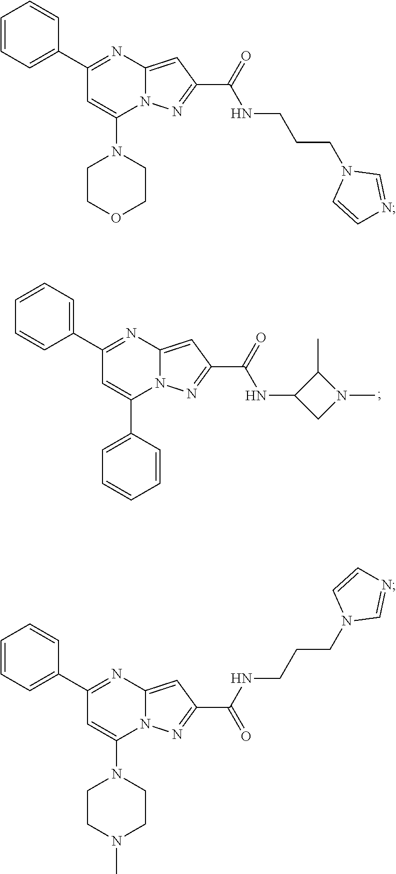 [SMILES: O=C(NCCCn1ccnc1)c1cc2nc(-c3ccccc3)cc(N3CCOCC3)n2n1 ; CC1C(NC(=O)c2cc3nc(-c4ccccc4)cc(-c4ccccc4)n3n2)CN1C ; CN1CCN(c2cc(-c3ccccc3)nc3cc(C(=O)NCCCn4ccnc4)nn23)CC1]

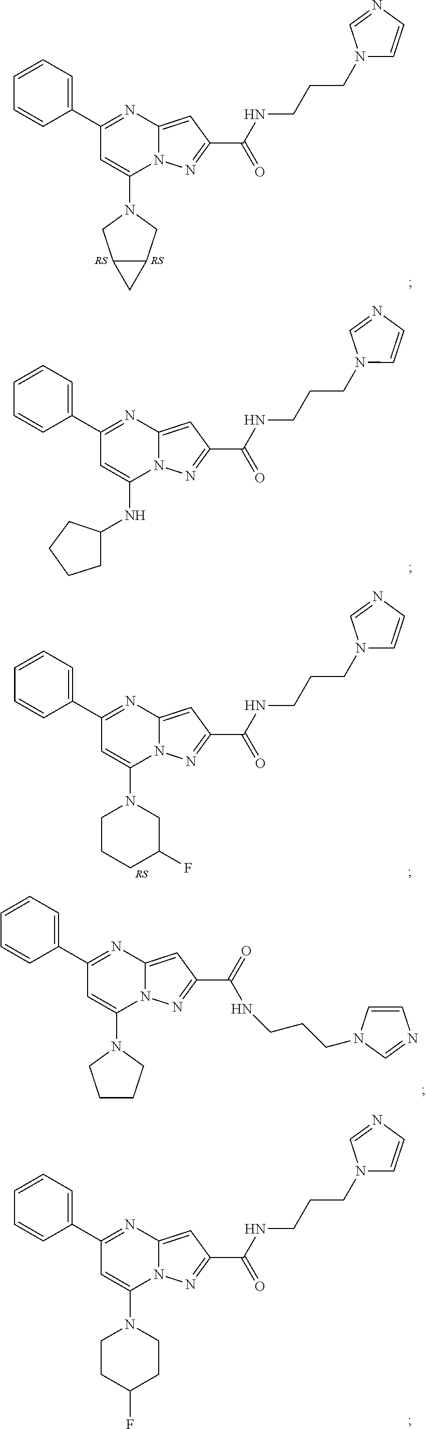 [SMILES: O=C(NCCCn1ccnc1)c1cc2nc(-c3ccccc3)cc(N3CC4CC4C3)n2n1 ; O=C(NCCCn1ccnc1)c1cc2nc(-c3ccccc3)cc(NC3CCCC3)n2n1 ; O=C(NCCCn1ccnc1)c1cc2nc(-c3ccccc3)cc(N3CCCC(F)C3)n2n1 ; O=C(NCCCn1ccnc1)c1cc2nc(-c3ccccc3)cc(N3CCCC3)n2n1 ; O=C(NCCCn1ccnc1)c1cc2nc(-c3ccccc3)cc(N3CCC(F)CC3)n2n1]

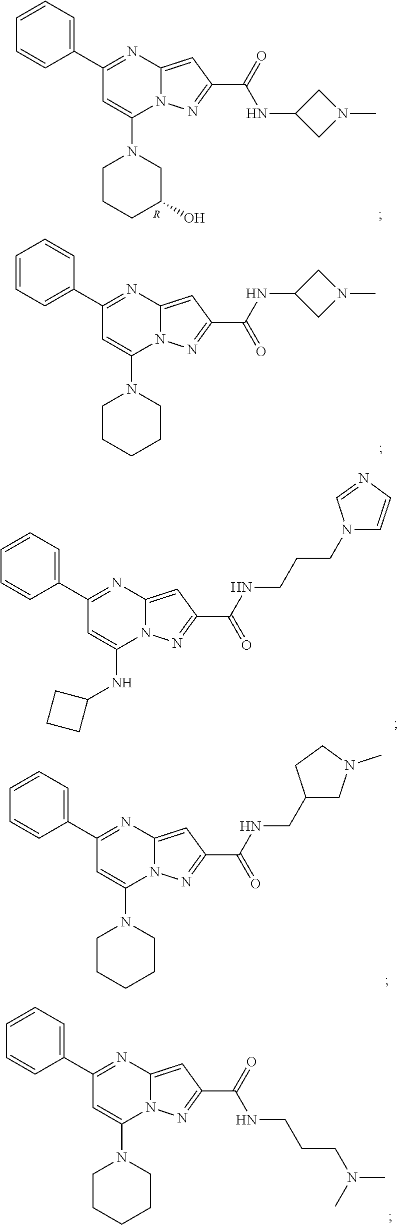

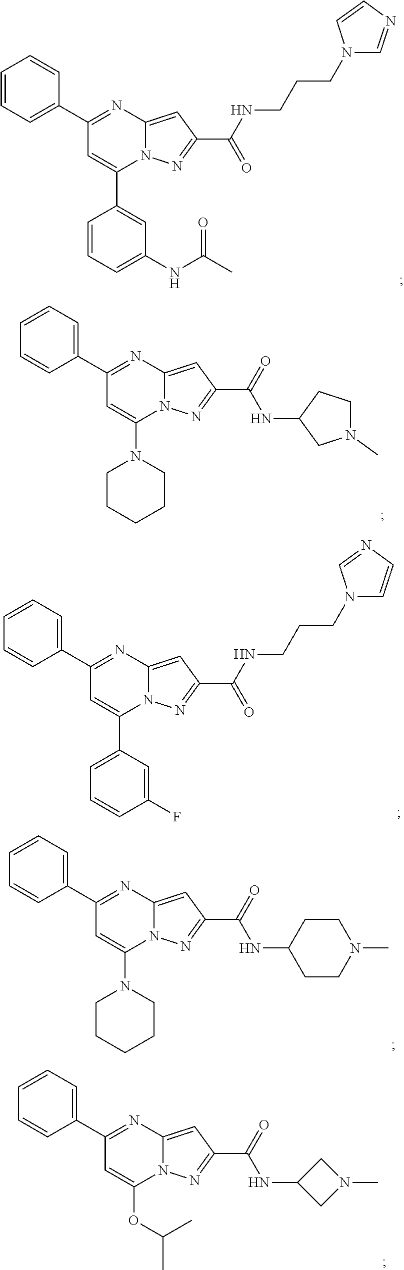 [SMILES: CC(=O)Nc1cccc(-c2cc(-c3ccccc3)nc3cc(C(=O)NCCCn4ccnc4)nn23)c1 ; CN1CCC(NC(=O)c2cc3nc(-c4ccccc4)cc(N4CCCCC4)n3n2)C1 ; O=C(NCCCn1ccnc1)c1cc2nc(-c3ccccc3)cc(-c3cccc(F)c3)n2n1 ; CN1CCC(NC(=O)c2cc3nc(-c4ccccc4)cc(N4CCCCC4)n3n2)CC1 ; CC(C)Oc1cc(-c2ccccc2)nc2cc(C(=O)NC3CN(C)C3)nn12]

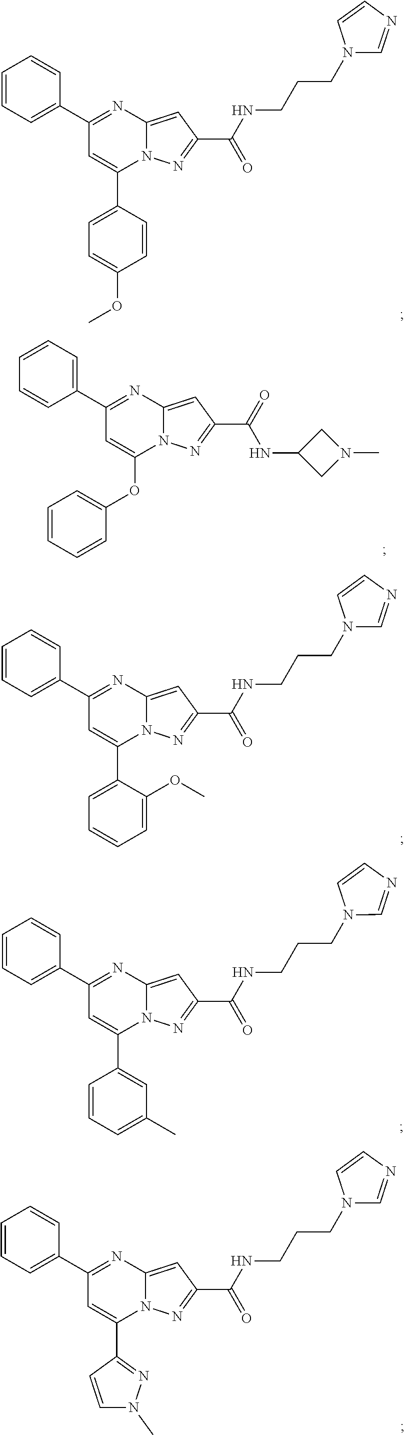

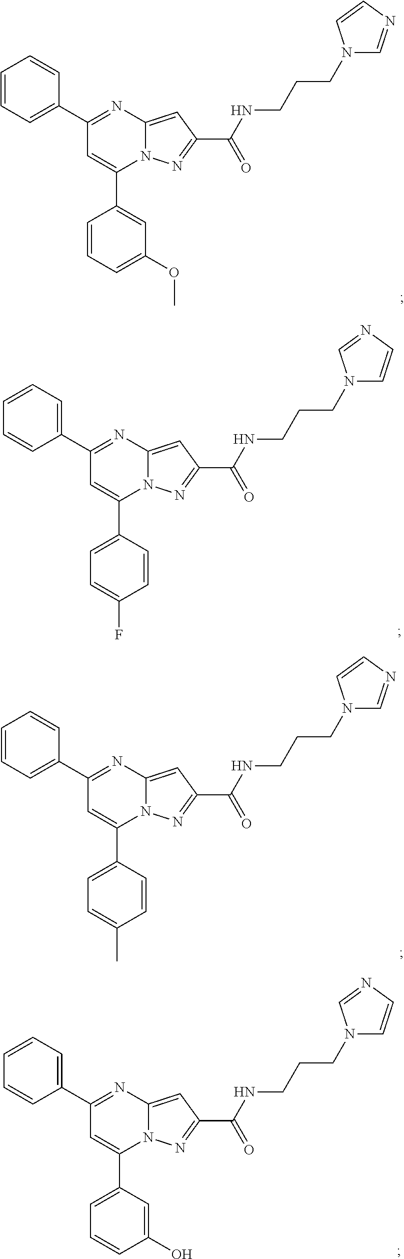 [SMILES: COc1cccc(-c2cc(-c3ccccc3)nc3cc(C(=O)NCCCn4ccnc4)nn23)c1 ; O=C(NCCCn1ccnc1)c1cc2nc(-c3ccccc3)cc(-c3ccc(F)cc3)n2n1 ; Cc1ccc(-c2cc(-c3ccccc3)nc3cc(C(=O)NCCCn4ccnc4)nn23)cc1 ; O=C(NCCCn1ccnc1)c1cc2nc(-c3ccccc3)cc(-c3cccc(O)c3)n2n1]

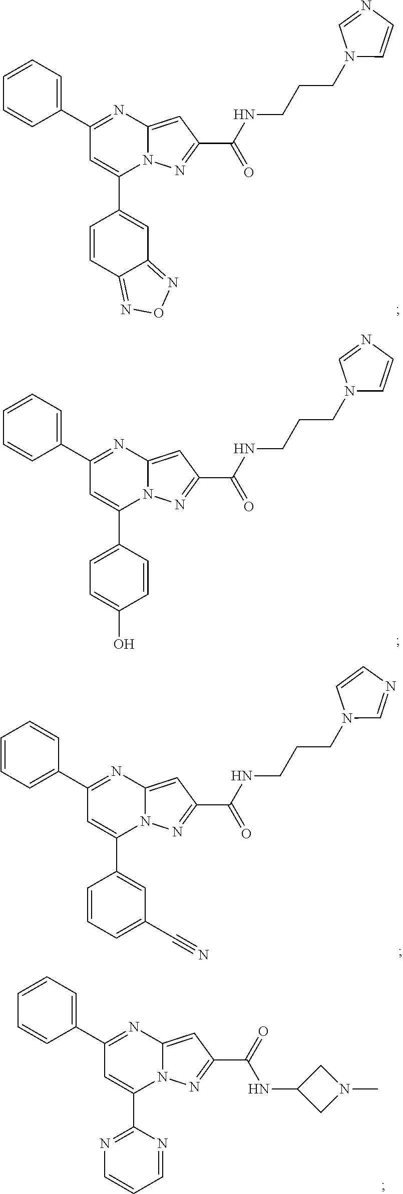 [SMILES: O=C(NCCCn1ccnc1)c1cc2nc(-c3ccccc3)cc(-c3ccc4nonc4c3)n2n1 ; O=C(NCCCn1ccnc1)c1cc2nc(-c3ccccc3)cc(-c3ccc(O)cc3)n2n1 ; N#Cc1cccc(-c2cc(-c3ccccc3)nc3cc(C(=O)NCCCn4ccnc4)nn23)c1 ; CN1CC(NC(=O)c2cc3nc(-c4ccccc4)cc(-c4ncccn4)n3n2)C1]

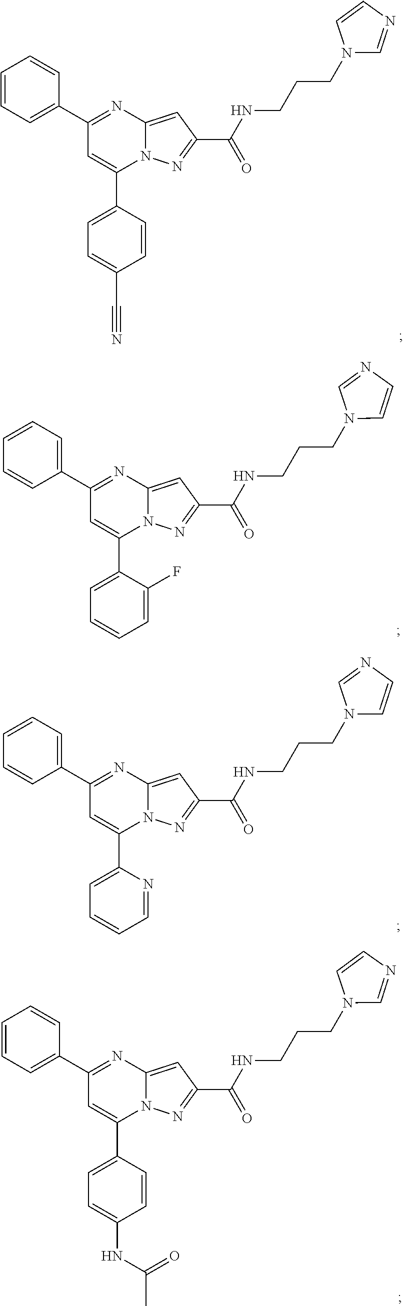 [SMILES: N#Cc1ccc(-c2cc(-c3ccccc3)nc3cc(C(=O)NCCCn4ccnc4)nn23)cc1 ; O=C(NCCCn1ccnc1)c1cc2nc(-c3ccccc3)cc(-c3ccccc3F)n2n1 ; O=C(NCCCn1ccnc1)c1cc2nc(-c3ccccc3)cc(-c3ccccn3)n2n1 ; CC(=O)Nc1ccc(-c2cc(-c3ccccc3)nc3cc(C(=O)NCCCn4ccnc4)nn23)cc1]

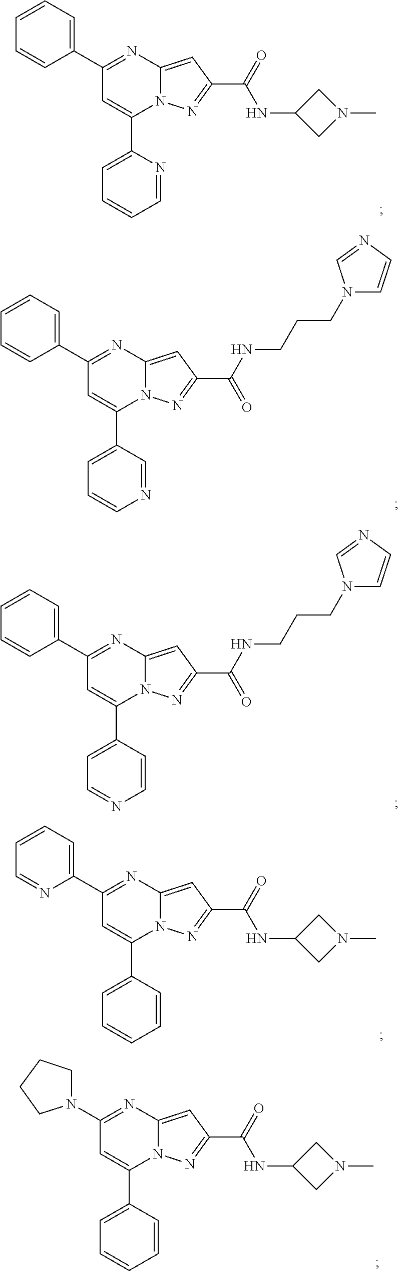 [SMILES: CN1CC(NC(=O)c2cc3nc(-c4ccccc4)cc(-c4ccccn4)n3n2)C1 ; O=C(NCCCn1ccnc1)c1cc2nc(-c3ccccc3)cc(-c3cccnc3)n2n1 ; O=C(NCCCn1ccnc1)c1cc2nc(-c3ccccc3)cc(-c3ccncc3)n2n1 ; CN1CC(NC(=O)c2cc3nc(-c4ccccn4)cc(-c4ccccc4)n3n2)C1 ; CN1CC(NC(=O)c2cc3nc(N4CCCC4)cc(-c4ccccc4)n3n2)C1]

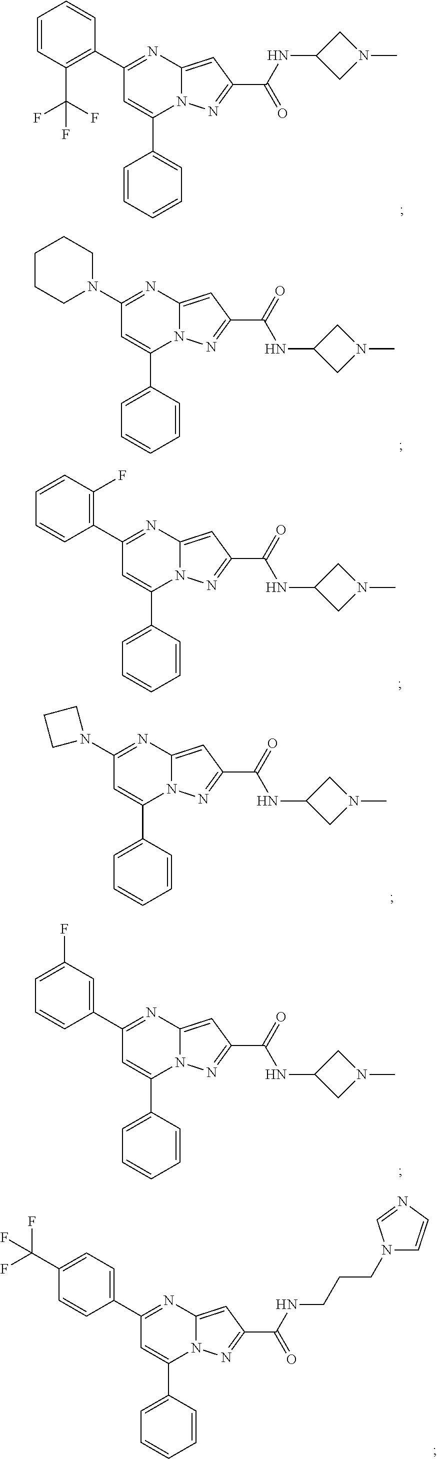

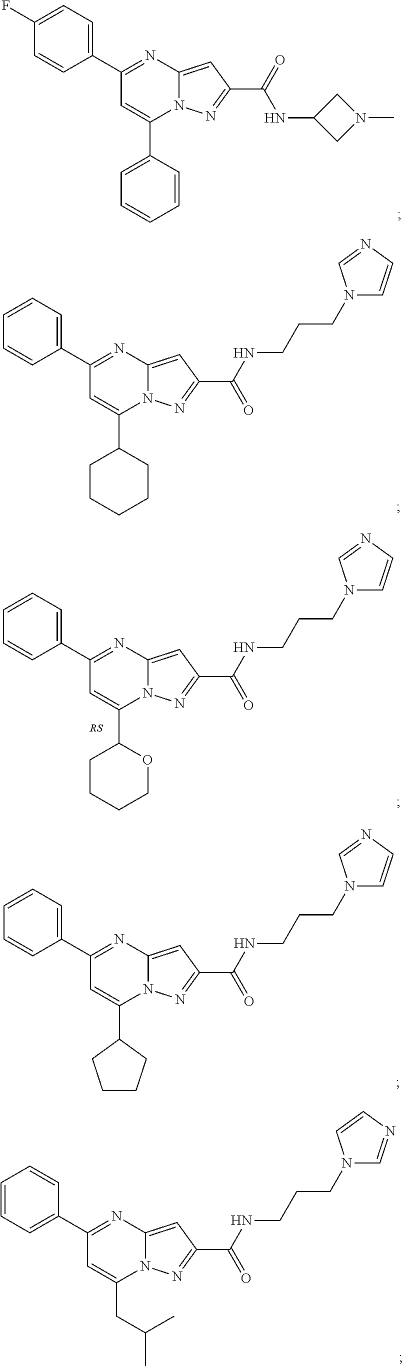 [SMILES: CN1CC(NC(=O)c2cc3nc(-c4ccc(F)cc4)cc(-c4ccccc4)n3n2)C1 ; O=C(NCCCn1ccnc1)c1cc2nc(-c3ccccc3)cc(C3CCCCC3)n2n1 ; O=C(NCCCn1ccnc1)c1cc2nc(-c3ccccc3)cc(C3CCCCO3)n2n1 ; O=C(NCCCn1ccnc1)c1cc2nc(-c3ccccc3)cc(C3CCCC3)n2n1 ; CC(C)Cc1cc(-c2ccccc2)nc2cc(C(=O)NCCCn3ccnc3)nn12]

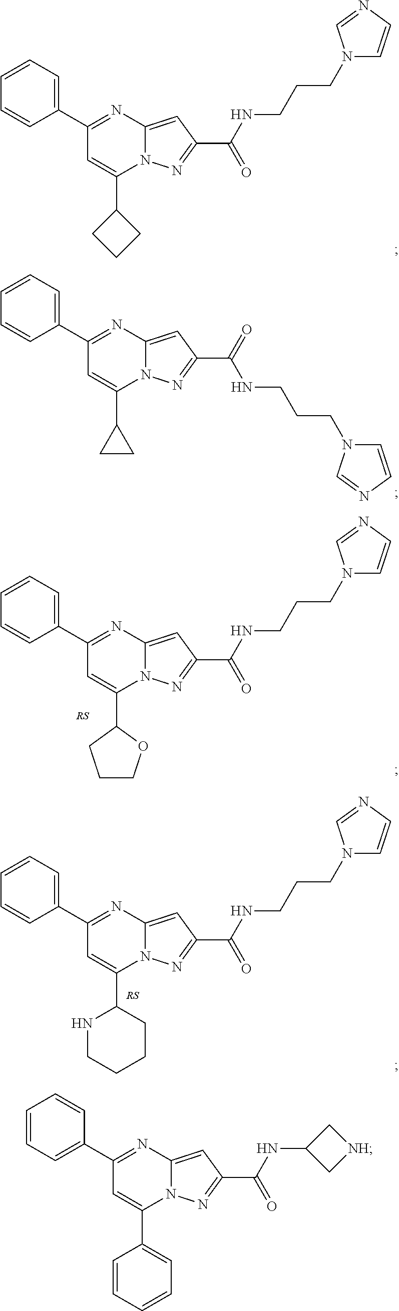

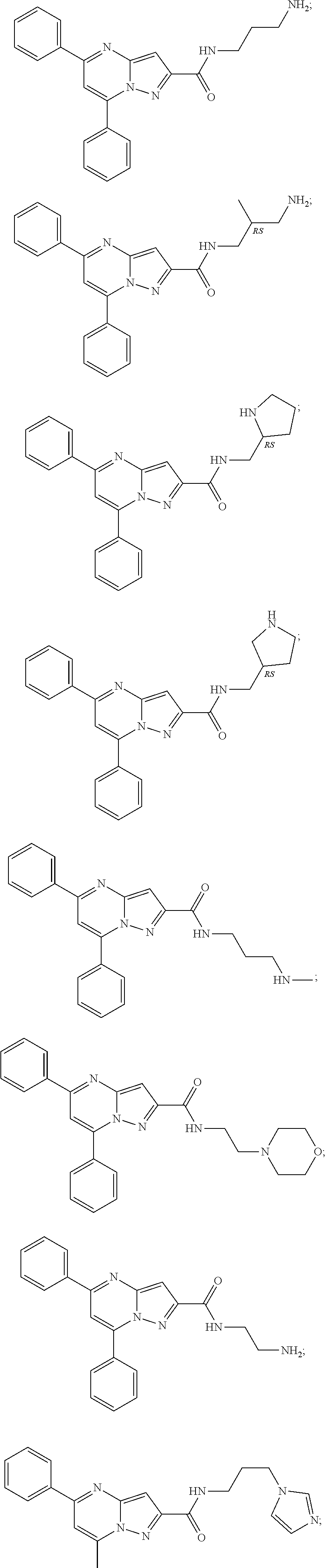

and pharmaceutically acceptable salts thereof.

Another embodiment of the invention is a compound of Formula I

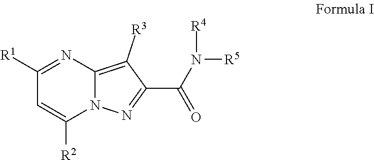

wherein:
R¹ is phenyl,

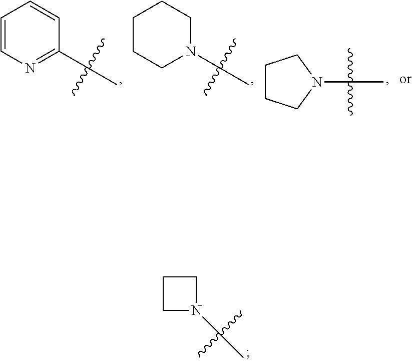

wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl,

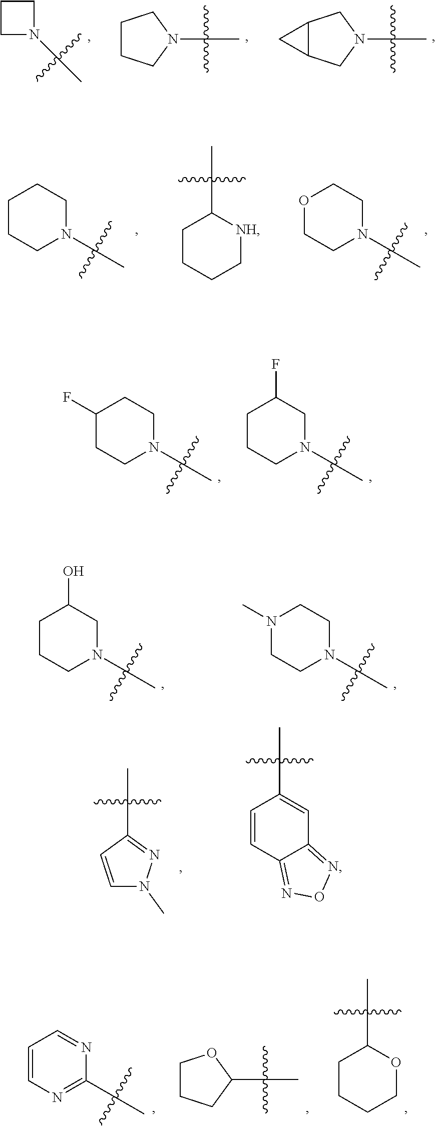

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H —I, —Cl, —F, —CN, or —CH₃;
R⁴ is —H, or —CH₃;
R⁵ is —C₍₂₋₃₎alkyl-R^{a}, cyclobutyl

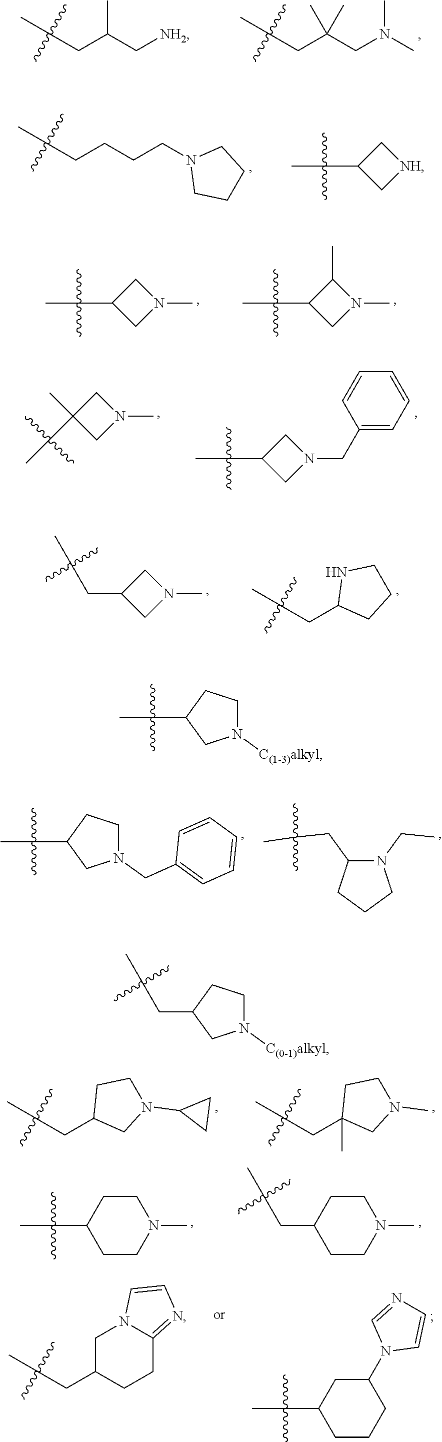

R^{a} is pyridinyl, azetidinyl, —NH₂, —NHCH₃, —N(CH₃)₂, —NHpyridinyl, or —N(CH₃)pyridinyl;
and pharmaceutically acceptable salts thereof.

Another embodiment of the invention is the compound of Formula I,

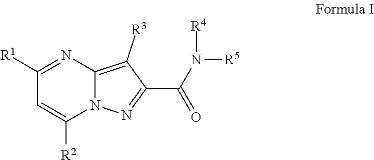

wherein:
R¹ is phenyl;
R² is phenyl, pyridinyl, —CH₃, —CH₂CH(CH₃)₂, —C₍₃₋₆₎cycloalkyl, —NHC₍₄₋₅₎cycloalkyl,

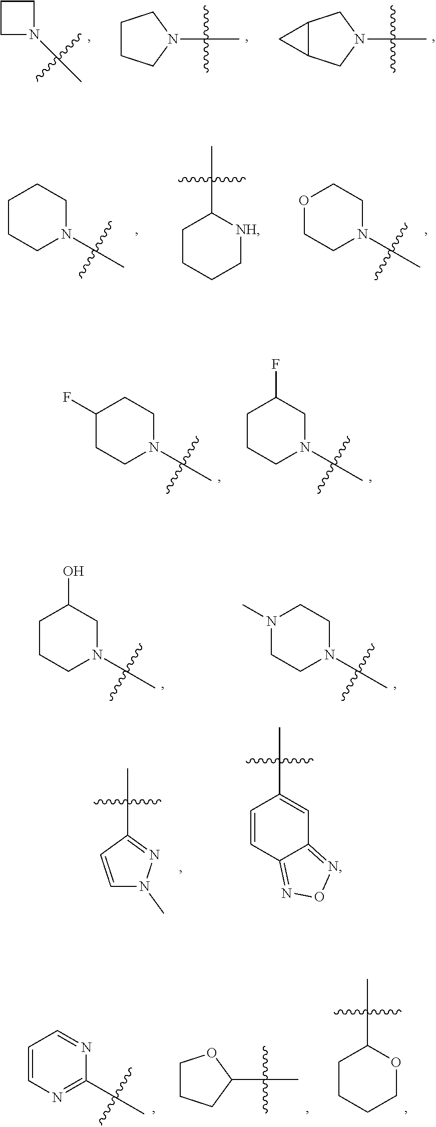

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H, —I, —Cl, —F, —CN, or —CH₃;
R⁴ is —H, or —CH₃;
R⁵ is —C₍₂₋₃₎alkyl-R^{a}, cyclobutyl,

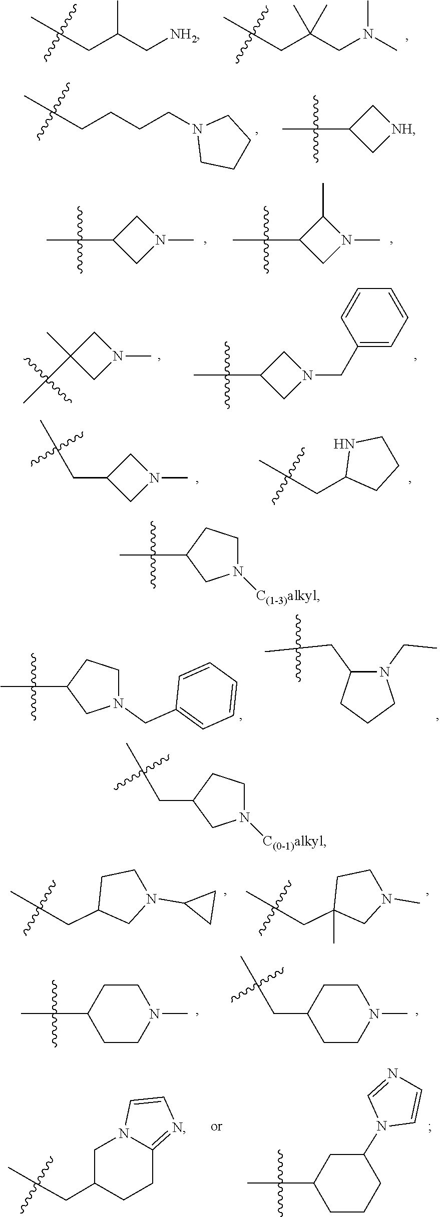

R^{a} is pyridinyl, azetidinyl, —NH₂, —NHCH₃, —N(CH₃)₂, —NHpyridinyl, or —N(CH₃)pyridinyl;
and pharmaceutically acceptable salts thereof.

Another embodiment of the invention is the compound of Formula I,

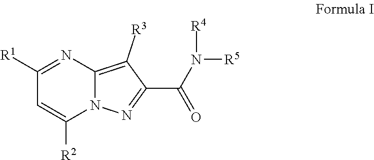

wherein:
R¹ is phenyl,

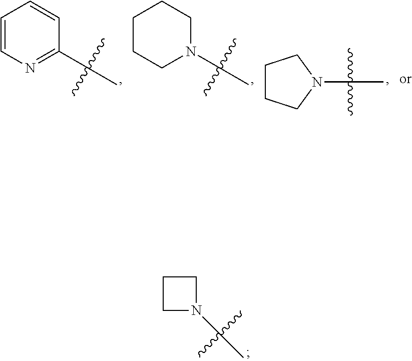

wherein said phenyl is optionally substituted with —F, or —CF₃;
R² is phenyl;
R³ is —H —I, —Cl, —F, —CN, or —CH₃;
R⁴ is —H, or —CH₃;
R⁵ is —C₍₂₋₃₎alkyl-R^{a}, cyclobutyl,

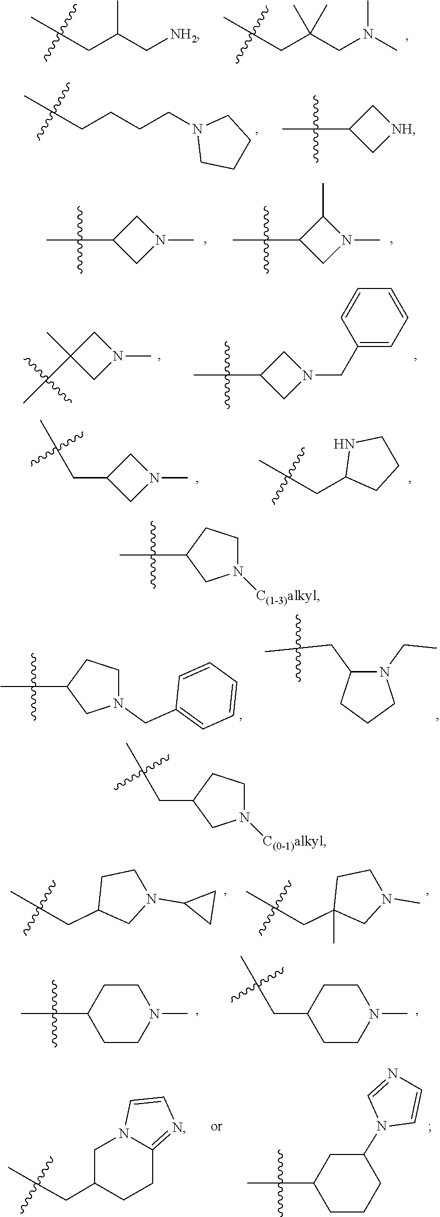

R^{a} is pyridinyl, azetidinyl, —NH₂, —NHCH₃, —N(CH₃)₂, —NHpyridinyl, or —N(CH₃)pyridinyl;
and pharmaceutically acceptable salts thereof.

Another embodiment of the invention is the compound of Formula I,

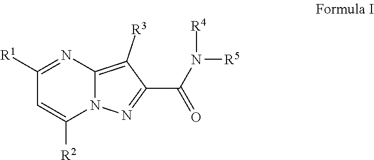

wherein:
R¹ is phenyl

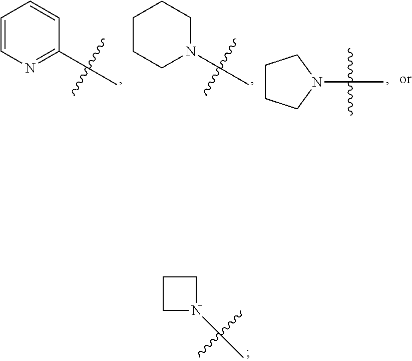

wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl,

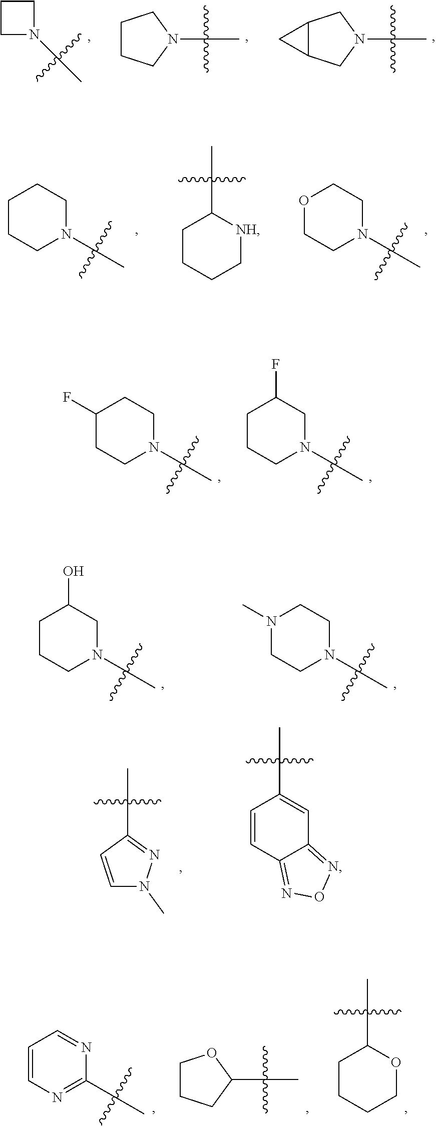

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H;
R⁴ is —H, or —CH₃;
R⁵ is —C₍₂₋₃₎alkyl-R^{a}, cyclobutyl,

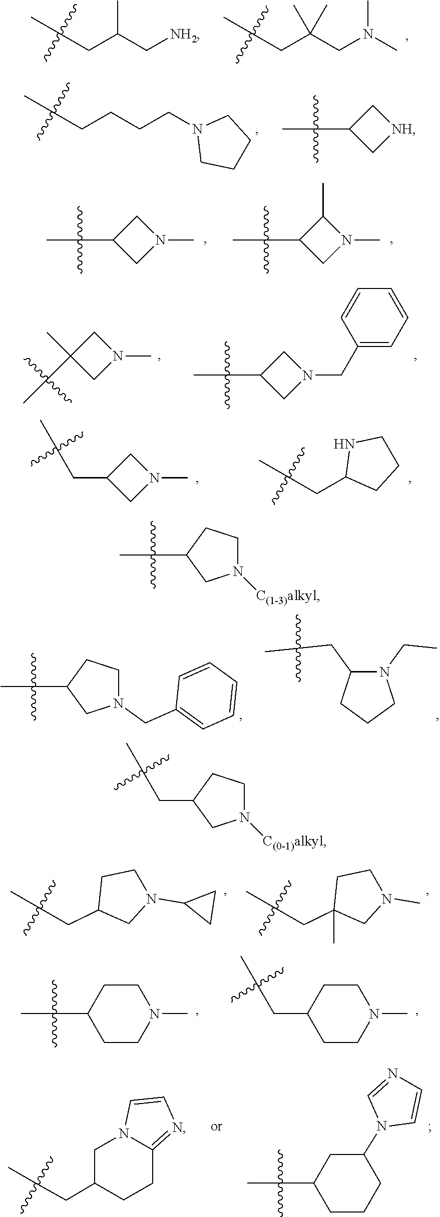

R^{a} is pyridinyl, azetidinyl, —NH₂, —NHCH₃, —N(CH₃)₂, —NHpyridinyl, or —N(CH₃)pyridinyl;
and pharmaceutically acceptable salts thereof.

Another embodiment of the invention is the compound of Formula I,

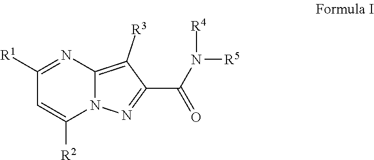

wherein:
R¹ is phenyl,

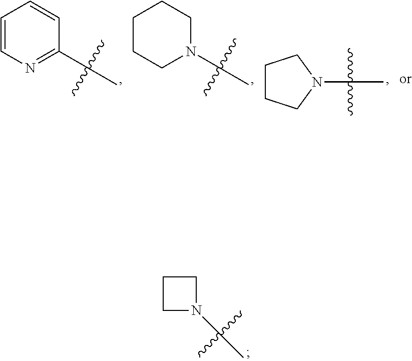

wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl,

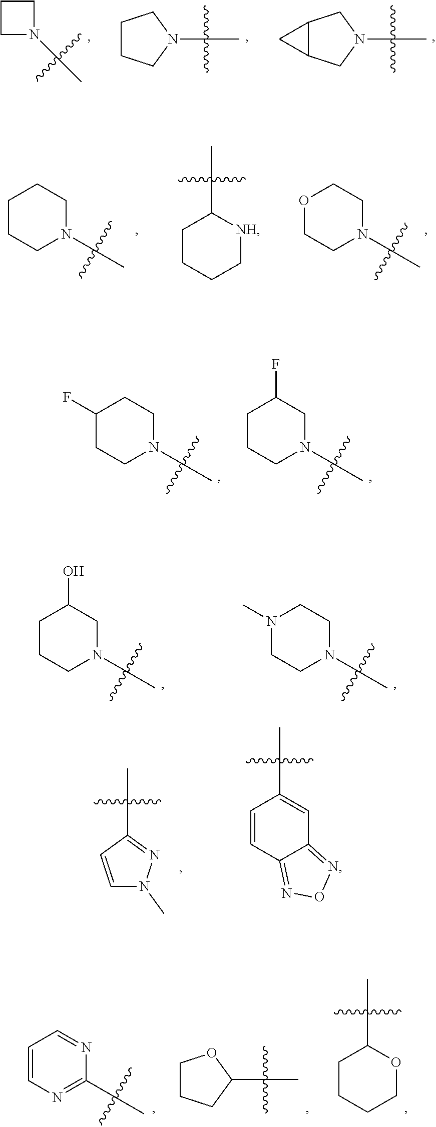

—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H —I, —Cl, —F, —CN, or —CH₃;
R⁴ is —H;
R⁵ is —C₍₂₋₃₎alkyl-R^{a}, cyclobutyl,

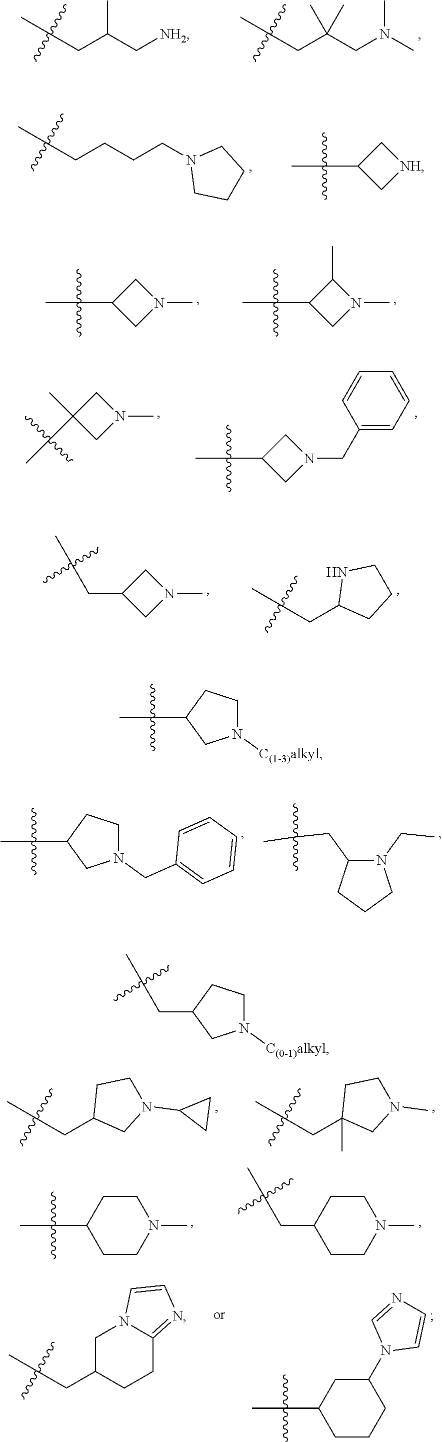

R^{a} is pyridinyl, azetidinyl, —NH₂, —NHCH₃, —N(CH₃)₂, —NHpyridinyl, or —N(CH₃)pyridinyl;
and pharmaceutically acceptable salts thereof.

Another embodiment of the invention is a compound selected from the group consisting of:

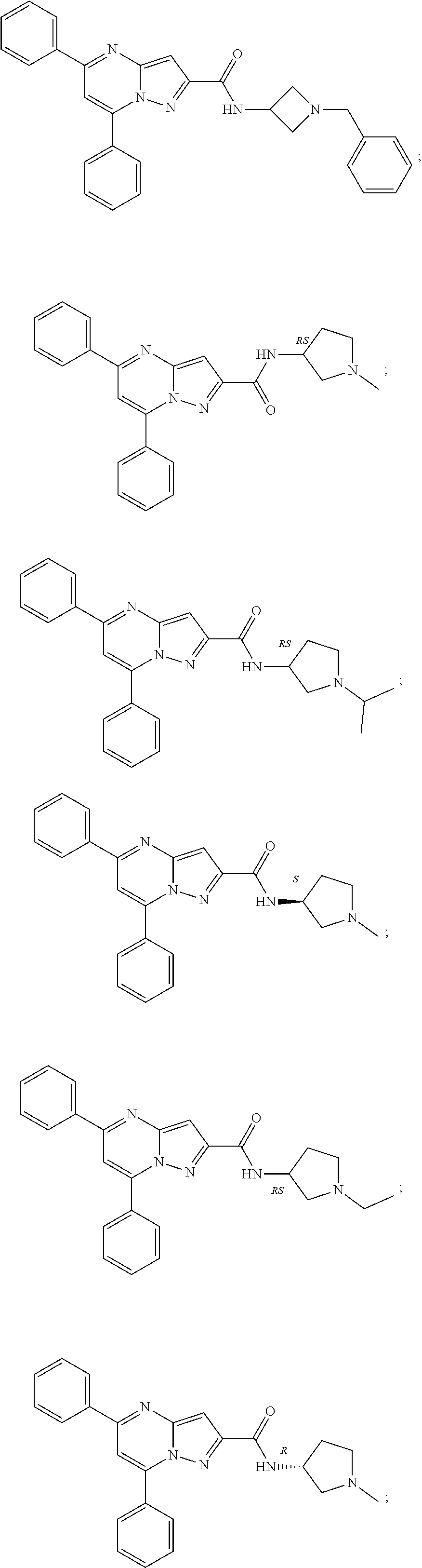

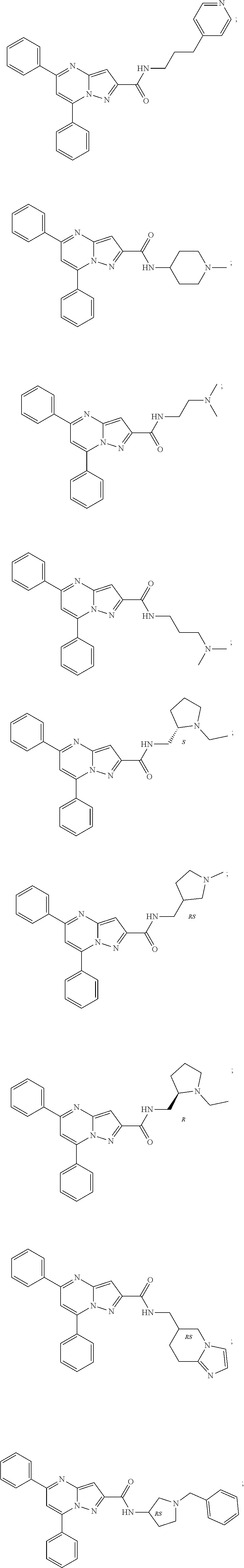

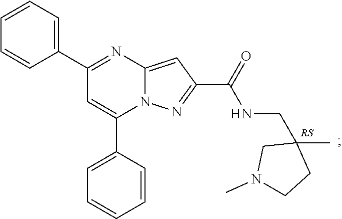 [SMILES: CN1CCC(C)(CNC(=O)c2cc3nc(-c4ccccc4)cc(-c4ccccc4)n3n2)C1]

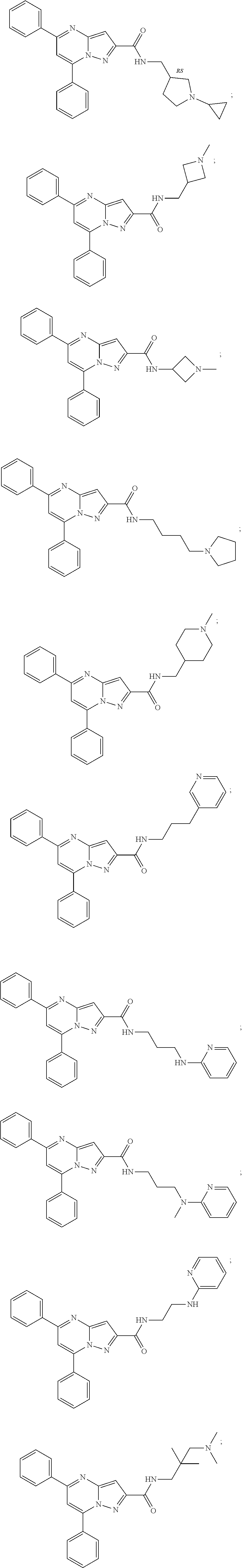

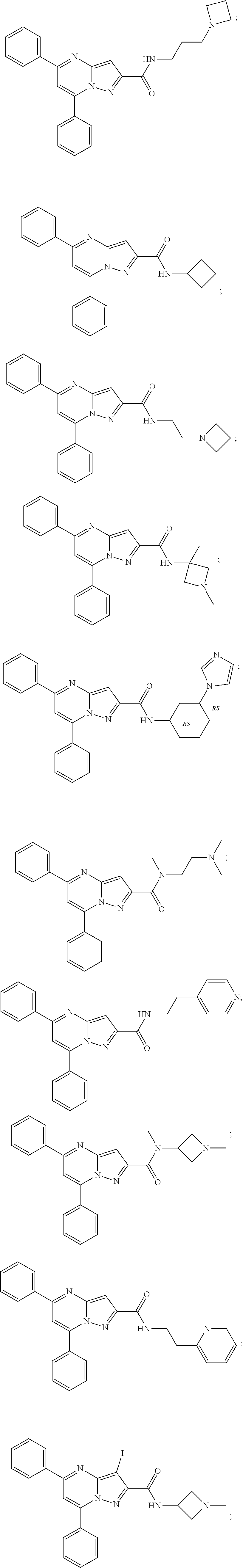

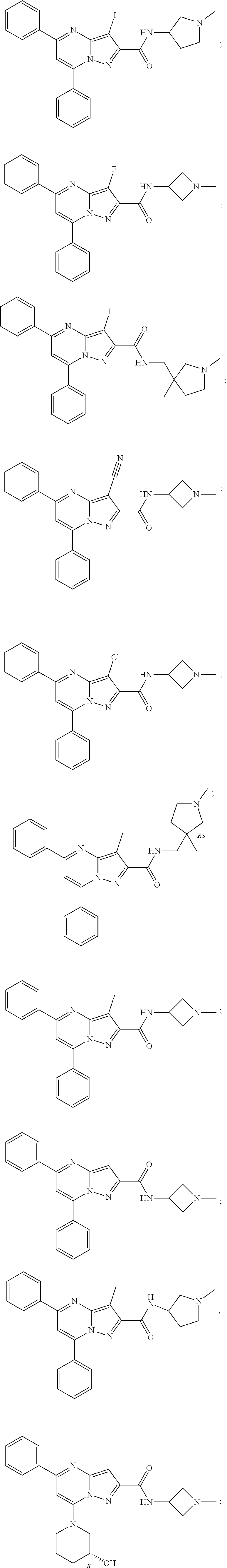

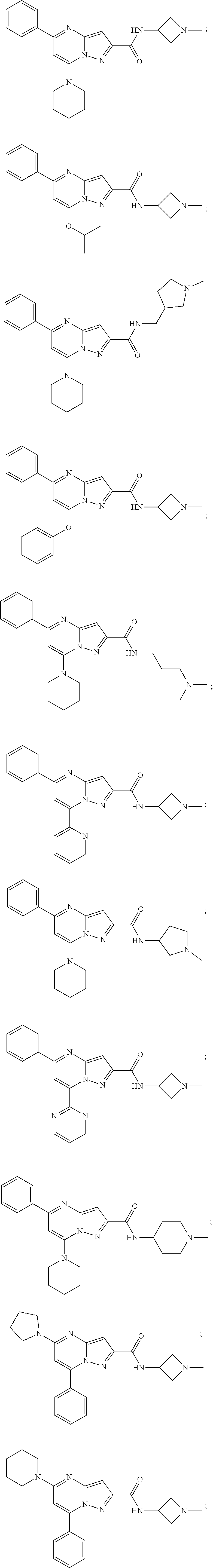

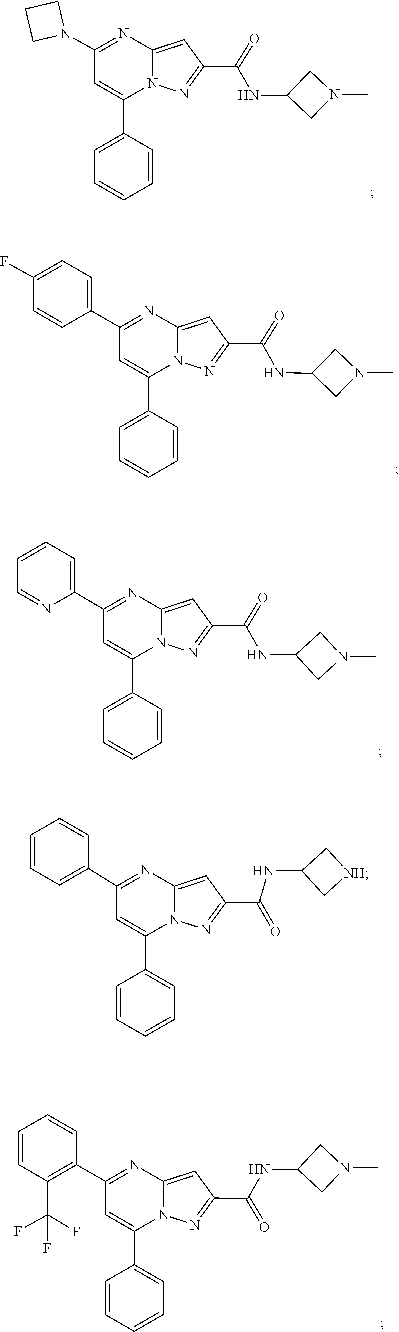 [SMILES: CN1CC(NC(=O)c2cc3nc(N4CCC4)cc(-c4ccccc4)n3n2)C1 ; CN1CC(NC(=O)c2cc3nc(-c4ccc(F)cc4)cc(-c4ccccc4)n3n2)C1 ; CN1CC(NC(=O)c2cc3nc(-c4ccccn4)cc(-c4ccccc4)n3n2)C1 ; O=C(NC1CNC1)c1cc2nc(-c3ccccc3)cc(-c3ccccc3)n2n1 ; CN1CC(NC(=O)c2cc3nc(-c4ccccc4C(F)(F)F)cc(-c4ccccc4)n3n2)C1]

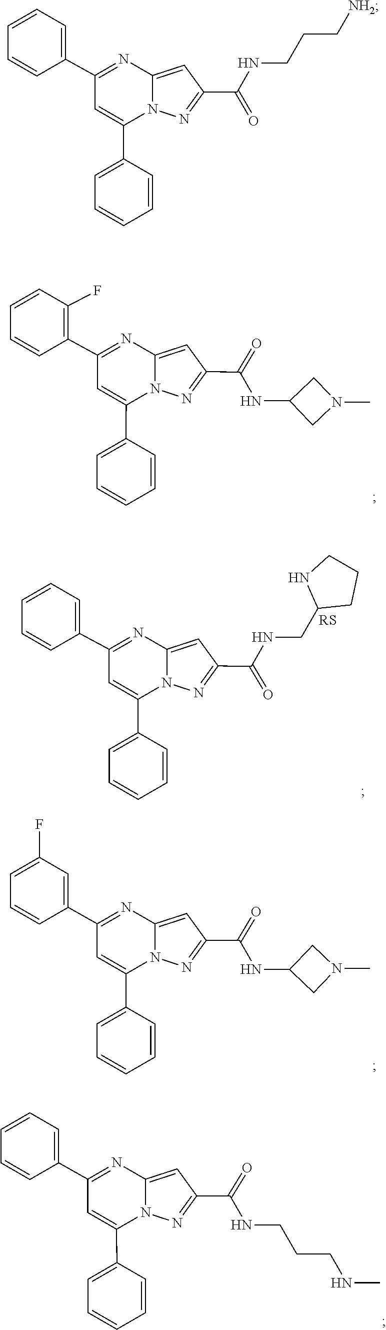 [SMILES: NCCCNC(=O)c1cc2nc(-c3ccccc3)cc(-c3ccccc3)n2n1 ; CN1CC(NC(=O)c2cc3nc(-c4ccccc4F)cc(-c4ccccc4)n3n2)C1 ; O=C(NCC1CCCN1)c1cc2nc(-c3ccccc3)cc(-c3ccccc3)n2n1 ; CN1CC(NC(=O)c2cc3nc(-c4cccc(F)c4)cc(-c4ccccc4)n3n2)C1 ; CNCCCNC(=O)c1cc2nc(-c3ccccc3)cc(-c3ccccc3)n2n1]

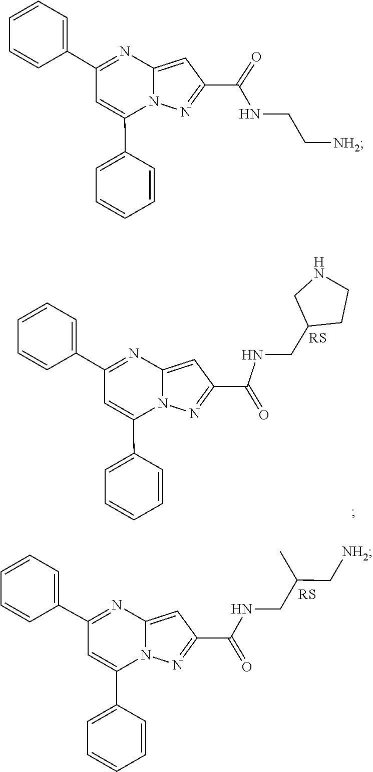 [SMILES: NCCNC(=O)c1cc2nc(-c3ccccc3)cc(-c3ccccc3)n2n1 ; O=C(NCC1CCNC1)c1cc2nc(-c3ccccc3)cc(-c3ccccc3)n2n1 ; CC(CN)CNC(=O)c1cc2nc(-c3ccccc3)cc(-c3ccccc3)n2n1]

and pharmaceutically acceptable salts thereof.

Another embodiment of the invention is a process for making a pharmaceutical composition comprising mixing a compound of Formula I and a pharmaceutically acceptable carrier.

Another embodiment of the invention is a method for treating or ameliorating a SMOX mediated syndrome, disorder or disease comprising administering to a subject in need thereof an effective amount of a compound of Formula I.

Another embodiment of the invention is a method for treating or ameliorating a SMOX mediated syndrome, disorder or disease comprising inhibiting spermine oxidase with a compound of Formula I as described herein by administering said compound to a subject in need thereof.

Another embodiment of the invention is a method for treating or ameliorating a SMOX mediated syndrome, disorder or disease comprising administering to a subject in need thereof an effective amount of a compound of Formula I, wherein the disease is selected from the group consisting of: colorectal cancer and vision loss.

Another embodiment of the invention is a method for treating or ameliorating a SMOX mediated syndrome, disorder or disease comprising administering to a subject in need thereof an effective amount of a compound of Formula I, wherein the disease is colorectal cancer.

Another embodiment of the invention is a method for treating or ameliorating a SMOX mediated syndrome, disorder or disease comprising administering to a subject in need thereof an effective amount of a compound of Formula I, wherein the disease is vision loss.

Another embodiment of the invention is a method of treating or ameliorating a syndrome, disorder or disease, in a subject in need thereof comprising administering to the subject an effective amount of a compound of Formula I or composition or medicament thereof in a combination therapy with one or more anti-inflammatory agents, or immunosuppressive agents, wherein said syndrome, disorder or disease is selected from the group consisting of: colorectal cancer and vision loss.

Another embodiment of the invention is a method of treating or ameliorating a syndrome, disorder or disease, in a subject in need thereof comprising administering to the subject an effective amount of a compound of Formula I or composition or medicament thereof in a combination therapy with one or more anti-inflammatory agents, or immunosuppressive agents, wherein said syndrome, disorder or disease is colorectal cancer.

The present invention provides a method of preventing, treating or ameliorating a syndrome, disorder or disease, wherein said syndrome, disorder or disease is selected from the group consisting of: ophthalmic disorders, including delay or prevention of vision loss in diabetic patients, and cancers, including colorectal cancer comprising administering to a subject in need thereof an effective amount of a compound of Formula I or a form, composition or medicament thereof.

The present invention provides a method of treating or ameliorating a syndrome, disorder or disease, wherein said syndrome, disorder or disease is colorectal cancer.

The present invention provides a method of treating or ameliorating a syndrome, disorder or disease, in a subject in need thereof comprising administering to the subject an effective amount of the compound of Formula I or composition or medicament thereof in a combination therapy with one or more anti-inflammatory agents, or immunosuppressive agents, wherein said syndrome, disorder or disease is colorectal cancer.

The invention also relates to methods of inhibiting SMOX activity in a mammal by administration of an effective amount of at least one compound of Formula I.

### Definitions

The term “administering” with respect to the methods of the invention, means a method for therapeutically or prophylactically preventing, treating or ameliorating a syndrome, disorder or disease as described herein by using a compound of Formula I or a form, composition or medicament thereof. Such methods include administering an effective amount of said compound, compound form, composition or medicament at different times during the course of a therapy or concurrently in a combination form. The methods of the invention are to be understood as embracing all known therapeutic treatment regimens.

The term “subject” refers to a patient, which may be an animal, typically a mammal, typically a human, which has been the object of treatment, observation or experiment and is at risk of (or susceptible to) developing a syndrome, disorder or disease that is associated with abberant SMOX expression or SMOX overexpression, or a patient with an inflammatory condition that accompanies syndromes, disorders or diseases associated with abberant SMOX expression or SMOX overexpression.

The term “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

As used herein, the term “composition” is intended to encompass a product comprising the specified ingredients in the specified amounts, as well as any product which results, directly or indirectly, from combinations of the specified ingredients in the specified amounts.

The term “alkyl” refers to both linear and branched chain radicals of up to 12 carbon atoms, preferably up to 6 carbon atoms, unless otherwise indicated, and includes, but is not limited to, methyl, ethyl, propyl, isopropyl, butyl, isobutyl, sec-butyl, tert-butyl, pentyl, isopentyl, hexyl, isohexyl, heptyl, octyl, 2,2,4-trimethylpentyl, nonyl, decyl, undecyl and dodecyl. Any alkyl group may be optionally substituted with one OCH₃, one OH, or up to two fluorine atoms.

The term “C_{(a-b)}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

### Pharmaceutically Acceptable Salts

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate, sodium hydroxide, triethanolamine, or zinc.

### Methods of Use

When employed as SMOX inhibitors, the compounds of the invention may be administered in an effective amount within the dosage range of about 0.5 mg to about 10 g, preferably between about 0.5 mg to about 5 g, in single or divided daily doses. The dosage administered will be affected by factors such as the route of administration, the health, weight and age of the recipient, the frequency of the treatment and the presence of concurrent and unrelated treatments.

It is also apparent to one skilled in the art that the therapeutically effective dose for compounds of the present invention or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

The compounds of Formula I may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutically-acceptable salts of the compounds of Formula I include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamino salts and salts with amino acids such as arginine. Also, the basic nitrogen-containing groups may be quaternized with, for example, alkyl halides.

The pharmaceutical compositions of the invention may be administered by any means that accomplish their intended purpose. Examples include administration by parenteral, subcutaneous, intravenous, intramuscular, intraperitoneal, transdermal, buccal or ocular routes. Alternatively or concurrently, administration may be by the oral route. Suitable formulations for parenteral administration include aqueous solutions of the active compounds in water-soluble form, for example, water-soluble salts, acidic solutions, alkaline solutions, dextrose-water solutions, isotonic carbohydrate solutions and cyclodextrin inclusion complexes.

The present invention also encompasses a method of making a pharmaceutical composition comprising mixing a pharmaceutically acceptable carrier with any of the compounds of the present invention. Additionally, the present invention includes pharmaceutical compositions made by mixing a pharmaceutically acceptable carrier with any of the compounds of the present invention.

### Polymorphs and Solvates

Furthermore, the compounds of the present invention may have one or more polymorph or amorphous crystalline forms and as such are intended to be included in the scope of the invention. In addition, the compounds may form solvates, for example with water (i.e., hydrates) or common organic solvents. As used herein, the term “solvate” means a physical association of the compounds of the present invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include ethanolates, methanolates, and the like.

It is intended that the present invention include within its scope polymorphs and solvates of the compounds of the present invention. Thus, in the methods of treatment of the present invention, the term “administering” shall encompass the means for treating, ameliorating or preventing a syndrome, disorder or disease described herein with the compounds of the present invention or a polymorph or solvate thereof, which would obviously be included within the scope of the invention albeit not specifically disclosed.

In another embodiment, the invention relates to a compound as described in Formula I for use as a medicament.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, Ed. H. Bundgaard, Elsevier, 1985.

Furthermore, it is intended that within the scope of the present invention, any element, in particular when mentioned in relation to a compound of Formula I, shall comprise all isotopes and isotopic mixtures of said element, either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radiolabelled compounds of Formula I may comprise a radioactive isotope selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

Some compounds of the present invention may exist as atropisomers. Atropisomers are stereoisomers resulting from hindered rotation about single bonds where the steric strain barrier to rotation is high enough to allow for the isolation of the conformers. It is to be understood that all such conformers and mixtures thereof are encompassed within the scope of the present invention.

Where the compounds according to this invention have at least one stereo center, they may accordingly exist as enantiomers or diastereomers. It is to be understood that all such isomers and mixtures thereof are encompassed within the scope of the present invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J.F.W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

### Abbreviations

Herein and throughout the application, the following abbreviations may be used.
4CzIPN 1,2,3,5-Tetrakis(carbazol-9-yl)-4,6-dicyanobenzene, 2,4,5,6-Tetrakis(9H-carbazol-9-yl) isophthalonitrile
Ac acetyl
ACN acetonitrile
aq sat, or sat. aq saturated aqueous
bu butyl
DCE dichloroethane
DCM dichloromethane
DEA diethylamine
dppf 1,1′-bis(diphenylphosphino) ferrocine
DIPEA N,N-diisopropylethylamine (HUnig's base)
DME 1,2-dimethoxyethane
DMA N,N-dimethylacetamide
DMF N,N-dimethylformamide
DMSO dimethyl sulfoxide
EDCI 1-ethyl-3-(3-dimethylaminopropyl) carbodiimide
Et₃N triethylamine
Et₂O diethyl ether
EtOAc ethyl acetate
EtOH ethyl alcohol
ESI electrospray ionization
Et ethyl
h hour(s)
HATU O-(7-azabenzotriazol-1-yl)-N,N,N′,N′-tetramethyluronium hexafluorophosphate
Hex hexanes
HOBt 1-hydroxybenzotriazole
HPLC high pressure liquid chromatography
Hz Hertz
IPA isopropylalcohol
LHMDS, or LiHMDS lithium hexamethyldisilazane
MS mass spectrometry
Me methyl
MeCN acetonitrile
NBS N-bromosuccinimide
NIS N-iodosuccinimide
NMR nuclear magnetic resonance
Pd(PPh₃)₄ Tetrakis(triphenylphosphine)palladium(0)
PE petroleum ether
Ph phenyl
iPrOH isopropyl alcohol
ppm parts per million
rt room temperature
t-bu tertiary butyl
TEA triethylamine
TFA trifluoroacetic acid
THF tetrahydrofuran
RuPhos 2-dicyclohexylphosphino-2′,6′-diisopropoxybiphenyl
SELECTFLUOR 1-chloromethyl-4-fluoro-1,4-diazoniabicyclo[2.2.2]octane bis(tetrafluoroborate)

### General Schemes:

Compounds of Formula I in the present invention can be synthesized in accordance with the general synthetic methods known to those who are skilled in the art. The following reaction schemes are only meant to represent examples of the invention and are in no way meant to be a limit of the invention.

### General Schemes:

Compounds of Formula I wherein R¹ and R² are Ph and R³ is H can be prepared according to Scheme 1. Condensation of commercially available A-I with 1,3-diphenylpropane-1,3-dione under acidic media can give pyrazolopyrimidines A-II. Then, ester hydrolysis using aqueous hydroxide solution and a cosolvent such as 1,4-dioxane or THF can give carboxylic acids A-III. Amides of Formula I can be formed by reaction of A-III with amines or amine salts of formula NHR⁴R⁵ promoted by a reagent such as HATU or EDCI and a base such as DIPEA, TEA in a solvent such as DMF, MeCN, or DCM.

In certain embodiments of the invention, R⁵ could contain a reactive amine group, such that the compound NHR⁴R⁵ contains at least two nitrogen atoms. Those skilled in the art will recognize that the reactive amine group in R⁵ may be protected, with any amine protecting group known in the art, for example Boc, and this protecting group is removed after NHR⁴R⁵ coupling to produce the compound of Formula I.

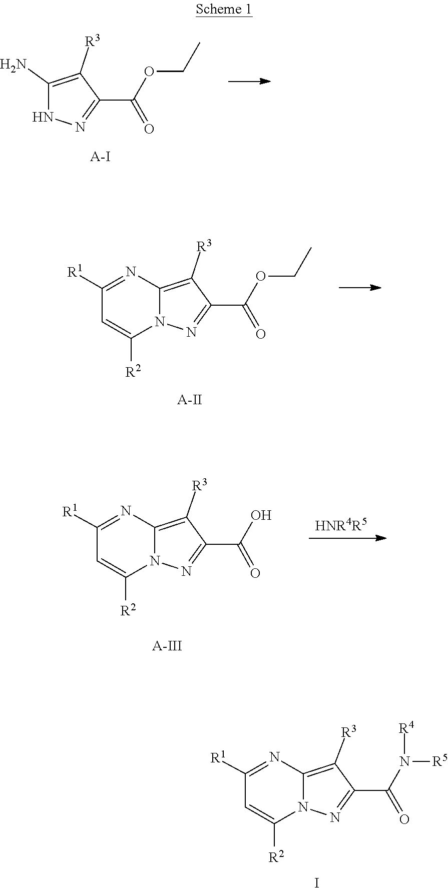

Compounds of Formula I wherein R¹ is Ph, R³ is H, and R² is alkyl, hetero-aryl, heterocyclyl, NH-alkyl, N-cycloalkyl, O-alkyl and O-aryl, in the present invention can be prepared according to Scheme 2. Compound B-II can be obtained by treatment of B-I (see PCT Int. Appl., 2003101993, 11 Dec. 2003) with POCl₃ in solvent like DCE in presence of a base such as N,N-diethylaniline or without the presence of a base. Palladium-catalyzed cross-coupling reaction between B-II and a boron compound, for example, boronic esters or boronic acids, can deliver B-III (R² is aryl). Alternatively, B-II can react with amines or alcohols, optionally in the presence of a base, to deliver B-III (where R² is hetero-aryl, heterocyclyl, NH-alkyl, N-cycloalkyl, O-alkyl and O-aryl). Alternatively, B-II can react with Zn-alkyl catalyzed by palladium-catalyzed cross-coupling reaction using catalytic systems such as Pd(OAc)₂ and RuPhos to give B-III (R² is alkyl). B-II can also react with acids using Ni-catalysts and photocatalyst under light irradiation to deliver compound B-III.

Ester hydrolysis using aqueous hydroxide solution and a cosolvent such as 1,4-dioxane or THF can give carboxylic acids B-IV. Amides of Formula I can be formed by reaction of B-IV with amines or amine salts of formula NHR⁴R⁵ promoted by a reagent such as HATU or EDCI and a base such as DIPEA, TEA in a solvent such as DMF, MeCN, or DCM. Alternatively, amides of Formula I can be formed by reaction of B-III with amines or amine salts of formula NHR⁴R⁵ promoted by LHMDS, as described in Chem Commun. 2014, 50, 15094.

In certain embodiments of the invention, R⁵ could contain a reactive amine group, such that the compound NHR⁴R⁵ contains at least two nitrogen atoms. Those skilled in the art will recognize that the reactive amine group in R⁵ may be protected, with any amine protecting group known in the art, for example Boc, and this protecting group is removed after NHR⁴R⁵ coupling to produce the compound of Formula I. Those skilled in the art will recognize that further derivitization of the unprotected amine in possible at this stage of the synthesis.

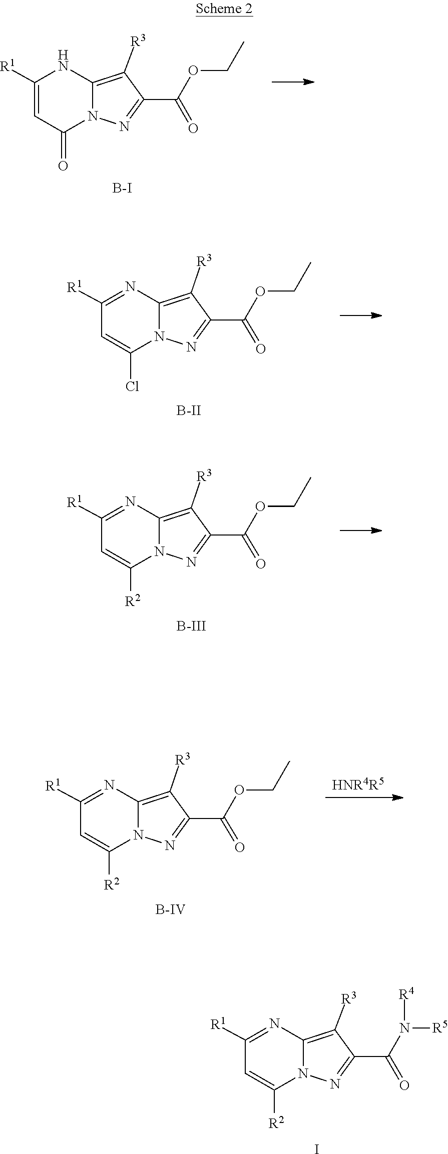

If the ester of formula B-I is hydrolyzed prior to the installation of R², conversion to Formula I can be accomplished as shown in Scheme 3. Amides of Formula B-VI can be formed by reaction of B-V (see PCT Int. Appl., 2003101993, 11 Dec. 2003) with amines or amine salts of formula NHR⁴R⁵ promoted by a reagent such as HATU or EDCI and a base such as DIPEA or TEA in a solvent such as DMF, MeCN, or DCM. B-VII can be obtained treating B-VI with phosphoryl chloride, optionally in the presence of a base such as N,N-diethylaniline, in solvents like DCE. Subsequent installation of R² can be accomplished by reaction SNAr reaction of B-VII with amines or salts in solvents such as iPrOH in the presence of a base like DIPEA to deliver I.

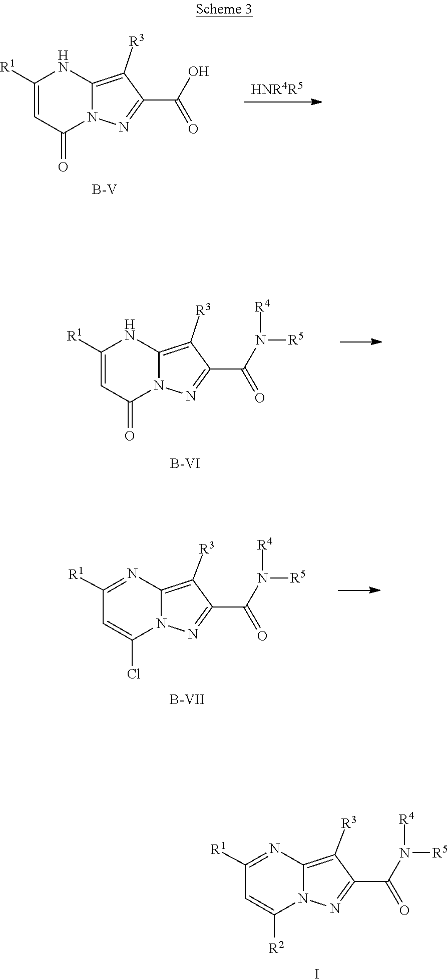

Compounds of Formula I wherein R³ is not H can be prepared according to Scheme 4. C-I (wherein R³ is H) can be treated with SELECTFLUOR in CH₃CN to give C-II (wherein R³ is F). Moreover, C-I can be halogenated at R³ by treatment C-I with NIS or NCS in solvents like DMF to give compounds C-II (wherein R³ is Cl, or I).

In cases where R³ is CN compound C-II is treated with Zn(CN)₂ in the presence of a palladium catalyst, such as bis(tri-tert-butylphosphine)palladium (0) to give the corresponding C-II wherein R³ is CN. Subsequent ester hydrolysis using aqueous hydroxide solution and a cosolvent such as 1,4-dioxane or THF can give carboxylic acids C-III. Amides of Formula I can be formed by reaction of C-III with amines or amine salts of formula NHR⁴R⁵ promoted by a reagent such as HATU or EDCI and a base such as DIPEA, TEA in a solvent such as DMF, MeCN, or DCM. In cases where R³ is alkyl, compound C-II (wherein R³ is I, Br, or Cl) is treated with Zn(C₍₁₋₄₎alkyl)₂, in the presence of a palladium catalyst, such as bis(tri-tert-butylphosphine)palladium (0) and solvent such as THF to give the corresponding I wherein R³ is alkyl.

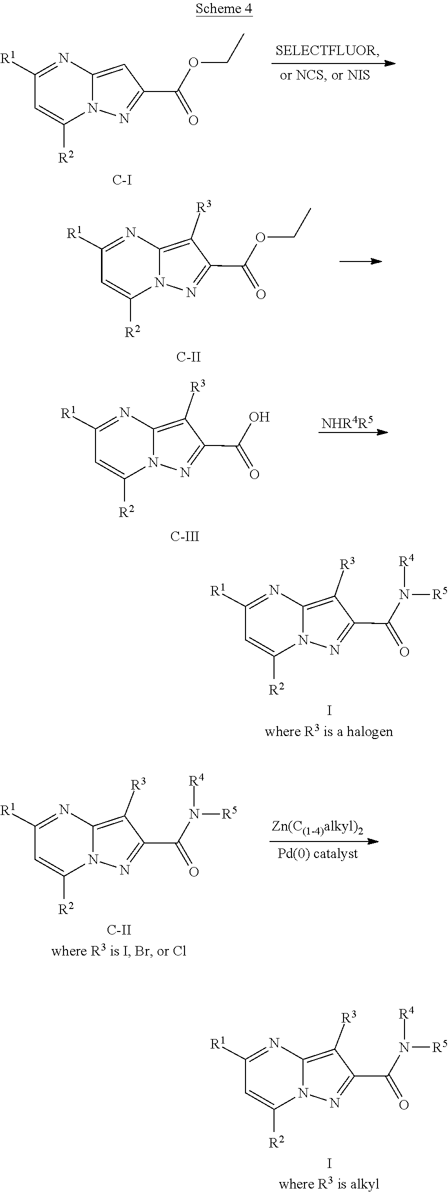

The compounds of Formula I wherein R¹ is N-alkyl heteroaryl, or aryl, R² is Ph, and R³ is H can be prepared according to Scheme 5. Amide coupling between aminopyrazole D-I and mono-ethyl malonate in a presence of N,N-diisopropylcarbodiimide or any other amide coupling reagent can give D-II. Treatment of D-II with DMAP can be converted to D-III. Heating D-III in a presence of POCl₃ can deliver D-IV. Palladium-catalyzed cross-coupling reaction between D-IV and boronic esters or boronic acids can selectively deliver D-V (where R² is Ph). Subsequent treatment of D-V with an amine and a base can give D-VI (R¹ is N-alkyl). Alternatively, D-V could deliver D-VI by treatment of with a boronic acid or boronic ester in a presence of a palladium as a catalyst (R¹ is aryl or heteroaryl) Hydrolysis of the D-VI ester and installation of the —NR⁴R⁵ amide moiety to yield I can be performed as described in the preceding schemes.

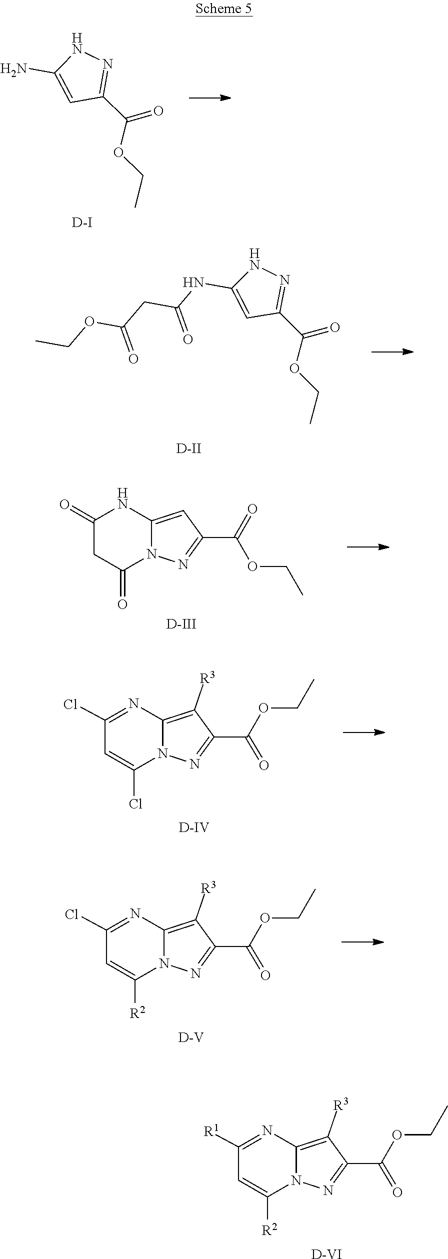

An alternative method for installation of the —NR⁴R⁵ amide moiety is described Scheme 6. Treatment of acid E-I (where R¹ is aryl, R² is Ph, and R³ is H), prepared as described in the preceeding schemes, with diphenyl phosphoryl azide can deliver E-II. Azide E-II can be treated with an amine of formula HNR⁴R⁵ to deliver compounds of Formula I

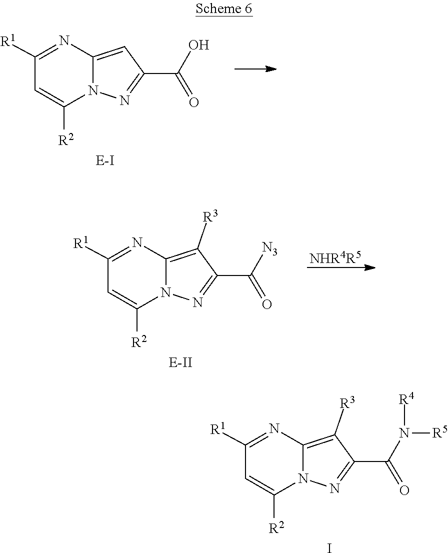

### EXAMPLES

Compounds of the present invention can be prepared by methods known to those who are skilled in the art. The following examples are only meant to represent examples of the invention and are in no way meant to be a limit of the invention.

### Intermediate 1. Ethyl 5,7-diphenylpyrazolo[1,5,a]pyrimidine-2-carboxylate

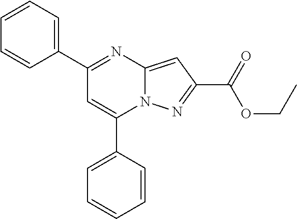 [SMILES: CCOC(=O)c1cc2nc(-c3ccccc3)cc(-c3ccccc3)n2n1]

A mixture of 1,3-diphenylpropane-1,3-dione (10 g, 44.6 mmol) and ethyl 5,7-diphenylpyrazolo[1,5-a]pyrimidine-2-carboxylate (8.5 g, 54.8 mmol) was dissolved in acetic acid (250 mL). The mixture was stirred for 24 h at reflux. The mixture was cooled to ambient temperature and concentrated in vacuo. The residue was dissolved in EtOAc and neutralized with aq NaHCO₃ sol. The layers were separated, and the aqueous phase was extracted with EtOAc. The combined organic layers were washed with brine and dried over MgSO₄. The crude product was purified by flash chromatography (SiO₂, EtOAc in heptane 0/100 to 75/25) to yield Intermediate 1 as a beige solid. MS (ESI+) calculated for C₂₁H₁₇N₃O₂: 343.1, found: 344.1 [M+H]⁺.

### Intermediate 2. 5,7-Diphenylpyrazolo[1,5,a]pyrimidine-2-carboxylic acid

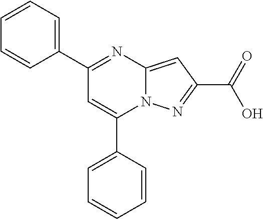 [SMILES: O=C(O)c1cc2nc(-c3ccccc3)cc(-c3ccccc3)n2n1]

An aqueous solution of LiOH (5 mL, 2 M, 10 mmol) was added to a solution of ethyl 5,7-diphenylpyrazolo[1,5,a]pyrimidine-2-carboxylate (Intermediate 1) in THF (10 mL). The mixture was stirred at room temperature for 18 h. Then, EtOAc was added to the mixture and the layers were separated. The aqueous phase was acidified with aq 1N HCl and extracted with EtOAc (×2). Combined organic layers were dried over MgSO₄ and the solvent was removed in vacuo to yield Intermediate 2 as a pale solid. MS (ESI+) calculated for C₁₉H₁₃N₃O₂: 315.1, found: 316.1 [M+H]⁺.

### Intermediate 3. Ethyl 7-chloro-5-phenylpyrazolo[1,5-a]pyrimidine-2-carboxylate

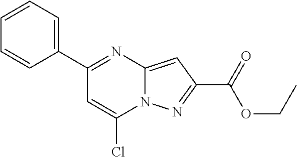 [SMILES: CCOC(=O)c1cc2nc(-c3ccccc3)cc(Cl)n2n1]

In a EasyMax vessel, phosphoryl chloride (50 mL, 1.64 g/mL, 534 mmol) was added at 0° C. to a solution of ethyl 7-hydroxy-5-phenylpyrazolo[1,5-a]pyrimidine-2-carboxylate (12 g, 41 mmol) and N,N-diethylaniline (25 mL, 0.93 g/mL, 156 mmol) in DCE (450 mL). The reaction mixture was stirred at 40° C. for 38 h. The, the reaction mixture was concentrated and dissolved in DCM. H₂O was added dropwise, followed by aq Na₂CO₃. The layers were separated, and the aqueous phase was extracted with DCM. The combined organic layers were washed with brine, dried over MgSO₄, and concentrated. The residue was purified by flash chromatography (SiO₂, EtOAc in heptane 0/100 to 50/50). The desired fractions were combined and concentrated in vacuo to yield Intermediate 3 as beige solid. MS (ESI+) calculated for C₁₅H₁₂ClN₃O₂: 301.1, found: 302.1 [M+H]⁺.

### Intermediate 4. Ethyl 3-iodo-5,7-diphenylpyrazolo[1,5-a]pyrimidine-2-carboxylate

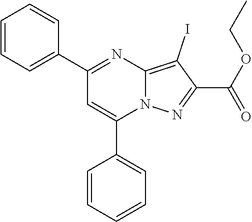 [SMILES: CCOC(=O)c1nn2c(-c3ccccc3)cc(-c3ccccc3)nc2c1I]

NIS (607 mg, 2.7 mmol) was added to a stirred solution of ethyl 5,7-diphenylpyrazolo[1,5,a]pyrimidine-2-carboxylate (Intermediate 1) (859 mg, 2.5 mmol) in DMF (12 mL). The mixture was stirred at rt for 16 h. Then, additionally NIS (60 mg, 0.27 mmol) was added and the mixture was stirred at rt for 5 h. The mixture was diluted with EtOAc and washed with sat. aq. Na₂S₂O₃ solution and brine. The organic layer was separated, dried (MgSO₄), filtered and concentrated in vacuo. The crude product was purified by flash column chromatography (SiO₂, EtOAc in heptane 10/90 to 100/0). The desired fractions were collected and concentrated in vacuo to yield Intermediate 4 as a yellow solid. MS (ESI+) calculated for C₂₁H₁₆IN₃O₂: 469.0, found: 470.0 [M+H]⁺.

### Intermediate 5. Ethyl 3-chloro-5,7-diphenylpyrazolo[1,5-a]pyrimidine-2-carboxylate

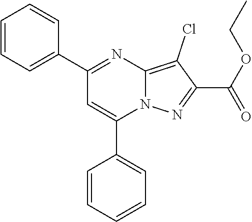 [SMILES: CCOC(=O)c1nn2c(-c3ccccc3)cc(-c3ccccc3)nc2c1Cl]

N-Chlorosuccinimide (65 mg, 0.49 mmol) was added to a stirred solution of ethyl 5,7-diphenylpyrazolo[1,5,a]pyrimidine-2-carboxylate (Intermediate 1) (172 mg, 0.5 mmol) in DMF (2.5 mL) in a sealed tube. The mixture was stirred at 70° C. for 60 h. The mixture was diluted with EtOAc and washed with aq sat Na₂S₂O₃ and brine. The organic layer was separated, dried (MgSO₄), filtered and concentrated in vacuo. The crude product was purified by flash column chromatography (SiO₂, EtOAc in heptane 10/90 to 100/0). The desired fractions were collected and concentrated in vacuo to yield Intermediate 5 as a yellow solid. MS (ESI+) calculated for C₂₁H₁₆ClN₃O₂: 377.1, found: 378.1 [M+H]⁺.

### Intermediate 6. Ethyl 3-fluoro-5,7-diphenylpyrazolo[1,5-a]pyrimidine-2-carboxylate

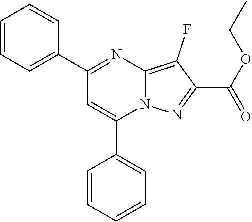 [SMILES: CCOC(=O)c1nn2c(-c3ccccc3)cc(-c3ccccc3)nc2c1F]

SELECTFLUOR (85 mg, 0.24 mmol) was added to a stirred suspension of ethyl 5,7-diphenylpyrazolo[1,5,a]pyrimidine-2-carboxylate (Intermediate 1) (69 mg, 0.2 mmol) in ACN (1 mL). The mixture was stirred at 80° C. for 16 h. The solvent was evaporated in vacuo and the crude product was purified by flash chromatography (SiO₂, EtOAc in heptane 10/90 to 100/0). The desired fractions were collected and concentrated in vacuo to yield Intermediate 6 as a yellow solid. MS (ESI+) calculated for C₂₁H₁₆FN₃O₂: 361.1, found: 362.2 [M+H]⁺.

### Intermediate 7. Ethyl 3-cyano-5,7-diphenylpyrazolo[1,5-a]pyrimidine-2-carboxylate

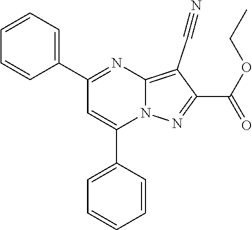 [SMILES: CCOC(=O)c1nn2c(-c3ccccc3)cc(-c3ccccc3)nc2c1C#N]

DMF (1 mL) was added to a stirred mixture of ethyl 3-iodo-5,7-diphenylpyrazolo[1,5-a]pyrimidine-2-carboxylate (94 mg, 0.2 mmol) (Intermediate 4), Zn(CN)₂ (28 mg, 0.24 mmol) and Pd(PPh₃)₄ (23 mg, 0.02 mmol) in a sealed tube. The mixture was stirred at 100° C. for 60 h. Then more Zn(CN)₂ (28 mg, 0.24 mmol) and Pd(PPh₃)₄ (23 mg, 0.02 mmol) were added and the mixture was stirred at 100° C. further for 24 h. The mixture was treated with water and DCM and stirred for 15 min. The layers were separated, and the organic layer was concentrated in vacuo. The residue was purified by flash column chromatography (SiO₂, EtOAc in heptane 0/100 to 50/50). The desired fractions were collected and concentrated in vacuo to yield Intermediate 7 as a yellow solid. MS (ESI+) calculated for C₂₂H₁₆N₄O₂: 368.10, found: 369.1 [M+H]⁺.

### Intermediate 8. 3-Iodo-5,7-diphenylpyrazolo[1,5-a]pyrimidine-2-carboxylic acid

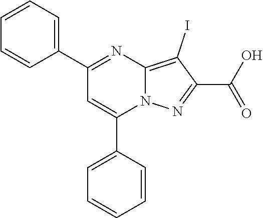 [SMILES: O=C(O)c1nn2c(-c3ccccc3)cc(-c3ccccc3)nc2c1I]

LiOH solution in H₂O (0.8 mL, 1 M, 0.8 mmol) was added to a stirred solution of ethyl 3-iodo-5,7-diphenylpyrazolo[1,5-a]pyrimidine-2-carboxylate (Intermediate 4) (230 mg, 0.49 mmol) in THF (4 mL). The mixture was stirred at rt for 16 h. The mixture was acidified with 1N HCl to pH 2 and extracted with DCM. The organic layer was separated, dried (MgSO₄), filtered and concentrated in vacuo to yield Intermediate 8 as a yellow solid. MS (ESI+) calculated for C₁₉H₁₂IN₃O₂: 441.00, found: 442.3 [M+H]+.

### Intermediate 9. 3-Chloro-5,7-diphenylpyrazolo[1,5-a]pyrimidine-2-carboxylic acid

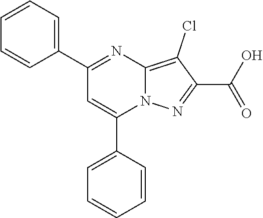 [SMILES: O=C(O)c1nn2c(-c3ccccc3)cc(-c3ccccc3)nc2c1Cl]

Example 9 was synthetized following the method described for Intermediate 8 using ethyl 3-chloro-5,7-diphenylpyrazolo[1,5-a]pyrimidine-2-carboxylate (Intermediate 5) (144 mg, 0.38 mmol) yielding Intermediate 9 as yellow solid. MS (ESI+) calculated for C₁₉H₁₂ClN₃O₂: 349.10, found: 350.0 [M+H]⁺.

### Intermediate 10. 3-Fluoro-5,7-diphenylpyrazolo[1,5-a]pyrimidine-2-carboxylic acid

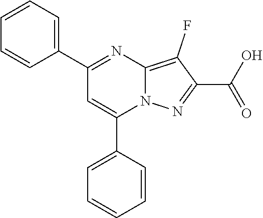 [SMILES: O=C(O)c1nn2c(-c3ccccc3)cc(-c3ccccc3)nc2c1F]

Intermediate 10 was synthetized following the method described for Intermediate 8 using ethyl 3-fluoro-5,7-diphenylpyrazolo[1,5-a]pyrimidine-2-carboxylate (Intermediate 6) (160 mg, 0.44 mmol) yielding Intermediate 10 as a yellow solid. MS (ESI+) calculated for C₁₉H₁₂FN₃O₂: 333.1, found: 334.1 [M+H]⁺.

### Intermediate 11. 3-Cyano-5,7-diphenylpyrazolo[1,5-a]pyrimidine-2-carboxylic acid

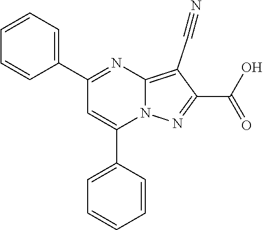 [SMILES: N#Cc1c(C(=O)O)nn2c(-c3ccccc3)cc(-c3ccccc3)nc12]

Intermediate 11 was synthetized following the method described for Intermediate 8 using ethyl 3-cyano-5,7-diphenylpyrazolo[1,5-a]pyrimidine-2-carboxylate (Intermediate 7) (48 mg, 0.082 mmol) yielding Intermediate 11 as a yellow solid. MS (ESI+) calculated for C₂₀H₁₂N₄O₂: 340.2, found: 341.1 [M+H]⁺.

### Intermediate 12. N-(3-(1H-imidazol-1-yl)propyl)-7-oxo-5-phenyl-4,7-dihydropyrazolo[1,5-a]pyrimidine-2-carboxamide

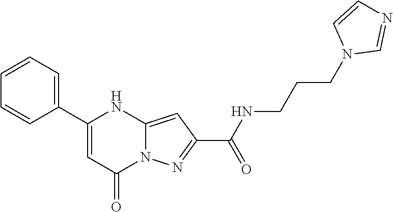 [SMILES: O=C(NCCCn1ccnc1)c1cc2[nH]c(-c3ccccc3)cc(=O)n2n1]

1-(3-aminopropyl)imidazole (2.27 mL, 0.95 g/mL, 17.24 mmol) was added to a mixture containing Et₃N (6.5 mL, 0.73 g/mL, 47.0 mmol), pyrazolo[1,5-a]pyrimidine-2-carboxylic acid, 4,7-dihydro-7-oxo-5-phenyl-(4 g, 15.67 mmol) and HATU (7.45 g, 19.6 mmol) in DMF (100 mL) and stirred at rt for 25 h. Aq sat NaHCO₃ solution and DCM were added and the layers were separated. The aqueous phase was extracted with a mixture iPrOH:CHCl₃ (1:7). Combined organic layers were dried in vacuo and acetonitrile was added. The white solid was filtered to yield Intermediate 12. MS (ESI+) calculated for C₁₉H₁₈N₆O₂: 362.2, found: 363.2 [M+H]⁺.

### Intermediate 13. N-(3-(1H-imidazol-1-yl)propyl)-7-chloro-5-phenylpyrazolo[1,5-a]pyrimidine-2-carboxamide

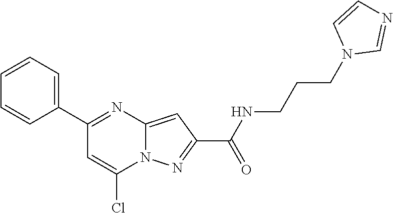 [SMILES: O=C(NCCCn1ccnc1)c1cc2nc(-c3ccccc3)cc(Cl)n2n1]

Phosphoryl chloride (13 mL, 1.64 g/mL, 139 mmol) was added at 0° C. to a solution of Intermediate 12 (2.5 g, 6.9 mmol) and N,N-diethylaniline (5.5 mL, 0.93 g/mL, 34.3 mmol) in DCE (30 mL). The reaction mixture was stirred at reflux (90° C.) for 7 h. The reaction mixture cooled down to r.t. and concentrated in vacuo. The residue was dissolved in DCM and slightly basified with aq NaHCO₃ solution, and extracted with EtOAc (3×). The combined organic layers were dried over MgSO₄ and concentrated in vacuo. The residue was purified by flash chromatography (SiO₂, MeOH in DCM 1/99 to 25/75) to yield Intermediate 13 as a yellowish syrup. MS (ESI+) calculated for C₁₉H₁₇ClN₆O: 380.1, found: 381.2 [M+H]⁺.

### Intermediate 14. Ethyl 5-(3-ethoxy-3-oxopropanamido)-1H-pyrazole-3-carboxylate

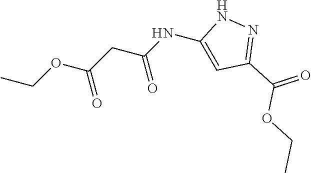 [SMILES: CCOC(=O)CC(=O)Nc1cc(C(=O)OCC)n[nH]1]

N,N-Diisopropylcarbodiimide (7.8 mL, 0.815 g/mL, 50.228 mmol) was added to a mixture of ethyl 5-amino-1H-pyrazole-3-carboxylate (6 g, 38.671 mmol) and mono-ethyl malonate (4.8 mL, 1.119 gr/mL, 40.597 mmol) in pyridine (193 mL) at 0° C. The mixture was stirred at 0° C. for 1 h and then at rt for 18 h. The mixture was diluted with EtOAc and washed with brine. The organic layer was dried over MgSO₄, filtered, and concentrated to dryness. The crude product was purified by flash column chromatography (SiO₂, MeOH in DCM 0/100 to 5/95) and triturated with diisopropyl ether to yield Intermediate 14 as a white solid. MS (ESI+) calculated for C₁H₁₅N₃O₅: 269.1, found: 270 [M+H]⁺.

### Intermediate 15: Ethyl 5,7-dioxo-4,5,6,7-tetrahydropyrazolo[1,5-a]pyrimidine-2-carboxylate

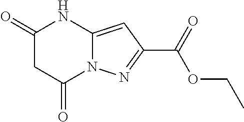 [SMILES: CCOC(=O)c1cc2n(n1)C(=O)CC(=O)N2]

4-Dimethylaminopyridine (12 g, 98.3 mmol) was added to a mixture of Intermediate 14 (9 g, 32.7 mmol) in ethanol (170 mL) and water (170 mL) at rt. The mixture was stirred at rt for 60 h. The reaction mixture was concentrated in vacuo. The crude was triturated with EtOAc/EtOH and the solid was dried in vacuo to yield Intermediate 15 as white solid. MS (ESI+) calculated for C₉H₉N₃O₄: 223.1, found: 224 [M+H]⁺.

### Intermediate 16: Ethyl 5,7-dichloropyrazolo[1,5-a]pyrimidine-2-carboxylate

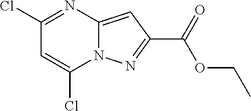 [SMILES: CCOC(=O)c1cc2nc(Cl)cc(Cl)n2n1]

A mixture of Intermediate 15 (5.55 g, 11.688 mmol) and phosphoryl chloride (8.3 mL, 1.64 g/mL, 89.314 mmol) was stirred at 100° C. for 1 h. Then, the reaction was quenched with water and ice at 0° C. The mixture was extracted with EtOAc (×3). The combined organic layers were basified with Na₂CO₃ (pH 7-8), dried with MgSO₄, and concentrated in vacuo. The combined organic layers were basified with Na₂CO₃ to pH 7-8, dried with MgSO₄, filtered and concentrated in vacuo. The crude was purified by flash column chromatography (SiO₂, EtOAc in heptane from 0/100 to 50/50). The desired fractions were collected and concentrated in vacuo to yield Intermediate 16 as white solid. MS (ESI+) calculated for C₉H₇Cl₂N₃O₂: 259.0, found: 260.0 [M+H]⁺.

### Intermediate 17: Ethyl 5-chloro-7-phenylpyrazolo[1,5-a]pyrimidine-2-carboxylate

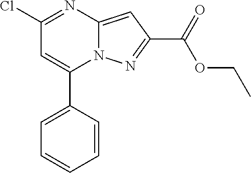 [SMILES: CCOC(=O)c1cc2nc(Cl)cc(-c3ccccc3)n2n1]

Ethyl 5,7-dichloropyrazolo[1,5-a]pyrimidine-2-carboxylate (Intermediate 16) (2.56 g, 9.84 mmol) and phenyl boronic acid (1 g, 8.2 mmol) and Na₂CO₃ (1.74 g, 16.4 mmol) were dissolved in dioxane (65 mL) and H₂O (10 mL) was added. The reaction was degassed for 10 min before Pd(dppf)Cl₂·CH₂Cl₂ (671 mg, 0.82 mmol) was added. The mixture was stirred at 50° C. for 8 h. EtOAc was added, and the layers were separated. The aqueous phase was extracted with EtOAc. The combined organic layers were dried over MgSO₄ and concentrated in vacuo. The residue was purified by flash chromatography (SiO₂, EtOAc in heptane 0/100 to 75/25). The desired fractions were collected and concentrated to yield Intermediate 17 as a yellowish solid. MS (ESI+) calculated for C₁₅H₁₂ClN₃O₂: 301.1, found: 302.1 [M+H]⁺.

### Intermediate 18. 7-Phenoxy-5-phenylpyrazolo[1,5-a]pyrimidine-2-carboxylic acid

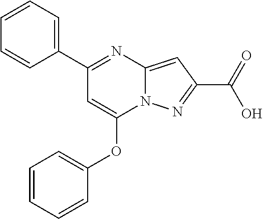 [SMILES: O=C(O)c1cc2nc(-c3ccccc3)cc(Oc3ccccc3)n2n1]

Ethyl 7-chloro-5-phenylpyrazolo[1,5-a]pyrimidine-2-carboxylate (Intermediate 3) (50 mg, 0.146 mmol), phenol (32.0 mg, 0.34 mmol) and Cs₂CO₃ (53 mg, 0.163 mmol) were suspended in THF (1 mL). The reaction was stirred at 55° C. for 18 h. Then, LiOH (0.35 mL, 0.5 M in water, 0.175 mmol) was added and stirred at 50° C. for 1 h. Solvents were removed in vacuo and water (3 mL) and DCM (3 mL) were added. The aqueous layer was separated, concentrated in vacuo, and redissolved in a mixture of DMSO and water (4 mL, 1:1 ratio). The resulting solution was submitted to RP HPLC (Stationary phase: C18 XBridge, column with 100 mm length, 5 μm. Mobile phase: Gradient using NH₄HCO₃ 0.25% solution in water and CH₃CN). The desired fractions were concentrated in vacuo to afford Intermediate 18 as a solid. MS (ESI+) calculated for C₁₉H₁₃N₃O₃: 331.1; found: 332.1 [M+H]⁺.

### Intermediate 19. Ethyl 5-(2-fluorophenyl)-7-phenylpyrazolo[1,5-a]pyrimidine-2-carboxylate

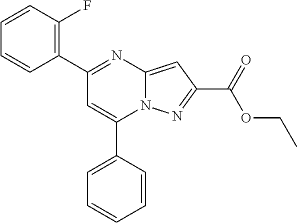 [SMILES: CCOC(=O)c1cc2nc(-c3ccccc3F)cc(-c3ccccc3)n2n1]

Ethyl 5-chloro-7-phenylpyrazolo[1,5-a]pyrimidine-2-carboxylate (Intermediate 17) (50 mg, 0.128 mmol), 2-fluorophenylboronic acid (27 mg, 0.193 mmol) and Pd(dppf)Cl₂·CH₂Cl₂ 10.5 mg, 0.0128 mmol) were suspended in aq Na₂CO₃ (270 μL, 0.1 g/mL in water, 0.255 mmol) and 1,4-dioxane (730 μL). The reaction was heated to 55° C. for 18 h. Water (2 mL) was added, and the product was extracted with EtOAc (3×2 mL). The combined organic layers were concentrated and purified by RP HPLC (Stationary phase: C18 XBridge 30×100 mm 5 μm, Mobile phase: NH₄HCO₃ 0.25% solution in water and CH₃CN). The desired fractions were concentrated in vacuo to afford Intermediate 19 as a solid. MS (ESI+) calculated for C₂₁H₁₆FN₃O₂: 361.1; found: 362.2 [M+H]⁺.

### Intermediate 20. Ethyl 5-(3-fluorophenyl)-7-phenylpyrazolo[1,5-a]pyrimidine-2-carboxylate

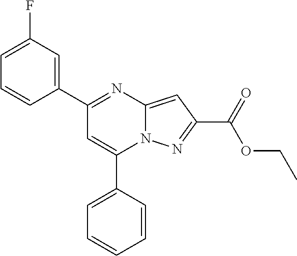 [SMILES: CCOC(=O)c1cc2nc(-c3cccc(F)c3)cc(-c3ccccc3)n2n1]

Intermediate 20 was synthetized following the method described for Intermediate 19 using ethyl 5-chloro-7-phenylpyrazolo[1,5-a]pyrimidine-2-carboxylate (Intermediate 17) (50 mg, 0.128 mmol) and 3-fluorophenylboronic acid (27 mg, 0.193 mmol) yielding Intermediate 20 as a solid. MS (ESI+) calculated for C₂₁H₁₆FN₃O₂: 361.1; found: 362.1 [M+H]⁺.

### Intermediate 21. Ethyl 5-(4-fluorophenyl)-7-phenylpyrazolo[1,5-a]pyrimidine-2-carboxylate

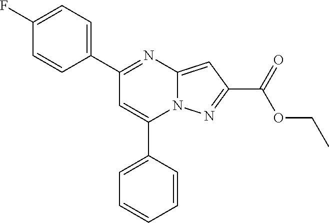 [SMILES: CCOC(=O)c1cc2nc(-c3ccc(F)cc3)cc(-c3ccccc3)n2n1]

Intermediate 21 was synthetized following the method described for Intermediate 19 using ethyl 5-chloro-7-phenylpyrazolo[1,5-a]pyrimidine-2-carboxylate (Intermediate 17) (50 mg, 0.128 mmol) and 4-fluorophenylboronic acid (27 mg, 0.193 mmol) yielding Intermediate 21 as a solid. MS (ESI+) calculated for C₂₁H₁₆FN₃O₂: 361.1; found: 362.1 [M+H]⁺.

### Intermediate 22. Ethyl 5-phenyl-7-(piperidin-1-yl)pyrazolo[1,5-a]pyrimidine-2-carboxylate

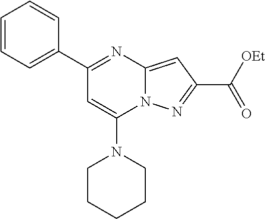 [SMILES: CCOC(=O)c1cc2nc(-c3ccccc3)cc(N3CCCCC3)n2n1]

To a suspension of ethyl 7-chloro-5-phenylpyrazolo[1,5-a]pyrimidine-2-carboxylate (Intermediate 3) (1.00 g, 1.55 mmol) in N,N-dimethylformamide (100 mL) was added potassium carbonate (810 mg, 5.86 mmol) and piperidine (0.35 mL, 3.82 mmol) at rt. After stirring at 60° C. for 16 h, the reaction mixture was diluted with water (200 mL) and extracted with EtOAc (3×200 mL). The combined organic layer was washed with brine (200 mL), dried over anhydrous Na₂SO₄, filtered and concentrated. The product was purified by column chromatography (SiO₂, petroleum ether:EtOAc 10:1) to give Intermediate 22 as a light yellow solid. MS (ESI+) calculated for C₂₀H₂₂N₄O₂: 350.2, found 351.1 [M+H]⁺.

### Intermediate 23. 5-Phenyl-7-(piperidin-1-yl)pyrazolo[1,5-a]pyrimidine-2-carboxylic acid

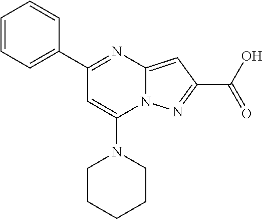 [SMILES: O=C(O)c1cc2nc(-c3ccccc3)cc(N3CCCCC3)n2n1]

To a solution of ethyl 5-phenyl-7-(piperidin-1-yl)pyrazolo[1,5-a]pyrimidine-2-carboxylate (Intermediate 22) (1.00 g, 2.71 mmol) in THF (20 mL) and water (20 mL) was added lithium hydroxide monohydrate (260 mg, 6.2 mmol) at rt and stirred for 12 h. The reaction mixture was adjusted pH to 5-6 with aq 1 M HCl sol. The precipitated solid was collected by filtration, washed with water and dried to give Intermediate 23 as a light yellow solid. MS (ESI+) calculated for C₁₈H₁₈N₄O₂ 322.1, m/z found 323.0 [M+H]⁺.

### Intermediate 24. 5-Phenyl-7-(pyridin-2-yl)pyrazolo[1,5-a]pyrimidine-2-carboxylic acid

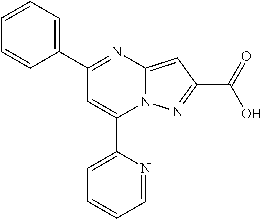 [SMILES: O=C(O)c1cc2nc(-c3ccccc3)cc(-c3ccccn3)n2n1]

Tetrakis(triphenylphosphine)palladium(0) (24 mg, 0.021 mmol) was added to a degassed solution of 7-chloro-5-phenylpyrazolo[1,5-a]pyrimidine-2-carboxylate (Intermediate 3) (100 mg, 0.33 mmol) and 2-(tributylstannyl)pyridine (136 mg, 0.37 mmol) in toluene (3 mL). The mixture was stirred in a microwave for 20 min at 150° C. The reaction mixture was filtered over a pad of Celite and rinsed with DCM. The organic solvent was removed in vacuo and the resulting crude was dissolved in THF (3 mL) and LiOH in H₂O (1 mL, 2 M, 2 mmol) was added. The mixture was stirred for 3 h at rt. EtOAc and H₂O were added to the reaction mixture and the layers were separated. The aqueous phase was acidified with aq 2N HCl and extracted with EtOAc. The aqueous phase was neutralized with aq sat NaHCO₃ and extracted with EtOAc (2×) and extracted again with a mixture of CHCl₃:iPrOH (7:1). The combined organic layers were dried over MgSO₄ and concentrated in vacuo to yield Intermediate 24 as a solid. MS (ESI+) calculated for C₁₈H₁₂N₄O₂: 316.1, found: 317.4 [M+H]⁺.

### Intermediate 25. Ethyl 5-phenyl-7-(pyrimidin-2-yl)pyrazolo[1,5-a]pyrimidine-2-carboxylate

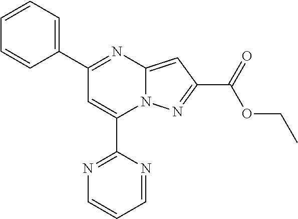 [SMILES: CCOC(=O)c1cc2nc(-c3ccccc3)cc(-c3ncccn3)n2n1]

Tetrakis(triphenylphosphine)palladium(0) (60 mg, 0.021 mmol) was added to a degassed solution of 7-chloro-5-phenylpyrazolo[1,5-a]pyrimidine-2-carboxylate (Intermediate 3) (300 mg, 0.99 mmol) and 2-(tributylstannyl)pyrimidine (0.27 mL, 1.5 g/mL, 1.1 mmol) in toluene (14 mL). The mixture was stirred in a microwave for 20 min at 150° C. Then, 2-(tributylstannyl)pyrimidine (0.32 mL, 1.5 g/mL, 1.3 mmol) and tetrakis(triphenylphosphine)palladium(0) (60 mg, 0.052 mmol) were added and the reaction mixture was stirred at 110° C. for 2 days. The mixture was concentrated, and the crude purified by flash chromatography (SiO₂, EtOAc in heptane 20:80 to 100:0) to yield Intermediate 25 as a white solid. MS (ESI+) calculated for C₁₉H₁₅N₅O₂: 345.1, found: 346.2 [M+H]⁺.

### Intermediate 26. 5-Phenyl-7-(pyrimidin-2-yl)pyrazolo[1,5-a]pyrimidine-2-carboxylic acid

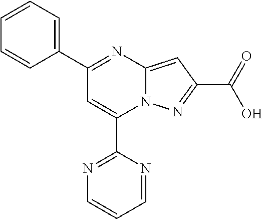 [SMILES: O=C(O)c1cc2nc(-c3ccccc3)cc(-c3ncccn3)n2n1]

5-Phenyl-7-(pyrimidin-2-yl)pyrazolo[1,5-a]pyrimidine-2-carboxylic acid (Intermediate 25) (100 mg, 0.29 mmol) was dissolved in THF (5 mL). A aqueous solution of LiOH (2 mL, 0.89 g/mL, 4 mmol) was added and the mixture was stirred at rt for 30 h. EtOAc and H₂O was added and the layers were separated. The aqueous phase was neutralized with aq 0.1 N HCl solution and extracted with EtOAc. The organic phase was concentrated and dried in vacuo at 50° C. to yield Intermediate 26 as a white solid. MS (ESI+) calculated for C₁₇H₁₁N₅O₂: 317.1, found: 318.0 [M+H]⁺.

### Intermediate 27. 7-(2-Fluorophenyl)-5-phenylpyrazolo[1,5-a]pyrimidine-2-carboxylic acid

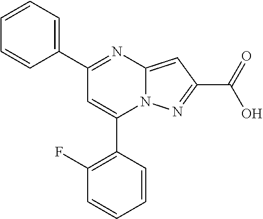 [SMILES: O=C(O)c1cc2nc(-c3ccccc3)cc(-c3ccccc3F)n2n1]

In a microwave vial, 7-chloro-5-phenylpyrazolo[1,5-a]pyrimidine-2-carboxylate (Intermediate 3) (100 mg, 0.33 mmol) and 2-fluorophenylboronic acid (83 mg, 0.6 mmol) were dissolved in dioxane (3 mL). Aq NaHCO₃ solution (0.7 mL, 1.65 M, 1.16 mmol) was added and the mixture was degassed for 2 min. Pd(PPh₃)₄ (19 mg, 0.017 mmol) was added and the mixture was stirred under microwave radiation for 20 min at 150° C. LiOH (1 mL, 2 M in water, 2 mmol) was added and the reaction mixture was stirred at rt for 20 h. EtOAc and aq 1N NaOH were added and the layers were separated. The aqueous phase was acidified with aq 2N HCl solution and extracted with EtOAc (2×). The combined organic layers were dried over MgSO₄ and concentrated in vacuo to yield Intermediate 27. MS (ESI+) calculated for C₁₉H₁₂FN₃O₂: 333.1, found: 334.1 [M+H]⁺.

### Intermediate 28. 5-Phenyl-7-(pyridin-3-yl)pyrazolo[1,5-a]pyrimidine-2-carboxylic acid

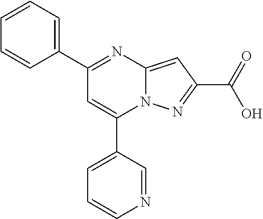 [SMILES: O=C(O)c1cc2nc(-c3ccccc3)cc(-c3cccnc3)n2n1]

Intermediate 28 was synthetized following the method described for Intermediate 27 using 7-chloro-5-phenylpyrazolo[1,5-a]pyrimidine-2-carboxylate (Intermediate 3) (100 mg, 0.33 mmol) and pyridine-3-boronic acid (81 mg, 0.66 mmol) yielding Intermediate 28 as a solid. MS (ESI+) calculated for C₁₈H₁₂N₄O₂: 316.1, found: 317.1 [M+H]⁺.

### Intermediate 29. 5-Phenyl-7-(pyridin-4-yl)pyrazolo[1,5-a]pyrimidine-2-carboxylic acid

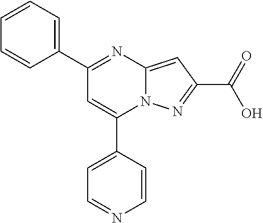 [SMILES: O=C(O)c1cc2nc(-c3ccccc3)cc(-c3ccncc3)n2n1]

Intermediate 29 was synthetized following the method described for Intermediate 27 using 7-chloro-5-phenylpyrazolo[1,5-a]pyrimidine-2-carboxylate (Intermediate 3) (100 mg, 0.33 mmol) and pyridine-4-boronic acid (81 mg, 0.66 mmol) yielding Intermediate 29 as a solid. MS (ESI−) calculated for C₁₈H₁₂N₄O₂: 316.1, found: 315.1 [M−H]⁺.

### Intermediate 30. 5,7-Diphenylpyrazolo[1,5-a]pyrimidine-2-carbonyl azide

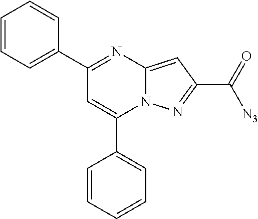 [SMILES: [N-]=[N+]=NC(=O)c1cc2nc(-c3ccccc3)cc(-c3ccccc3)n2n1]

To a solution of 5,7-diphenylpyrazolo[1,5,a]pyrimidine-2-carboxylic acid (Intermediate 2) (2 g, 6.3 mmol) in DCM (30 mL) was added diphenyl phosphoryl azide (2.05 mL, 1.28 g/mL, 9.5 mmol) and DIPEA (2.2 mL, 0.75 g/mL, 12.7 mmol). The reaction was stirred at rt for 16 h. The mixture was evaporated in vacuo and purified by flash column chromatography (SiO₂, MeOH in DCM 0/100 to 5/95) to afford an oil, which was treated with Et₂O to yield Intermediate 30 as a solid. MS (ESI+) calculated for C₁₉H₂₁N₆O: 340.1, found: 241.1 [M+H]⁺.

### Intermediate 31. 7-Phenyl-5-(pyrrolidin-1-yl)pyrazolo[1,5-a]pyrimidine-2-carboxylic acid

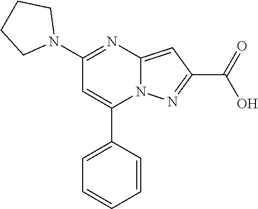 [SMILES: O=C(O)c1cc2nc(N3CCCC3)cc(-c3ccccc3)n2n1]

To a mixture of ethyl 5-chloro-7-phenylpyrazolo[1,5-a]pyrimidine-2-carboxylate (Intermediate 17) (200 mg, 0.57 mmol) and TEA (0.32 mL, 0.73 g/mL, 2.28 mmol) in iPrOH (5 mL), pyrrolidine (0.15 mL, 1.97 mmol) was added. The mixture was stirred at 50° C. for 4 h. The mixture was concentrated in vacuo and diluted with THF (4 mL). LiOH (3 mL, 2 M in water, 6 mmol) was added and the rm was stirred at rt overnight. H₂O and DCM were added, and layers separated. The aqueous phase was acidified with aq 1N HCl and extracted with DCM. The aqueous phase was concentrated in vacuo and dried in vacuo at 50° C. to yield Intermediate 31 as a solid. MS (ESI+) calculated for C₁₇H₁₆N₄O₂: 308.1, found: 309.1[M+H]⁺.

### Intermediate 32. 7-Phenyl-5-(piperidin-1-yl)pyrazolo[1,5-a]pyrimidine-2-carboxylic acid

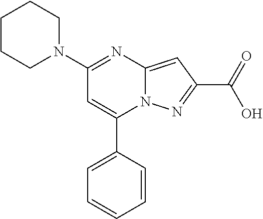 [SMILES: O=C(O)c1cc2nc(N3CCCCC3)cc(-c3ccccc3)n2n1]

Intermediate 32 was synthetized following the method described for Intermediate 31 using ethyl 5-chloro-7-phenylpyrazolo[1,5-a]pyrimidine-2-carboxylate (Intermediate 17) (200 mg, 0.57 mmol) and piperidine (0.16 mL mg, 1.58 mmol) yielding Intermediate 32. MS (ESI+) calculated for C₁₈H₁₈N₄O₂: 322.1, found: 323.1 [M+H]⁺.

### Intermediate 33. 5-(azetidin-1-yl)-7-phenylpyrazolo[1,5-a]pyrimidine-2-carboxylic acid

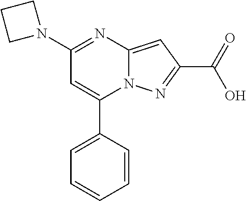 [SMILES: O=C(O)c1cc2nc(N3CCC3)cc(-c3ccccc3)n2n1]

Intermediate 33 was synthetized following the method described for Intermediate 31 using ethyl 5-chloro-7-phenylpyrazolo[1,5-a]pyrimidine-2-carboxylate (Intermediate 17) (200 mg, 0.57 mmol) and azetidine hydrochoride (148 mg, 1.58 mmol) to yield Intermediate 33 as a solid. MS (ESI+) calculated for C₁₆H₁₄N₄O₂: 294.1, found: 295.1 [M+H]⁺.

### Intermediate 34. 7-Phenyl-5-(4-(trifluoromethyl)phenyl)pyrazolo[1,5-a]pyrimidine-2-carboxylic acid

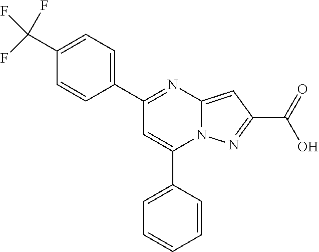 [SMILES: O=C(O)c1cc2nc(-c3ccc(C(F)(F)F)cc3)cc(-c3ccccc3)n2n1]

Pd(dppf)Cl₂·CH₂Cl₂ was added to a mixture of ethyl 5-chloro-7-phenylpyrazolo[1,5-a]pyrimidine-2-carboxylate (Intermediate 17) (110 mg, 9.29 mmol), 4-(trifluoromethyl)phenylboronic acid (82 mg, 0.43 mmol), K₃PO₄ (0.18 g, 0.86 mmol) in dioxane (8 mL) and water (3 mL). The mixture was stirred at 110° C. in a sealed tube for 16 h. The mixture was diluted with water and extracted with DCM. The aqueous phase was acidified with aq 1M HCl until pH 6 and extracted with DCM. The combined organic layers dried over MgSO₄, filtered, and concentrated in vacuo to yield Intermediate 34. MS (ESI+) calculated for C₂₀H₁₂F₃N₃O₂: 383.1, found: 384.1 [M+H]⁺.

### Intermediate 35. Ethyl 7-phenyl-5-(pyridin-2-yl)pyrazolo[1,5-a]pyrimidine-2-carboxylate

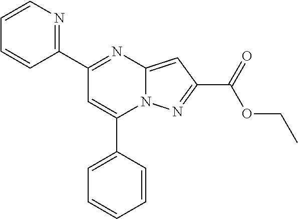 [SMILES: CCOC(=O)c1cc2nc(-c3ccccn3)cc(-c3ccccc3)n2n1]

Tetrakis(triphenylphosphine)palladium (0) (60 mg, 0.05 mmol) was added to a degassed solution of ethyl 5-chloro-7-phenylpyrazolo[1,5-a]pyrimidine-2-carboxylate (Intermediate 17) (200 mg, 0.57 mmol) and 2-(tributylstannyl)pyridine (0.36 mL, 1.14 mmol) in toluene (5 mL). The reaction mixture was stirred in a microwave for 20 min at 150° C. The reaction mixture was filtered over a pad of Celite and rinsed with DCM. The organic solvent was removed in vacuo and the residue was purified by flash chromatography (SiO₂, EtOAc in heptane 0/100 to 50/50). The desired fractions were collected and concentrated in vacuo to yield Intermediate 35 as a pale solid. MS (ESI+) calculated for C₂₀H₁₆N₄O₂: 344.2, found: 345.2 [M+H]⁺.

### Intermediate 36. 7-Phenyl-5-(pyridin-2-yl)pyrazolo[1,5-a]pyrimidine-2-carboxylic acid

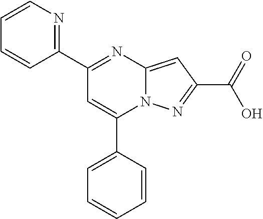 [SMILES: O=C(O)c1cc2nc(-c3ccccn3)cc(-c3ccccc3)n2n1]

LiOH (3 mL, 2 M in water, 6 mmol) was added to a suspension of ethyl 7-phenyl-5-(pyridin-2-yl)pyrazolo[1,5-a]pyrimidine-2-carboxylate (Intermediate 35) (200 mg, 0.58 mmol) in THF (5 mL). The reaction was stirred at rt for 18 h. EtOAc and H₂O were added. The layers were separated and the aqueous phase was acidified with aq 1N HCl solution and extracted with EtOAc. The organic layers were concentrated to yield Intermediate 36 as a pale solid. MS (ESI+) calculated for C₁₈H₁₂N₄O₂: 316.1, found: 317.0 [M+H]⁺.

### Intermediate 37. Ethyl 7-cyclohexyl-5-phenylpyrazolo[1,5-a]pyrimidine-2-carboxylate

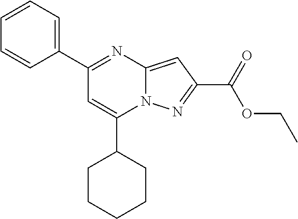 [SMILES: CCOC(=O)c1cc2nc(-c3ccccc3)cc(C3CCCCC3)n2n1]

Cyclohexylzine bromide (2.66 mL, 0.5 M in THF, 1.33 mmol) was added to a mixture of ethyl 7-chloro-5-phenylpyrazolo[1,5-a]pyrimidine-2-carboxylate (Intermediate 3) (80 mg, 0.27 mmol), Pd(OAc)₂ (3 mg, 0.013 mmol) and RuPhos (12.5 mg, 0.027 mmol) under N₂. The reaction was stirred at rt for 40 min. Then, sat aq NH₄Cl (1.5 mL) and EtOAc (1.5 mL) were added. Phases were separated and the aqueous phase was extracted with EtOAc (2×2 mL). The combined organic layers were dried over Na₂SO₄, filtered, and concentrated in vacuo. The residue was purified by flash column chromatography (SiO₂, EtOAc in DCM 0/100 to 50/50) The desired fractions were concentrated in vacuo to afford Intermediate 37 as a colorless oil. MS (ESI+) calculated for C₂₁H₂₃N₃O₂: 349.18; found: 350.2 [M+H]⁺.

### Intermediate 38. Ethyl 7-cyclopentyl-5-phenylpyrazolo[1,5-a]pyrimidine-2-carboxylate

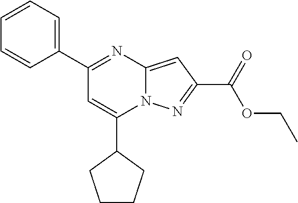 [SMILES: CCOC(=O)c1cc2nc(-c3ccccc3)cc(C3CCCC3)n2n1]

Intermediate 38 was synthetized following the method described for Intermediate 36 using ethyl 7-chloro-5-phenylpyrazolo[1,5-a]pyrimidine-2-carboxylate (Intermediate 3) (80 mg, 0.27 mmol) and cyclopentylzinc bromide (2.66 mL, 0.5 M in THF, 1.33 mmol) yielding Intermediate 38 as an oil. MS (ESI+) calculated for C₂₀H₂₁N₃O₂: 335.41; found: 336.2 [M+H]⁺.

### Intermediate 39. Ethyl 7-cyclobutyl-5-phenylpyrazolo[1,5-a]pyrimidine-2-carboxylate

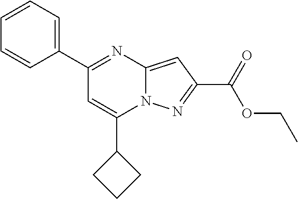 [SMILES: CCOC(=O)c1cc2nc(-c3ccccc3)cc(C3CCC3)n2n1]

Intermediate 39 was synthetized following the method described for Intermediate 36 using ethyl 7-chloro-5-phenylpyrazolo[1,5-a]pyrimidine-2-carboxylate (Intermediate 3) (60 mg, 0.20 mmol) and cyclobutylzinc bromide (0.8 mL, 0.5 M in THF, 0.4 mmol) yielding Intermediate 39 as a colorless oil. MS (ESI+) calculated for C₁₉H₁₉N₃O₂: 321.15; found: 322.1 [M+H]⁺.

### Intermediate 40. Ethyl 7-cyclopropyl-5-phenylpyrazolo[1,5-a]pyrimidine-2-carboxylate

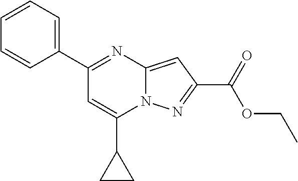 [SMILES: CCOC(=O)c1cc2nc(-c3ccccc3)cc(C3CC3)n2n1]

Intermediate 40 was synthetized following the method described for Intermediate 36 using ethyl 7-chloro-5-phenylpyrazolo[1,5-a]pyrimidine-2-carboxylate (Intermediate 3) (60 mg, 0.20 mmol) and cyclopropylzinc bromide (0.8 mL, 0.5 M in THF, 0.4 mmol) yielding Intermediate 40. MS (ESI+) calculated for C₁₈H₁₇N₃O₂: 307.0, found: 308.2 [M+H]⁺.

### Intermediate 41. Ethyl 7-isobutyl-5-phenylpyrazolo[1,5-a]pyrimidine-2-carboxylate

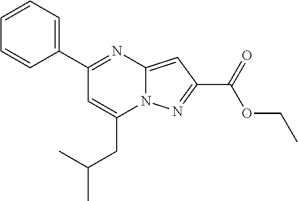 [SMILES: CCOC(=O)c1cc2nc(-c3ccccc3)cc(CC(C)C)n2n1]

Intermediate 41 was synthetized following the method described for Intermediate 36 using ethyl 7-chloro-5-phenylpyrazolo[1,5-a]pyrimidine-2-carboxylate (Intermediate 3) (60 mg, 0.20 mmol) and isobutylzinc bromide (0.8 mL, 0.5 M in THF, 0.4 mmol) yielding Intermediate 41 as a yellow oil. MS (ESI+) calculated for C₁₉H₂₁N₃O₂: 323.2; found: 324.23 [M+H]⁺.

### Intermediate 42. Ethyl-5-phenyl-7-(tetrahydrofuran-2-yl) pyrazolo[1,5-a]pyrimidine-2-carboxylate

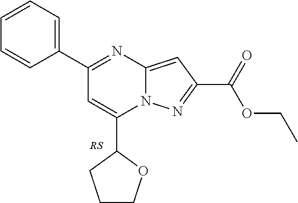 [SMILES: CCOC(=O)c1cc2nc(-c3ccccc3)cc(C3CCCO3)n2n1]

A solution of 2-tetrahydrofuroic acid (48 mg, 0.41 mmol), ethyl 7-chloro-5-phenylpyrazolo[1,5-a]pyrimidine-2-carboxylate (Intermediate 3) (80 mg, 0.27 mmol), nickel(II) chloride ethylene glycol dimethyl ether complex (12.2 mg, 0.056 mmol), 4,4′-di-tert-butyl-2,2′-dipyridyl (18.5 mg, 0.069 mmol), 4CzIPN (4.8 mg, 0.0061 mmol) and 2-tert-butyl-1,1,3,3-tetramethylguanidine (0.1 mL, 0.85 g/mL, 0.5 mmol) in DMA (2.6 mL) was degassed by bubbling N₂ for 5 min. The mixture was placed in a Penn reactor (450 nm, 6800 FAN, 100% LED) for 20 h. Sat aq NH₄Cl (1.5 mL) and EtOAc (2.5 mL) were added and the aqueous phase was extracted with EtOAc (2×2 mL). The combined organic layers were dried over Na₂SO₄, filtered, and concentrated in vacuo. The residue was purified by flash column chromatography (SiO₂, EtOAc in DCM 0/100 to 30/70) to yield Intermediate 42 as a light-yellow oil. MS (ESI+) calculated for C₁₉H₁₉N₃O₃: 337.0; found: 338.3 [M+H]⁺.

### Intermediate 43. Ethyl-5-phenyl-7-(tetrahydro-2H-pyran-2-yl) pyrazolo[1,5-a]pyrimidine-2-carboxylate

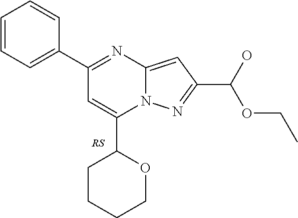 [SMILES: CCOC(O)c1cc2nc(-c3ccccc3)cc(C3CCCCO3)n2n1]

Intermediate 43 was synthetized following the method described for Intermediate 42 using ethyl 7-chloro-5-phenylpyrazolo[1,5-a]pyrimidine-2-carboxylate (Intermediate 3) (93.31 mg, 0.31 mmol) and tetrahydropyran-2-carboxylic acid (62 mg, 0.48 mmol) yielding Intermediate 43 as an oil. MS (ESI+) calculated for C₂₀H₂₁N₃O₃: 351.0; found: 352.2 [M+H]⁺.

### Intermediate 44. Ethyl-7-(1-(tert-butoxycarbonyl)piperidin-2-yl)-5-phenylpyrazolo[1,5-a]pyrimidine-2-carboxylate

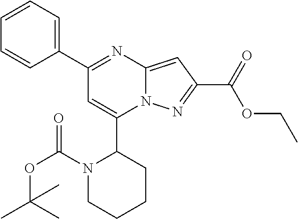 [SMILES: CCOC(=O)c1cc2nc(-c3ccccc3)cc(C3CCCCN3C(=O)OC(C)(C)C)n2n1]

Intermediate 44 was synthetized following the method described for Intermediate 42 using ethyl 7-chloro-5-phenylpyrazolo[1,5-a]pyrimidine-2-carboxylate (Intermediate 3) (200 mg, 0.66 mmol) and N-Boc-2-piperidinecarboxylic acid (234 mg, 1.02 mmol). MS (ESI+) calculated for C₂₅H₃₀N₄O₄: 450.2; found: 451.20 [M+H]⁺.

### Intermediate 45. 7-Cyclohexyl-5-phenylpyrazolo[1,5-a]pyrimidine-2-carboxylic acid

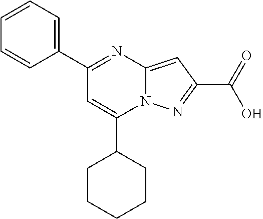 [SMILES: O=C(O)c1cc2nc(-c3ccccc3)cc(C3CCCCC3)n2n1]

Lithium hydroxide (21 mg, 0.88 mmol) in water (0.2 mL) was added to a stirred solution of ethyl 7-cyclohexyl-5-phenylpyrazolo[1,5-a]pyrimidine-2-carboxylate (Intermediate 37) (60 mg, 0.17 mmol) in 1,4-dioxane (0.3 mL). The mixture was stirred at rt for 4 h. Aq 2N HCl (1.5 mL) and EtOAc (2 mL) were added and phases were separated. The aqueous phase was extracted with EtOAc (2×1.5 mL). The combined organic layers were dried over Na₂SO₄, filtered, and concentrated in vacuo to yield Intermediate 45 as a clear wax. MS (ESI+) calculated for C₁₉H₁₉N₃O₂: 321.2; found: 322.2 [M+H]⁺.

### Intermediate 46. 7-Cyclopentyl-5-phenylpyrazolo[1,5-a]pyrimidine-2-carboxylic acid

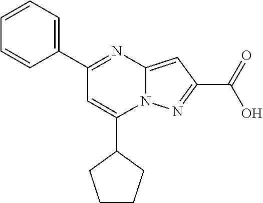 [SMILES: O=C(O)c1cc2nc(-c3ccccc3)cc(C3CCCC3)n2n1]

Intermediate 46 was synthetized following the method described for Intermediate 45 using ethyl 7-cyclopentyl-5-phenylpyrazolo[1,5-a]pyrimidine-2-carboxylate (Intermediate 38) yielding Intermediate 46 as a yellow oil. MS (ESI+) calculated for C₁₈H₁₇N₃O₂: 307.1; found: 308.1 [M+H]⁺.

### Intermediate 47. 7-Cyclobutyl-5-phenylpyrazolo[1,5-a]pyrimidine-2-carboxylic acid

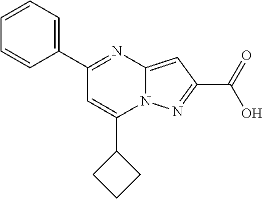 [SMILES: O=C(O)c1cc2nc(-c3ccccc3)cc(C3CCC3)n2n1]

Intermediate 47 was synthetized following the method described for Intermediate 45 using ethyl 7-cyclobutyl-5-phenylpyrazolo[1,5-a]pyrimidine-2-carboxylate (Intermediate 39) (60 mg, 0.20 mmol) yielding Intermediate 47 as a colourless oil. MS (ESI+) calculated for C₁₇H₁₅N₃O₂: 293.1, found: 294.0 [M+H]⁺.

### Intermediate 48. 5-Phenyl-7-(tetrahydrofuran-2-yl) pyrazolo[1,5-a]pyrimidine-2-carboxylic acid

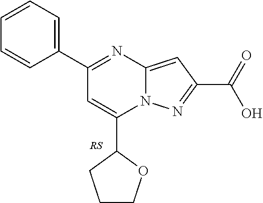 [SMILES: O=C(O)c1cc2nc(-c3ccccc3)cc(C3CCCO3)n2n1]

Intermediate 48 was synthetized following the method described for Intermediate 45 using ethyl-5-phenyl-7-(tetrahydrofuran-2-yl) pyrazolo[1,5-a]pyrimidine-2-carboxylate (Intermediate 42) (69 mg, 0.07 mmol) yielding Intermediate 48 as a yellow oil. MS (ESI+) calculated for C₁₇H₁₅N₃O₃: 309.1; found: 310.1 [M+H]⁺.

### Intermediate 49. 5-Phenyl-7-(tetrahydro-2H-pyran-2-yl) pyrazolo[1,5-a]pyrimidine-2-carboxylic acid

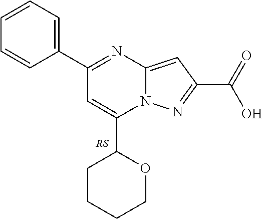 [SMILES: O=C(O)c1cc2nc(-c3ccccc3)cc(C3CCCCO3)n2n1]

Intermediate 49 was synthetized following the method described for Intermediate 45 using ethyl-5-phenyl-7-(tetrahydro-2H-pyran-2-yl) pyrazolo[1,5-a]pyrimidine-2-carboxylate (Intermediate 43) (96 mg, 0.19 mmol) yielding Intermediate 49 as a yellow oil. MS (ESI+) calculated for C₁₈H₁₇N₃O₃: 323.1; found: 324.1 [M+H]⁺.

### Intermediate 50. 7-(1-(tert-Butoxycarbonyl)piperidin-2-yl)-5-phenylpyrazolo[1,5-a]pyrimidine-2-carboxylic acid

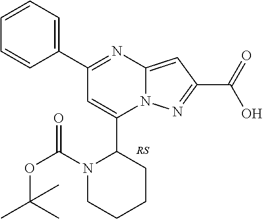 [SMILES: CC(C)(C)OC(=O)N1CCCCC1c1cc(-c2ccccc2)nc2cc(C(=O)O)nn12]

Intermediate 50 was synthetized following the method described for Intermediate 45 using ethyl-7-(1-(tert-butoxycarbonyl)piperidin-2-yl)-5-phenylpyrazolo[1,5-a]pyrimidine-2-carboxylate (Intermediate 44) yielding Intermediate 50 as a yellow solid. MS (ESI+) calculated for C₂₃H₂₆N₄O₄: 422.2; found: 423.2 [M+H]⁺.

### Intermediate 51. tert-Butyl-2-(2-((3-(1H-imidazo-1-yl)propyl)carbamoyl)-5-phenylpyrazolo[1,5-a]pyrimidin-7-yl)piperidine-1-carboxylate

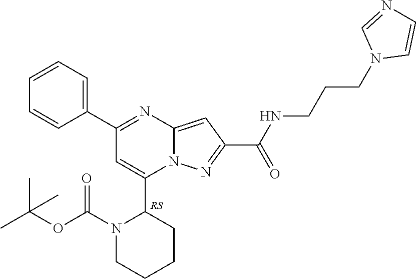 [SMILES: CC(C)(C)OC(=O)N1CCCCC1c1cc(-c2ccccc2)nc2cc(C(=O)NCCCn3ccnc3)nn12]

DIPEA (0.43 mL, 0.75 g/mL, 2.52 mmol) was added to a stirred suspension of 7-(1-(tert-butoxycarbonyl)piperidin-2-yl)-5-phenylpyrazolo[1,5-a]pyrimidine-2-carboxylic acid (Intermediate 50) (329.8 mg, 0.47 mmol), 1-(3-aminopropyl)imidazole (94 mg, 0.75 mmol) and HATU (357 mg, 0.94 mmol) in DMF (4.7 mL). The mixture was stirred at rt for 2 h. NaHCO₃ (4 mL) and EtOAc (4 mL) were added, and phases were separated. The aqueous phase was extracted with EtOAc (4 mL) and EtOAc/THF (7/3) (4 mL). The combined organic layers were dried over Na₂SO₄, filtered, and concentrated in vacuo. The residue was purified by flash column chromatography (SiO₂, EtOAc in DCM 0/100 to 100/0, followed by MeOH in DCM 0/100 to 10/90). The desired fractions were collected and concentrated in vacuo to yield Intermediate 51 as a yellow foam. MS (ESI+) calculated for C₂₉H₃₅N₇O₃: 529.3; found: 530.4 [M+H]⁺.

### Intermediate 52. tert-Butyl 3-(5,7-diphenylpyrazolo[1,5-a]pyrimidine-2-carboxamido)azetidine-1-carboxylate

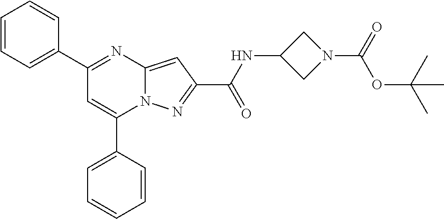 [SMILES: CC(C)(C)OC(=O)N1CC(NC(=O)c2cc3nc(-c4ccccc4)cc(-c4ccccc4)n3n2)C1]

To a solution of 5,7-diphenylpyrazolo[1,5-a]pyrimidine-2-carboxylic acid (135 mg, 0.41 mmol) in N,N-dimethylformamide (5 mL) was added tert-butyl 3-aminoazetidine-1-carboxylate (0.1 mL, 0.639 mmol), triethylamine (0.17 mL, 1.22 mmol) and O-(7-azabenzotriazol-1-yl)-N,N,N′,N′-tetramethyluronium hexafluorophosphate (230 mg, 0.605 mmol) at rt. After stirring at rt overnight, the reaction mixture was diluted with water (20 mL) and extracted with EtOAc (3×20 mL). The combined organic layer was dried over Na₂SO₄ and concentrated to give the Intermediate 52 as a yellow solid. MS (ESI+) calculated for: C₂₇H₂₇N₅O₃ 469.2, found 470.1 [M+H]⁺.

### Intermediate 53. tert-Butyl (3-(5,7-diphenylpyrazolo[1,5-a]pyrimidine-2-carboxamido)propyl)carbamate

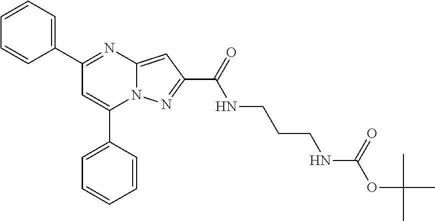 [SMILES: CC(C)(C)OC(=O)NCCCNC(=O)c1cc2nc(-c3ccccc3)cc(-c3ccccc3)n2n1]

HATU (54 mg, 0.14 mmol) and DIPEA (49 μL, 0.75 g/mL, 0.29 mmol) were added to a suspension of 5,7-diphenylpyrazolo[1,5,a]pyrimidine-2-carboxylic acid (Intermediate 2) (30 mg, 0.095 mmol) in DCM (1 mL). After 5 min, tert-butyl (3-aminopropyl)carbamate (25 mg, 0.14 mmol) was added, and the reaction mixture was stirred at rt for 18 h. Then, the mixture was treated with sat. aq Na₂CO₃ and stirred at rt for 5 min. Then, the aqueous phase was extracted with EtOAc and the organic layer was separated, dried over MgSO₄, filtered and concentrated evaporated in vacuo. The product was purified by RP HPLC (Stationary phase: C18 XBridge, column with 100 mm length, 5 μm. Mobile phase: Gradient using NH₄HCO₃ 0.25% solution in water and CH₃CN as organic solvent), yielding Intermediate 53 as solid. MS (ESI+) calculated for C₂₇H₂₉N₅O₃ 471.2, found: 472.2 [M+H]⁺.

### Intermediate 54. tert-Butyl 2-((5,7-diphenylpyrazolo[1,5-a]pyrimidine-2-carboxamido)methyl)pyrrolidine-1-carboxylate

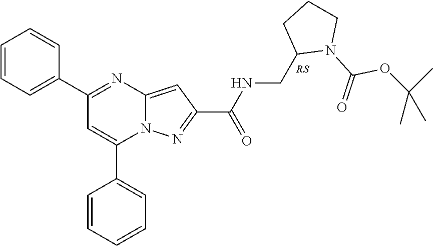 [SMILES: CC(C)(C)OC(=O)N1CCCC1CNC(=O)c1cc2nc(-c3ccccc3)cc(-c3ccccc3)n2n1]

Intermediate 23 was synthetized following the method described for Intermediate 53 using 5,7-diphenylpyrazolo[1,5,a]pyrimidine-2-carboxylic acid (Intermediate 2) (30 mg, 0.095 mmol) and tert-butyl 2-(aminomethyl)pyrrolidine-1-carboxylate (1.5 eq., 0.143 mmol), yielding Intermediate 54.

### Intermediate 55. tert-Butyl (3-(5,7-diphenylpyrazolo[1,5-a]pyrimidine-2-carboxamido)propyl)(methyl)carbamate

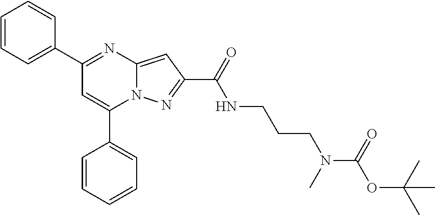 [SMILES: CN(CCCNC(=O)c1cc2nc(-c3ccccc3)cc(-c3ccccc3)n2n1)C(=O)OC(C)(C)C]

Intermediate 55 was synthetized following the method described for Intermediate 53 using 5,7-diphenylpyrazolo[1,5,a]pyrimidine-2-carboxylic acid (Intermediate 2) (30 mg, 0.095 mmol) and tert-butyl (3-aminopropyl)(methyl)carbamate (1.5 eq., 0.143 mmol), yielding Intermediate 55 as a solid. MS (ESI+) calculated for C₂₈H₃₁N₅O₃ 485.2 found 486.3 [M+H]⁺.

### Intermediate 56. tert-Butyl (2-(5,7-diphenylpyrazolo[1,5-a]pyrimidine-2-carboxamido)ethyl)carbamate

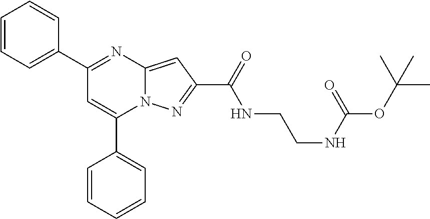 [SMILES: CC(C)(C)OC(=O)NCCNC(=O)c1cc2nc(-c3ccccc3)cc(-c3ccccc3)n2n1]

Intermediate 56 was synthetized following the method described for Intermediate 53 using 5,7-diphenylpyrazolo[1,5,a]pyrimidine-2-carboxylic acid (Intermediate 2) (30 mg, 0.095 mmol) and tert-butyl (2-aminoethyl)carbamate (1.5 eq., 0.143 mmol), yielding Intermediate 56 as a solid. MS (ESI+) calculated for C₂₆H₂₇N₅O₃ 457.2, found 458.3 [M+H]⁺.

### Intermediate 57. tert-Butyl (3-(5,7-diphenylpyrazolo[1,5-a]pyrimidine-2-carboxamido)-2-methylpropyl)carbamate

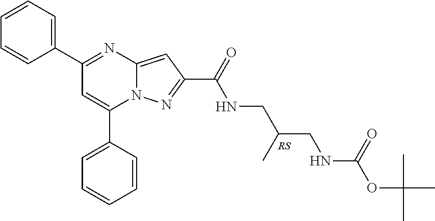 [SMILES: CC(CNC(=O)OC(C)(C)C)CNC(=O)c1cc2nc(-c3ccccc3)cc(-c3ccccc3)n2n1]

Intermediate 57 was synthetized following the method described for Intermediate 53 using 5,7-diphenylpyrazolo[1,5,a]pyrimidine-2-carboxylic acid (Intermediate 2) (30 mg, 0.095 mmol) and tert-butyl (3-amino-2-methylpropyl)carbamate (1.5 eq., 0.143 mmol), yielding Intermediate 57 as solid. MS (ESI+) calculated for C₂₈H₃₁N₅O₃ 485.2, found 486.3 [M+H]⁺.

### Intermediate 58. tert-Butyl 3-((5,7-diphenylpyrazolo[1,5-a]pyrimidine-2-carboxamido)methyl)pyrrolidine-1-carboxylate

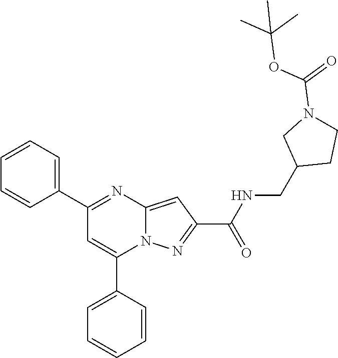 [SMILES: CC(C)(C)OC(=O)N1CCC(CNC(=O)c2cc3nc(-c4ccccc4)cc(-c4ccccc4)n3n2)C1]

Intermediate 57 was synthetized following the method described for Intermediate 53 using 5,7-diphenylpyrazolo[1,5,a]pyrimidine-2-carboxylic acid (Intermediate 2) (30 mg, 0.095 mmol) and tert-butyl (3-amino-2-methylpropyl)carbamate (1.5 eq., 0.143 mmol), yielding Intermediate 58 as solid. MS (ESI+) calculated for C₂₉H₃₁N₅O₃ 497.2, found 398.2 [M-Boc+H]⁺.

### Intermediate 59. tert-Butyl 3-(5,7-diphenylpyrazolo[1,5-a]pyrimidine-2-carboxamido)-2-methylazetidine-1-carboxylate

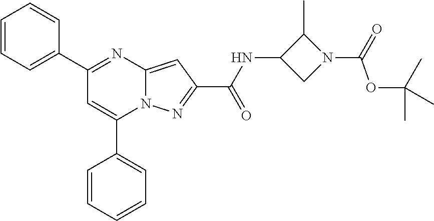 [SMILES: CC1C(NC(=O)c2cc3nc(-c4ccccc4)cc(-c4ccccc4)n3n2)CN1C(=O)OC(C)(C)C]

DIPEA (0.42 mL, 0.75 g/mL, 2.44 mmol) followed by tert-butyl 3-amino-2-methylazetidine-1-carboxylate (0.18 mL, 1.05 g/mL, 1.02 mmol) were added to a stirred mixture of 5,7-diphenylpyrazolo[1,5,a]pyrimidine-2-carboxylic acid (Intermediate 2) (263 mg, 0.83 mmol) and HATU (494 mg, 1.3 mmol) in DMF (9 mL). The mixture was stirred at rt for 16 h. The mixture was treated with 10% Na₂CO₃ aq and DCM and stirred for 10 min. The layers were separated, the organic phase was concentrated in vacuo and the crude product was purified by RP flash chromatography (C18, MeOH in NH₄HCO₃ 0.25% solution in water 50/50 to 100/0) yielding Intermediate 59 as a yellow solid. MS (ESI+) calculated for C₂₈H₂₉N₅O₃ 483.2, found: 484.1 [M+H]⁺.

### Intermediate 60. N-(2-Methylazetidin-3-yl)-5,7-diphenylpyrazolo[1,5-a]pyrimidine-2-carboxamide

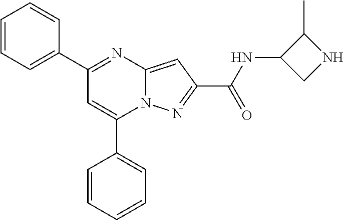 [SMILES: CC1NCC1NC(=O)c1cc2nc(-c3ccccc3)cc(-c3ccccc3)n2n1]

TFA (0.24 mL, 1.49 g/mL, 3.1 mmol) was added to a stirred solution of tert-butyl 3-(5,7-diphenylpyrazolo[1,5-a]pyrimidine-2-carboxamido)-2-methylazetidine-1-carboxylate (Intermediate 59) (300 mg, 0.62 mmol) in DCM (6.2 mL). The mixture was stirred at rt for 16 h. Then more TFA (0.48 mL, 1.49 g/mL, 6.27 mmol) was added and the mixture was stirred at rt for 24 h more. The mixture was basified with 10% Na₂CO₃ aq. and DCM and stirred for 15 min. The layers were separated and the organic layers were concentrated under vacuo. The crude product was purified by flash column chromatography (SiO₂, 7N solution of NH₃ in MeOH in DCM 0.5/99.5 to 10/90) yielding Intermediate 60 as an off white solid. 1H NMR (400 MHz, CHLOROFORM-d) δ ppm 1.39 (d, J=6.24 Hz, 1H) 1.23 (d, J=6.70 Hz, 2H) 3.37-3.57 (m, 1H) 3.71-5.04 (m, 3H) 7.32 (d, J=1.16 Hz, 1H) 7.44-7.46 (m, 1H) 7.51-7.59 (m, 4H) 7.61-7.67 (m, 3H) 8.01-8.10 (m, 2H) 8.12-8.19 (m, 2H). MS (ESI+) calculated for C₂₃H₂₁N₅O 383.2, found 384.1 [M+H]⁺.

### Example 1. N-(3-(1-Methyl-1H-imidazol-2-yl)propyl)-5,7-diphenylpyrazolo[1,5-a]pyrimidine-2-carboxamide

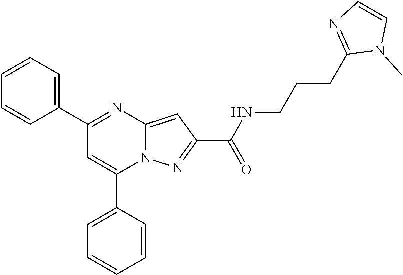 [SMILES: Cn1ccnc1CCCNC(=O)c1cc2nc(-c3ccccc3)cc(-c3ccccc3)n2n1]

HATU (54 mg, 0.14 mmol) and DIPEA (49 μL, 0.75 g/mL, 0.29 mmol) were added to a suspension of 5,7-diphenylpyrazolo[1,5,a]pyrimidine-2-carboxylic acid (Intermediate 2) (30 mg, 0.095 mmol) in DMF (1 mL). After 5 min, 3-(1-methyl-1H-imidazol-2-yl)propan-1-amine dihydrochloride (30 mg, 0.14 mmol) was added, and the reaction mixture was stirred at rt for 18 h. Then, the mixture was treated with sat aq Na₂CO₃ solution and stirred at rt for 5 min. Then, the product was extracted with EtOAc. The organic layer was separated, dried over MgSO₄, filtered and the solvents evaporated in vacuo. The product was purified by RP HPLC (Stationary phase: C18 XBridge, column with 100 mm length, 5 μm. Mobile phase: Gradient using NH₄HCO₃ 0.25% solution in water and CH₃CN), yielding Example 1 as a solid. ¹H NMR (500 MHz, DMSO-d₆) δ ppm 1.94 (quin, J=7.21 Hz, 2H) 2.66 (t, J=7.48 Hz, 2H) 3.38 (q, J=6.56 Hz, 2H) 3.54 (s, 3H) 6.68 (d, J=1.22 Hz, 1H) 6.98 (d, J=1.22 Hz, 1H) 7.15 (s, 1H) 7.54-7.61 (m, 3H) 7.62-7.71 (m, 3H) 7.92 (s, 1H) 8.27-8.39 (m, 4H) 8.59 (t, J=5.80 Hz, 1H). MS (ESI+) calculated for C₂₆H₂₄N₆O: 436.2, found: 437.3 [M+H]⁺.

### Example 2. N-(3-(2-Methyl-1H-imidazol-1-yl)propyl)-5,7-diphenylpyrazolo[1,5-a]pyrimidine-2-carboxamide

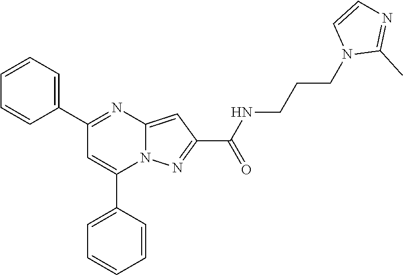 [SMILES: Cc1nccn1CCCNC(=O)c1cc2nc(-c3ccccc3)cc(-c3ccccc3)n2n1]

Example 2 was synthetized analogously to the method in Example 1 using 5,7-diphenylpyrazolo[1,5,a]pyrimidine-2-carboxylic acid (Intermediate 2) (30 mg, 0.095 mmol) and 3-(2-methyl-1H-imidazol-1-yl)propan-1-amine (1.5 eq., 0.143 mmol) yielding Example 2 as a solid. ¹H NMR (500 MHz, DMSO-d₆) δ ppm 1.95 (t, J=7.02 Hz, 2H) 2.27 (s, 3H) 3.33-3.34 (m, 2H) 3.92 (t, J=7.10 Hz, 2H) 6.71 (d, J=1.22 Hz, 1H) 7.10 (d, J=1.22 Hz, 1H) 7.16 (s, 1H) 7.56-7.60 (m, 3H) 7.63-7.69 (m, 3H) 7.93 (s, 1H) 8.30 (dd, J=7.63, 1.98 Hz, 2H) 8.33-8.36 (m, 2H) 8.47 (t, J=5.87 Hz, 1H). MS (ESI+) calculated for C₂₆H₂₄N₆O: 436.5, found: 437.3 [M+H]⁺.

### Example 3. N-(1-Benzylazetidin-3-yl)-5,7-diphenylpyrazolo[1,5-a]pyrimidine-2-carboxamide

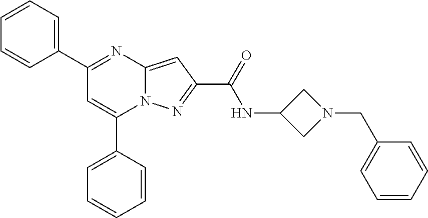 [SMILES: O=C(NC1CN(Cc2ccccc2)C1)c1cc2nc(-c3ccccc3)cc(-c3ccccc3)n2n1]

Example 3 was synthetized analogously to the method in Example 1 using 5,7-diphenylpyrazolo[1,5,a]pyrimidine-2-carboxylic acid (Intermediate 2) (30 mg, 0.095 mmol) and 1-benzylazetidin-3-amine (1.5 eq., 0.143 mmol), yielding Example 3 as a solid. ¹H NMR (500 MHz, DMSO-d₆) δ ppm 3.18 (br s, 2H) 3.58 (br t, J=6.87 Hz, 2H) 3.64 (s, 2H) 4.52 (q, J=7.02 Hz, 1H) 7.18 (s, 1H) 7.22-7.34 (m, 5H) 7.55-7.61 (m, 3H) 7.63-7.69 (m, 3H) 7.92 (s, 1H) 8.26-8.31 (m, 2H) 8.32-8.36 (m, 2H) 8.74 (d, J=6.87 Hz, 1H). MS (ESI+) calculated for C₂₉H₂₅N₅O: 459.2, found: 460.3 [M+H]⁺.

### Example 4. N-(1-Isopropylpyrrolidin-3-yl)-5,7-diphenylpyrazolo[1,5-a]pyrimidine-2-carboxamide

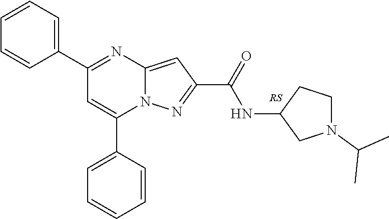 [SMILES: CC(C)N1CCC(NC(=O)c2cc3nc(-c4ccccc4)cc(-c4ccccc4)n3n2)C1]

Example 4 was synthetized analogously to the method in Example 1 using 5,7-diphenylpyrazolo[1,5,a]pyrimidine-2-carboxylic acid (Intermediate 2) (30 mg, 0.095 mmol) and 1-isopropylpyrrolidin-3-amine (1.5 eq., 0.143 mmol), yielding Example 4 as solid. ¹H NMR (500 MHz, DMSO-d₆) δ ppm 1.03 (dd, J=7.17, 6.41 Hz, 6H) 1.74-1.84 (m, 1H) 2.09-2.40 (m, 4H) 2.71 (td, J=8.58, 4.96 Hz, 1H) 2.78 (dd, J=9.23, 7.25 Hz, 1H) 4.35-4.45 (m, 1H) 7.19 (s, 1H) 7.56-7.60 (m, 3H) 7.64-7.68 (m, 3H) 7.92 (s, 1H) 8.25 (d, J=7.63 Hz, 1H) 8.29 (dd, J=7.71, 1.91 Hz, 2H) 8.33-8.36 (m, 2H). MS (ESI+) calculated for C₂₆H₂₇N₅O: 425.2, found: 426.3 [M+H]⁺.

### Example 5. N-(1-Ethylpyrrolidin-3-yl)-5,7-diphenylpyrazolo[1,5-a]pyrimidine-2-carboxamide

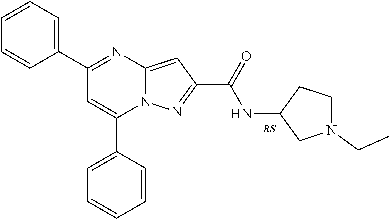 [SMILES: CCN1CCC(NC(=O)c2cc3nc(-c4ccccc4)cc(-c4ccccc4)n3n2)C1]

Example 5 was synthetized analogously to the method in Example 1 using 5,7-diphenylpyrazolo[1,5,a]pyrimidine-2-carboxylic acid (Intermediate 2) (30 mg, 0.095 mmol) and 1-ethylpyrrolidin-3-amine, di-hydrogen chloride (1.5 eq., 0.14 mmol), yielding Example 5 as a solid. ¹H NMR (500 MHz, DMSO-d₆) δ ppm 1.03 (t, J=7.25 Hz, 3H) 1.72-1.84 (m, 1H) 2.09-2.23 (m, 1H) 2.34-2.46 (m, 4H) 2.61-2.76 (m, 2H) 4.35-4.47 (m, 1H) 7.19 (s, 1H) 7.54-7.61 (m, 3H) 7.63-7.69 (m, 3H) 7.91 (s, 1H) 8.25-8.31 (m, 3H) 8.32-8.36 (m, 2H). MS (ESI+) calculated for C₂₅H₂₅N₅O: 411.2, found: 412.3 [M+H]⁺.

### Example 6. N-(1-Methylpyrrolidin-3-yl)-5,7-diphenylpyrazolo[1,5-a]pyrimidine-2-carboxamide

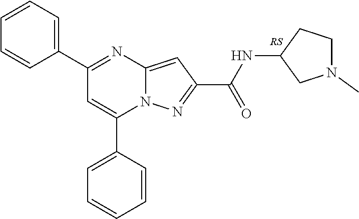 [SMILES: CN1CCC(NC(=O)c2cc3nc(-c4ccccc4)cc(-c4ccccc4)n3n2)C1]

Example 6 was synthetized analogously to the method in Example 1 using 5,7-diphenylpyrazolo[1,5,a]pyrimidine-2-carboxylic acid (Intermediate 2) (30 mg, 0.095 mmol) and 1-methylpyrrolidin-3-amine (1.5 eq., 0.143 mmol), yielding Example 6 as a solid. ¹H NMR (500 MHz, DMSO-d₆) δ ppm 1.73-1.84 (m, 1H) 2.13-2.22 (m, 1H) 2.25 (s, 3H) 2.35-2.39 (m, 1H) 2.60-2.69 (m, 3H) 4.43 (dddd, J=9.38, 7.10, 4.81, 2.37 Hz, 1H) 7.19 (s, 1H) 7.53-7.61 (m, 3H) 7.63-7.69 (m, 3H) 7.91 (s, 1H) 8.23-8.31 (m, 3H) 8.31-8.37 (m, 2H). MS (ESI+) calculated for C₂₄H₂₃N₅O: 397.2, found: 398.3 [M+H]⁺.

### Example 7. (S)—N-(1-Methylpyrrolidin-3-yl)-5,7-diphenylpyrazolo[1,5-a]pyrimidine-2-carboxamide

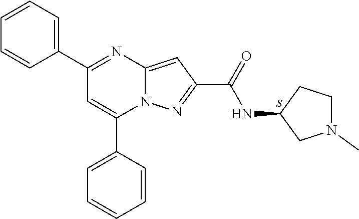

Example 7 was synthesized according to the procedure of Example 6, using (S)-3-amino-1-methyl pyrrolidine.

### Example 8. (R)—N-(1-Methylpyrrolidin-3-yl)-5,7-diphenylpyrazolo[1,5-a]pyrimidine-2-carboxamide

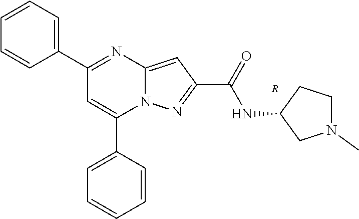

Example 8 was synthesized according to the procedure of Example 6, using (R)-3-amino-1-methyl pyrrolidine.

### Example 9. 5,7-Diphenyl-N-(3-(pyridin-4-yl)propyl)pyrazolo[1,5-a]pyrimidine-2-carboxamide

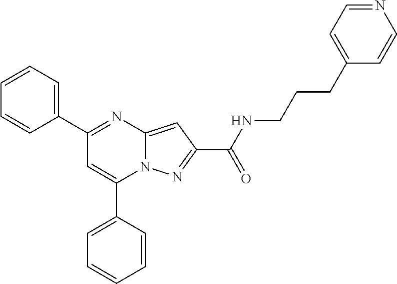 [SMILES: O=C(NCCCc1ccncc1)c1cc2nc(-c3ccccc3)cc(-c3ccccc3)n2n1]

Example 9 was synthetized analogously to the method in Example 1 using 5,7-diphenylpyrazolo[1,5,a]pyrimidine-2-carboxylic acid (Intermediate 2) (30 mg, 0.095 mmol) and 3-(pyridin-4-yl)propan-1-amine (1.5 eq., 0.143 mmol), yielding Example 9 as a solid. ¹H NMR (500 MHz, DMSO-d₆) δ ppm 1.88 (t, J=7.32 Hz, 2H) 2.63-2.68 (m, 2H) 3.34 (br s, 2H) 7.16 (s, 1H) 7.24-7.30 (m, 2H) 7.54-7.61 (m, 3H) 7.63-7.69 (m, 3H) 7.92 (s, 1H) 8.30 (dd, J=7.48, 2.14 Hz, 2H) 8.33-8.36 (m, 2H) 8.42-8.48 (m, 3H). MS (ESI+) calculated for C₂₇H₂₃N₅O: 433.2, found: 434.3 [M+H]⁺.

### Example 10. N-(2-(Dimethylamino)ethyl)-5,7-diphenylpyrazolo[1,5-a]pyrimidine-2-carboxamide

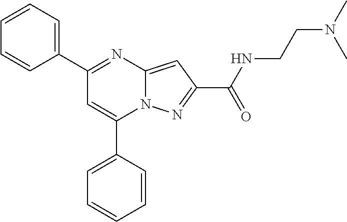 [SMILES: CN(C)CCNC(=O)c1cc2nc(-c3ccccc3)cc(-c3ccccc3)n2n1]

Example 10 was synthetized analogously to the method in Example 1 using 5,7-diphenylpyrazolo[1,5,a]pyrimidine-2-carboxylic acid (Intermediate 2) (30 mg, 0.095 mmol) and N1,N1-dimethylethane-1,2-diamine (1.5 eq., 0.143 mmol), yielding Example 10 as a solid. ¹H NMR (500 MHz, DMSO-d₆) δ ppm 2.18 (s, 6H) 2.42 (t, J=6.79 Hz, 2H) 3.39 (br d, J=6.10 Hz, 2H) 7.15 (s, 1H) 7.55-7.62 (m, 3H) 7.63-7.70 (m, 3H) 7.93 (s, 1H) 8.24 (t, J=5.80 Hz, 1H) 8.27-8.31 (m, 2H) 8.32-8.37 (m, 2H). MS (ESI+) calculated for C₂₃H₂₃N₅O: 385.2, found: 386.3 [M+H]⁺.

### Example 11. N-(3-(1H-Imidazol-1-yl)propyl)-N-methyl-5,7-diphenylpyrazolo[1,5-a]pyrimidine-2-carboxamide

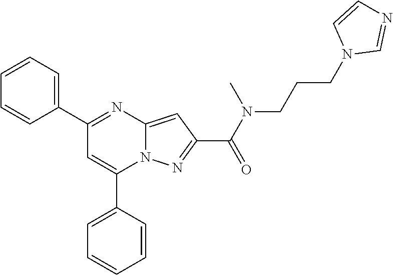 [SMILES: CN(CCCn1ccnc1)C(=O)c1cc2nc(-c3ccccc3)cc(-c3ccccc3)n2n1]

Example 11 was synthetized analogously to the method in Example 1 using 5,7-diphenylpyrazolo[1,5,a]pyrimidine-2-carboxylic acid (Intermediate 2) (30 mg, 0.095 mmol) and 3-(1H-imidazol-1-yl)-N-methylpropan-1-amine (1.5 eq., 0.143 mmol), yielding Example 11 as a solid. ¹H NMR (500 MHz, DMSO-d₆) δ ppm 2.06 (quin, J=6.71 Hz, 2H) 2.92-3.21 (m, 3H) 3.43-3.52 (m, 2H) 3.80 (t, J=7.17 Hz, 1H) 4.03 (t, J=7.02 Hz, 1H) 6.68-6.91 (m, 1H) 6.99-7.09 (m, 1H) 7.22-7.44 (m, 1H) 7.45-7.78 (m, 7H) 7.84-7.94 (m, 1H) 8.14 (dd, J=7.86, 1.60 Hz, 1H) 8.19-8.24 (m, 1H) 8.30-8.38 (m, 2H). MS (ESI+) calculated for C₂₆H₂₄N₆O: 436.2, found: 337.2 [M+H]⁺.

### Example 12. N-(2-(1H-Imidazol-1-yl)ethyl)-5,7-diphenylpyrazolo[1,5-a]pyrimidine-2-carboxamide

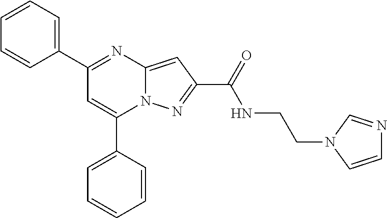 [SMILES: O=C(NCCn1ccnc1)c1cc2nc(-c3ccccc3)cc(-c3ccccc3)n2n1]

Example 12 was synthetized analogously to the method in Example 1 using 5,7-diphenylpyrazolo[1,5,a]pyrimidine-2-carboxylic acid (Intermediate 2) (30 mg, 0.095 mmol) and 2-(1H-imidazol-1-yl)ethan-1-amine (1.5 eq., 0.143 mmol), yielding Example 12 as a solid. ¹H NMR (500 MHz, DMSO-d₆) δ ppm 3.63 (q, J=6.05 Hz, 2H) 4.20 (t, J=6.26 Hz, 2H) 6.88 (t, J=0.99 Hz, 1H) 7.15 (s, 1H) 7.17 (t, J=1.22 Hz, 1H) 7.56-7.60 (m, 3H) 7.61 (s, 1H) 7.63-7.69 (m, 3H) 7.93 (s, 1H) 8.25-8.31 (m, 2H) 8.34 (dd, J=6.79, 2.98 Hz, 2H) 8.45 (t, J=5.87 Hz, 1H). MS (ESI+) calculated for C₂₄H₂₀N₆O: 408.2, found: 409.3 [M+H]⁺.

### Example 13. N-(3-Morpholinopropyl)-5,7-diphenylpyrazolo[1,5-a]pyrimidine-2-carboxamide

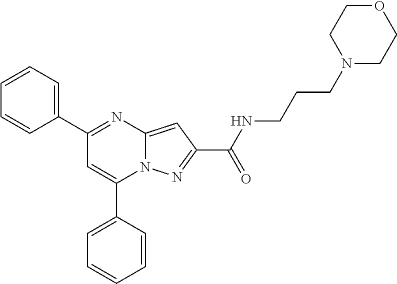 [SMILES: O=C(NCCCN1CCOCC1)c1cc2nc(-c3ccccc3)cc(-c3ccccc3)n2n1]

Example 13 was synthetized analogously to the method in Example 1 using 5,7-diphenylpyrazolo[1,5,a]pyrimidine-2-carboxylic acid (Intermediate 2) (30 mg, 0.095 mmol) and 3-morpholinopropan-1-amine (1.5 eq., 0.143 mmol), yielding Example 13 as a solid. ¹H NMR (500 MHz, DMSO-d₆) δ ppm 1.70 (quin, J=7.02 Hz, 2H) 2.32 (br d, J=6.87 Hz, 4H) 3.32 (s, 4H) 3.49 (t, J=4.58 Hz, 4H) 7.15 (s, 1H) 7.54-7.60 (m, 3H) 7.62-7.68 (m, 3H) 7.91 (s, 1H) 8.23-8.29 (m, 2H) 8.32-8.36 (m, 2H) 8.41 (t, J=5.87 Hz, 1H). MS (ESI) 441. MS (ESI+) calculated for C₂₆H₂₇N₅O₂: 441.1, found: 442.3 [M+H]⁺.

### Example 14. (S)—N-((1-Ethylpyrrolidin-2-yl)methyl)-5,7-diphenylpyrazolo[1,5-a]pyrimidine-2-carboxamide

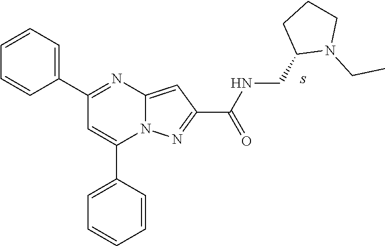

Example 14 was synthetized analogously to the method in Example 1 using 5,7-diphenylpyrazolo[1,5,a]pyrimidine-2-carboxylic acid (Intermediate 2) (30 mg, 0.095 mmol) and (S)-(1-ethylpyrrolidin-2-yl)methanamine (1.5 eq., 0.143 mmol), yielding Example 14 as a solid. ¹H NMR (500 MHz, DMSO-d₆) δ ppm 1.04 (t, J=7.17 Hz, 3H) 1.51-1.59 (m, 1H) 1.60-1.69 (m, 2H) 1.75-1.85 (m, 1H) 2.14 (td, J=8.96, 7.40 Hz, 1H) 2.26 (dq, J=12.00, 7.04 Hz, 1H) 2.64-2.67 (m, 1H) 2.83 (dq, J=11.98, 7.30 Hz, 1H) 3.06 (ddd, J=8.96, 6.29, 2.90 Hz, 1H) 3.18 (ddd, J=13.16, 6.37, 5.04 Hz, 1H) 3.44 (ddd, J=13.24, 6.68, 3.59 Hz, 1H) 7.15 (s, 1H) 7.55-7.61 (m, 3H) 7.62-7.70 (m, 3H) 7.95 (s, 1H) 8.11 (t, J=5.72 Hz, 1H) 8.26-8.31 (m, 2H) 8.33-8.37 (m, 2H). MS (ESI) 441. MS (ESI+) calculated for C₂₆H₂₇N₅O₂: 425.2, found: 426.3 [M+H]⁺.

### Example 15. (R)—N-((1-Ethylpyrrolidin-2-yl)methyl)-5,7-diphenylpyrazolo[1,5-a]pyrimidine-2-carboxamide

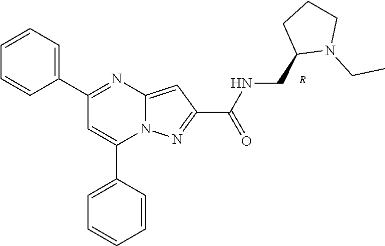

The example 15 was synthesized according to the procedure of Example 14, using (R)-(1-ethylpyrrolidin-2-yl)methanamine.

### Example 16. N-(1-Benzylpyrrolidin-3-yl)-5,7-diphenylpyrazolo[1,5-a]pyrimidine-2-carboxamide

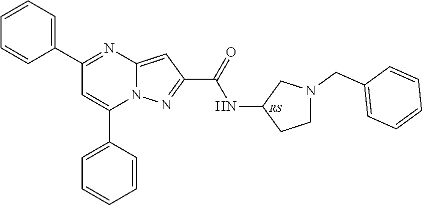 [SMILES: O=C(NC1CCN(Cc2ccccc2)C1)c1cc2nc(-c3ccccc3)cc(-c3ccccc3)n2n1]

Example 16 was synthetized analogously to the method in Example 1 using 5,7-diphenylpyrazolo[1,5,a]pyrimidine-2-carboxylic acid (Intermediate 2) (30 mg, 0.095 mmol) and 1-benzylpyrrolidin-3-amine (1.5 eq., 0.143 mmol), yielding Example 16 as a solid. ¹H NMR (500 MHz, DMSO-d₆) δ ppm 1.77-1.86 (m, 1H) 2.14-2.23 (m, 1H) 2.42-2.47 (m, 2H) 2.66-2.76 (m, 2H) 3.55-3.64 (m, 2H) 4.38-4.46 (m, 1H) 7.18 (s, 1H) 7.22-7.27 (m, 1H) 7.31 (d, J=4.58 Hz, 4H) 7.56-7.60 (m, 3H) 7.63-7.70 (m, 3H) 7.91 (s, 1H) 8.26 (d, J=7.48 Hz, 1H) 8.27-8.30 (m, 2H) 8.32-8.36 (m, 2H). MS (ESI+) calculated for C₃₀H₂₇N₅O: 473.2, found: 474.3 [M+H]⁺.

### Example 17. N-(1-Methylpiperidin-4-yl)-5,7-diphenylpyrazolo[1,5-a]pyrimidine-2-carboxamide

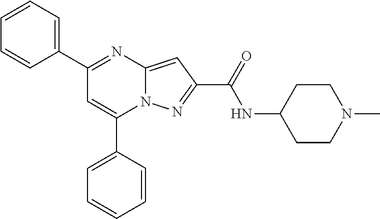 [SMILES: CN1CCC(NC(=O)c2cc3nc(-c4ccccc4)cc(-c4ccccc4)n3n2)CC1]

Example 17 was synthetized analogously to the method in Example 1 using 5,7-diphenylpyrazolo[1,5,a]pyrimidine-2-carboxylic acid (Intermediate 2) (30 mg, 0.095 mmol) and 1-methylpiperidin-4-amine (1.5 eq., 0.143 mmol), yielding Example 17 as a solid. ¹H NMR (500 MHz, DMSO-d₆) δ ppm 1.57-1.72 (m, 2H) 1.73-1.81 (m, 2H) 1.91-2.01 (m, 2H) 2.16 (s, 3H) 2.75 (br d, J=11.75 Hz, 2H) 3.71-3.85 (m, 1H) 7.17 (s, 1H) 7.53-7.62 (m, 3H) 7.63-7.70 (m, 3H) 7.91 (s, 1H) 8.12 (d, J=8.09 Hz, 1H) 8.25-8.31 (m, 2H) 8.32-8.36 (m, 2H). MS (ESI+) calculated for C₂₅H₂₅N₅O: 411.2, found: 4712.3 [M+H]⁺.

### Example 18. N-(3-(Dimethylamino)propyl)-5,7-diphenylpyrazolo[1,5-a]pyrimidine-2-carboxamide

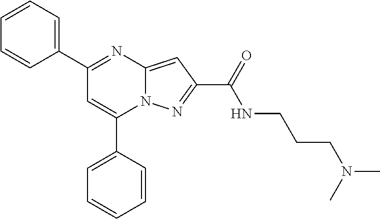 [SMILES: CN(C)CCCNC(=O)c1cc2nc(-c3ccccc3)cc(-c3ccccc3)n2n1]

Example 18 was synthetized analogously to the method in Example 1 using 5,7-diphenylpyrazolo[1,5,a]pyrimidine-2-carboxylic acid (Intermediate 2) (30 mg, 0.095 mmol) and N,N-dimethylpropane-1,3-diamine (1.5 eq., 0.143 mmol), yielding Example 18 as a solid. ¹H NMR (500 MHz, DMSO-d₆) δ ppm 1.66 (quin, J=6.79 Hz, 2H) 2.10 (s, 6H) 2.28 (t, J=6.71 Hz, 2H) 3.34-3.37 (m, 2H) 7.14 (s, 1H) 7.53-7.61 (m, 3H) 7.62-7.70 (m, 3H) 7.91 (s, 1H) 8.21-8.30 (m, 2H) 8.32-8.37 (m, 2H) 8.57 (t, J=5.72 Hz, 1H). MS (ESI+) calculated for C₂₄H₂₅N₅O: 399.2, found: 400.3 [M+H]⁺.

### Example 19. N-((1-Methylpyrrolidin-3-yl)methyl)-5,7-diphenylpyrazolo[1,5-a]pyrimidine-2-carboxamide

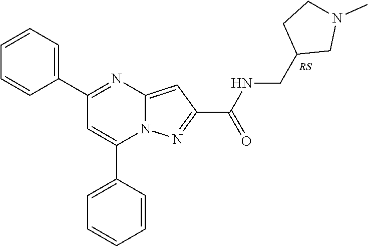 [SMILES: CN1CCC(CNC(=O)c2cc3nc(-c4ccccc4)cc(-c4ccccc4)n3n2)C1]

Example 19 was synthetized analogously to the method in Example 1 using 5,7-diphenylpyrazolo[1,5,a]pyrimidine-2-carboxylic acid (Intermediate 2) (30 mg, 0.095 mmol) and (1-methylpyrrolidin-3-yl)methanamine (1.5 eq., 0.143 mmol), yielding Example 19 as a solid. ¹H NMR (500 MHz, DMSO-d₆) δ ppm 1.44-1.54 (m, 1H) 1.80-1.93 (m, 1H) 2.22 (s, 3H) 2.28-2.36 (m, 2H) 2.41-2.58 (m, 3H) 3.22-3.30 (m, 2H) 7.15 (s, 1H) 7.53-7.62 (m, 3H) 7.63-7.70 (m, 3H) 7.92 (s, 1H) 8.27-8.32 (m, 2H) 8.32-8.37 (m, 2H) 8.49 (t, J=5.80 Hz, 1H). MS (ESI+) calculated for C₂₅H₂₅N₅O: 411.2, found: 412.3 [M+H]⁺.

### Example 20. 5,7-Diphenyl-N-((5,6,7,8-tetrahydroimidazo[1,2-a]pyridin-6-yl)methyl)pyrazolo[1,5-a]pyrimidine-2-carboxamide

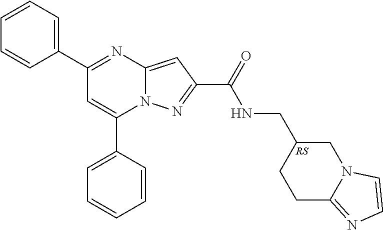 [SMILES: O=C(NCC1CCc2nccn2C1)c1cc2nc(-c3ccccc3)cc(-c3ccccc3)n2n1]

Example 20 was synthetized analogously to the method in Example 1 using 5,7-diphenylpyrazolo[1,5,a]pyrimidine-2-carboxylic acid (Intermediate 2) (30 mg, 0.095 mmol) and (5,6,7,8-tetrahydroimidazo[1,2-a]pyridin-6-yl)methanamine (1.5 eq., 0.143 mmol), yielding Example 20 as a solid. ¹H NMR (500 MHz, DMSO-d₆) δ ppm 1.61 (dtd, J=13.20, 10.76, 10.76, 5.72 Hz, 1H) 1.95-2.04 (m, 1H) 2.24-2.35 (m, 1H) 2.61-2.70 (m, 1H) 2.79-2.88 (m, 1H) 3.40 (t, J=6.56 Hz, 2H) 3.65 (dd, J=12.36, 9.92 Hz, 1H) 4.06 (dd, J=12.36, 4.88 Hz, 1H) 6.78 (d, J=1.22 Hz, 1H) 6.97 (d, J=1.22 Hz, 1H) 7.19 (s, 1H) 7.54-7.62 (m, 3H) 7.63-7.71 (m, 3H) 7.93 (s, 1H) 8.26-8.32 (m, 2H) 8.33-8.38 (m, 2H) 8.58 (t, J=6.10 Hz, 1H). MS (ESI+) calculated for C₂₇H₂₄N₆O: 448.2, found: 449.3 [M+H]⁺.

### Example 21. N-((1,3-Dimethylpyrrolidin-3-yl)methyl)-5,7-diphenylpyrazolo[1,5-a]pyrimidine-2-carboxamide

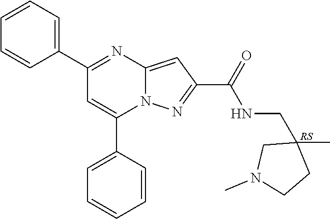 [SMILES: CN1CCC(C)(CNC(=O)c2cc3nc(-c4ccccc4)cc(-c4ccccc4)n3n2)C1]

Example 21 was synthetized analogously to the method in Example 1 using 5,7-diphenylpyrazolo[1,5,a]pyrimidine-2-carboxylic acid (Intermediate 2) (30 mg, 0.095 mmol) and (1,3-dimethylpyrrolidin-3-yl)methanamine (1.5 eq., 0.143 mmol), yielding Example 21 as a solid. ¹H NMR (500 MHz, DMSO-d₆) δ ppm 1.07 (s, 3H) 1.48 (ddd, J=12.78, 8.43, 4.58 Hz, 1H) 1.79 (dt, J=12.47, 7.80 Hz, 1H) 2.05 (d, J=9.00 Hz, 1H) 2.15 (s, 3H) 2.28-2.35 (m, 1H) 2.62 (d, J=9.46 Hz, 1H) 2.64-2.70 (m, 1H) 3.19-3.26 (m, 1H) 3.27-3.35 (m, 1H) 7.02-7.21 (m, 1H) 7.53-7.61 (m, 3H) 7.62-7.71 (m, 3H) 7.93 (s, 1H) 8.26-8.32 (m, 2H) 8.32-8.38 (m, 2H) 8.60 (t, J=5.49 Hz, 1H). MS (ESI+) calculated for C₂₆H₂₇N₅O: 425.2, found: 426.3 [M+H]⁺.

### Example 22. N-((1-Cyclopropylpyrrolidin-3-yl)methyl)-5,7-diphenylpyrazolo[1,5-a]pyrimidine-2-carboxamide

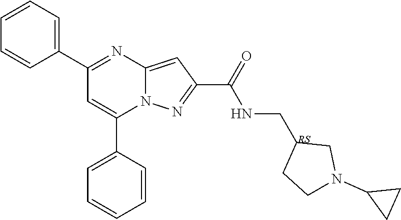 [SMILES: O=C(NCC1CCN(C2CC2)C1)c1cc2nc(-c3ccccc3)cc(-c3ccccc3)n2n1]

Example 22 was synthetized analogously to the method in Example 1 using 5,7-diphenylpyrazolo[1,5,a]pyrimidine-2-carboxylic acid (Intermediate 2) (30 mg, 0.095 mmol) and (1-cyclopropylpyrrolidin-3-yl)methanamine (1.5 eq., 0.143 mmol), yielding Example 22 as a solid. ¹H NMR (500 MHz, DMSO-d₆) δ ppm 0.19-0.29 (m, 2H) 0.29-0.38 (m, 2H) 1.41-1.52 (m, 1H) 1.57 (tt, J=6.56, 3.51 Hz, 1H) 1.77-1.88 (m, 1H) 2.38-2.47 (m, 2H) 2.55 (br d, J=6.87 Hz, 1H) 2.62-2.70 (m, 2H) 3.23-3.30 (m, 2H) 7.15 (s, 1H) 7.55-7.61 (m, 3H) 7.63-7.69 (m, 3H) 7.92 (s, 1H) 8.26-8.32 (m, 2H) 8.32-8.37 (m, 2H) 8.42 (t, J=5.87 Hz, 1H). MS (ESI+) calculated for C₂₇H₂₇N₅O: 437.2, found: 438.3 [M+H]⁺.

### Example 23. N-(1-Methylazetidin-3-yl)-5,7-diphenylpyrazolo[1,5-a]pyrimidine-2-carboxamide

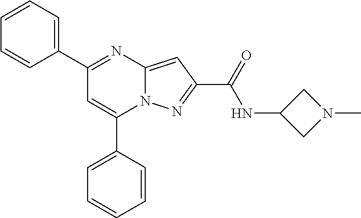 [SMILES: CN1CC(NC(=O)c2cc3nc(-c4ccccc4)cc(-c4ccccc4)n3n2)C1]

Example 23 was synthetized analogously to the method in Example 1 using 5,7-diphenylpyrazolo[1,5,a]pyrimidine-2-carboxylic acid (Intermediate 2) (30 mg, 0.095 mmol) and 1-methylazetidin-3-amine (1.5 eq., 0.143 mmol), yielding Example 23 as a solid. ¹H NMR (500 MHz, DMSO-d₆) δ ppm 2.33 (s, 3H) 3.19 (br s, 2H) 3.64 (br t, J=7.17 Hz, 2H) 4.45-4.53 (m, 1H) 7.18 (s, 1H) 7.54-7.61 (m, 3H) 7.64-7.69 (m, 3H) 7.92 (s, 1H) 8.26-8.31 (m, 2H) 8.31-8.37 (m, 2H) 8.73 (d, J=7.02 Hz, 1H). MS (ESI+) calculated for C₂₃H₂₁N₅O: 383.2, found: 384.2 [M+H]⁺.

### Example 24. N-((1-Methylpiperidin-4-yl)methyl)-5,7-diphenylpyrazolo[1,5-a]pyrimidine-2-carboxamide

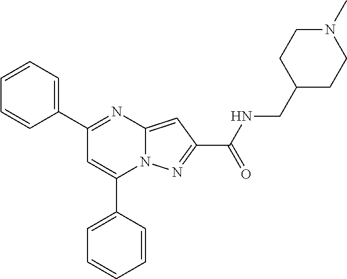 [SMILES: CN1CCC(CNC(=O)c2cc3nc(-c4ccccc4)cc(-c4ccccc4)n3n2)CC1]

Example 24 was synthetized analogously to the method in Example 1 using 5,7-diphenylpyrazolo[1,5,a]pyrimidine-2-carboxylic acid (Intermediate 2) (30 mg, 0.095 mmol) and 1-methylpiperidin-4-yl)methanamine (1.5 eq., 0.143 mmol), yielding Example 24 as a solid. ¹H NMR (500 MHz, DMSO-d₆) δ ppm 1.18 (br dd, J=12.13, 3.28 Hz, 2H) 1.52 (td, J=7.36, 3.74 Hz, 1H) 1.61 (br d, J=11.29 Hz, 2H) 1.78 (td, J=11.56, 2.06 Hz, 2H) 2.12 (s, 3H) 2.73 (br d, J=11.44 Hz, 2H) 3.19 (t, J=6.56 Hz, 2H) 7.16 (s, 1H) 7.55-7.60 (m, 3H) 7.62-7.69 (m, 3H) 7.92 (s, 1H) 8.29-8.32 (m, 2H) 8.32-8.36 (m, 2H) 8.37 (t, J=6.10 Hz, 1H). MS (ESI+) calculated for C₂₆H₂₇N₅O: 425.2, found: 426.4 [M+H]⁺.

### Example 25. N-(2-(1H-Imidazol-2-yl)ethyl)-5,7-diphenylpyrazolo[1,5-a]pyrimidine-2-carboxamide

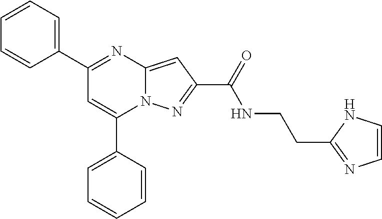 [SMILES: O=C(NCCc1ncc[nH]1)c1cc2nc(-c3ccccc3)cc(-c3ccccc3)n2n1]

Example 25 was synthetized analogously to the method in Example 1 using 5,7-diphenylpyrazolo[1,5,a]pyrimidine-2-carboxylic acid (Intermediate 2) (30 mg, 0.095 mmol) and 2-(1H-imidazol-2-yl)ethan-1-amine di-hydrochloride (1.5 eq., 0.143 mmol), yielding Example 25 as a solid. ¹H NMR (500 MHz, DMSO-d₆) δ ppm 2.89 (t, J=7.25 Hz, 2H) 3.59-3.69 (m, 2H) 6.73-6.91 (m, 1H) 7.01 (br s, 1H) 7.16 (s, 1H) 7.54-7.62 (m, 3H) 7.63-7.74 (m, 3H) 7.94 (s, 1H) 8.29-8.33 (m, 2H) 8.33-8.37 (m, 2H) 8.70 (br t, J=5.65 Hz, 1H) 11.81 (br s, 1H). MS (ESI+) calculated for C₂₄H₂₀N₆O: 408.2, found: 409.3 [M+H]⁺.

### Example 26. 5,7-Diphenyl-N-(3-(pyridin-2-ylamino)propyl)pyrazolo[1,5-a]pyrimidine-2-carboxamide

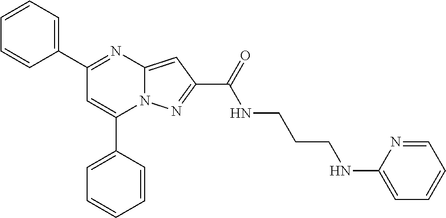 [SMILES: O=C(NCCCNc1ccccn1)c1cc2nc(-c3ccccc3)cc(-c3ccccc3)n2n1]

Example 26 was synthetized analogously to the method in Example 1 using 5,7-diphenylpyrazolo[1,5,a]pyrimidine-2-carboxylic acid (Intermediate 2) (30 mg, 0.095 mmol) and N1-(pyridin-2-yl)propane-1,3-diamine (1.5 eq., 0.143 mmol), example 26 as a solid. ¹H NMR (500 MHz, DMSO-d₆) δ ppm 1.76 (quin, J=6.71 Hz, 2H) 3.29 (br d, J=6.10 Hz, 2H) 3.35 (br d, J=6.56 Hz, 2H) 6.39 (ddd, J=6.94, 5.11, 0.76 Hz, 1H) 6.43 (d, J=8.39 Hz, 1H) 6.52 (t, J=5.80 Hz, 1H) 7.16 (s, 1H) 7.32 (ddd, J=8.54, 6.87, 1.98 Hz, 1H) 7.54-7.61 (m, 3H) 7.62-7.70 (m, 3H) 7.82 (dd, J=5.04, 1.22 Hz, 1H) 7.92 (s, 1H) 8.25-8.31 (m, 2H) 8.32-8.37 (m, 2H) 8.59 (t, J=6.03 Hz, 1H). MS (ESI+) calculated for C₂₇H₂₄N₆O: 448.2, found: 449.4 [M+H]⁺.

### Example 27. N-(3-(1H-Imidazol-4-yl)propyl)-5,7-diphenylpyrazolo[1,5-a]pyrimidine-2-carboxamide

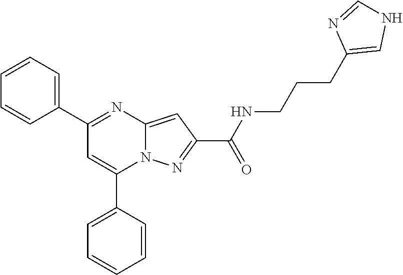 [SMILES: O=C(NCCCc1c[nH]cn1)c1cc2nc(-c3ccccc3)cc(-c3ccccc3)n2n1]

Example 27 was synthetized analogously to the method in Example 1 using 5,7-diphenylpyrazolo[1,5,a]pyrimidine-2-carboxylic acid (Intermediate 2) (30 mg, 0.095 mmol) and 3-(1H-imidazol-4-yl)propan-1-amine di-hydrochloride (1.5 eq., 0.143 mmol), yielding Example 27 as a solid. ¹H NMR (500 MHz, DMSO-d₆) δ ppm 1.83 (quin, J=7.32 Hz, 2H) 2.58 (br t, J=7.63 Hz, 2H) 3.34-3.40 (m, 2H) 6.57-6.93 (m, 1H) 7.07-7.23 (m, 1H) 7.47 (d, J=2.90 Hz, 1H) 7.54-7.63 (m, 3H) 7.64-7.70 (m, 3H) 7.92 (s, 1H) 8.27-8.36 (m, 4H) 8.42-8.54 (m, 1H) 11.60-11.95 (m, 1H). MS (ESI+) calculated for C₂₅H₂₂N₆O: 422.2, found: 423.4 [M+H]⁺.

### Example 28. 5,7-Diphenyl-N-(2-(pyridin-2-ylamino)ethyl)pyrazolo[1,5-a]pyrimidine-2-carboxamide

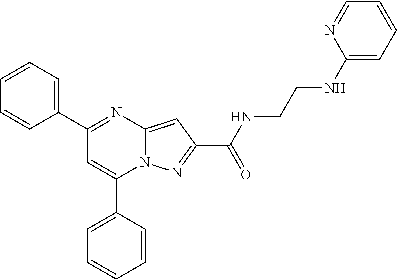 [SMILES: O=C(NCCNc1ccccn1)c1cc2nc(-c3ccccc3)cc(-c3ccccc3)n2n1]

Example 28 was synthetized analogously to the method in Example 1 using 5,7-diphenylpyrazolo[1,5,a]pyrimidine-2-carboxylic acid (Intermediate 2) (30 mg, 0.095 mmol) and N1-(pyridin-2-yl)ethane-1,2-diamine (1.5 eq., 0.143 mmol), yielding Example 28 as a solid. ¹H NMR (500 MHz, DMSO-d₆) δ ppm 3.45 (dt, J=18.08, 5.61 Hz, 4H) 6.41 (ddd, J=6.98, 5.07, 0.92 Hz, 1H) 6.47 (d, J=8.39 Hz, 1H) 6.68 (t, J=5.49 Hz, 1H) 7.16 (s, 1H) 7.35 (ddd, J=8.54, 6.87, 1.98 Hz, 1H) 7.52-7.62 (m, 3H) 7.62-7.71 (m, 3H) 7.74-7.83 (m, 1H) 7.91 (s, 1H) 8.22-8.30 (m, 2H) 8.32-8.38 (m, 2H) 8.69 (t, J=5.42 Hz, 1H). MS (ESI+) calculated for C₂₆H₂₂N₆O: 434.2, found: 435.3 [M+H]⁺.

### Example 29. N-((1-Methylazetidin-3-yl)methyl)-5,7-diphenylpyrazolo[1,5-a]pyrimidine-2-carboxamide

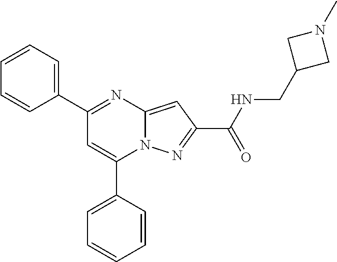 [SMILES: CN1CC(CNC(=O)c2cc3nc(-c4ccccc4)cc(-c4ccccc4)n3n2)C1]

Example 29 was synthetized analogously to the method in Example 1 using 5,7-diphenylpyrazolo[1,5,a]pyrimidine-2-carboxylic acid (Intermediate 2) (30 mg, 0.095 mmol) and (1-methylazetidin-3-yl)methanamine (1.5 eq., 0.143 mmol), yielding Example 29 as a solid. ¹H NMR (500 MHz, DMSO-d₆) δ ppm 2.16 (s, 3H) 2.54-2.59 (m, 1H) 2.87-2.94 (m, 2H) 3.17 (t, J=7.32 Hz, 2H) 3.46 (t, J=6.41 Hz, 2H) 7.15 (s, 1H) 7.53-7.61 (m, 3H) 7.63-7.71 (m, 3H) 7.92 (s, 1H) 8.28-8.32 (m, 2H) 8.33-8.36 (m, 2H) 8.52 (t, J=5.95 Hz, 1H). MS (ESI+) calculated for C₂₄H₂₃N₅O: 397.2, found: 398.3 [M+H]⁺.

### Example 30. 5,7-Diphenyl-N-(4-(pyrrolidin-1-yl)butyl)pyrazolo[1,5-a]pyrimidine-2-carboxamide

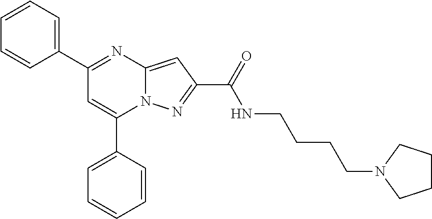 [SMILES: O=C(NCCCCN1CCCC1)c1cc2nc(-c3ccccc3)cc(-c3ccccc3)n2n1]

Example 30 was synthetized analogously to the method in Example 1 using 5,7-diphenylpyrazolo[1,5,a]pyrimidine-2-carboxylic acid (Intermediate 2) (30 mg, 0.095 mmol) and 4-(pyrrolidin-1-yl)butan-1-amine (1.5 eq., 0.143 mmol), yielding Example 30 as a solid. ¹H NMR (500 MHz, DMSO-d₆) δ ppm 1.43-1.51 (m, 2H) 1.57 (dt, J=14.61, 7.42 Hz, 2H) 1.63 (dt, J=6.71, 3.20 Hz, 4H) 2.33-2.42 (m, 6H) 3.28-3.31 (m, 2H) 7.14 (s, 1H) 7.54-7.61 (m, 3H) 7.62-7.69 (m, 3H) 7.91 (s, 1H) 8.27-8.32 (m, 2H) 8.32-8.36 (m, 2H) 8.41 (t, J=5.87 Hz, 1H). MS (ESI+) calculated for C₂₇H₂₉N₅O: 439.2, found: 440.4 [M+H]⁺.

### Example 31. 5,7-Diphenyl-N-(3-(pyridin-3-yl)propyl)pyrazolo[1,5-a]pyrimidine-2-carboxamide

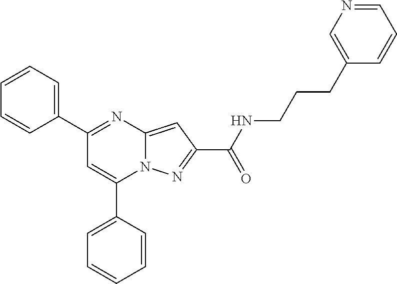 [SMILES: O=C(NCCCc1cccnc1)c1cc2nc(-c3ccccc3)cc(-c3ccccc3)n2n1]

Example 31 was synthetized analogously to the method in Example 1 using 5,7-diphenylpyrazolo[1,5,a]pyrimidine-2-carboxylic acid (Intermediate 2) (30 mg, 0.095 mmol) and 3-(pyridin-3-yl)propan-1-amine di-hydrochloride (1.5 eq., 0.143 mmol), yielding Example 31 a solid. ¹H NMR (500 MHz, DMSO-d₆) δ ppm 1.83-1.93 (m, 2H) 2.51-2.53 (m, 2H) 2.61-2.69 (m, 2H) 7.16 (s, 1H) 7.31 (ddd, J=7.78, 4.81, 0.69 Hz, 1H) 7.55-7.61 (m, 3H) 7.62-7.69 (m, 4H) 7.92 (s, 1H) 8.29-8.32 (m, 2H) 8.32-8.36 (m, 2H) 8.40 (dd, J=4.81, 1.60 Hz, 1H) 8.43-8.48 (m, 2H). MS (ESI+) calculated for C₂₇H₂₃N₅O: 433.2, found: 434.3 [M+H]⁺.

### Example 32. N-(3-(Methyl(pyridin-2-yl)amino)propyl)-5,7-diphenylpyrazolo[1,5-a]pyrimidine-2-carboxamide

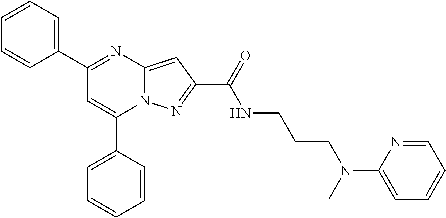 [SMILES: CN(CCCNC(=O)c1cc2nc(-c3ccccc3)cc(-c3ccccc3)n2n1)c1ccccn1]

Example 32 was synthetized analogously to the method in Example 1 using 5,7-diphenylpyrazolo[1,5,a]pyrimidine-2-carboxylic acid (Intermediate 2) (30 mg, 0.095 mmol) and N1-methyl-N1-(pyridin-2-yl)propane-1,3-diamine di-hydrochloride, (1.5 eq., 0.143 mmol), yielding Example 32 as a solid. ¹H NMR (500 MHz, DMSO-d₆) δ ppm 1.78 (t, J=6.79 Hz, 2H) 2.96 (s, 3H) 3.28 (br d, J=6.41 Hz, 2H) 3.58 (t, J=6.79 Hz, 2H) 6.43 (ddd, J=6.52, 5.53, 0.61 Hz, 1H) 6.58 (d, J=8.54 Hz, 1H) 7.15 (s, 1H) 7.44 (ddd, J=8.70, 6.94, 2.06 Hz, 1H) 7.54-7.60 (m, 3H) 7.61-7.70 (m, 3H) 7.81-7.86 (m, 1H) 7.92 (s, 1H) 8.24-8.29 (m, 2H) 8.31-8.37 (m, 2H) 8.62 (t, J=6.03 Hz, 1H). MS (ESI+) calculated for C₂₈H₂₆N₆O: 462.2, found: 463.4 [M+H]⁺.

### Example 33. N-(3-(Dimethylamino)-2,2-dimethylpropyl)-5,7-diphenylpyrazolo[1,5-a]pyrimidine-2-carboxamide

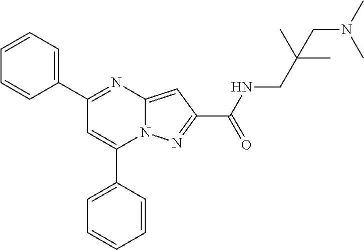 [SMILES: CN(C)CC(C)(C)CNC(=O)c1cc2nc(-c3ccccc3)cc(-c3ccccc3)n2n1]

Example 33 was synthetized analogously to the method in Example 1 using 5,7-diphenylpyrazolo[1,5,a]pyrimidine-2-carboxylic acid (Intermediate 2) (30 mg, 0.095 mmol) and N1,N1,2,2-tetramethylpropane-1,3-diamine (1.5 eq., 0.143 mmol), yielding Example 33 as a solid. ¹H NMR (500 MHz, DMSO-d₆) δ ppm 0.88 (s, 6H) 2.16 (s, 6H) 2.21 (s, 2H) 3.26 (d, J=5.65 Hz, 2H) 7.15 (s, 1H) 7.55-7.60 (m, 3H) 7.61-7.69 (m, 3H) 7.91 (s, 1H) 8.14-8.26 (m, 2H) 8.29-8.39 (m, 2H) 8.86 (t, J=5.57 Hz, 1H). MS (ESI+) calculated for C₂₆H₂₉N₅O: 427.2, found: 428.4 [M+H]⁺.

### Example 34. N-(2-(1H-Imidazol-4-yl)ethyl)-5,7-diphenylpyrazolo[1,5-a]pyrimidine-2-carboxamide

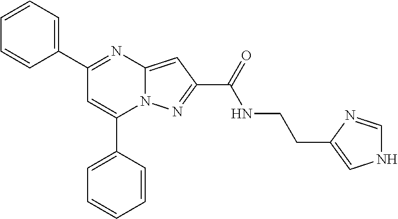 [SMILES: O=C(NCCc1c[nH]cn1)c1cc2nc(-c3ccccc3)cc(-c3ccccc3)n2n1]

Example 34 was synthetized analogously to the method in Example 1 using 5,7-diphenylpyrazolo[1,5,a]pyrimidine-2-carboxylic acid (Intermediate 2) (30 mg, 0.095 mmol) and 2-(1H-imidazol-4-yl)ethan-1-amine di-hydrochloride (1.5 eq., 0.143 mmol), yielding Example 34 as a solid. ¹H NMR (500 MHz, DMSO-d₆) δ ppm 2.75 (br t, J=6.79 Hz, 2H) 3.54 (q, J=6.87 Hz, 2H) 6.58-7.00 (m, 1H) 7.15 (s, 1H) 7.47-7.62 (m, 4H) 7.64-7.72 (m, 3H) 7.94 (s, 1H) 8.26-8.39 (m, 4H) 8.60 (br s, 1H) 11.49-12.24 (m, 1H). MS (ESI+) calculated for C₂₄H₂₀N₆O: 408.2, found: 409.3 [M+H]⁺.

### Example 35. N-(3-(Azetidin-1-yl)propyl)-5,7-diphenylpyrazolo[1,5-a]pyrimidine-2-carboxamide

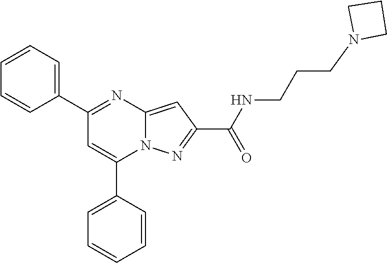 [SMILES: O=C(NCCCN1CCC1)c1cc2nc(-c3ccccc3)cc(-c3ccccc3)n2n1]

Example 35 was synthetized analogously to the method in Example 1 using 5,7-diphenylpyrazolo[1,5,a]pyrimidine-2-carboxylic acid (Intermediate 2) (30 mg, 0.095 mmol) and 3-(azetidin-1-yl)propan-1-amine (1.5 eq., 0.143 mmol), yielding Example 35 as a solid. ¹H NMR (500 MHz, DMSO-d₆) δ ppm 1.50 (quin, J=6.71 Hz, 2H) 1.76 (quin, J=6.90 Hz, 2H) 2.39 (t, J=6.64 Hz, 2H) 3.01 (t, J=6.94 Hz, 4H) 3.25-3.35 (m, 2H) 7.14 (s, 1H) 7.53-7.61 (m, 3H) 7.62-7.69 (m, 3H) 7.91 (s, 1H) 8.24-8.30 (m, 2H) 8.31-8.38 (m, 2H) 8.61 (t, J=5.72 Hz, 1H). MS (ESI+) calculated for C₂₅H₂₅N₅O: 411.2, found: 412.3 [M+H]⁺.

### Example 36. N-(2-(Azetidin-1-yl)ethyl)-5,7-diphenylpyrazolo[1,5-a]pyrimidine-2-carboxamide

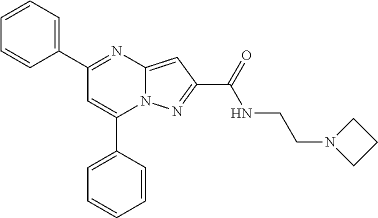 [SMILES: O=C(NCCN1CCC1)c1cc2nc(-c3ccccc3)cc(-c3ccccc3)n2n1]

Example 36 was synthetized analogously to the method in Example 1 using 5,7-diphenylpyrazolo[1,5,a]pyrimidine-2-carboxylic acid (Intermediate 2) (30 mg, 0.095 mmol) and 2-(azetidin-1-yl)ethan-1-amine (1.5 eq., 0.143 mmol), yielding Example 36 as a solid. ¹H NMR (500 MHz, DMSO-d₆) δ ppm 1.95 (quin, J=6.94 Hz, 2H) 2.47-2.60 (m, 2H) 3.13 (t, J=6.87 Hz, 4H) 3.24 (q, J=6.56 Hz, 2H) 7.15 (s, 1H) 7.53-7.62 (m, 3H) 7.62-7.71 (m, 3H) 7.93 (s, 1H) 8.24 (t, J=5.87 Hz, 1H) 8.27-8.38 (m, 4H). MS (ESI+) calculated for C₂₄H₂₃N₅O: 397.2, found: 398.3 [M+H]⁺.

### Example 37. N-(3-(1H-Imidazol-1-yl)cyclohexyl)-5,7-diphenylpyrazolo[1,5-a]pyrimidine-2-carboxamide

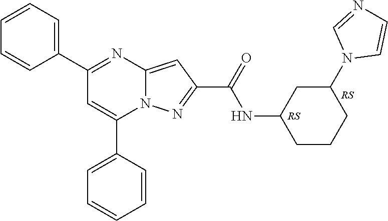 [SMILES: O=C(NC1CCCC(n2ccnc2)C1)c1cc2nc(-c3ccccc3)cc(-c3ccccc3)n2n1]

Example 37 was synthetized analogously to the method in Example 1 using 5,7-diphenylpyrazolo[1,5,a]pyrimidine-2-carboxylic acid (Intermediate 2) (30 mg, 0.095 mmol) and 3-(1H-imidazol-1-yl)cyclohexan-1-amine, (1.5 eq., 0.143 mmol), yielding Example 37 as a solid. ¹H NMR (500 MHz, DMSO-d₆) δ ppm 1.33-1.75 (m, 3H) 1.78-2.04 (m, 4H) 2.11-2.27 (m, 1H) 3.88-4.51 (m, 2H) 6.87-6.94 (m, 1H) 7.18 (s, 1H) 7.22-7.30 (m, 1H) 7.54-7.61 (m, 3H) 7.62-7.69 (m, 3H) 7.69-7.76 (m, 1H) 7.88-7.95 (m, 1H) 8.10-8.23 (m, 1H) 8.25-8.38 (m, 4H). MS (ESI+) calculated for C₂₈H₂₆N₆O: 462.2, found: 463.4 [M+H]⁺.

### Example 38. 5,7-Diphenyl-N-(2-(pyridin-4-yl)ethyl)pyrazolo[1,5-a]pyrimidine-2-carboxamide

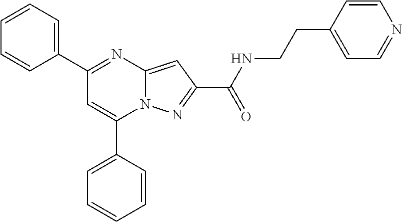 [SMILES: O=C(NCCc1ccncc1)c1cc2nc(-c3ccccc3)cc(-c3ccccc3)n2n1]

Example 38 was synthetized analogously to the method in Example 1 using 5,7-diphenylpyrazolo[1,5,a]pyrimidine-2-carboxylic acid (Intermediate 1) (30 mg, 0.095 mmol) and 2-(pyridin-4-yl)ethan-1-amine (1.5 eq., 0.143 mmol), yielding Example 38 as a solid. ¹H NMR (500 MHz, DMSO-d₆) δ ppm 2.90 (t, J=7.25 Hz, 2H) 3.57 (q, J=7.02 Hz, 2H) 7.14 (s, 1H) 7.24-7.33 (m, 2H) 7.54-7.61 (m, 3H) 7.63-7.72 (m, 3H) 7.92 (s, 1H) 8.28 (dd, J=7.86, 1.75 Hz, 2H) 8.32-8.37 (m, 2H) 8.41-8.52 (m, 3H). MS (ESI+) calculated for C₂₆H₂₁N₅O: 419.2, found: 420.3 [M+H]⁺.

### Example 39. 5,7-Diphenyl-N-(2-(pyridin-2-yl)ethyl)pyrazolo[1,5-a]pyrimidine-2-carboxamide

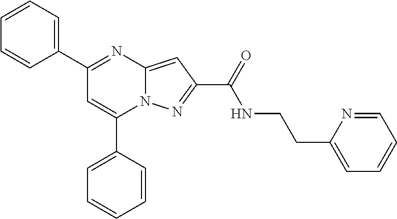 [SMILES: O=C(NCCc1ccccn1)c1cc2nc(-c3ccccc3)cc(-c3ccccc3)n2n1]

Example 39 was synthetized analogously to the method in Example 1 using 5,7-diphenylpyrazolo[1,5,a]pyrimidine-2-carboxylic acid (Intermediate 1) (30 mg, 0.095 mmol) and 2-(pyridin-2-yl)ethan-1-amine (1.5 eq., 0.1427 mmol), yielding Example 39 as a solid. ¹H NMR (500 MHz, DMSO-d₆) δ ppm 3.03 (t, J=7.25 Hz, 2H) 3.64-3.71 (m, 2H) 7.15 (s, 1H) 7.25 (ddd, J=7.48, 4.88, 1.07 Hz, 1H) 7.30 (d, J=7.78 Hz, 1H) 7.55-7.60 (m, 3H) 7.63-7.75 (m, 4H) 7.93 (s, 1H) 8.27-8.30 (m, 2H) 8.32-8.36 (m, 2H) 8.48-8.50 (m, 1H) 8.54 (t, J=5.80 Hz, 1H). MS (ESI+) calculated for C₂₆H₂₁N₅O: 419.2, found: 420.2 [M+H]⁺.

### Example 40. N-Phenethyl-5,7-diphenylpyrazolo[1,5-a]pyrimidine-2-carboxamide

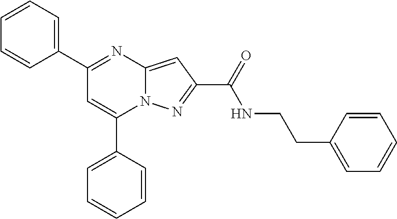 [SMILES: O=C(NCCc1ccccc1)c1cc2nc(-c3ccccc3)cc(-c3ccccc3)n2n1]

Example 40 was synthetized analogously to the method in Example 1 using 5,7-diphenylpyrazolo[1,5,a]pyrimidine-2-carboxylic acid (Intermediate 1) (30 mg, 0.095 mmol) and 2-phenylethan-1-amine (1.5 eq., 0.143 mmol), yielding Example 40 as a solid. ¹H NMR (500 MHz, DMSO-d₆) δ ppm 2.87 (t, J=7.55 Hz, 2H) 3.50-3.56 (m, 2H) 7.15 (s, 1H) 7.19-7.24 (m, 1H) 7.25-7.33 (m, 4H) 7.56-7.60 (m, 3H) 7.63-7.71 (m, 3H) 7.92 (s, 1H) 8.27-8.30 (m, 2H) 8.32-8.36 (m, 2H) 8.40 (t, J=5.87 Hz, 1H). MS (ESI+) calculated for C₂₇H₂₂N₄O: 418.2, found: 419.3 [M+H]⁺.

### Example 41. N-Cyclobutyl-5,7-diphenylpyrazolo[1,5-a]pyrimidine-2-carboxamide

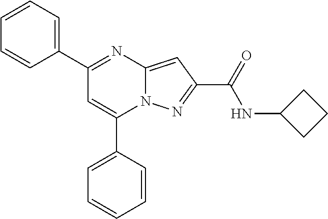 [SMILES: O=C(NC1CCC1)c1cc2nc(-c3ccccc3)cc(-c3ccccc3)n2n1]

Example 41 was synthetized analogously to the method in Example 1 using 5,7-diphenylpyrazolo[1,5,a]pyrimidine-2-carboxylic acid (Intermediate 1) (30 mg, 0.095 mmol) and commercially available cyclobutanamine, (1.5 eq., 0.1427 mmol), yielding Compound 41 as a solid. ¹H NMR (500 MHz, DMSO-d₆) δ ppm 1.56-1.75 (m, 2H) 2.10-2.28 (m, 4H) 4.38-4.52 (m, 1H) 7.16 (s, 1H) 7.54-7.60 (m, 3H) 7.64-7.70 (m, 3H) 7.91 (s, 1H) 8.27-8.32 (m, 2H) 8.32-8.36 (m, 2H) 8.51 (d, J=7.93 Hz, 1H). MS (ESI+) calculated for C₂₃H₂₀N₄O: 368.2, found: 369.3 [M+H]⁺.

### Example 42. N-(1,3-Dimethylazetidin-3-yl)-5,7-diphenylpyrazolo[1,5-a]pyrimidine-2-carboxamide

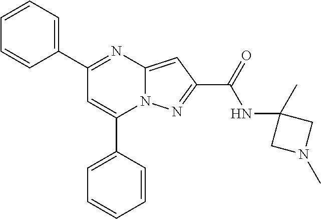 [SMILES: CN1CC(C)(NC(=O)c2cc3nc(-c4ccccc4)cc(-c4ccccc4)n3n2)C1]

5,7-diphenylpyrazolo[1,5,a]pyrimidine-2-carboxylic acid (Intermediate 2) (75 mg, 0.24 mmol) was dissolved in DMF (5 mL), and HATU (0.14 g, 0.36 mmol), Et₃N (0.1 mL, 0.73 g/mL, 0.28 mmol) and 1,3-dimethylazetidine-3-amine dihydrochloride (82 mg, 0.48 mmol) were added. The mixture was stirred at rt for 27 h. aq Na₂CO₃ solution was added and stirred for 10 min. Then, DCM and H₂O were added, the layers were separated, and the organic layer was concentrated in vacuo. The product was purified by RP HPLC (Stationary phase: C18 XBridge 30×100 mm 5 μm, Mobile phase: NH₄HCO₃ 0.25% solution in water and MeOH), yielding Example 42 as a solid. ¹H NMR (500 MHz, DMSO-d₆) δ ppm 1.56 (s, 3H) 2.24 (s, 3H) 3.11 (d, J=7.8 Hz, 2H) 3.30-3.31 (m, 2H) 7.15 (s, 1H) 7.55-7.61 (m, 3H) 7.62-7.70 (m, 3H) 7.91 (s, 1H) 8.27-8.32 (m, 2H) 8.32-8.37 (m, 2H) 8.60 (s, 1H). MS (ESI+) calculated for C₂₄H₂₃N₅O: 397.2, found: 398.3 [M+H]⁺.

### Example 43. N-(3-(1H-Imidazol-1-yl)propyl)-5,7-diphenylpyrazolo[1,5-a]pyrimidine-2-carboxamide

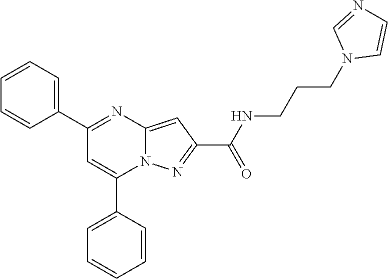 [SMILES: O=C(NCCCn1ccnc1)c1cc2nc(-c3ccccc3)cc(-c3ccccc3)n2n1]

To a mixture of 5,7-diphenylpyrazolo[1,5,a]pyrimidine-2-carboxylic acid (Intermediate 2) (400 mg, 1.269 mmol) in THF (10 mL) at rt was added 1-(3-dimethylaminopropyl)-3-ethylcarbodiimide hydrochloride (268 mg, 1.395 mmol), 1-hydroxybenzotriazole (189 mg, 1.395 mmol) and DIPEA (180 mg, 13.95 mmol). The mixture was stirred at rt for 30 min, then the mixture was added with N-(3-aminopropyl)-imidazole (175 mg, 1.395 mmol) and stirred at rt for 16 h. The mixture was quenched with water (10 mL) and extracted with EtOAc. The combined organic phase was washed with aq. NaHCO₃ (3×10 mL) and brine (30 mL), dried over Na₂SO₄, filtered, and concentrated in vacuo. The crude product was triturated with MeOH (5 mL) and filtered to give example 43 as white solid. ¹H (300 MHz, DMSO-d₆) δ ppm 1.95-2.01 (m, 2H) 3.15-3.29 (m, 2H), 4.00-4.07 (m, 2H), 6.90 (s, 1H), 7.17 (s, 1H) 7.21 (s, 1H), 7.57-7.59 (m, 3H) 7.66-7.69 (m, 4H) 7.94 (s, 1H) 8.29-8.38 (m, 4H) 8.50 (m, 1H). MS (ESI+) calculated for C₂₅H₂₂N₆O: 422.2, found: 423.2 [M+H]⁺.

### Example 44. N-(2-(Dimethylamino)ethyl)-N-methyl-5,7-diphenylpyrazolo[1,5-a]pyrimidine-2-carboxamide

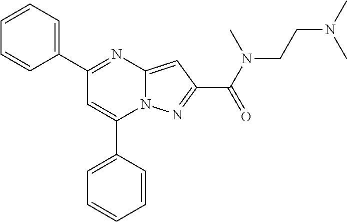 [SMILES: CN(C)CCN(C)C(=O)c1cc2nc(-c3ccccc3)cc(-c3ccccc3)n2n1]

To a mixture of 5,7-diphenylpyrazolo[1,5-a]pyrimidine-2-carbonyl azide (Intermediate 30) (150 mg, 0.44 mmol) in toluene (4 mL) was heated at 90° C. for 2 h. Then the reaction was cooled at rt and concentrated in vacuo. The crude was dissolved in DCM (4 mL), N,N′-trimethylethylenediamine (50 mg, 0.489 mmol) and DIPEA (0.11 mL, 0.75 g/mL, 0.661 mmol) were added at rt. The mixture was stirred for 5 h at rt. The reaction mixture was evaporated in vacuo and the crude was purified by flash column chromatography (SiO₂; MeOH in DCM 0/100 to 10/90) and then RP HPLC (stationary phase: C18 XBridge 30×100 mm 5 μm. Mobile phase: NH₄HCO₃ 0.25% solution in water and MeOH), yielding Example 44. ¹H NMR (500 MHz, CDCl₃) δ ppm 2.39 (s, 6H) 2.58-2.67 (m, 2H) 3.05 (s, 3H) 3.37-3.44 (m, 2H) 7.05-7.12 (m, 1H) 7.25 (s, 1H) 7.46-7.58 (m, 6H) 8.01-8.23 (m, 4H) 10.65-12.47 (m, 1H). MS (ESI+) calculated for C₂₄H₂₅N₅O: 399.2, found: 400.2 [M+H]⁺.

### Example 45. N-Methyl-N-(1-methylazetidin-3-yl)-5,7-diphenylpyrazolo[1,5-a]pyrimidine-2-carboxamide

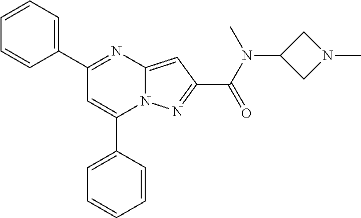 [SMILES: CN1CC(N(C)C(=O)c2cc3nc(-c4ccccc4)cc(-c4ccccc4)n3n2)C1]

Example 45 was synthetized analogously to the method in Example 42 using 5,7-diphenylpyrazolo[1,5,a]pyrimidine-2-carboxylic acid (Intermediate 2) (75 mg, 0.24 mmol) and N,1-dimethylazetidine-3-amine (36 mg, 0.36 mmol) yielding Example 45 as a solid. ¹H NMR (500 MHz, DMSO-d₆) δ ppm 2.20 (s, 1.8H) 2.25 (s, 1.2H) 3.09 (br t, J=7.1 Hz, 0.8H) 3.13-3.23 (m, 4.2H) 3.27-3.32 (m, 1.2H) 3.54 (br t, J=7.2 Hz, 0.8H) 4.73 (quin, J=6.8 Hz, 0.4H) 4.87 (quin, J=6.7 Hz, 0.6H) 7.00 (s, 0.6H) 7.04 (s, 0.4H) 7.54-7.61 (m, 3H) 7.61-7.69 (m, 3H) 7.90 (br s, 0.4H) 7.91 (s, 0.6H) 8.17-8.25 (m, 2H) 8.29-8.40 (m, 2H). MS (ESI+) calculated for C₂₄H₂₃N₅O: 397.2, found: 398.3 [M+H]⁺.

### Example 46. 3-Iodo-N-(1-methylazetidin-3-yl)-5,7-diphenylpyrazolo[1,5-a]pyrimidine-2-carboxamide

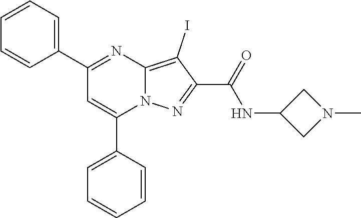 [SMILES: CN1CC(NC(=O)c2nn3c(-c4ccccc4)cc(-c4ccccc4)nc3c2I)C1]

Example 46 was synthetized analogously to the method in Example 42 using 3-iodo-5,7-diphenylpyrazolo[1,5-a]pyrimidine-2-carboxylic acid (Intermediate 8) (176 mg, 0.4 mmol) and 1-methylazetidin-3-amine (0.057 mL, 0.96 g/mL, 0.6 mmol) yielding Example 46 as a solid. ¹H NMR (500 MHz, DMSO-d₆) δ ppm 2.25 (s, 3H) 3.01 (t, J=7.32 Hz, 2H) 3.54-3.59 (m, 2H) 4.43 (q, J=7.02 Hz, 1H) 7.54-7.74 (m, 7H) 7.98 (s, 1H) 8.25-8.31 (m, 2H) 8.38-8.44 (m, 2H) 8.73 (d, J=7.02 Hz, 1H). MS (ESI+) m/z calculated for C₂₃H₂₀IN₅O: 509.1; found: 510.1[M+H]⁺.

### Example 47. 3-Iodo-N-(1-methylpyrrolidin-3-yl)-5,7-diphenylpyrazolo[1,5-a]pyrimidine-2-carboxamide

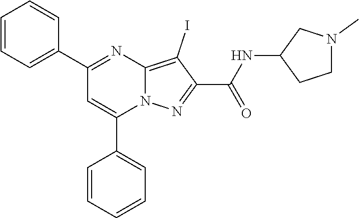 [SMILES: CN1CCC(NC(=O)c2nn3c(-c4ccccc4)cc(-c4ccccc4)nc3c2I)C1]

Example 47 was synthetized analogously to the method in Example 42 using 3-iodo-5,7-diphenylpyrazolo[1,5-a]pyrimidine-2-carboxylic acid (Intermediate 8) (176 mg, 0.4 mmol) and 1-methylpyrrolidin-3-amine (0.064 mL, 0.93 g/mL, 0.6 mmol) yielding Example 47 as a solid. ¹H NMR (500 MHz, DMSO-d₆) δ ppm 1.66-1.83 (m, 1H) 2.12-2.22 (m, 1H) 2.25 (s, 3H) 2.34-2.47 (m, 2H) 2.59-2.75 (m, 2H) 4.33-4.45 (m, 1H) 7.54-7.72 (m, 6H) 7.97 (s, 1H) 8.20-8.28 (m, 2H) 8.34 (d, J=7.32 Hz, 1H) 8.38-8.44 (m, 2H). MS (ESI+) calculated for C₂₄H₂₂IN₅O: 523.1, found: 524.1 [M+H]⁺.

### Example 48. N-((1,3-Dimethylpyrrolidin-3-yl)methyl)-3-iodo-5,7-diphenylpyrazolo[1,5-a]pyrimidine-2-carboxamide

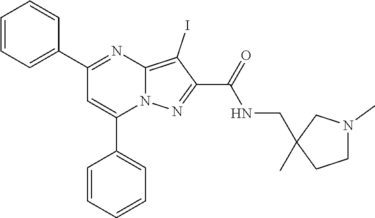 [SMILES: CN1CCC(C)(CNC(=O)c2nn3c(-c4ccccc4)cc(-c4ccccc4)nc3c2I)C1]

Example 48 was synthetized analogously to the method in Example 42 using 3-iodo-5,7-diphenylpyrazolo[1,5-a]pyrimidine-2-carboxylic acid (Intermediate 8) (176 mg, 0.4 mmol) and (1,3-dimethylpyrrolidin-3-yl)methanamine (0.086 mL, 0.9 g/mL, 0.6 mmol) yielding Example 48 as a solid. ¹H NMR (400 MHz, DMSO-d₆) δ ppm 1.09 (s, 3H) 1.47 (ddd, J=12.83, 8.32, 4.74 Hz, 1H) 1.79 (dt, J=12.54, 7.60 Hz, 1H) 2.06 (d, J=9.25 Hz, 1H) 2.14 (s, 3H) 2.29-2.40 (m, 1H) 2.56-2.69 (m, 3H) 3.18-3.28 (m, 2H) 7.54-7.77 (m, 6H) 7.99 (s, 1H) 8.27 (dd, J=7.74, 1.73 Hz, 2H) 8.36-8.46 (m, 2H) 8.57 (t, J=5.55 Hz, 1H). MS (ESI+) calculated for C₂₆H₂₆IN₅O: 551.1, found: 552.1 [M+H]⁺.

### Example 49. 3-Chloro-N-(1-methylazetidin-3-yl)-5,7-diphenylpyrazolo[1,5-a]pyrimidine-2-carboxamide

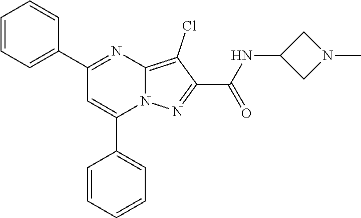 [SMILES: CN1CC(NC(=O)c2nn3c(-c4ccccc4)cc(-c4ccccc4)nc3c2Cl)C1]

Example 49 was synthetized analogously to the method in Example 42 using 3-chloro-5,7-diphenylpyrazolo[1,5-a]pyrimidine-2-carboxylic acid (Intermediate 9) (67 mg, 0.19 mmol) and 1-methylazetidin-3-amine (0.026 mL, 0.96 g/mL, 0.29 mmol) yielding Example 49 as a solid. ¹H NMR (500 MHz, DMSO-d₆) δ ppm 2.26 (br s, 3H) 3.02-3.10 (m, 2H) 3.58 (br s, 2H) 4.09 (q, J=5.19 Hz, 1H) 7.56-7.74 (m, 6H) 8.01 (s, 1H) 8.24-8.30 (m, 2H) 8.37-8.43 (m, 2H) 8.81 (br d, J=6.71 Hz, 1H). MS (ESI+) calculated for C₂₃H₂₀ClN₅O: 417.1, found: 418.2 [M+H]⁺.

### Example 50. 3-Methyl-N-(1-methylazetidin-3-yl)-5,7-diphenylpyrazolo[1,5-a]pyrimidine-2-carboxamide

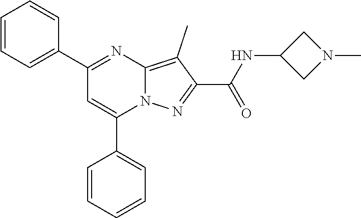 [SMILES: Cc1c(C(=O)NC2CN(C)C2)nn2c(-c3ccccc3)cc(-c3ccccc3)nc12]

A solution of dimethylzinc (0.13 mL, 2 M in toluene, 0.26 mmol) was added dropwise to a stirred solution of 3-iodo-N-(1-methylazetidin-3-yl)-5,7-diphenylpyrazolo[1,5-a]pyrimidine-2-carboxamide (Example 46) (45 mg, 0.088 mmol) and bis(tri-tert-butylphosphine)palladium (0) (2 mg, 0.004 mmol) in THF (1 mL) at 0° C. The mixture was stirred at 0° C. for 2 min and at 70° C. for 1 h. aq sat NH₄Cl solution was added to the mixture and diluted with DCM. The layers were separated, and the organic layers were concentrated in vacuo. The crude product was purified by RP HPLC (Conditions: Stationary phase: C18 XBridge 30×100 mm 10 μm. Mobile phase: NH₄HCO₃ 0.25% solution in water and CH₃CN) yielding Example 50 as a solid. ¹H NMR (500 MHz, DMSO-d₆) δ ppm 2.24 (s, 3H) 2.55 (s, 3H) 3.02 (br t, J=7.10 Hz, 2H) 3.55 (t, J=7.10 Hz, 2H) 4.45 (sxt, J=7.02 Hz, 1H) 7.52-7.63 (m, 3H) 7.64-7.72 (m, 3H) 7.88 (s, 1H) 8.27-8.39 (m, 4H) 8.51 (d, J=7.17 Hz, 1H). MS (ESI+) calculated for C₂₄H₂₃N₅O: 397.2, found: 398.2 [M+H]⁺.

### Example 51. 3-Methyl-N-(1-methylpyrrolidin-3-yl)-5,7-diphenylpyrazolo[1,5-a]pyrimidine-2-carboxamide

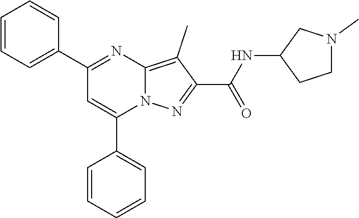 [SMILES: Cc1c(C(=O)NC2CCN(C)C2)nn2c(-c3ccccc3)cc(-c3ccccc3)nc12]

Example 51 was synthetized analogously to the method in Example 50 using dimethylzinc 2M in toluene (0.26 mL, 0.52 mmol) and 3-iodo-N-(1-methylpyrrolidin-3-yl)-5,7-diphenylpyrazolo[1,5-a]pyrimidine-2-carboxamide (Example 47) (90 mg, 0.17 mmol) yielding Example 54 as a solid. ¹H NMR (500 MHz, DMSO-d₆) δ ppm 1.69-1.80 (m, 1H) 2.08-2.23 (m, 2H) 2.25 (s, 3H) 2.56 (s, 3H) 2.59-2.72 (m, 3H) 4.35-4.48 (m, 1H) 7.55-7.61 (m, 3H) 7.63-7.67 (m, 3H) 7.87 (s, 1H) 8.06 (d, J=7.48 Hz, 1H) 8.27-8.33 (m, 2H) 8.34-8.39 (m, 2H). MS (ESI+) calculated for C₂₅H₂₅N₅O: 411.2, found: 412.2 [M+H]⁺.

### Example 52. 3-Fluoro-N-(1-methylazetidin-3-yl)-5,7-diphenylpyrazolo[1,5-a]pyrimidine-2-carboxamide

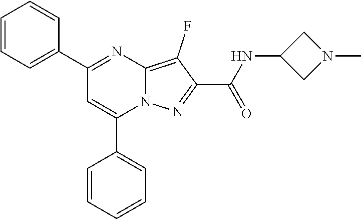 [SMILES: CN1CC(NC(=O)c2nn3c(-c4ccccc4)cc(-c4ccccc4)nc3c2F)C1]

Example 52 was synthetized analogously to the method in Example 42 using 3-fluoro-5,7-diphenylpyrazolo[1,5-a]pyrimidine-2-carboxylic acid (Intermediate 10) (104 mg, 0.31 mmol) and 1-methylazetidin-3-amine (42 μL, 0.96 g/mL, 0.47 mmol) yielding Example 52 as a solid. ¹H NMR (500 MHz, DMSO-d₆) δ ppm 2.24 (s, 3H) 2.99-3.08 (m, 2H) 3.48-3.63 (m, 2H) 4.38-4.48 (m, 1H) 7.54-7.63 (m, 3H) 7.64-7.72 (m, 3H) 7.95 (s, 1H) 8.23-8.30 (m, 2H) 8.33-8.41 (m, 2H) 8.75 (d, J=7.02 Hz, 1H). MS (ESI+) calculated for C₂₃H₂₀FN₅O: 401.2, found: 402.2 [M+H]⁺.

### Example 53. 3-Cyano-N-(1-methylazetidin-3-yl)-5,7-diphenylpyrazolo[1,5-a]pyrimidine-2-carboxamide

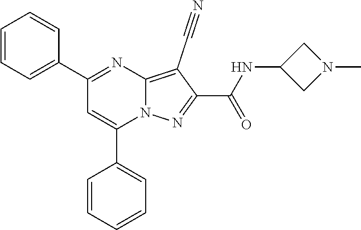 [SMILES: CN1CC(NC(=O)c2nn3c(-c4ccccc4)cc(-c4ccccc4)nc3c2C#N)C1]

Example 53 was synthetized analogously to the method in Example 42 using 3-cyano-5,7-diphenylpyrazolo[1,5-a]pyrimidine-2-carboxylic acid (Intermediate 11) (34 mg, 0.045 mmol) and 1-methylazetidin-3-amine (6 μL, 0.96 g/mL, 0.067 mmol) yielding Example 53 as a solid. ¹H NMR (500 MHz, DMSO-d₆) δ ppm 2.26 (s, 3H) 3.07 (br t, J=6.41 Hz, 2H) 3.57 (br t, J=6.64 Hz, 2H) 4.44 (sxt, J=6.96 Hz, 1H) 7.61-7.66 (m, 4H) 7.68-7.71 (m, 2H) 8.20 (s, 1H) 8.29 (dd, J=7.93, 1.68 Hz, 2H) 8.41-8.46 (m, 2H) 9.01 (d, J=6.87 Hz, 1H) MS (ESI+) calculated for C₂₄H₂₀N₆O: 408.2, found: 409.2 [M+H]⁺.

### Example 54. N-((1,3-Dimethylpyrrolidin-3-yl)methyl)-3-methyl-5,7-diphenylpyrazolo[1,5-a]pyrimidine-2-carboxamide

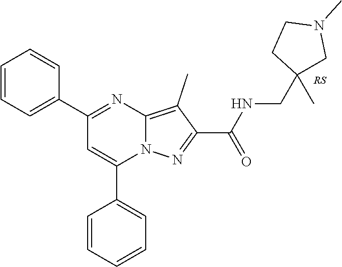 [SMILES: Cc1c(C(=O)NCC2(C)CCN(C)C2)nn2c(-c3ccccc3)cc(-c3ccccc3)nc12]

Example 54 was synthetized analogously to the method in Example 50 using dimethylzinc 2M in (0.41 mmol, 2M in toluene) and N-((1,3-dimethylpyrrolidin-3-yl)methyl)-3-iodo-5,7-diphenylpyrazolo[1,5-a]pyrimidine-2-carboxamide (Example 48) (75 mg, 0.14 mmol), yielding Example 54 as a solid. ¹H NMR (500 MHz, DMSO-d₆) δ ppm 1.08 (d, J=1.37 Hz, 3H) 1.40-1.56 (m, 2H) 1.79 (dt, J=12.86, 7.46 Hz, 1H) 2.05 (br d, J=8.85 Hz, 1H) 2.14 (s, 3H) 2.32 (br d, J=7.78 Hz, 1H) 2.54-2.66 (m, 4H) 3.18-3.25 (m, 1H) 3.29 (br s, 1H) 7.55-7.60 (m, 3H) 7.63-7.67 (m, 3H) 7.89 (d, J=1.98 Hz, 1H) 8.28-8.32 (m, 2H) 8.35-8.40 (m, 2H) 8.48 (br t, J=4.81 Hz, 1H). MS (ESI+) calculated for C₂₇H₂₉N₅O: 439.2, found: 440.2 [M+H]⁺.

### Example 55. N-(1,2-Dimethylazetidin-3-yl)-5,7-diphenylpyrazolo[1,5-a]pyrimidine-2-carboxamide

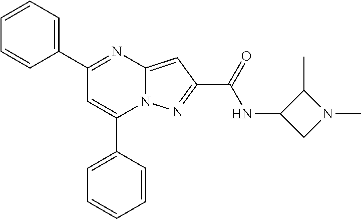 [SMILES: CC1C(NC(=O)c2cc3nc(-c4ccccc4)cc(-c4ccccc4)n3n2)CN1C]

Paraformaldehyde (40 mg, 0.44 mmol) followed by NaBH₄ (33 mg, 0.87 mmol) were added to a stirred solution of N-(2-methylazetidin-3-yl)-5,7-diphenylpyrazolo[1,5-a]pyrimidine-2-carboxamide (Intermediate 60) (114 mg, 0.3 mmol) in 2,2,2-trifluoroethanol (3 mL, 1.37 g/mL, 13.72 mmol). The mixture was stirred at 70° C. for 15 min. The mixture was treated with water and DCM and stirred for 5 min. The layers were separated, and the organic layers were concentrated in vacuo. The crude product was purified by RP HPLC (Conditions: Stationary phase: C18 XBridge 30×100 mm 10 μm. Mobile phase: NH₄HCO₃ 0.25% solution in water and CH₃CN) yielding Example 129 as an off white solid. ¹H NMR (500 MHz, DMSO-d₆) δ ppm 0.89-1.20 (m, 3H) 2.17-2.29 (m, 3H) 2.77-3.30 (m, 2H) 3.37-3.61 (m, 1H) 4.01-4.55 (m, 1H) 7.09-7.32 (m, 1H) 7.49-7.62 (m, 3H) 7.63-7.72 (m, 3H) 7.89-7.96 (m, 1H) 8.24-8.39 (m, 4H) 8.42-8.62 (m, 1H). MS (ESI+) calculated for C₂₄H₂₄N₅O: 397.1; found: 398.2 [M+H]⁺.

### Example 56. N-(3-(1H-Imidazol-1-yl)propyl)-7-(azetidin-1-yl)-5-phenylpyrazolo[1,5-a]pyrimidine-2-carboxamide

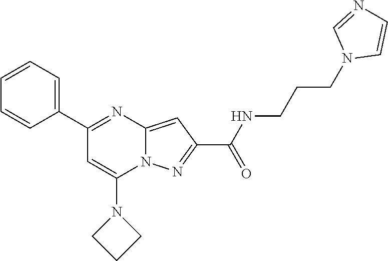 [SMILES: O=C(NCCCn1ccnc1)c1cc2nc(-c3ccccc3)cc(N3CCC3)n2n1]

N-(3-(1H-imidazol-1-yl)propyl)-7-chloro-5-phenylpyrazolo[1,5-a]pyrimidine-2-carboxamide (Intermediate 13) (37.5 mg, 0.1 mmol), DIPEA (50.9 μL, 0.75 g/mL, 0.3 mmol) and azetidine hydrochloride (18.42 mg, 0.197 mmol) were suspended in iPrOH (1 mL). The reaction was stirred at 50° C. for 1 h. The solvent was removed in vacuo, and the crude was purified by RP HPLC (Stationary phase: C18 XBridge, column with 100 mm length, 5 μm. Mobile phase: Gradient using NH4HCO3 0.25% solution in water and CH₃CN). The desired fractions were collected and concentrated in vacuo yielding Example 56 as a solid. ¹H NMR (500 MHz, DMSO-d₆) δ ppm 1.95-2.02 (m, 2H) 2.46 (m, 2H) 3.28 (m, 2H) 4.03 (t, J=6.87 Hz, 2H) 4.63 (br m, 4H) 6.41 (s, 1H) 6.70 (s, 1H) 6.92 (s, 1H) 7.24 (s, 1H) 7.47-7.53 (m, 3H) 7.71 (s, 1H) 8.11-8.16 (m, 2H) 8.35 (t, J=5.95 Hz, 1H). MS (ESI+) calculated for C₂₂H₂₃N₇O: 401.2, found: 402.2 [M+H]⁺.

### Example 57. N-(3-(1H-Imidazol-1-yl)propyl)-7-(isopropylamino)-5-phenylpyrazolo[1,5-a]pyrimidine-2-carboxamide

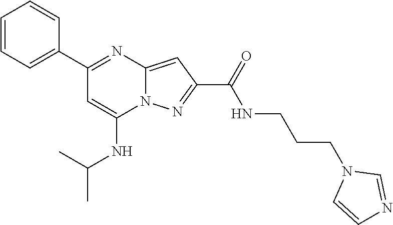 [SMILES: CC(C)Nc1cc(-c2ccccc2)nc2cc(C(=O)NCCCn3ccnc3)nn12]

Example 57 was synthetized analogously to the method in Example 56 with N-(3-(1H-imidazol-1-yl)propyl)-7-chloro-5-phenylpyrazolo[1,5-a]pyrimidine-2-carboxamide (Intermediate 13) (37.5 mg, 0.1 mmol) and propan-2-amine (11.64 mg, 0.197 mmol) yielding Example 57 as solid. ¹H NMR (500 MHz, DMSO-d₆) δ ppm 1.37 (d, J=6.41 Hz, 6H) 1.95-2.05 (m, 2H) 3.31 (br s, 2H) 4.05 (t, J=6.94 Hz, 2H) 4.22-4.31 (m, 1H) 6.78 (s, 1H) 6.86 (s, 1H) 6.91 (t, J=0.99 Hz, 1H) 7.24 (t, J=1.22 Hz, 1H) 7.34 (d, J=8.85 Hz, 1H) 7.46-7.54 (m, 3H) 7.69 (s, 1H) 8.16-8.22 (m, 2H) 8.41 (t, J=6.03 Hz, 1H). MS (ESI+) calculated for C₂₂H₂₅N₇O: 403.2; found: 404.3 [M+H]⁺.

### Example 58. N-(3-(1H-Imidazol-1-yl)propyl)-5-phenyl-7-(piperidin-1-yl)pyrazolo[1,5-a]pyrimidine-2-carboxamide

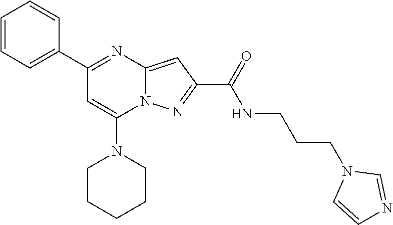 [SMILES: O=C(NCCCn1ccnc1)c1cc2nc(-c3ccccc3)cc(N3CCCCC3)n2n1]

Example 58 was synthetized analogously to the method in Example 56 with N-(3-(1H-imidazol-1-yl)propyl)-7-chloro-5-phenylpyrazolo[1,5-a]pyrimidine-2-carboxamide (Intermediate 13) and piperidine (16.77 mg, 0.197 mmol) yielding Example 58 as a solid. ¹H NMR (500 MHz, DMSO-d₆) δ ppm 1.69-1.81 (m, 6H) 1.96-2.05 (m, 2H) 3.29 (m, 2H) 3.81-3.90 (m, 4H) 4.04 (t, J=6.94 Hz, 2H) 6.87 (s, 1H) 6.91 (s, 2H) 7.24 (t, J=1.14 Hz, 1H) 7.49-7.56 (m, 3H) 7.69 (s, 1H) 8.17-8.22 (m, 2H) 8.44 (t, J=6.03 Hz, 1H). MS (ESI+) calculated for C₂₄H₂₇N₇O: 429.5, found: 430.2 [M+H]⁺.

### Example 59. N-(3-(1H-Imidazol-1-yl)propyl)-7-morpholino-5-phenylpyrazolo[1,5-a]pyrimidine-2-carboxamide

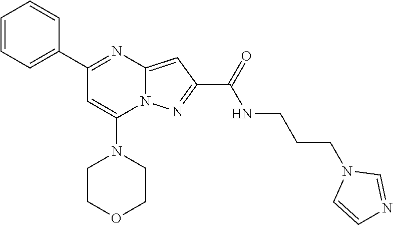 [SMILES: O=C(NCCCn1ccnc1)c1cc2nc(-c3ccccc3)cc(N3CCOCC3)n2n1]

Example 59 was synthetized analogously to the method in Example 56 with N-(3-(1H-imidazol-1-yl)propyl)-7-chloro-5-phenylpyrazolo[1,5-a]pyrimidine-2-carboxamide (Intermediate 13) (37.5 mg, 0.1 mmol) and morpholine (17.16 mg, 0.197 mmol) yielding Example 59. ¹H NMR (500 MHz, DMSO-d₆) δ ppm 1.96-2.05 (m, 2H) 3.28-3.31 (m, 2H) 3.88 (br m, 8H) 4.04 (t, J=6.87 Hz, 2H) 6.89-6.93 (m, 2H) 6.96 (s, 1H) 7.24 (t, J=1.14 Hz, 1H) 7.47-7.57 (m, 3H) 7.69 (s, 1H) 8.18-8.25 (m, 2H) 8.52 (t, J=6.03 Hz, 1H). MS (ESI+) C₂₃H₂₅N₇O₂: 431.2, found 432.3 [M+H]⁺.

### Example 60. N-(3-(1H-Imidazol-1-yl)propyl)-7-(4-methylpiperazin-1-yl)-5-phenylpyrazolo[1,5-a]pyrimidine-2-carboxamide

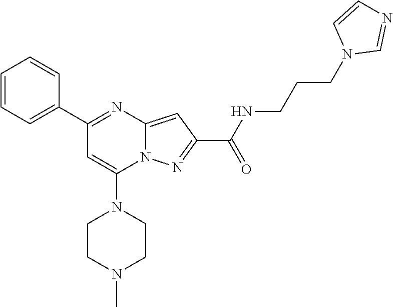 [SMILES: CN1CCN(c2cc(-c3ccccc3)nc3cc(C(=O)NCCCn4ccnc4)nn23)CC1]

A solution of ethyl 7-chloro-5-phenylpyrazolo[1,5-a]pyrimidine-2-carboxylate (Intermediate 3) (75 mg, 0.25 mmol) in ACN (1.25 mL) and THF (0.25 mL) was added to the 1-methylpiperazine (26 mg, 0.26 mmol) TEA (207 μL, 0.728 g/mL, 1.49 mmol). The reaction was stirred for 16 h at rt. Then, LiOH (1.5 mL, 0.5 M in water, 0.75 mmol) was added, and the reaction was stirred at 50° C. for 3 h. Solvents were removed in vacuo. Then, HCl (4M in dioxane) (188 μL, 4 M, 0.75 mmol) was added followed by DCM (1 mL) and THF (1 mL) and the reaction mixture was stirred for 5 min at rt. TEA (207 μL, 0.728 g/mL, 1.49 mmol), 1-propanephosphonic anhydride solution (0.63 mL, 0.5 g/mL, 0.99 mmol, 50 wt % in EtOAc) and 1-(3-aminopropyl)imidazole (74 μL, 1.049 g/mL, 0.62 mmol) were added. The reaction was stirred at rt for 6 h. The product was purified by RP HPLC (Stationary phase: C18 XBridge, column with 100 mm length, 5 μm. Mobile phase: Gradient using NH₄HCO₃ 0.25% solution in water and CH₃CN), yielding Example 60 as a solid. ¹H NMR (500 MHz, chloroform-d) δ ppm 2.17 (quin, J=6.90 Hz, 2H) 2.43 (s, 3H) 2.74 (br t, J=4.81 Hz, 4H) 3.52 (q, J=6.71 Hz, 2H) 3.80 (br s, 4H) 4.09 (t, J=7.02 Hz, 2H) 6.66 (s, 1H) 7.00 (s, 1H) 7.05-7.12 (m, 2H) 7.16 (s, 1H) 7.44-7.58 (m, 4H) 8.04 (dd, J=7.71, 1.60 Hz, 2H). MS (ESI+) C₂₄H₂₈N₈O: 444.2, found 445.3[M+H]⁺.

### Example 61. N-(3-(1H-Imidazol-1-yl)propyl)-7-(3-azabicyclo[3.1.0]hexan-3-yl)-5-phenylpyrazolo[1,5-a]pyrimidine-2-carboxamide

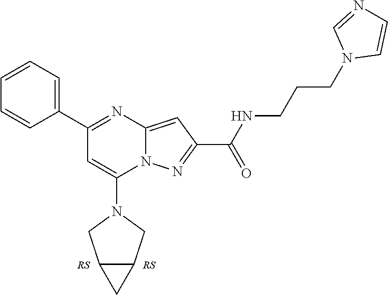 [SMILES: O=C(NCCCn1ccnc1)c1cc2nc(-c3ccccc3)cc(N3CC4CC4C3)n2n1]

Example 61 was synthetized analogously to the method in Example 60 with ethyl 7-chloro-5-phenylpyrazolo[1,5-a]pyrimidine-2-carboxylate (Intermediate 3) (75 mg, 0.25 mmol) and 3-azabicyclo[3.1.0]hexan-3-ium chloride (31 mg, 0.26 mmol), yielding Example 61 as a solid. ¹H NMR (500 MHz, DMSO-d₆) δ ppm 0.37 (q, J=4.2 Hz, 1H) 0.81 (td, J=7.7, 4.7 Hz, 1H) 1.75-1.86 (m, 2H) 2.00 (quin, J=7.0 Hz, 2H) 3.25-3.32 (m, 2H) 3.89 (br d, J=11.0 Hz, 2H) 4.03 (t, J=6.9 Hz, 2H) 4.61 (br d, J=10.7 Hz, 2H) 6.53 (s, 1H) 6.73 (s, 1H) 6.90 (t, J=1.1 Hz, 1H) 7.23 (t, J=1.1 Hz, 1H) 7.45-7.57 (m, 3H) 7.68 (s, 1H) 8.11-8.17 (m, 2H) 8.49 (t, J=6.0 Hz, 1H). MS (ESI+) calculated for C₂₄H₂₅N₇O₂: 427.2, found 428.3 [M+1]⁺.

### Example 62. N-(3-(1H-Imidazol-1-yl)propyl)-7-(3-fluoropiperidin-1-yl)-5-phenylpyrazolo[1,5-a]pyrimidine-2-carboxamide

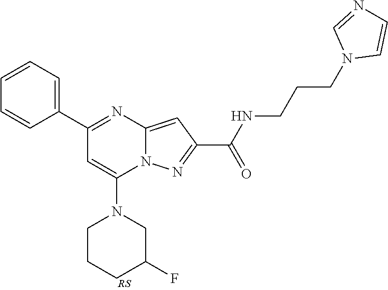 [SMILES: O=C(NCCCn1ccnc1)c1cc2nc(-c3ccccc3)cc(N3CCCC(F)C3)n2n1]

Example 62 was synthetized analogously to the method in Example 60 with ethyl 7-chloro-5-phenylpyrazolo[1,5-a]pyrimidine-2-carboxylate (Intermediate 3) (75 mg, 0.25 mmol) and 3-fluoropiperidine hydrochloride (36 mg, 0.26 mmol), yielding Example 62 as a solid. ¹H NMR (500 MHz, DMSO-d₆) δ ppm 1.66-1.77 (m, 1H) 1.93-2.11 (m, 5H) 3.27-3.31 (m, 2H) 3.58-3.66 (m, 1H) 3.88-4.00 (m, 1H) 4.04 (t, J=6.87 Hz, 2H) 4.14 (br d, J=13.12 Hz, 1H) 4.47-4.58 (m, 1H) 4.87-5.04 (m, 1H) 6.83-6.93 (m, 2H) 6.96 (s, 1H) 7.23 (t, J=1.07 Hz, 1H) 7.47-7.58 (m, 3H) 7.68 (s, 1H) 8.14-8.28 (m, 2H) 8.48 (t, J=5.95 Hz, 1H). MS (ESI+) calculated for C₂₄H₂₆FN₇O: 447.2, found 448.3 [M+H]⁺.

### Example 63. N-(3-(1H-Imidazol-1-yl)propyl)-7-(4-fluoropiperidin-1-yl)-5-phenylpyrazolo[1,5-a]pyrimidine-2-carboxamide

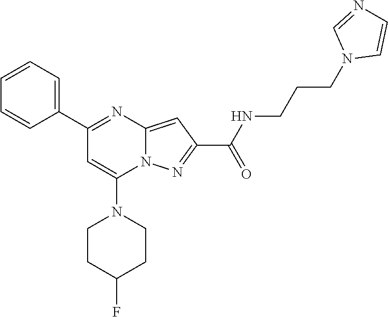 [SMILES: O=C(NCCCn1ccnc1)c1cc2nc(-c3ccccc3)cc(N3CCC(F)CC3)n2n1]

Example 63 was synthetized analogously to the method in Example 60 with ethyl 7-chloro-5-phenylpyrazolo[1,5-a]pyrimidine-2-carboxylate (Intermediate 3) (75 mg, 0.25 mmol) and 4-fluoropiperidine (27 mg, 0.26 mmol) yielding Example 63 as a solid. ¹H NMR (500 MHz, DMSO-d₆) δ ppm 1.92-2.05 (m, 4H) 2.15 (tdd, J=17.70, 17.70, 8.93, 4.20 Hz, 2H) 3.28-3.31 (m, 2H) 3.87-3.94 (m, 2H) 3.94-4.01 (m, 2H) 4.04 (t, J=6.87 Hz, 2H) 4.92-5.10 (m, 1H) 6.81-6.93 (m, 2H) 6.98 (s, 1H) 7.23 (t, J=1.22 Hz, 1H) 7.48-7.57 (m, 3H) 7.68 (s, 1H) 8.15-8.25 (m, 2H) 8.47 (t, J=5.95 Hz, 1H). MS (ESI+) calculated for C₂₄H₂₆FN₇O: 447.2, found 448.3 [M+H]⁺.

### Example 64. N-(3-(1H-Imidazol-1-yl)propyl)-7-(cyclobutylamino)-5-phenylpyrazolo[1,5-a]pyrimidine-2-carboxamide

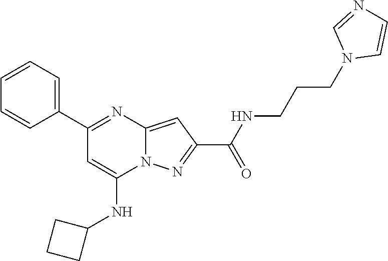 [SMILES: O=C(NCCCn1ccnc1)c1cc2nc(-c3ccccc3)cc(NC3CCC3)n2n1]

Example 64 was synthetized analogously to the method in Example 60 with ethyl 7-chloro-5-phenylpyrazolo[1,5-a]pyrimidine-2-carboxylate (Intermediate 3) (75 mg, 0.25 mmol) and 4-cyclobutylamine (19 mg, 0.27 mmol) yielding Example 64 as a solid. ¹H NMR (500 MHz, DMSO-d₆) δ ppm 1.73-1.86 (m, 2H) 2.01 (quin, J=6.90 Hz, 2H) 2.25 (quind, J=9.31, 9.31, 9.31, 9.31, 2.59 Hz, 2H) 2.42-2.48 (m, 2H) 3.28-3.31 (m, 2H) 4.05 (t, J=6.87 Hz, 2H) 4.44-4.55 (m, 1H) 6.75 (s, 1H) 6.79 (s, 1H) 6.91 (t, J=1.07 Hz, 1H) 7.24 (t, J=1.22 Hz, 1H) 7.48-7.55 (m, 3H) 7.70 (s, 1H) 7.85 (d, J=7.48 Hz, 1H) 8.15-8.22 (m, 2H) 8.36 (t, J=5.95 Hz, 1H). MS (ESI+) calculated for C₂₃H₂₅FN₇O: 415.2, found 416.3 [M+H]⁺.

### Example 65. N-(3-(1H-imidazol-1-yl)propyl)-7-(cyclopentylamino)-5-phenylpyrazolo[1,5-a]pyrimidine-2-carboxamide

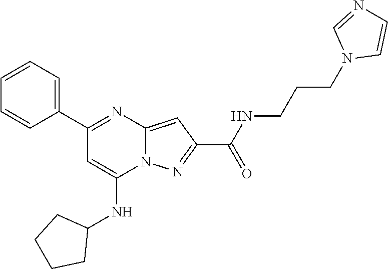 [SMILES: O=C(NCCCn1ccnc1)c1cc2nc(-c3ccccc3)cc(NC3CCCC3)n2n1]

Example 65 was synthetized analogously to the method in Example 60 with ethyl 7-chloro-5-phenylpyrazolo[1,5-a]pyrimidine-2-carboxylate (Intermediate 3) (75 mg, 0.25 mmol) and cyclopentylamine (22 mg, 0.26 mmol) yielding Example 65 as a solid. ¹H NMR (500 MHz, DMSO-d₆) δ ppm 1.61-1.83 (m, 6H) 2.00 (quin, J=6.94 Hz, 2H) 2.12-2.22 (m, 2H) 3.27-3.31 (m, 2H) 4.05 (t, J=6.87 Hz, 2H) 4.35 (sxt, J=6.96 Hz, 1H) 6.78 (s, 1H) 6.85 (s, 1H) 6.90 (t, J=0.99 Hz, 1H) 7.24 (t, J=1.22 Hz, 1H) 7.37 (d, J=7.93 Hz, 1H) 7.48-7.55 (m, 3H) 7.69 (s, 1H) 8.17-8.23 (m, 2H) 8.41 (t, J=5.95 Hz, 1H). MS (ESI+) calculated for C₂₄H₂₇FN₇O: 429.2, found 430.3 [M+H]⁺.

### Example 66. N-(3-(1H-imidazol-1-yl)propyl)-5-phenyl-7-(pyrrolidin-1-yl)pyrazolo[1,5-a]pyrimidine-2-carboxamide

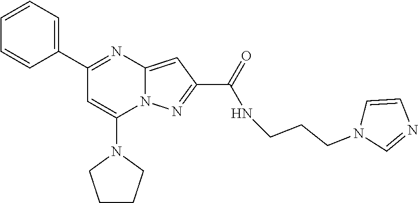 [SMILES: O=C(NCCCn1ccnc1)c1cc2nc(-c3ccccc3)cc(N3CCCC3)n2n1]

Example 66 was synthetized analogously to the method in Example 60 with ethyl 7-chloro-5-phenylpyrazolo[1,5-a]pyrimidine-2-carboxylate (Intermediate 3) (15 mg, 0.05 mmol) and 4-pyrrolidine (0.004 mL, 0.05 mmol) yielding Example 66 as a solid. ¹H NMR (500 MHz, DMSO-d₆) δ ppm 1.94-2.06 (m, 6H) 3.26-3.31 (m, 2H) 4.03 (t, J=6.9 Hz, 2H) 4.03-4.13 (m, 4H) 6.51 (s, 1H) 6.70 (s, 1H) 6.90 (t, J=1.1 Hz, 1H) 7.23 (t, J=1.2 Hz, 1H) 7.45-7.54 (m, 3H) 7.67 (s, 1H) 8.11-8.18 (m, 2H) 8.40 (t, J=6.0 Hz, 1H). MS (ESI+) calculated for C₂₃H₂₅N₇O: 415.1, found 416.3 [M+H]⁺.

### Example 67. (R)-7-(3-hydroxypiperidin-1-yl)-N-(1-methylazetidin-3-yl)-5-phenylpyrazolo[1,5-a]pyrimidine-2-carboxamide

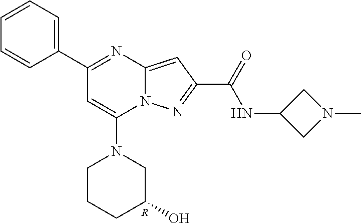

Ethyl 7-chloro-5-phenylpyrazolo[1,5-a]pyrimidine-2-carboxylate (Intermediate 3) (50 mg, 0.146 mmol), Et₃N (41 μL, 0.728 g/mL, 0.295 mmol) and (R)-(+)-3-hydroxypiperidine hydrochloride (21 mg, 0.153 mmol) were suspended in THF (1 mL) and stirred for 1 h at rt. Then, 1-methylazetidin-3-amine dihydrochloride (58 mg, 0.365 mmol) and LHMDS (1M in THF) (1 mL, 1 M, 1 mmol) were added and the reaction was stirred at rt for 1 h. DCM (2 mL) and water were added, and the layers were separated. The collected organic layer was concentrated in vacuo and purified by RP HPLC (Stationary phase: C18 XBridge (5 μm*100 mm); Mobile phase: gradient using NH₄HCO₃ 0.25% solution in water and CH₃CN), yielding Example 67 as a solid. ¹H NMR (500 MHz, DMSO-d₆) δ ppm 1.52-1.58 (m, 1H) 1.63-1.70 (m, 1H) 1.92-1.99 (m, 2H) 2.25-2.28 (m, 3H) 3.01-3.10 (m, 2H) 3.48-3.55 (m, 2H) 3.56-3.61 (m, 2H) 3.81 (td, J=7.74, 4.04 Hz, 1H) 4.10-4.17 (m, 1H) 4.21 (dd, J=12.36, 2.90 Hz, 1H) 4.45 (sxt, J=6.99 Hz, 1H) 4.94 (d, J=4.12 Hz, 1H) 6.88 (s, 1H) 6.92 (s, 1H) 7.50-7.55 (m, 3H) 8.18 (dd, J=7.78, 1.83 Hz, 2H) 8.58 (d, J=7.17 Hz, 1H). MS (ESI+) calculated for C₂₂H₂₆N₆O₂: 406.2, found: 407.3 [M+H]⁺.

### Example 68. N-(1-methylazetidin-3-yl)-5-phenyl-7-(piperidin-1-yl)pyrazolo[1,5-a]pyrimidine-2-carboxamide trifluoroacetate

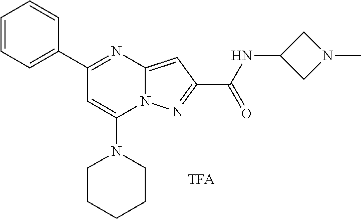 [SMILES: CN1CC(NC(=O)c2cc3nc(-c4ccccc4)cc(N4CCCCC4)n3n2)C1]

To a solution of 5-phenyl-7-(piperidin-1-yl)pyrazolo[1,5-a]pyrimidine-2-carboxylic acid (Intermediate 23) (100 mg, 0.279 mmol) in N,N-dimethylformamide (2 mL) was added 1-methylazetidin-3-amine dihydrochloride (70 mg, 0.44 mmol), triethylamine (0.25 mL, 1.8 mmol) and HATU (160 mg, 0.421 mmol) at rt. After stirred at rt overnight, the reaction mixture was diluted with water (10 mL) and extracted with EtOAc (3×10 mL). The combined organic layer was dried over Na₂SO₄ and concentrated to afford a crude product, which was purified by prep. HPLC (preparation method: SunFire® Prep C18 OBD™ (5 μm 19*150 mm), Mobile Phase A: water (0.01% trifluoroacetic acid), Mobile Phase B: acetonitrile, UV: 214 nm, fFlow rate: 15 mL/min, Gradient: 5-95% (% B)) yielding Example 65 as light yellow solid. ¹H NMR (400 MHz, DMSO-d₆) δ ppm 1.75 (s, 6H) 2.93 (d, J=5.2 Hz, 3H) 3.87 (s, 4H) 4.24-4.17 (m, 2H) 4.53-4.46 (m, 2H) 4.88-4.75 (m, 1H) 6.96-6.92 (m, 2H) 7.55-7.53 (m, 3H) 8.21-8.19 (m, 2H) 9.02-8.98 (m, 1H) 9.79 (br s, 1H). MS (ESI+) calculated for C₂₂H₂₆N₆O 390.2, found 391.2 [M+H]⁺.

### Example 69. N-((1-methylpyrrolidin-3-yl)methyl)-5-phenyl-7-(piperidin-1-yl)pyrazolo[1,5-a]pyrimidine-2-carboxamide

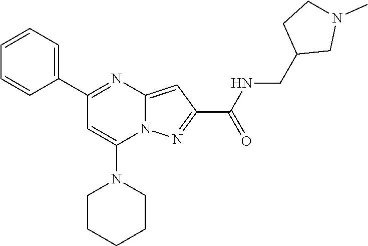 [SMILES: CN1CCC(CNC(=O)c2cc3nc(-c4ccccc4)cc(N4CCCCC4)n3n2)C1]

Example 69 was synthetized analogously to the method in Example 68 with 5-phenyl-7-(piperidin-1-yl)pyrazolo[1,5-a]pyrimidine-2-carboxylic acid (Intermediate 23) (200 mg, 0.279 mmol) and (1-methylpyrrolidin-3-yl)methanamine (0.1 mL, 0.771 mmol), yielding Example 69 as a solid. ¹H NMR (400 MHz, DMSO-d₆) δ ppm 1.55-1.47 (m, 1H) 1.81-1.74 (m, 6H), 1.92-1.84 (m, 1H), 2.23 (s, 3H), 2.37-2.29 (m, 2H), 2.45-2.42 (m, 2H), 2.57-2.53 (m, 1H), 3.30-3.24 (m, 2H), 3.85-3.84 (m, 4H), 6.86 (s, 1H), 6.90 (s, 1H), 7.54-7.51 (m, 3H), 8.20-8.18 (m, 2H), 8.39-8.36 (m, 1H). MS (ESI+) calculated for C₂₄H₃₀N₆O: 418.3, found: 419.3 [M+H]⁺.

### Example 70. N-(3-(dimethylamino)propyl)-5-phenyl-7-(piperidin-1-yl)pyrazolo[1,5-a]pyrimidine-2-carboxamide

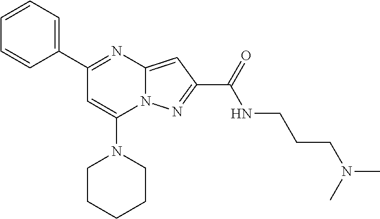 [SMILES: CN(C)CCCNC(=O)c1cc2nc(-c3ccccc3)cc(N3CCCCC3)n2n1]

Example 70 was synthetized analogously to the method in Example 68 with 5-phenyl-7-(piperidin-1-yl)pyrazolo[1,5-a]pyrimidine-2-carboxylic acid (Intermediate 23) (100 mg, 0.279 mmol) and N1,N1-dimethylpropane-1,3-diamine (0.05 mL, 0.397 mmol), yielding Example 70 as a solid. ¹H NMR (400 MHz, DMSO-d₆) δ ppm 1.74-1.70 (m, 8H), 2.15 (s, 6H), 2.30 (t, J=6.8 Hz, 2H), 3.35 (dd, J=12.8, 6.4 Hz, 2H), 3.85-3.84 (m, 4H), 6.85 (s, 1H), 6.90 (s, 1H), 7.55-7.51 (m, 3H), 8.20-8.18 (m, 2H), 8.43-8.40 (m, 1H). MS (ESI+) calculated for C₂₃H₃₀N₆O: 406.3, found: 407.2 [M+H]⁺.

### Example 71. N-(1-methylpyrrolidin-3-yl)-5-phenyl-7-(piperidin-1-yl)pyrazolo[1,5-a]pyrimidine-2-carboxamide trifluoroacetate

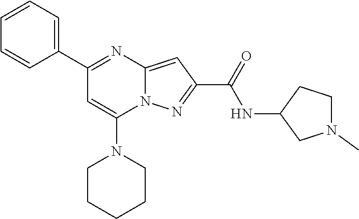 [SMILES: CN1CCC(NC(=O)c2cc3nc(-c4ccccc4)cc(N4CCCCC4)n3n2)C1]

Example 71 was synthetized analogously to the method in Example 68 with 5-phenyl-7-(piperidin-1-yl)pyrazolo[1,5-a]pyrimidine-2-carboxylic acid (Intermediate 23) (100 mg, 0.279 mmol) and 1-methylazetidin-3-amine dihydrochloride (74 mg, 0.466 mmol), yielding Example 71 as a solid. ¹H NMR (400 MHz, DMSO-d₆) δ ppm 1.74 (s, 6H), 2.34-1.91 (m, 2H), 2.93-2.88 (m, 3H), 3.16-3.07 (m, 1H), 3.43-3.26 (m, 1H), 3.76-3.61 (m, 2H), 3.92-3.86 (m, 4H), 4.70-4.59 (m, 1H), 6.94-6.92 (m, 2H), 7.55-7.53 (m, 3H), 8.21-8.19 (m, 2H), 8.70-8.61 (m, 1H), 9.97 (br s, 1H). MS (ESI+) calculated for C₂₃H₂₈N₆O: 404.2, found: 405.2 [M+H]⁺.

### Example 72. (*R)—N-(1-methylpyrrolidin-3-yl)-5-phenyl-7-(piperidin-1-yl)pyrazolo[1,5-a]pyrimidine-2-carboxamide trifluoroacetate

Example 72 was obtained by chiral Prep. HPLC of Example 71 (separation condition: Column: Chiralpak OJ-H 5 μm 20*250 mm; Mobile Phase: Hex:EtOH:DEA=80:20:0.2 at 30 mL/min; Temp: 30° C.; Wavelength: 254 nm) to afford example 72.

SFC: 99.9% ee at Rt=8.01 min

### Example 73. (*S)—N-(1-methylpyrrolidin-3-yl)-5-phenyl-7-(piperidin-1-yl)pyrazolo[1,5-a]pyrimidine-2-carboxamide trifluoroacetate

Example 73 was obtained by chiral Prep. HPLC of Example 71 (separation condition: Column: Chiralpak OJ-H 5 μm 20*250 mm; Mobile Phase: Hex:EtOH:DEA=80:20:0.2 at 30 mL/min; Temp: 30° C.; Wavelength: 254 nm) to afford compound of Example 73.

SFC: 97.5% ee at Rt=10.19 min

### Example 74. N-(1-methylpiperidin-4-yl)-5-phenyl-7-(piperidin-1-yl)pyrazolo[1,5-a]pyrimidine-2-carboxamide

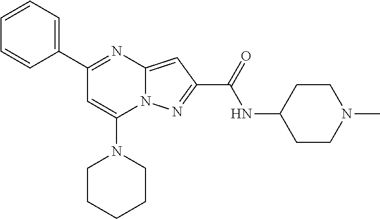 [SMILES: CN1CCC(NC(=O)c2cc3nc(-c4ccccc4)cc(N4CCCCC4)n3n2)CC1]

Example 74 was synthetized analogously to the method in Example 68 with 5-phenyl-7-(piperidin-1-yl)pyrazolo[1,5-a]pyrimidine-2-carboxylic acid (Intermediate 23) (100 mg, 0.279 mmol) and 1-methylpiperidin-4-amine (0.05 mL, 0.4 mmol), yielding Example 74 as a solid. ¹H NMR (400 MHz, DMSO-d₆) δ ppm 1.82-1.67 (m, 10H) 2.08-2.03 (m, 2H), 2.20 (s, 3H), 2.78-2.75 (m, 2H), 3.84-3.79 (m, 5H), 6.91-6.89 (m, 2H), 7.54-7.51 (m, 3H), 8.03-8.01 (m, 1H), 8.20-8.18 (m, 2H). MS (ESI+) calculated for C₂₄H₃₀N₆O: 418.2, found: 419.2 [M+H]⁺.

### Example 75. 7-isopropoxy-N-(1-methylazetidin-3-yl)-5-phenylpyrazolo[1,5-a]pyrimidine-2-carboxamide

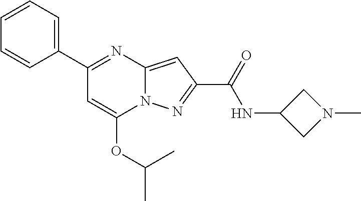 [SMILES: CC(C)Oc1cc(-c2ccccc2)nc2cc(C(=O)NC3CN(C)C3)nn12]

Ethyl 7-chloro-5-phenylpyrazolo[1,5-a]pyrimidine-2-carboxylate (Intermediate 3) (50 mg, 0.146 mmol), iPrOH (26 μL, 0.785 g/mL, 0.34 mmol) and Cs₂CO₃ (53 mg, 0.163 mmol) were suspended in THF (1 mL) and stirred at 55° C. for 16 h. Then, a solution of 1-methylazetidin-3-amine dihydrochloride (58 mg, 0.365 mmol) and LHMDS (1.06 M in THF) (1.5 mL, 1.06 M in THF, 1.5 mmol) were added and the reaction was stirred for 15 min at rt. Water (2 mL) was added, and the product was extracted with EtOAc (3×2 mL). The combined organic layers were concentrated in vacuo and purified by RP HPLC (Stationary phase: C18 XBridge 30×100 mm 5 μm, Mobile phase: NH₄HCO₃ 0.25% solution in water and CH₃CN). The desired fractions were collected and concentrated in vacuo to yield example 75 as a solid. ¹H NMR (400 MHz, DMSO-d₆) δ ppm 1.53 (d, J=6.24 Hz, 6H) 2.27 (s, 3H) 3.11 (t, J=7.28 Hz, 2H) 3.54-3.59 (m, 2H) 4.48 (sxt, J=7.07 Hz, 1H) 5.47 (spt, J=6.09 Hz, 1H) 6.97 (s, 1H) 7.24-7.33 (m, 1H) 7.52-7.59 (m, 3H) 8.22-8.29 (m, 2H) 8.79 (d, J=7.40 Hz, 1H). MS (ESI+) calculated for C₂₀H₂₃N₅O₂: 365.2; found: 366.2 [M+H]⁺.

### Example 76. N-(1-methylazetidin-3-yl)-7-phenoxy-5-phenylpyrazolo[1,5-a]pyrimidine-2-carboxamide

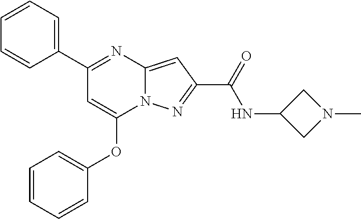 [SMILES: CN1CC(NC(=O)c2cc3nc(-c4ccccc4)cc(Oc4ccccc4)n3n2)C1]

1-Methylazetidin-3-amine (10 mg, 0.116 mmol) was added to a mixture of HATU (44 mg, 0.116 mmol), 7-phenoxy-5-phenylpyrazolo[1,5-a]pyrimidine-2-carboxylic acid (Intermediate 18) (31.9 mg, 0.1 mmol), and DIPEA (20 μL, 0.75 g/mL, 0.116 mmol) in DMF (0.5 mL). The reaction was stirred at rt for 20 h. Then, water (2 mL) was added, and the product was extracted in EtOAc (3×2 mL). The combined organic extracts were concentrated in vacuo and the resulting crude was purified by RP HPLC (Stationary phase: C18 XBridge, column with 100 mm length, 5 μm. Mobile phase: Gradient using NH₄HCO₃ 0.25% solution in water and CH₃CN). The desired fractions were collected and concentrated in vacuo yielding Example 76 as a white solid. ¹H NMR (500 MHz, DMSO-d₆) δ ppm 2.27 (s, 3H), 3.07-3.14 (m, 2H) 3.57 (br t, J=6.2 Hz, 2H) 4.51 (sxt, J=7.2 Hz, 1H) 6.60 (s, 1H) 7.11 (s, 1H) 7.45-7.57 (m, 6H) 7.60-7.66 (m, 2H) 7.94-7.99 (m, 2H) 8.96 (d, J=7.5 Hz, 1H). MS (ESI+) calculated for C₂₃H₂₁N₅O₂: 399.2; found: 400.2 [M+H]⁺.

### Example 77. N-(3-(1H-imidazol-1-yl)propyl)-7-(3-acetamidophenyl)-5-phenylpyrazolo[1,5-a]pyrimidine-2-carboxamide

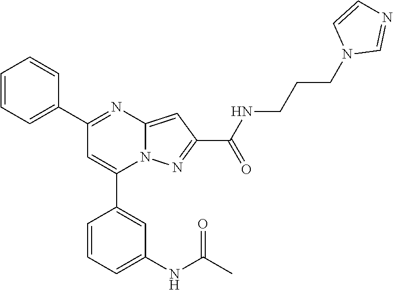 [SMILES: CC(=O)Nc1cccc(-c2cc(-c3ccccc3)nc3cc(C(=O)NCCCn4ccnc4)nn23)c1]

Ethyl 7-chloro-5-phenylpyrazolo[1,5-a]pyrimidine-2-carboxylate (Intermediate 3) (60.3 mg, 0.2 mmol), Pd(PPh₃)₄ (23 mg, 0.02 mmol) and (3-acetamidophenyl)boronic acid (53.7 mg, 0.3 mmol) were suspended in a mixture of 1,4-dioxane (1.3 mL) and aq Na₂CO₃ solution (0.7 mL, 100 g/L). The reaction was stirred at 60° C. for 3 h. Water (2 mL) was added and the aqueous layer was extracted in EtOAc (3×2 mL). The combined organic extracts were concentrated in vacuo. Then, LiOH (5.7 mg, 0.24 mmol), water (0.1 mL) and THF (1 mL) were added, and the reaction was stirred at 60° C. for 1 h. The solvents were distilled in vacuo. Then, a solution of HATU (152 mg, 0.4 mmol), 1-(3-aminopropyl)imidazole (75 mg, 0.599 mmol) and DIPEA (150 μL, 0.75 g/mL, 0.87 mmol) in DMF (1 mL) was added, and the reaction was stirred at 50° C. for 30 min. Then, water was added (2 mL), and the product was extracted with EtOAc (3×2 mL). The combined organic extracts were concentrated in vacuo. The resulting crude was purified by RP HPLC (Stationary phase: C18 XBridge 100 mm×5 μm; Mobile phase: Gradient using NH₄HCO₃ 0.25% solution in water and CH₃CN). The desired fractions were collected and concentrated in vacuo yielding Example 77 as a solid. ¹H NMR (500 MHz, DMSO-d₆) δ ppm 2.02 (quin, J=6.9 Hz, 2H) 2.09 (s, 3H) 3.29-3.34 (m, 2H) 4.04 (t, J=6.9 Hz, 2H) 6.89 (br s, 1H) 7.16 (s, 1H) 7.21 (t, J=1.1 Hz, 1H) 7.54-7.61 (m, 4H) 7.68 (s, 1H) 7.78 (dd, J=8.2, 1.1 Hz, 1H) 7.90-7.93 (m, 1H) 7.94 (s, 1H) 8.30-8.36 (m, 2H) 8.42 (t, J=6.0 Hz, 1H) 8.75 (t, J=1.8 Hz, 1H) 10.33 (s, 1H). MS (ESI+) calculated for C₂₇H₂₅N₇O₂: 479.2, found: 480.2 [M+H]⁺.

### Example 78. N-(3-(1H-imidazol-1-yl)propyl)-7-(3-fluorophenyl)-5-phenylpyrazolo[1,5-a]pyrimidine-2-carboxamide

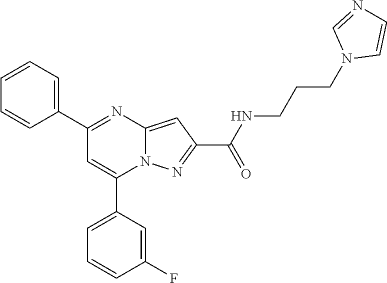 [SMILES: O=C(NCCCn1ccnc1)c1cc2nc(-c3ccccc3)cc(-c3cccc(F)c3)n2n1]

Example 78 was synthetized analogously to the method in Example 77 using ethyl 7-chloro-5-phenylpyrazolo[1,5-a]pyrimidine-2-carboxylate (Intermediate 3) (60.3 mg, 0.2 mmol) and (3-fluorophenyl)boronic acid (41.98 mg, 0.3 mmol) yielding Example 78 as a solid. ¹H NMR (500 MHz, DMSO-d₆) δ ppm 1.99 (quin, J=6.94 Hz, 2H) 3.29 (br d, J=6.41 Hz, 2H) 4.02 (t, J=6.94 Hz, 2H) 6.87-6.91 (m, 1H) 7.18 (s, 1H) 7.22 (t, J=1.14 Hz, 1H) 7.49-7.55 (m, 1H) 7.56-7.61 (m, 3H) 7.66 (s, 1H) 7.70 (td, J=8.01, 6.26 Hz, 1H) 7.99 (s, 1H) 8.15-8.22 (m, 2H) 8.33-8.39 (m, 2H) 8.54 (t, J=6.03 Hz, 1H); MS (ESI+) calculated for C₂₅H₂₁FN₆O: 440.2; found: 441.3 [M+H]⁺.

### Example 79. N-(3-(1H-imidazol-1-yl)propyl)-7-(4-methoxyphenyl)-5-phenylpyrazolo[1,5-a]pyrimidine-2-carboxamide

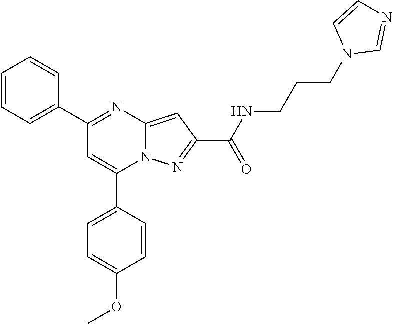 [SMILES: COc1ccc(-c2cc(-c3ccccc3)nc3cc(C(=O)NCCCn4ccnc4)nn23)cc1]

Example 79 was synthetized analogously to the method in Example 77 using ethyl 7-chloro-5-phenylpyrazolo[1,5-a]pyrimidine-2-carboxylate (Intermediate 3) (60.3 mg, 0.2 mmol) and (4-methoxyphenyl)boronic acid (45.6 mg, 0.3 mmol) yielding Example 79 as a white solid. ¹H NMR (500 MHz, DMSO-d₆) δ ppm 1.96-2.04 (m, 2H) 3.31 (br s, 2H) 3.91 (s, 3H) 4.03 (t, J=6.94 Hz, 2H) 6.89 (t, J=0.99 Hz, 1H) 7.13 (s, 1H) 7.19 (s, 1H) 7.20 (s, 1H) 7.22 (t, J=1.22 Hz, 1H) 7.56-7.60 (m, 3H) 7.67 (s, 1H) 7.90 (s, 1H) 8.32-8.35 (m, 2H) 8.37-8.40 (m, 2H) 8.50 (t, J=5.95 Hz, 1H). MS (ESI+) calculated for C₂₆H₂₄N₆O₂: 452.2, found: 453.3 [M+H]⁺.

### Example 80. N-(3-(1H-imidazol-1-yl)propyl)-7-(2-methoxyphenyl)-5-phenylpyrazolo[1,5-a]pyrimidine-2-carboxamide

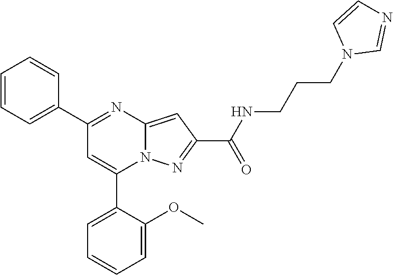 [SMILES: COc1ccccc1-c1cc(-c2ccccc2)nc2cc(C(=O)NCCCn3ccnc3)nn12]

Example 80 was synthetized analogously to the method in Example 77 using ethyl 7-chloro-5-phenylpyrazolo[1,5-a]pyrimidine-2-carboxylate (Intermediate 3) (60.3 mg, 0.2 mmol) and (2-methoxyphenyl)boronic acid (45.6 mg, 0.3 mmol) yielding Example 80 as a solid. ¹H NMR (500 MHz, DMSO-d₆) δ ppm 1.94 (quin, J=6.90 Hz, 2H) 3.25 (q, J=6.66 Hz, 2H) 3.74-3.83 (m, 3H) 3.98 (t, J=6.94 Hz, 2H) 6.87 (s, 1H) 7.14 (s, 1H) 7.16-7.20 (m, 2H) 7.30 (d, J=8.24 Hz, 1H) 7.54-7.58 (m, 3H) 7.60-7.65 (m, 2H) 7.67 (dd, J=7.63, 1.68 Hz, 1H) 7.79 (s, 1H) 8.28 (td, J=3.97, 1.83 Hz, 2H) 8.33 (t, J=5.95 Hz, 1H). MS (ESI+) calculated for C₂₆H₂₄N₆O₂: 452.2, found: 453.3[M+H]⁺.

### Example 81. N-(3-(1H-imidazol-1-yl)propyl)-5-phenyl-7-(m-tolyl)pyrazolo[1,5-a]pyrimidine-2-carboxamide

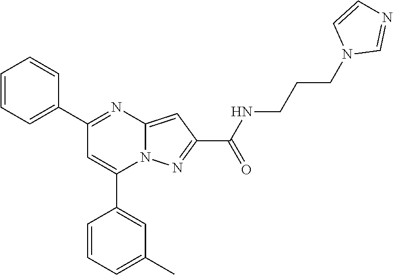 [SMILES: Cc1cccc(-c2cc(-c3ccccc3)nc3cc(C(=O)NCCCn4ccnc4)nn23)c1]

Example 81 was synthetized analogously to the method in Example 77 using ethyl 7-chloro-5-phenylpyrazolo[1,5-a]pyrimidine-2-carboxylate (Intermediate 3) (60.3 mg, 0.2 mmol) and m-tolylboronic acid (40.8 mg, 0.3 mmol) yielding Example 81 as a white solid. ¹H NMR (500 MHz, DMSO-d₆) δ ppm 1.99 (quin, J=6.94 Hz, 2H) 2.47 (s, 3H) 3.26-3.31 (m, 2H) 4.02 (t, J=6.94 Hz, 2H) 6.88 (t, J=0.99 Hz, 1H) 7.16 (s, 1H) 7.21 (t, J=1.22 Hz, 1H) 7.46-7.50 (m, 1H) 7.52-7.56 (m, 1H) 7.56-7.61 (m, 3H) 7.66 (s, 1H) 7.89 (s, 1H) 8.05 (s, 1H) 8.12 (d, J=7.63 Hz, 1H) 8.28-8.38 (m, 2H) 8.47 (t, J=5.95 Hz, 1H). MS (ESI+) calculated for C₂₆H₂₄N₆O: 436.2, found: 437.3 [M+H]⁺.

### Example 82. N-(3-(1H-imidazol-1-yl)propyl)-7-(3-methoxyphenyl)-5-phenylpyrazolo[1,5-a]pyrimidine-2-carboxamide

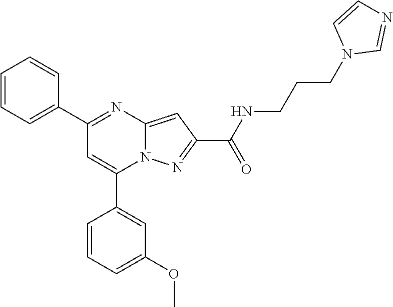 [SMILES: COc1cccc(-c2cc(-c3ccccc3)nc3cc(C(=O)NCCCn4ccnc4)nn23)c1]

Example 82 was synthetized analogously to the method in Example 77 using ethyl 7-chloro-5-phenylpyrazolo[1,5-a]pyrimidine-2-carboxylate (Intermediate 3) (60.3 mg, 0.2 mmol) and (3-methoxyphenyl)boronic acid (45.6 mg, 0.3 mmol) yielding Example 82 as a solid. ¹H NMR (500 MHz, DMSO-d₆) δ ppm 1.95-2.02 (m, 2H) 3.26-3.31 (m, 2H) 3.89 (s, 3H) 4.01 (t, J=6.94 Hz, 2H) 6.88 (t, J=0.99 Hz, 1H) 7.17 (s, 1H) 7.21 (t, J=1.22 Hz, 1H) 7.23 (ddd, J=8.32, 2.59, 0.69 Hz, 1H) 7.54-7.61 (m, 4H) 7.66 (s, 1H) 7.80 (s, 1H) 7.85 (dt, J=7.74, 1.16 Hz, 1H) 7.92 (s, 1H) 8.31-8.37 (m, 2H) 8.48 (t, J=5.87 Hz, 1H). MS (ESI+) calculated for C₂₆H₂₄N₆O₂: 452.2, found: 453.2 [M+H]⁺.

### Example 83. N-(3-(1H-imidazol-1-yl)propyl)-5-phenyl-7-(p-tolyl)pyrazolo[1,5-a]pyrimidine-2-carboxamide

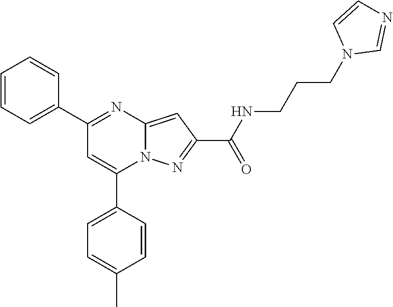 [SMILES: Cc1ccc(-c2cc(-c3ccccc3)nc3cc(C(=O)NCCCn4ccnc4)nn23)cc1]

Example 83 was synthetized analogously to the method in Example 77 using ethyl 7-chloro-5-phenylpyrazolo[1,5-a]pyrimidine-2-carboxylate (Intermediate 3) p-tolylboronic acid (40.8 mg, 0.3 mmol) yielding Example 83 as a solid. ¹H NMR (500 MHz, DMSO-d₆) δ ppm 1.99 (quin, J=6.90 Hz, 2H) 2.46 (s, 3H) 3.30 (br d, J=6.71 Hz, 2H) 4.02 (t, J=6.94 Hz, 2H) 6.89 (t, J=0.99 Hz, 1H) 7.14 (s, 1H) 7.22 (t, J=1.22 Hz, 1H) 7.47 (d, J=7.93 Hz, 2H) 7.54-7.61 (m, 3H) 7.67 (s, 1H) 7.89 (s, 1H) 8.19-8.27 (m, 2H) 8.29-8.38 (m, 2H) 8.48 (t, J=5.95 Hz, 1H). MS (ESI+) calculated for C₂₆H₂₄N₆O: 436.2, found: 437.3 [M+H]⁺.

### Example 84. N-(3-(1H-imidazol-1-yl)propyl)-7-(benzo[c][1,2,5]oxadiazol-5-yl)-5-phenylpyrazolo[1,5-a]pyrimidine-2-carboxamide

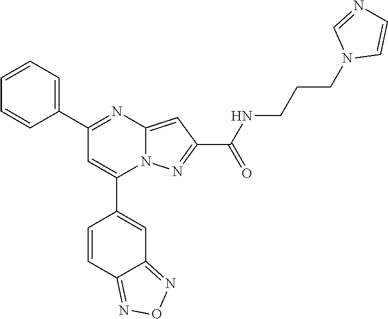 [SMILES: O=C(NCCCn1ccnc1)c1cc2nc(-c3ccccc3)cc(-c3ccc4nonc4c3)n2n1]

Example 84 was synthetized analogously to the method in Example 77 using ethyl 7-chloro-5-phenylpyrazolo[1,5-a]pyrimidine-2-carboxylate (Intermediate 3) (60.3 mg, 0.2 mmol) and 5-(4,4,5,5-tetramethyl-1,3,2-dioxaborolan-2-yl)benzo[c][1,2,5]oxadiazole (73.8 mg, 0.3 mmol) yielding Example 84 as a solid. ¹H NMR (500 MHz, DMSO-d₆) δ ppm 1.98 (quin, J=6.9 Hz, 2H) 3.22-3.31 (m, 2H) 4.01 (t, J=6.9 Hz, 2H) 6.88 (t, J=1.0 Hz, 1H) 7.21 (t, J=1.1 Hz, 1H) 7.23 (s, 1H) 7.51-7.64 (m, 3H) 7.64-7.67 (m, 1H) 8.18 (s, 1H) 8.28-8.32 (m, 1H) 8.33-8.36 (m, 1H) 8.36-8.40 (m, 2H) 8.57 (t, J=6.0 Hz, 1H) 9.08 (t, J=1.1 Hz, 1H). MS (ESI+) calculated for C₂₅H₂₀N₈O₂: 464.2, found: 465.2 [M+H]⁺.

### Example 85. N-(3-(1H-imidazol-1-yl)propyl)-7-(1-methyl-1H-pyrazol-3-yl)-5-phenylpyrazolo[1,5-a]pyrimidine-2-carboxamide

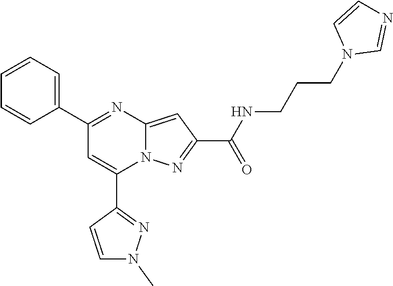 [SMILES: Cn1ccc(-c2cc(-c3ccccc3)nc3cc(C(=O)NCCCn4ccnc4)nn23)n1]

Example 85 was synthetized analogously to the method in Example 77 using ethyl 7-chloro-5-phenylpyrazolo[1,5-a]pyrimidine-2-carboxylate (Intermediate 3) (60.3 mg, 0.2 mmol) and 1-methyl-3-(4,4,5,5-tetramethyl-1,3,2-dioxaborolan-2-yl)-1H-pyrazole (62.4 mg, 0.3 mmol) yielding Example 85. ¹H NMR (500 MHz, DMSO-d₆) δ ppm 2.04 (quin, J=6.94 Hz, 2H) 3.35-3.39 (m, 2H) 4.03-4.11 (m, 5H) 6.91 (t, J=0.92 Hz, 1H) 7.14 (s, 1H) 7.25 (t, J=1.22 Hz, 1H) 7.51-7.64 (m, 3H) 7.69 (s, 1H) 8.06-8.11 (m, 2H) 8.19-8.25 (m, 3H) 8.75 (t, J=6.03 Hz, 1H). MS (ESI+) calculated for C₂₃H₂₂N₈O: 426.2; found: 427.3 [M+H]⁺.

### Example 86. N-(3-(1H-imidazol-1-yl)propyl)-7-(4-fluorophenyl)-5-phenylpyrazolo[1,5-a]pyrimidine-2-carboxamide

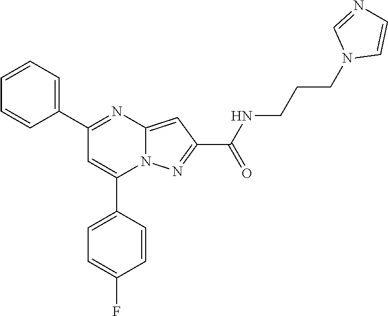 [SMILES: O=C(NCCCn1ccnc1)c1cc2nc(-c3ccccc3)cc(-c3ccc(F)cc3)n2n1]

Example 86 was synthetized analogously to the method in Example 77 using ethyl 7-chloro-5-phenylpyrazolo[1,5-a]pyrimidine-2-carboxylate (Intermediate 3) (60.3 mg, 0.2 mmol) and (4-fluorophenyl)boronic acid 41.98 mg, 0.3 mmol) yielding Example 86. ¹H NMR (500 MHz, DMSO-d₆) δ ppm 1.94-2.03 (m, 2H) 3.27-3.31 (m, 2H) 4.02 (t, J=6.87 Hz, 2H) 6.89 (t, J=0.99 Hz, 1H) 7.16 (s, 1H) 7.22 (t, J=1.22 Hz, 1H) 7.49 (t, J=8.93 Hz, 2H) 7.54-7.62 (m, 3H) 7.67 (s, 1H) 7.95 (s, 1H) 8.32-8.37 (m, 2H) 8.38-8.45 (m, 2H) 8.50 (t, J=5.95 Hz, 1H). MS (ESI+) calculated for C₂₅H₂₁FN₆O: 440.2; found: 441.3 [M+H]⁺.

### Example 87. N-(3-(1H-imidazol-1-yl)propyl)-7-(3-hydroxyphenyl)-5-phenylpyrazolo[1,5-a]pyrimidine-2-carboxamide

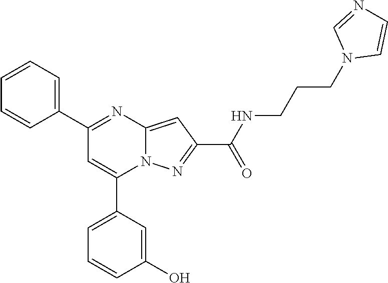 [SMILES: O=C(NCCCn1ccnc1)c1cc2nc(-c3ccccc3)cc(-c3cccc(O)c3)n2n1]

Example 87 was synthetized analogously to the method in Example 77 using ethyl 7-chloro-5-phenylpyrazolo[1,5-a]pyrimidine-2-carboxylate (Intermediate 3) (60.3 mg, 0.2 mmol) and (3-hydroxyphenyl)boronic acid (41.38 mg, 0.3 mmol) yielding Example 87 as a solid. ¹H NMR (500 MHz, DMSO-d₆) δ ppm 2.00 (quin, J=6.8 Hz, 2H) 3.25-3.37 (m, 2H) 4.03 (t, J=6.7 Hz, 2H) 6.90 (t, J=1.0 Hz, 1H) 7.04 (ddd, J=8.2, 2.4, 0.6 Hz, 1H) 7.14 (s, 1H) 7.22 (t, J=1.1 Hz, 1H) 7.43 (t, J=7.9 Hz, 1H) 7.54-7.60 (m, 3H) 7.63 (d, J=7.6 Hz, 1H) 7.68 (t, J=2.0 Hz, 1H) 7.72 (s, 1H) 7.85 (s, 1H) 8.28-8.35 (m, 3H) 10.19 (s, 1H). MS (ESI+) calculated for C₂₅H₂₂N₆O₂: 438.2; found: 439.2 [M+H]⁺.

### Example 88. N-(3-(1H-imidazol-1-yl)propyl)-7-(4-hydroxyphenyl)-5-phenylpyrazolo[1,5-a]pyrimidine-2-carboxamide

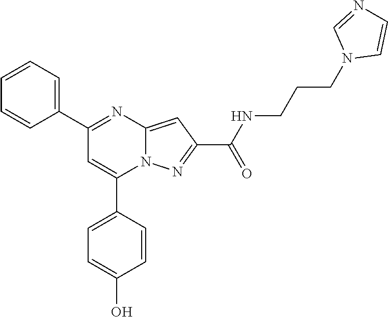 [SMILES: O=C(NCCCn1ccnc1)c1cc2nc(-c3ccccc3)cc(-c3ccc(O)cc3)n2n1]

Example 88 was synthetized analogously to the method in Example 77 using ethyl 7-chloro-5-phenylpyrazolo[1,5-a]pyrimidine-2-carboxylate (Intermediate 3) (60.3 mg, 0.2 mmol) and (4-hydroxyphenyl)boronic acid (41.38 mg, 0.3 mmol) yielding Example 88. ¹H NMR (500 MHz, chloroform-d) δ ppm 2.18 (quin, J=6.79 Hz, 2H) 3.48 (q, J=6.51 Hz, 2H) 4.10 (t, J=6.87 Hz, 2H) 7.01 (s, 1H) 7.09-7.14 (m, 3H) 7.27 (s, 2H) 7.41 (s, 1H) 7.49-7.55 (m, 3H) 7.61 (s, 1H) 7.96 (d, J=8.70 Hz, 2H) 8.12 (dd, J=7.48, 1.83 Hz, 2H) 9.89-11.87 (m, 1H). MS (ESI+) calculated for C₂₅H₂₂N₆O₂: 438.2; found: 439.2 [M+H]⁺.

### Example 89. N-(3-(1H-imidazol-1-yl)propyl)-7-(3-cyanophenyl)-5-phenylpyrazolo[1,5-a]pyrimidine-2-carboxamide

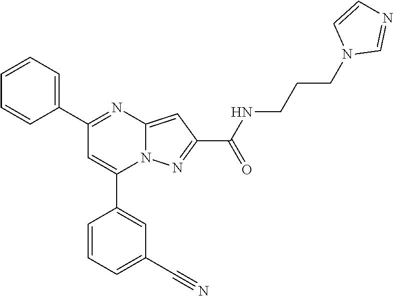 [SMILES: N#Cc1cccc(-c2cc(-c3ccccc3)nc3cc(C(=O)NCCCn4ccnc4)nn23)c1]

Example 89 was synthetized analogously to the method in Example 77 using ethyl 7-chloro-5-phenylpyrazolo[1,5-a]pyrimidine-2-carboxylate (Intermediate 3) (60.3 mg, 0.2 mmol) and 3-(4,4,5,5-tetramethyl-1,3,2-dioxaborolan-2-yl)benzonitrile (68.7 mg, 0.3 mmol) yielding Example 89. ¹H NMR (500 MHz, DMSO-d₆) δ ppm 1.99 (quin, J=6.94 Hz, 2H) 3.27-3.30 (m, 2H) 4.02 (t, J=6.87 Hz, 2H) 6.88 (t, J=0.99 Hz, 1H) 7.19 (s, 1H) 7.22 (t, J=1.14 Hz, 1H) 7.55-7.63 (m, 3H) 7.66 (s, 1H) 7.86 (t, J=7.93 Hz, 1H) 8.08 (s, 1H) 8.13 (dt, J=7.78, 1.30 Hz, 1H) 8.32-8.42 (m, 2H) 8.56 (t, J=5.95 Hz, 1H) 8.68-8.72 (m, 1H) 8.73 (t, J=1.45 Hz, 1H). MS (ESI+) calculated for C₂₆H₂₁N₇O: 447.2; found: 448.3 [M+H]⁺.

### Example 90. N-(3-(1H-imidazol-1-yl)propyl)-7-(4-cyanophenyl)-5-phenylpyrazolo[1,5-a]pyrimidine-2-carboxamide

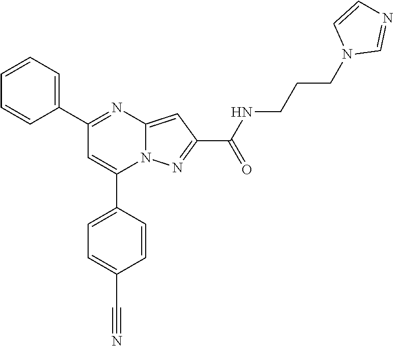 [SMILES: N#Cc1ccc(-c2cc(-c3ccccc3)nc3cc(C(=O)NCCCn4ccnc4)nn23)cc1]

Example 90 was synthetized analogously to the method in Example 77 using ethyl 7-chloro-5-phenylpyrazolo[1,5-a]pyrimidine-2-carboxylate (Intermediate 3) (60.3 mg, 0.2 mmol) and commercially available 4-(4,4,5,5-tetramethyl-1,3,2-dioxaborolan-2-yl)benzonitrile (68.7 mg, 0.3 mmol) yielding Example 90 as a solid. ¹H NMR (500 MHz, DMSO-d₆) δ ppm 1.95-2.02 (m, 2H) 3.25-3.31 (m, 2H) 4.02 (t, J=6.87 Hz, 2H) 6.89 (t, J=0.99 Hz, 1H) 7.20 (s, 1H) 7.21 (t, J=1.14 Hz, 1H) 7.57-7.61 (m, 3H) 7.66 (s, 1H) 8.04 (s, 1H) 8.11-8.15 (m, 2H) 8.33-8.38 (m, 2H) 8.47-8.54 (m, 3H). MS (ESI+) calculated for C₂₆H₂₁N₇O: 447.2; found: 448.3 [M+H]⁺.

### Example 91. N-(3-(1H-imidazol-1-yl)propyl)-5-phenyl-7-(pyridin-2-yl)pyrazolo[1,5-a]pyrimidine-2-carboxamide

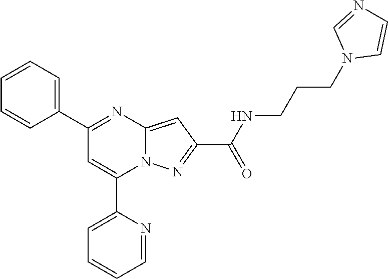 [SMILES: O=C(NCCCn1ccnc1)c1cc2nc(-c3ccccc3)cc(-c3ccccn3)n2n1]

5-Phenyl-7-(pyridin-2-yl)pyrazolo[1,5-a]pyrimidine-2-carboxylic acid (Intermediate 24) (50 mg, 0.095 mmol) was dissolved in DMF (2 mL) and then, Et₃N (0.04 mL, 0.73 g/mL, 0.28 mmol) and HATU (0.054 g, 0.14 mmol) were added followed by 1-(3-aminopropyl)imidazole (18 mg, 0.14 mmol). the mixture was stirred at rt for 12 h. aq Na₂CO₃ sol was added and stirred for 10 min, then DCM and H₂O were added, and the layers were separated. The organic layer was concentrated in vacuo and purified by RP HPLC (Stationary phase: C18 XBridge 30×(100 mm×5 μm), Mobile phase: NH₄HCO₃ 0.25% solution in water, CH₃CN) yielding Example 91 as a solid. ¹H NMR (500 MHz, DMSO-d₆) δ ppm 9.21 (dt, J=8.0, 1.0 Hz, 1H) 8.89-8.99 (m, 1H) 8.70 (t, J=6.0 Hz, 1H) 8.41 (s, 1H) 8.25-8.32 (m, 2H) 8.15 (td, J=7.8, 1.8 Hz, 1H) 7.71 (ddd, J=7.6, 4.6, 1.1 Hz, 1H) 7.69 (t, J=1.0 Hz, 1H) 7.53-7.65 (m, 3H) 7.22-7.26 (m, 2H) 6.90 (t, J=1.1 Hz, 1H) 4.05 (t, J=6.9 Hz, 2H) 2.02 (quin, J=6.9 Hz, 2H) (+2H under the solvent). MS (ESI+) calculated for C₂₄H₂₁N₇O: 423.2; found: 424.3 [M+H]⁺.

### Example 92. N-(1-methylazetidin-3-yl)-5-phenyl-7-(pyridin-2-yl)pyrazolo[1,5-a]pyrimidine-2-carboxamide

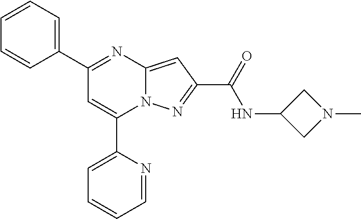 [SMILES: CN1CC(NC(=O)c2cc3nc(-c4ccccc4)cc(-c4ccccn4)n3n2)C1]

Example 92 was synthetized analogously to the method in Example 91 using 5-phenyl-7-(pyridin-2-yl)pyrazolo[1,5-a]pyrimidine-2-carboxylic acid (Intermediate 24) (50 mg, 0.095 mmol) and 1-methylazetidine-3-amine dihydrochloride (18 mg, 0.11 mmol) yielding Example 92 as a solid. ¹H NMR (500 MHz, DMSO-d₆) δ ppm 2.27 (s, 3H) 3.03-3.12 (m, 2H) 3.52-3.66 (m, 2H) 4.47 (sxt, J=7.0 Hz, 1H) 7.25 (s, 1H) 7.55-7.65 (m, 3H) 7.71 (ddd, J=7.6, 4.7, 1.1 Hz, 1H) 8.17 (td, J=7.9, 1.8 Hz, 1H) 8.25-8.32 (m, 2H) 8.39 (s, 1H) 8.88 (d, J=6.9 Hz, 1H) 8.91-8.95 (m, 1H) 9.18 (dt, J=8.0, 1.0 Hz, 1H). MS (ESI+) calculated for C₂₂H₂₀N₆O: 384.2, found: 385.22 [M+H]⁺.

### Example 93. N-(1-methylazetidin-3-yl)-5-phenyl-7-(pyrimidin-2-yl)pyrazolo[1,5-a]pyrimidine-2-carboxamide

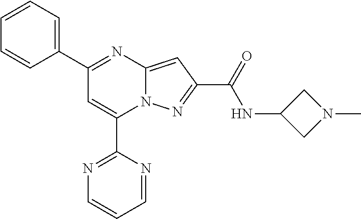 [SMILES: CN1CC(NC(=O)c2cc3nc(-c4ccccc4)cc(-c4ncccn4)n3n2)C1]

Example 93 was synthetized analogously to the method in Example 91 using 5-phenyl-7-(pyrimidin-2-yl)pyrazolo[1,5-a]pyrimidine-2-carboxylic acid (Intermediate 26) and 1-methylazetidine-3-amine dihydrochloride (56 mg, 0.11 mmol) yielding Example 93 as a solid. ¹H NMR (400 MHz, DMSO-d₆) δ ppm 2.23 (s, 3H) 2.97-3.08 (m, 2H) 3.48-3.58 (m, 2H) 4.43 (sxt, J=7.0 Hz, 1H) 7.26 (s, 1H) 7.55-7.62 (m, 3H) 7.82 (t, J=5.0 Hz, 1H) 8.06 (s, 1H) 8.22-8.37 (m, 2H) 8.67 (d, J=7.2 Hz, 1H) 9.17 (d, J=5.1 Hz, 2H). MS (ESI+) calculated for C₂₁H₁₉N₇O: 384.2, found: 385.22 [M+H]⁺.

### Example 94. N-(3-(1H-imidazol-1-yl)propyl)-7-(2-fluorophenyl)-5-phenylpyrazolo[1,5-a]pyrimidine-2-carboxamide

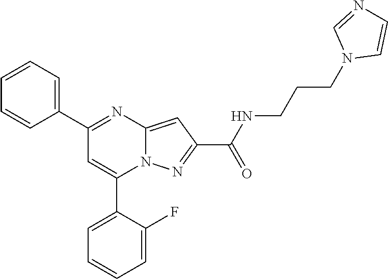 [SMILES: O=C(NCCCn1ccnc1)c1cc2nc(-c3ccccc3)cc(-c3ccccc3F)n2n1]

Example 94 was synthetized analogously to the method in Example 91 using 7-(2-fluorophenyl)-5-phenylpyrazolo[1,5-a]pyrimidine-2-carboxylic acid (Intermediate 27) (100 mg, 0.3 mmol) 1-(3-aminopropyl)imidazole (32 μL, 0.95 g/mL, 0.23 mmol) were added and stirred at rt for 29 h. 1-(3-aminopropyl)imidazole (15 μL, 0.95 g/mL, 0.11 mmol), yielding Example 94 as a solid. ¹H NMR (400 MHz, DMSO-d₆) δ ppm 1.95 (quin, J=7.0 Hz, 2H) 3.25 (br d, J=6.7 Hz, 2H) 3.99 (t, J=6.9 Hz, 2H) 6.87 (s, 1H) 7.15-7.23 (m, 2H) 7.46-7.54 (m, 2H) 7.55-7.60 (m, 3H) 7.64 (s, 1H) 7.69-7.78 (m, 1H) 7.89-7.98 (m, 2H) 8.27-8.35 (m, 2H) 8.39 (t, J=5.9 Hz, 1H). MS (ESI+) calculated for C₂₅H₂₁FN₆O: 440.2, found: 441.3 [M+H]⁺.

### Example 95. N-(3-(1H-imidazol-1-yl)propyl)-7-(4-acetamidophenyl)-5-phenylpyrazolo[1,5-a]pyrimidine-2-carboxamide

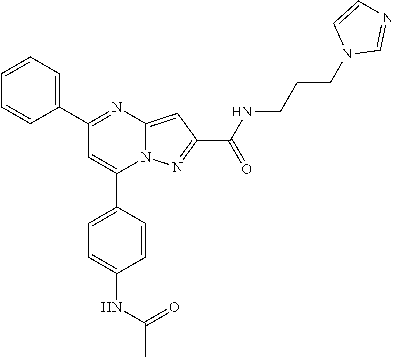 [SMILES: CC(=O)Nc1ccc(-c2cc(-c3ccccc3)nc3cc(C(=O)NCCCn4ccnc4)nn23)cc1]

Example 95 was synthetized analogously to the method in Example 77 using ethyl 7-chloro-5-phenylpyrazolo[1,5-a]pyrimidine-2-carboxylate (Intermediate 3) (60.3 mg, 0.2 mmol) and 4-acetamidobenzeneboronic acid (53.7 mg, 0.3 mmol) yielding Example 95 as a solid. ¹H NMR (500 MHz, DMSO-d₆) δ ppm 2.00 (quin, J=6.9 Hz, 2H) 2.13 (s, 3H) 3.26-3.34 (m, 2H) 4.03 (t, J=6.9 Hz, 2H) 6.89 (s, 1H) 7.13 (s, 1H) 7.23 (t, J=1.1 Hz, 1H) 7.53-7.61 (m, 3H) 7.67 (s, 1H) 7.86 (d, J=8.9 Hz, 2H) 7.92 (s, 1H) 8.30-8.36 (m, 2H) 8.37-8.42 (m, 2H) 8.53 (t, J=6.0 Hz, 1H) 10.32 (s, 1H). MS (ESI+) calculated for C₂₇H₂₅N₇O₂: 479.2, found: 480.3 [M+H]⁺.

### Example 96. N-(3-(1H-imidazol-1-yl)propyl)-5-phenyl-7-(pyridin-3-yl)pyrazolo[1,5-a]pyrimidine-2-carboxamide

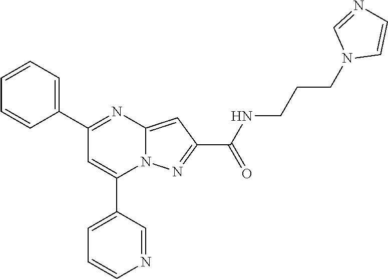 [SMILES: O=C(NCCCn1ccnc1)c1cc2nc(-c3ccccc3)cc(-c3cccnc3)n2n1]

5-Phenyl-7-(pyridin-3-yl)pyrazolo[1,5-a]pyrimidine-2-carboxylic acid (Intermediate 28) (100 mg, 0.32 mmol) and HATU (56 mg, 0.15 mmol) were dissolved in DMF (3 mL), Et₃N (0.04 mL, 0.73 g/mL, 0.29 mmol) and 1-(3-aminopropyl)imidazole (16 μL, 0.95 g/mL, 0.12 mmol) were added and stirred at rt for 29 h. aq Na₂CO₃ sol was added, and the mixture was stirred for 10 min. DCM were added and the layers were separated, and the organic layer was concentrated in vacuo. The product was purified by RP HPLC (Stationary phase: C18 XBridge 30×100 mm 5 μm, Mobile phase: Gradient from 90% NH₄HCO₃ 0.25% solution in water, 10% CH₃CN to 10% NH₄HCO₃ 0.25% solution in water, 90% CH₃CN), yielding Example 96 as a solid. ¹H NMR (400 MHz, DMSO-d₆) δ ppm 1.99 (quin, J=6.9 Hz, 2H) 3.26-3.29 (m, 2H) 4.02 (t, J=6.9 Hz, 2H) 6.89 (s, 1H) 7.18 (s, 1H) 7.21 (s, 1H) 7.56-7.62 (m, 3H) 7.66 (s, 1H) 7.70 (ddd, J=8.0, 4.9, 0.8 Hz, 1H) 8.08 (s, 1H) 8.32-8.42 (m, 2H) 8.55 (t, J=6.0 Hz, 1H) 8.75 (dt, J=8.3, 1.9 Hz, 1H) 8.84 (dd, J=4.9, 1.6 Hz, 1H) 9.45 (d, J=1.6 Hz, 1H). MS (ESI+) calculated for C₂₄H₂₁N₇O: 423.2, found: 424.2 [M+H]⁺.

### Example 97. N-(3-(1H-imidazol-1-yl)propyl)-5-phenyl-7-(pyridin-4-yl)pyrazolo[1,5-a]pyrimidine-2-carboxamide

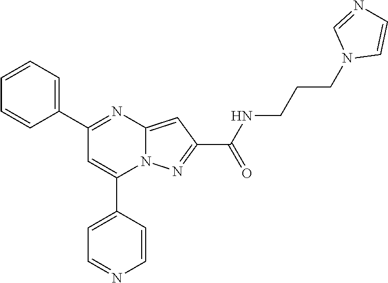 [SMILES: O=C(NCCCn1ccnc1)c1cc2nc(-c3ccccc3)cc(-c3ccncc3)n2n1]

Example 97 was synthetized analogously to the method in Example 96 using 5-phenyl-7-(pyridin-4-yl)pyrazolo[1,5-a]pyrimidine-2-carboxylic acid (Intermediate 29) and in and 1-(3-aminopropyl)imidazole (16 μL, 0.95 g/mL, 0.12 mmol) yielding Example 97 as a solid. ¹H NMR (500 MHz, DMSO-d₆) δ ppm 1.99 (quin, J=6.9 Hz, 2H) 3.27-3.31 (m, 2H) 4.02 (t, J=6.9 Hz, 2H) 6.89 (s, 1H) 7.21 (s, 1H) 7.22 (t, J=1.1 Hz, 1H) 7.56-7.63 (m, 3H) 7.67 (s, 1H) 8.10 (s, 1H) 8.31-8.33 (m, 2H) 8.33-8.38 (m, 2H) 8.56 (t, J=6.0 Hz, 1H) 8.86-8.92 (m, 2H). MS (ESI+) calculated for MS (ESI+) calculated for C₂₄H₂₁N₇O: 423.2, found: 424.2 [M+H]⁺.

### Example 98. N-(1-methylazetidin-3-yl)-7-phenyl-5-(pyrrolidin-1-yl)pyrazolo[1,5-a]pyrimidine-2-carboxamide

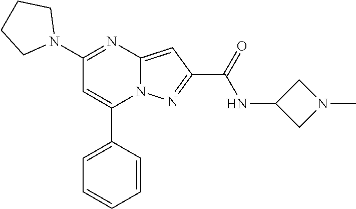 [SMILES: CN1CC(NC(=O)c2cc3nc(N4CCCC4)cc(-c4ccccc4)n3n2)C1]

Example 98 was synthetized analogously to the method in Example 96 using 7-phenyl-5-(pyrrolidin-1-yl)pyrazolo[1,5-a]pyrimidine-2-carboxylic acid (Intermediate 31) (50 mg, 0.16 mmol) and 1-methylazetidin-3-amine (28 mg, 0.32 mmol) yielding Example 98 as a solid. ¹H NMR (500 MHz, DMSO-d₆) δ ppm 1.97 (br s, 4H) 2.25 (s, 3H) 3.03 (br t, J=7.1 Hz, 2H) 3.49-3.67 (m, 6H) 4.39 (sxt, J=7.0 Hz, 1H) 6.40 (s, 1H) 6.58 (s, 1H) 7.51-7.68 (m, 3H) 7.99-8.14 (m, 2H) 8.34 (d, J=7.2 Hz, 1H). MS (ESI+) calculated for C₂₁H₂₄N₆O: 376.2, found: 377.2 [M+H]⁺.

### Example 99. N-(1-methylazetidin-3-yl)-7-phenyl-5-(piperidin-1-yl)pyrazolo[1,5-a]pyrimidine-2-carboxamide

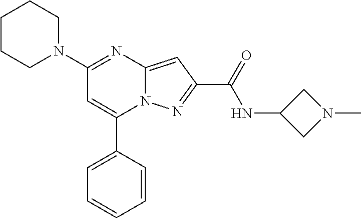 [SMILES: CN1CC(NC(=O)c2cc3nc(N4CCCCC4)cc(-c4ccccc4)n3n2)C1]

Example 99 was synthetized analogously to the method in Example 96 using 7-phenyl-5-(piperidin-1-yl)pyrazolo[1,5-a]pyrimidine-2-carboxylic acid (Intermediate 32) (100 mg, 0.31 mmol) and 1-methylazetidine-3-amine (56 mg, 0.64 mmol) yielding Example 99 as a solid. ¹H NMR (400 MHz, DMSO-d₆) δ ppm 1.58 (br d, J=3.7 Hz, 4H) 1.65 (br d, J=4.6 Hz, 2H) 2.22 (s, 3H) 2.91-3.04 (m, 2H) 3.44-3.57 (m, 2H) 3.67-3.79 (m, 4H) 4.38 (sxt, J=7.0 Hz, 1H) 6.40 (s, 1H) 6.91 (s, 1H) 7.47-7.67 (m, 3H) 7.98-8.13 (m, 2H) 8.32 (d, J=7.2 Hz, 1H). MS (ESI+) calculated for C₂₂H₂₆N₆O: 390.2, found: 391.2 [M+H]⁺.

### Example 100. 5-(azetidin-1-yl)-N-(1-methylazetidin-3-yl)-7-phenylpyrazolo[1,5-a]pyrimidine-2-carboxamide

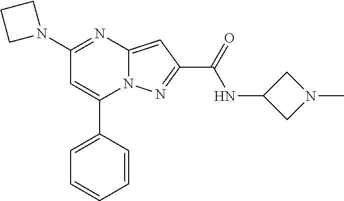 [SMILES: CN1CC(NC(=O)c2cc3nc(N4CCC4)cc(-c4ccccc4)n3n2)C1]

Example 100 was synthetized analogously to the method in Example 96 using 5-(azetidin-1-yl)-7-phenylpyrazolo[1,5-a]pyrimidine-2-carboxylic acid (Intermediate 33) and 1-methylazetidine-3-amine (39 mg, 0.45 mmol) yielding Example 100 as a solid. ¹H NMR (500 MHz, DMSO-d₆) δ ppm 2.22 (s, 3H) 2.35-2.40 (m, 2H) 2.94-3.01 (m, 2H) 3.45-3.56 (m, 2H) 4.15 (br t, J=7.5 Hz, 4H) 4.38 (sxt, J=7.0 Hz, 1H) 6.42 (d, J=0.9 Hz, 2H) 7.52-7.65 (m, 3H) 7.99-8.14 (m, 2H) 8.34 (d, J=7.3 Hz, 1H). MS (ESI+) calculated for C₂₀H₂₂N₆O: 362.2, found: 363.2 [M+H]⁺.

### Example 101. N-(3-(1H-imidazol-1-yl)propyl)-7-phenyl-5-(4-(trifluoromethyl)phenyl)pyrazolo[1,5-a]pyrimidine-2-carboxamide

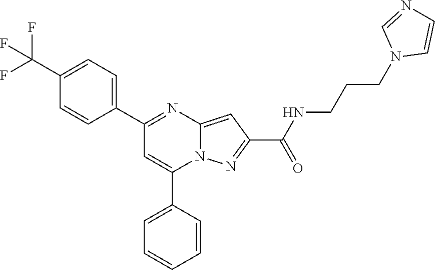 [SMILES: O=C(NCCCn1ccnc1)c1cc2nc(-c3ccc(C(F)(F)F)cc3)cc(-c3ccccc3)n2n1]

Example 101 was synthetized analogously to the method in Example 96 using 7-phenyl-5-(4-(trifluoromethyl)phenyl)pyrazolo[1,5-a]pyrimidine-2-carboxylic acid (Intermediate 34) (90 mg, 0.16 mmol) and 1-(3-aminopropyl)imidazole (0.039 mL, 0.33 mmol), yielding Example 101 as a solid. ¹H NMR (400 MHz, DMSO-d₆) δ ppm 2.02-1.95 (m, 2H) 4.02 (t, J=6.6 Hz, 2H) 6.89 (s, 1H) 7.23 (d, J=8.6 Hz, 2H) 7.67 (d, J=6.1 Hz, 3H) 7.94 (d, J=7.9 Hz, 2H) 8.04 (s, 1H) 8.32 (d, J=7.1 Hz, 2H) 8.52 (s, 1H) 8.57 (d, J=8.0 Hz, 2H), CH₂ signal is under the solvent). MS (ESI+) calculated for C₂₆H₂₁F₃N₆O: 490.2, found: 491.2 [M+H]⁺.

### Example 102. N-(1-methylazetidin-3-yl)-7-phenyl-5-(pyridin-2-yl)pyrazolo[1,5-a]pyrimidine-2-carboxamide

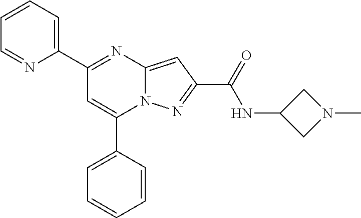 [SMILES: CN1CC(NC(=O)c2cc3nc(-c4ccccn4)cc(-c4ccccc4)n3n2)C1]

Example 102 was synthetized analogously to the method in Example 96 using 7-phenyl-5-(pyridin-2-yl)pyrazolo[1,5-a]pyrimidine-2-carboxylic acid (Intermediate 36) (50 mg, 0.16 mmol) and 1-methylazetidine-3-amine (27 mg, 0.32 mmol), yielding Example 102 as a solid. ¹H NMR (500 MHz, DMSO-d₆) δ ppm 2.29 (s, 3H) 3.11 (br t, J=6.7 Hz, 2H) 3.60 (br t, J=7.3 Hz, 2H) 4.40-4.55 (m, 1H) 7.26 (s, 1H) 7.60 (ddd, J=7.5, 4.8, 1.0 Hz, 1H) 7.65-7.72 (m, 3H) 8.06 (td, J=7.8, 1.7 Hz, 1H) 8.21 (s, 1H) 8.23-8.27 (m, 2H) 8.53 (d, J=7.9 Hz, 1H) 8.75 (d, J=7.0 Hz, 1H) 8.78-8.82 (m, 1H). MS (ESI+) calculated for C₂₂H₂₀N₆O: 384.2, found: 385.2 [M+H]⁺.

### Example 103. N-(1-methylazetidin-3-yl)-7-phenyl-5-(2-(trifluoromethyl)phenyl)pyrazolo[1,5-a]pyrimidine-2-carboxamide

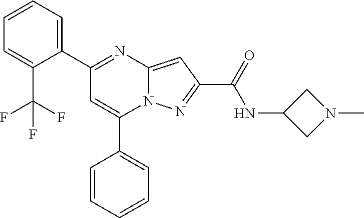 [SMILES: CN1CC(NC(=O)c2cc3nc(-c4ccccc4C(F)(F)F)cc(-c4ccccc4)n3n2)C1]

Pd(PPh₃)₄ (14 mg, 0.012 mmol) was added to a suspension of ethyl 5-chloro-7-phenylpyrazolo[1,5-a]pyrimidine-2-carboxylate (Intermediate 17) (40 mg, 0.1198 mmol) and (2-(trifluoromethyl)phenyl)boronic acid (38 mg, 0.20 mmol) in Na₂CO₃ (sat) (400 μL) and 1,4-dioxane (0.8 mL, 1.033 g/mL, 7.0347 mmol) and the reaction mixture was stirred at 90° C. for 18 h. The mixture was diluted with water and DCM and filtered over celite. The organic layer was separated, dried (MgSO₄), filtered and the solvents evaporated in vacuo. The residue was suspended in THF (1 mL). Then a solution of LiOH (16 mg, 0.6681 mmol) in water (0.1 mL) was added and the reaction mixture was stirred at 50° C. for 4 h. The reaction was cooled to room temperature and acidified with 1N HCl (1 mL). The solvents were removed in vacuo and compound was dissolved in DMF (1 mL). DIPEA (140 μL, 0.75 g/mL, 0.81 mmol) and HATU (103 mg, 0.27 mmol) were added. Resulting slurry was stirred at room temperature for 5 min. Then 1-methylazetidin-3-amine (24 μL, 0.27 mmol) was added and the reaction mixture was stirred at room temperature for 18 h. Then, the mixture was treated with sat. Na₂CO₃ and stirred at room temperature for 5 minutes. The product was extracted with ethyl acetate. The organic layer was separated, dried over MgSO₄, filtered and the solvents evaporated in vacuo. The product was redissolved in DMSO, (3 mL) and purified by RP HPLC (Stationary phase: C18 XBridge, column with 100 mm length, 5 μm. Mobile phase: Gradient using NH₄HCO₃ 0.25% solution in water and CH₃CN as organic solvent), yielding Example 103 as a solid. ¹H NMR (500 MHz, DMSO-d₆) δ ppm 2.26 (s, 3H) 3.06 (br t, J=6.87 Hz, 2H) 3.57 (t, J=6.79 Hz, 2H) 4.40-4.53 (m, 1H) 7.22 (s, 1H) 7.52 (s, 1H) 7.60-7.69 (m, 3H) 7.74-7.80 (m, 1H) 7.80-7.88 (m, 2H) 7.95 (d, J=7.93 Hz, 1H) 8.27 (dd, J=7.63, 1.68 Hz, 2H) 8.78 (d, J=7.02 Hz, 1H). MS (ESI+) calculated for C₂₄H₂₀F₃N₅O: 451.1; found; 452.2 [M+H]⁺.

### Example 104. 5-(2-fluorophenyl)-N-(1-methylazetidin-3-yl)-7-phenylpyrazolo[1,5-a]pyrimidine-2-carboxamide

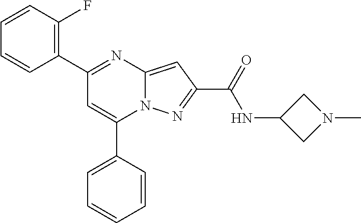 [SMILES: CN1CC(NC(=O)c2cc3nc(-c4ccccc4F)cc(-c4ccccc4)n3n2)C1]

LHMDS (120 μL, 1 M in THF) 0.12 mmol) was added to a solution of 1-methylazetidin-3-amine (5 mg, 0.058 mmol) in THF (0.5 mL), and stirred at rt for 15 min. This solution was added to ethyl 5-(2-fluorophenyl)-7-phenylpyrazolo[1,5-a]pyrimidine-2-carboxylate (Intermediate 19) (10.2 mg, 0.027 mmol) and stirred at rt for 1 h. Then, aq NH₄Cl sol (2 mL) was added, and the product was extracted in EtOAc (3×2 mL). The combined organic extracts were concentrated in vacuo, and purified by RP HPLC (Stationary phase: C18 XBridge, column with 100 mm length, 5 μm. Mobile phase: Gradient using NH₄HCO₃ 0.25% solution in water and CH₃CN). The desired fractions were collected and concentrated in vacuo yielding Example 104 as a solid. ¹H NMR (400 MHz, DMSO-d₆) δ ppm 2.25 (s, 3H) 2.99-3.08 (m, 2H) 3.52-3.60 (m, 2H) 4.45 (sxt, J=6.98 Hz, 1H) 7.24 (s, 1H) 7.39-7.49 (m, 2H) 7.57-7.71 (m, 5H) 8.08 (td, J=7.98, 1.39 Hz, 1H) 8.20-8.29 (m, 2H) 8.75 (d, J=7.17 Hz, 1H). MS (ESI+) calculated for C₂₃H₂₀FN₅O: 401.2; found: 402.2 [M+H]⁺.

### Example 105. 5-(3-fluorophenyl)-N-(1-methylazetidin-3-yl)-7-phenylpyrazolo[1,5-a]pyrimidine-2-carboxamide

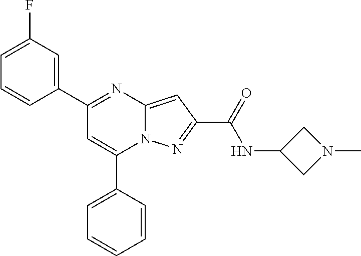 [SMILES: CN1CC(NC(=O)c2cc3nc(-c4cccc(F)c4)cc(-c4ccccc4)n3n2)C1]

Example 105 was synthetized analogously to the method in Example 104 using ethyl 5-(3-fluorophenyl)-7-phenylpyrazolo[1,5-a]pyrimidine-2-carboxylate (Intermediate 20) and 1-methylazetidin-3-amine (5 mg, 0.058 mmol) yielding Example 105. ¹H NMR (400 MHz, DMSO-d₆) δ ppm 2.26 (s, 3H) 3.00-3.09 (m, 2H) 3.53-3.58 (m, 2H) 4.45 (q, J=6.94 Hz, 1H) 7.22 (s, 1H) 7.42 (td, J=8.44, 2.08 Hz, 1H) 7.59-7.72 (m, 4H) 7.98 (s, 1H) 8.14-8.25 (m, 2H) 8.27-8.35 (m, 2H) 8.73 (d, J=7.17 Hz, 1H). MS (ESI+) m/z calculated for C₂₃H₂₀FN₅O: 401.2; found: 402.2 [M+H]⁺.

### Example 106. 5-(4-fluorophenyl)-N-(1-methylazetidin-3-yl)-7-phenylpyrazolo[1,5-a]pyrimidine-2-carboxamide

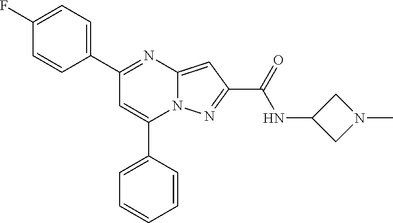 [SMILES: CN1CC(NC(=O)c2cc3nc(-c4ccc(F)cc4)cc(-c4ccccc4)n3n2)C1]

Example 106 was synthetized analogously to the method in Example 104 using ethyl 5-(4-fluorophenyl)-7-phenylpyrazolo[1,5-a]pyrimidine-2-carboxylate (Intermediate 21) (13.3 mg, 0.0368 mmol) and 1-methylazetidin-3-amine (5 mg, 0.058 mmol) yielding Example 106 as a solid. ¹H NMR (400 MHz, DMSO-d₆) δ ppm 2.25 (s, 3H) 3.04 (t, J=7.3 Hz, 2H) 3.49-3.62 (m, 2H) 4.45 (sxt, J=7.0 Hz, 1H) 7.42 (t, J=8.3 Hz, 2H) 7.18 (s, 1H) 7.64-7.70 (m, 3H) 7.93 (s, 1H) 8.27-8.32 (m, 2H) 8.39-8.45 (m, 2H) 8.71 (d, J=7.2 Hz, 1H). MS (ESI+) calculated for C₂₃H₂₀FN₅O: 401.2, found: 402.2 [M+H]⁺.

### Example 107. N-(3-(1H-imidazol-1-yl)propyl)-7-cyclohexyl-5-phenylpyrazolo[1,5-a]pyrimidine-2-carboxamide

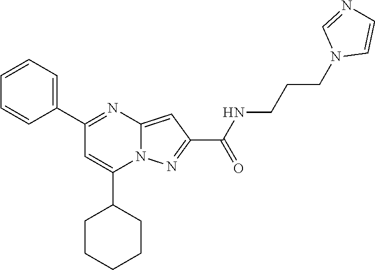 [SMILES: O=C(NCCCn1ccnc1)c1cc2nc(-c3ccccc3)cc(C3CCCCC3)n2n1]

DIPEA (0.17 mL, 0.75 g/mL, 0.98 mmol) was added to a stirred suspension of 7-cyclohexyl-5-phenylpyrazolo[1,5-a]pyrimidine-2-carboxylic acid (Intermediate 45) (63.8 mg, 0.18 mmol), [1,2,4]triazolo[4,3-a]pyridin-7-amine (35 mg, 0.28 mmol) and HATU (140 mg, 0.37 mmol) in DMF (1.85 mL). The mixture was stirred at rt for 2 h. aq sat NaHCO₃ sol (4 mL) and EtOAc (4 mL) were added and the phases were separated. The aqueous phase was extracted with EtOAc (2×4 mL) and EtOAc/THF (7/3) (1×4 mL). The combined organic layers were dried over Na₂SO₄, filtered and concentrated in vacuo. The residue was purified by RP HPLC (Stationary phase: C18 XBridge 30×100 mm 5 μm, Mobile phase: Gradient from 70% NH₄HCO₃ 0.25% solution in water, 30% CH₃CN to 35% NH₄HCO₃ 0.25% solution in water, 65% CH₃CN) yielding Example 107 as an off white solid. ¹H NMR (400 MHz, DMSO-d₆) δ ppm 1.29-1.63 (m, 3H) 1.66-1.97 (m, 5H) 2.02 (quin, J=6.94 Hz, 2H) 2.07-2.18 (m, 2H) 2.52-2.55 (m, 2H) 3.67 (tt, J=11.91, 3.12 Hz, 1H) 4.04 (t, J=6.94 Hz, 2H) 6.90 (s, 1H) 7.07 (s, 1H) 7.24 (t, J=1.16 Hz, 1H) 7.52-7.59 (m, 3H) 7.61 (s, 1H) 7.68 (s, 1H) 8.22-8.37 (m, 2H) 8.56 (t, J=5.90 Hz, 1H). MS (ESI+) calculated for C₂₅H₂₈N₆O: 428.2; found: 429.3 [M+H]⁺.

### Example 108. N-(3-(1H-imidazol-1-yl)propyl)-7-cyclopentyl-5-phenylpyrazolo[1,5-a]pyrimidine-2-carboxamide

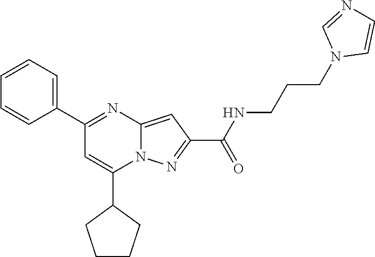 [SMILES: O=C(NCCCn1ccnc1)c1cc2nc(-c3ccccc3)cc(C3CCCC3)n2n1]

Example 108 was synthetized analogously to the method in Example 107 using 7-cyclopentyl-5-phenylpyrazolo[1,5-a]pyrimidine-2-carboxylic acid (Intermediate 46) (31.2 mg, 0.1 mmol) and 1-(3-aminopropyl)imidazole yielding Example 108 as a solid. ¹H NMR (400 MHz, DMSO-d₆) δ ppm 1.70-1.84 (m, 2H) 1.84-1.96 (m, 4H) 1.97-2.09 (m, 2H) 2.24-2.42 (m, 2H) 2.51-2.55 (m, 2H) 3.89-4.00 (m, 1H) 4.04 (t, J=6.82 Hz, 2H) 6.90 (s, 1H) 7.07 (s, 1H) 7.20-7.25 (m, 1H) 7.53-7.61 (m, 3H) 7.63 (s, 1H) 7.68 (s, 1H) 8.20-8.37 (m, 2H) 8.56 (t, J=6.01 Hz, 1H). MS (ESI+) calculated for C₂₄H₂₆N₆O: 414.2; found: 415.3 [M+H]⁺.

### Example 109. N-(3-(1H-imidazol-1-yl)propyl)-7-cyclobutyl-5-phenylpyrazolo[1,5-a]pyrimidine-2-carboxamide

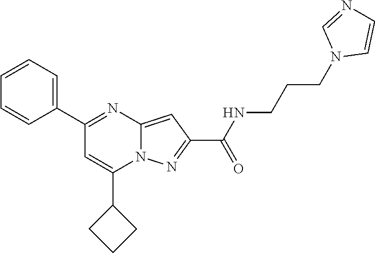 [SMILES: O=C(NCCCn1ccnc1)c1cc2nc(-c3ccccc3)cc(C3CCC3)n2n1]

Example 109 was synthetized analogously to the method Example 107 using 7-cyclobutyl-5-phenylpyrazolo[1,5-a]pyrimidine-2-carboxylic acid (Intermediate 47) (37.5 mg, 0.1 mmol) and 1-(3-aminopropyl)imidazole (19.06 mg, 0.15 mmol) yielding Example 109. ¹H NMR (400 MHz, DMSO-d₆) δ ppm 1.90-2.06 (m, 3H) 2.11-2.25 (m, 1H) 2.19-2.20 (m, 1H) 2.36-2.49 (m, 2H) 2.52-2.67 (m, 3H) 4.04 (t, J=6.94 Hz, 2H) 4.19-4.45 (m, 1H) 6.90 (s, 1H) 7.06 (s, 1H) 7.23 (s, 1H) 7.53-7.63 (m, 3H) 7.64-7.72 (m, 2H) 8.23-8.39 (m, 2H) 8.51 (t, J=6.01 Hz, 1H). MS (ESI+) calculated for C₂₃H₂₄N₆O: 400.2; found: 401.2 [M+H]⁺.

### Example 110. N-(3-(1H-imidazol-1-yl)propyl)-5-phenyl-7-(tetrahydrofuran-2-yl)pyrazolo[1,5-a]pyrimidine-2-carboxamide

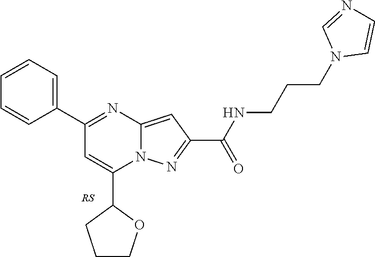 [SMILES: O=C(NCCCn1ccnc1)c1cc2nc(-c3ccccc3)cc(C3CCCO3)n2n1]

Example 110 was synthetized analogously to the method in Example 107 using 5-phenyl-7-(tetrahydrofuran-2-yl) pyrazolo[1,5-a]pyrimidine-2-carboxylic acid (Intermediate 48) (63.2 mg, 0.065 mmol) and 1-(3-aminopropyl)imidazole (11.0 mg, 0.088 mmol) yielding Example 110 as a solid. ¹H NMR (400 MHz, chloroform-d) δ ppm 1.93-2.30 (m, 5H) 2.70-2.95 (m, 1H) 3.44-3.67 (m, 2H) 4.02-4.19 (m, 3H) 4.20-4.29 (m, 1H) 5.53-5.65 (m, 1H) 6.96-7.12 (m, 2H) 7.16 (br t, J=6.13 Hz, 1H) 7.45-7.71 (m, 5H) 8.06-8.22 (m, 2H). MS (ESI+) calculated for C₂₃H₂₄N₆O₂: 416.2; found: 417.2 [M+H]⁺.

### Example 111. N-(3-(1H-imidazol-1-yl)propyl)-5-phenyl-7-(tetrahydro-2H-pyran-2-yl)pyrazolo[1,5-a]pyrimidine-2-carboxamide

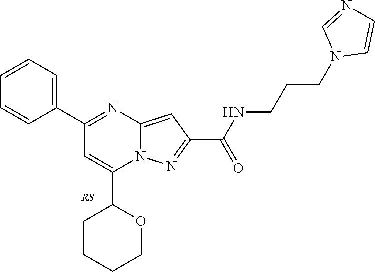 [SMILES: O=C(NCCCn1ccnc1)c1cc2nc(-c3ccccc3)cc(C3CCCCO3)n2n1]

Example 111 was synthetized analogously to the method in Example 107 using 5-phenyl-7-(tetrahydro-2H-pyran-2-yl) pyrazolo[1,5-a]pyrimidine-2-carboxylic acid (Intermediate 49) (88.2 mg, 0.12 mmol) and 1-(3-aminopropyl)imidazole (23.0 mg, 0.18 mmol) yielding Example 111. ¹H NMR (500 MHz, DMSO-d₆) δ ppm 1.42-1.59 (m, 1H) 1.64-1.89 (m, 3H) 1.92-2.05 (m, 3H) 3.20-3.44 (m, 3H) 3.73 (td, J=11.25, 3.43 Hz, 1H) 4.04 (t, J=6.87 Hz, 2H) 4.16-4.28 (m, 1H) 5.21 (dd, J=11.06, 1.75 Hz, 1H) 6.90 (t, J=0.99 Hz, 1H) 7.11 (s, 1H) 7.24 (t, J=1.22 Hz, 1H) 7.55-7.60 (m, 3H) 7.61 (d, J=0.61 Hz, 1H) 7.68 (s, 1H) 8.07-8.29 (m, 2H) 8.58 (t, J=5.95 Hz, 1H). MS (ESI+) calculated for C₂₄H₂₆N₆O₂: 430.2; found: 431.2 [M+H]⁺.

### Example 112. N-(3-(1H-imidazol-1-yl)propyl)-7-isobutyl-5-phenylpyrazolo[1,5-a]pyrimidine-2-carboxamide

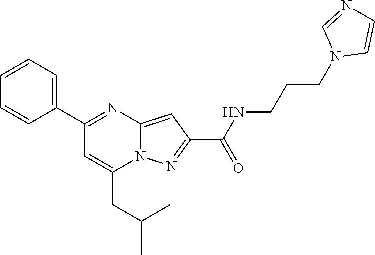 [SMILES: CC(C)Cc1cc(-c2ccccc2)nc2cc(C(=O)NCCCn3ccnc3)nn12]

LiHMDS (0.44 mL, 1 M, 0.44 mmol) was added to a stirred solution of 1-(3-aminopropyl)imidazole (25 mg, 0.2 mmol) in DMF (0.4 mL) at 0° C. The mixture was stirred at 0° C. for 10 min, and then a solution of ethyl 7-isobutyl-5-phenylpyrazolo[1,5-a]pyrimidine-2-carboxylate (Intermediate 41) (64.7 mg, 0.2 mmol) in THF (0.4 mL) was added. The mixture was stirred at 0° C. for 1 h. Aq. NH₄Cl (1.5 mL) and EtOAc (2 mL) were added and the phases were separated. The aqueous phase was extracted with EtOAc-THF (8/2) (2×2 mL). The combined organic layers were dried over Na₂SO₄, filtered and concentrated in vacuo. The residue was purified by RP HPLC (Stationary phase: C18 XBridge 30×100 mm 5 μm), Mobile phase: Gradient from 70% NH₄HCO₃ 0.25% solution in water, 30% CH₃CN to 35% NH₄HCO₃ 0.25% solution in water, 65% CH₃CN) yielding Example 112 as an off white solid. ¹H NMR (400 MHz, chloroform-d) δ ppm 1.09 (d, J=6.70 Hz, 6H) 2.19 (quin, J=6.94 Hz, 2H) 2.44 (dt, J=13.58, 6.73 Hz, 1H) 3.10 (d, J=7.17 Hz, 2H) 3.54 (q, J=6.70 Hz, 2H) 4.10 (t, J=7.05 Hz, 2H) 7.01 (t, J=1.16 Hz, 1H) 7.09 (s, 1H) 7.23 (d, J=14.56 Hz, 2H) 7.49-7.63 (m, 4H) 8.04-8.17 (m, 2H); 1H exchangeable. MS (ESI+) calculated for C₂₃H₂₆N₆O: 402.22; found: 403.20 [M+H]⁺.

### Example 113. N-(3-(1H-imidazol-1-yl)propyl)-7-cyclopropyl-5-phenylpyrazolo[1,5-a]pyrimidine-2-carboxamide

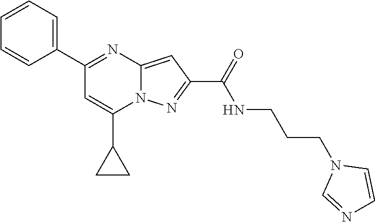 [SMILES: O=C(NCCCn1ccnc1)c1cc2nc(-c3ccccc3)cc(C3CC3)n2n1]

Example 113 was synthetized analogously to the method in Example 112 using ethyl 7-cyclopropyl-5-phenylpyrazolo[1,5-a]pyrimidine-2-carboxylate (Intermediate 40) (67.47 mg, 0.20 mmol) and 1-(3-aminopropyl)imidazole (25 mg, 0.20 mmol) yielding Example 113 as a solid. ¹H NMR (500 MHz, DMSO-d₆) δ ppm 1.31-1.48 (m, 4H) 1.83-2.09 (m, 2H) 2.85-3.06 (m, 1H) 3.28-3.32 (m, 2H) 4.04 (t, J=6.94 Hz, 2H) 6.90 (t, J=0.99 Hz, 1H) 7.05 (s, 1H) 7.20-7.38 (m, 2H) 7.51-7.61 (m, 3H) 7.68 (s, 1H) 8.20-8.30 (m, 2H) 8.62 (t, J=5.95 Hz, 1H). MS (ESI+) calculated for C₂₂H₂₂N₆O: 386.2; found: 387.2 [M+H]⁺.

### Example 114. N-(3-(1H-imidazo-1-yl)propyl)-5-phenyl-7-(piperidin-2.yl)pyrazolo[1,5-a]pyrimidine-2-carboxamide

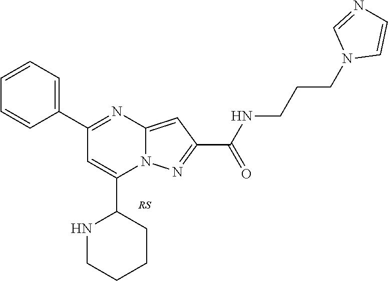 [SMILES: O=C(NCCCn1ccnc1)c1cc2nc(-c3ccccc3)cc(C3CCCCN3)n2n1]

TFA (712 μL, 1.49 g/mL, 9.3 mmol) was added to a stirred solution of tert-butyl-2-(2-((3-(1H-imidazo-1-yl)propyl)carbamoyl)-5-phenylpyrazolo[1,5-a]pyrimidin-7-yl)piperidine-1-carboxylate (Intermediate 45) (120 mg, 0.23 mmol) in DCM (1.5 mL). The mixture was stirred at rt for 1 h. The solvent was concentrated in vacuo. The residue was passed through a SCX-2 cartridge eluting with 7 N ammonia solution in MeOH. The solvent was concentrated in vacuo to yield example 114 as a light-yellow oil. ¹H NMR (400 MHz, methanol-d₄) δ ppm 1.42-1.86 (m, 4H) 1.92 (br d, J=9.48 Hz, 1H) 2.07 (quin, J=6.88 Hz, 2H) 2.20-2.30 (m, 1H) 2.82-2.94 (m, 1H) 3.21 (dt, J=3.24, 1.62 Hz, 2H) 3.33-3.45 (m, 2H) 4.06 (t, J=6.94 Hz, 2H) 4.54 (dd, J=10.40, 2.31 Hz, 1H) 6.89 (t, J=1.16 Hz, 1H) 7.03 (s, 1H) 7.11 (t, J=1.27 Hz, 1H) 7.42-7.51 (m, 3H) 7.62 (d, J=19.42 Hz, 2H) 8.00-8.17 (m, 2H); MS (ESI+) calculated for C₂₄H₂₇N₇O: 429.2; found: 430.3 [M+H]⁺.

### Example 115. N-(azetidin-3-yl)-5,7-diphenylpyrazolo[1,5-a]pyrimidine-2-carboxamide

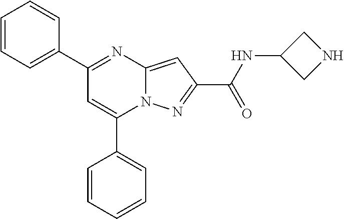 [SMILES: O=C(NC1CNC1)c1cc2nc(-c3ccccc3)cc(-c3ccccc3)n2n1]

A solution of tert-butyl 3-(5,7-diphenylpyrazolo[1,5-a]pyrimidine-2-carboxamido)azetidine-1-carboxylate (Intermediate 52) (160 mg, 0.307 mmol) and 4M hydrochloric acid in 1,4-dioxane (3 mL) was stirred at room temperature for 1 hour. The reaction was concentrated to give residue, which was purified by prep. HPLC (preparation method: SunFire® Prep C₁₈ OBD™ (5 μm 19*150 mm), Mobile Phase A: water (0.01% trifluoroacetic acid), Mobile Phase B: acetonitrile, UV: 214 nm, Flow rate: 15 mL/min, Gradient: 5-95% (% B)) to give example 115 as solid. ¹H NMR (400 MHz, DMSO-d₆) δ 4.21-4.19 (m, 4H), 4.89-4.83 (m, 1H), 7.21 (s, 1H), 7.59-7.58 (m, 3H), 7.68-7.66 (m, 3H), 7.95 (s, 1H), 8.29-8.27 (m, 2H), 8.36-8.34 (m, 2H), 8.74 (br s, 2H), 9.07 (d, J=6.8 Hz, 1H). MS (ESI+) calculated for C₂₂H₁₉N₅O₂: 369.1, found: 370.2 [M+H]⁺.

### Example 116. N-(3-aminopropyl)-5,7-diphenylpyrazolo[1,5-a]pyrimidine-2-carboxamide

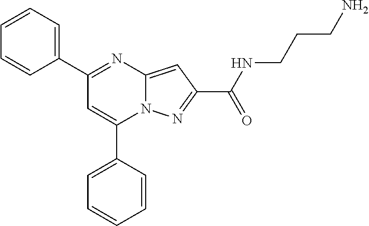 [SMILES: NCCCNC(=O)c1cc2nc(-c3ccccc3)cc(-c3ccccc3)n2n1]

HCl (4M in dioxane) (250 μL) was added to a suspension of tert-butyl (3-(5,7-diphenylpyrazolo[1,5-a]pyrimidine-2-carboxamido)propyl)carbamate (Intermediate 53) (21 mg, 0.04454 mmol) in 1,4-dioxane (100 μL), and the reaction mixture was stirred at rt for 18 h. Then, the solvents were concentrated in vacuo and the residue was redissolved in methanol and the solution loaded onto an isolute SCX2 cartridge. The product was eluted with 5% ammonia in methanol. The desired fractions were collected and concentrated in vacuo yielding Example 116 as a solid. ¹H NMR (400 MHz, CDCl₃) δ ppm 1.76 (t, J=6.59 Hz, 2H) 2.85 (t, J=6.47 Hz, 2H) 3.58 (q, J=6.40 Hz, 2H) 7.32 (s, 1H) 7.44 (s, 1H) 7.50-7.58 (m, 3H) 7.58-7.68 (m, 4H) 8.04-8.10 (m, 2H) 8.12-8.18 (m, 2H), 2H exchangeable. MS (ESI+) calculated for C₂₂H₂₁N₅O: 371.4, found: 372.2 [M+H]⁺.

### Example 117. 5,7-diphenyl-(pyrrolidin-2-ylmethyl)pyrazolo[1,5-a]pyrimidine-2-carboxamide

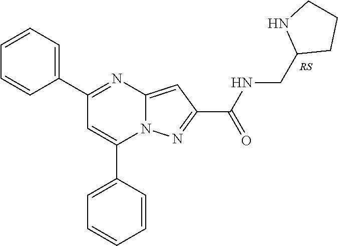 [SMILES: O=C(NCC1CCCN1)c1cc2nc(-c3ccccc3)cc(-c3ccccc3)n2n1]

Example 117 was synthetized analogously of Example 116 with 2-((5,7-diphenylpyrazolo[1,5-a]pyrimidine-2-carboxamido)methyl)pyrrolidine-1-carboxylate (Intermediate 54) (63 mg, 0.1903 mmol), yielding Example 117 as solid. ¹H NMR (500 MHz, DMSO-d₆) δ ppm 1.34-1.44 (m, 1H) 1.54-1.80 (m, 3H) 2.71-2.86 (m, 2H) 3.33 (s, 4H) 7.16 (s, 1H) 7.54-7.62 (m, 3H) 7.63-7.71 (m, 3H) 7.93 (s, 1H) 8.22 (t, J=5.65 Hz, 1H) 8.27-8.31 (m, 2H) 8.31-8.37 (m, 2H). MS (ESI+) calculated for C₂₄H₂₃N₅O: 397.2, found: 398.3 [M+H]⁺.

### Example 118. N-(3-(methylamino)propyl)-5,7-diphenylpyrazolo[1,5-a]pyrimidine-2-carboxamide

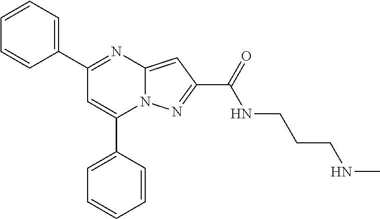 [SMILES: CNCCCNC(=O)c1cc2nc(-c3ccccc3)cc(-c3ccccc3)n2n1]

Example 118 was synthetized analogously to the method in Example 116 with tert-butyl (3-(5,7-diphenylpyrazolo[1,5-a]pyrimidine-2-carboxamido)propyl)(methyl)carbamate (Intermediate 55) (31 mg, 0.06388 mmol) yielding Example 118 as a solid. ¹H NMR (400 MHz, chloroform-d) δ ppm 1.79 (quin, J=6.42 Hz, 2H) 2.39 (s, 3H) 2.73 (t, J=6.36 Hz, 2H) 3.57 (q, J=6.01 Hz, 2H) 7.32 (s, 1H) 7.44 (s, 1H) 7.50-7.58 (m, 3H) 7.58-7.65 (m, 3H) 7.88-7.98 (m, 1H) 8.04-8.10 (m, 2H) 8.12-8.18 (m, 2H), 1H exchangeable. MS (ESI+) calculated for C₂₃H₂₃N₅O: 385.2, found: 386.3 [M+H]⁺.

### Example 119. N-(2-aminoethyl)-5,7-diphenylpyrazolo[1,5-a]pyrimidine-2-carboxamide hydrochloride

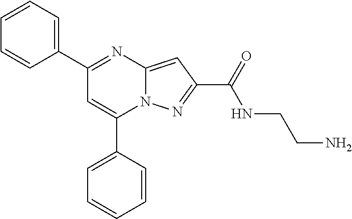 [SMILES: NCCNC(=O)c1cc2nc(-c3ccccc3)cc(-c3ccccc3)n2n1]

HCl (4M in dioxane) (250 μL, 4 M, 1 mmol) was added to a suspension of t tert-butyl (3-(5,7-diphenylpyrazolo[1,5-a]pyrimidine-2-carboxamido)propyl)(methyl)carbamate (Intermediate 56) (31 mg, 0.06388 mmol) in 1,4-dioxane (100 μL) and the reaction mixture was stirred at room temperature for 18 h. The solvents were concentrated in vacuo yielding Example 119 as solid. ¹H NMR (400 MHz, DMSO-d₆) δ ppm 3.01 (q, J=5.93 Hz, 2H) 3.58 (q, J=6.24 Hz, 2H) 7.23 (s, 1H) 7.55-7.63 (m, 3H) 7.64-7.71 (m, 3H) 7.90-8.06 (m, 4H) 8.29-8.33 (m, 2H) 8.33-8.38 (m, 2H) 8.63 (t, J=5.90 Hz, 1H). MS (ESI+) calculated for C₂₁H₁₉N₅O: 357.2, found: 358.2 [M+H]⁺.

### Example 120. N-(3-amino-2-methylpropyl)-5,7-diphenylpyrazolo[1,5-a]pyrimidine-2-carboxamide hydrochloride

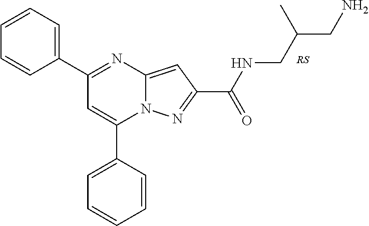 [SMILES: CC(CN)CNC(=O)c1cc2nc(-c3ccccc3)cc(-c3ccccc3)n2n1]

Example 120 was synthetized analogously to the method in Example 119, using tert-butyl (3-(5,7-diphenylpyrazolo[1,5-a]pyrimidine-2-carboxamido)-2-methylpropyl)carbamate (Intermediate 57) (20 mg, 0.0412 mmol) yielding Example 120 as a solid. ¹H NMR (400 MHz, DMSO-d₆) δ ppm 0.97 (d, J=6.70 Hz, 3H) 2.03-2.18 (m, 1H) 2.60-2.72 (m, 1H) 2.76-2.89 (m, 1H) 3.24-3.35 (m, 2H) 7.21 (s, 1H) 7.55-7.62 (m, 3H) 7.63-7.71 (m, 3H) 7.85-8.00 (m, 4H) 8.27-8.40 (m, 4H) 8.64 (t, J=6.13 Hz, 1H). MS (ESI+) calculated for C₂₃H₂₃N₅O: 385.2, found: 386.3 [M+H]⁺.

### Example 121. 5,7-diphenyl-N-(pyrrolidin-3-ylmethyl)pyrazolo[1,5-a]pyrimidine-2-carboxamide

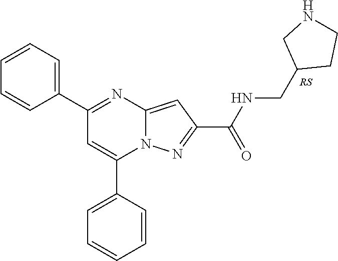 [SMILES: O=C(NCC1CCNC1)c1cc2nc(-c3ccccc3)cc(-c3ccccc3)n2n1]

Example 121 was synthetized analogously to the method in Example 119, using tert-butyl 3-((5,7-diphenylpyrazolo[1,5-a]pyrimidine-2-carboxamido)methyl)pyrrolidine-1-carboxylate (Intermediate 58) (47 mg, 0.0945 mmol) yielding Example 121 as a solid. ¹H NMR (400 MHz, CDCl₃) δ ppm 1.47-1.59 (m, 1H) 1.96 (dtd, J=13.00, 8.06, 8.06, 5.32 Hz, 1H) 2.37-2.50 (m, 1H) 2.74 (dd, J=10.87, 5.55 Hz, 1H) 2.92 (ddd, J=10.69, 7.92, 6.82 Hz, 1H) 3.00-3.11 (m, 2H) 3.47 (t, J=6.59 Hz, 2H) 7.29-7.40 (m, 2H) 7.45 (s, 1H) 7.50-7.58 (m, 3H) 7.58-7.66 (m, 3H) 8.03-8.11 (m, 2H) 8.11-8.18 (m, 2H), 1H exchangeable. MS (ESI+) calculated for C₂₄H₂₃N₅O: 397.2, found: 398.3 [M+H]⁺.

The following examples may be synthesized by methods known in the art or by the procedures proposed below.

### Example 122. N-(2-morpholinoethyl)-5,7-diphenylpyrazolo[1,5-a]pyrimidine-2-carboxamide

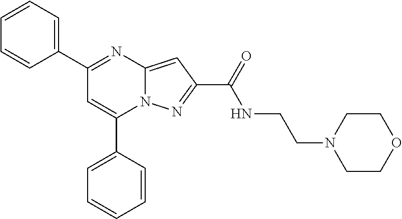 [SMILES: O=C(NCCN1CCOCC1)c1cc2nc(-c3ccccc3)cc(-c3ccccc3)n2n1]

Example 122 can be prepared analogously to the method in Example 1 using 5,7-diphenylpyrazolo[1,5,a]pyrimidine-2-carboxylic acid (Intermediate 2) and 2-morpholinoethan-1-amine.

### Example 123. N-(3-(1H-imidazol-1-yl)propyl)-7-methyl-5-phenylpyrazolo[1,5-a]pyrimidine-2-carboxamide

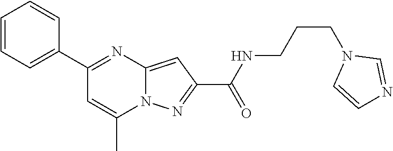 [SMILES: Cc1cc(-c2ccccc2)nc2cc(C(=O)NCCCn3ccnc3)nn12]

Example 123 can be prepared analogously to the method in Example 107 or 111 using 7-methyl-5-phenylpyrazolo[1,5-a]pyrimidine-2-carboxylic acid and 1-(3-aminopropyl)imidazole.

### In Vitro Biological Data

The direct detection of hydrogen peroxide produced from the enzymatic reaction of hSMOX or hPAOX in a biochemical assay format was measured using HyPerBlu (Lumigen, Southfield, MI), a small molecule boronate probe that reacts with hydrogen peroxide to generate a chemiluminescent signal. Inhibition of hSMOX or hPAOX enzyme results in a decrease in luminescence.

### Human SMOX Assay

Test compounds (75 nL) dissolved in DMSO were added to a 384-well white assay plate (Greiner Bio-one, catalog #784075) using an ECHO Acoustic Dispenser (Labcyte). Each compound was serially diluted 3-fold in DMSO to prepare an 11-point dose response curve (columns 1-22). Column 23 and 24 contained 75 nL of 2 mM chlorhexidine and DMSO, respectively, to represent the 100% inhibited versus the neutral control reactions.

Subsequently, 3 μL/well of the substrate solution prepared in assay buffer (10 mM Hepes, pH 7.4, 150 mM NaCl, 1 mM EGTA, 0.01% Pluronic F-127, 0.05% ovalbumin) was dispensed into the assay plate followed by 3 μL/well of full length hSMOX enzyme with an N-terminal His tag from E. coli prepared in assay buffer to initiate the reactions. After a 1 h incubation at room temperature, 6 μL/well of HyPerBlu was added. After a 30 min incubation, the hydrogen peroxide product generated from the hSMOX reactions was measured using an endpoint luminescence read on the PHERAStar FS/FSX microplate reader (BMG LABTECH).

The final assay conditions included 10 mM Hepes, pH 7.4, 150 mM NaCl, 1 mM EGTA, 0.01% Pluronic F-127, 0.05% ovalbumin, 2.5 nM hSMOX, 30 M spermine, and 1.25% DMSO.

The raw luminescence data was normalized to % inhibition using DMSO or 25 μM chlorhexidine to represent the 0 and 100% inhibited control reactions, respectively. Dose-response curves were fit and compound IC₅₀ values were determined using Genedata Screener (Genedata AG).

### Human PAOX Assay

The hPAOX assay was run and the data analyzed similarly to the hSMOX assay described above, except the reactions contained 60 pM recombinant PAOX with an N-terminal His tag expressed from sf9 insect cells and used 20 μM N-acetylspermine as the substrate.

### Cell Based Biological Data

The Cellular Thermal Shift Assay (CETSA) is a biophysical assay based on the principle of ligand-induced thermal stabilization of target proteins, meaning that a protein denaturing temperature will change upon ligand interaction. Thus, by heating samples (i.e. cells, ex vivo samples) to different temperatures, and quantifying conformationally intact proteins we can detect compound-protein interactions after compound treatment. CETSA allows direct monitoring of ligand binding to a specific target (target engagement). In our study, we have developed a suitable detection system that will detect intact SMOX using the alphaLISA. This PerkinElmer's bead-based assay is a highly selective and sensitive chemiluminescent, no-wash assay that allow us to distinguish intact from denatured SMOX. The detection of compound -SMOX ligand binding is performed using A549 cells. This adenocarcinoma human alveolar basal epithelial cell line expresses high levels of SMOX. Their use has been widely reported in the literature with respect to the investigation of the SMOX pathway. Using SMOX high-expressing cells with CETSA and alphaLISA systems provides the optimal platform for screening small molecules for SMOX inhibition.

### Protocol for Intracellular Detection of Ligand Binding of SMOX

### Cell Culture and Cell Preparation:

A549 cells were seeded at a density of 0.05-0.1×10⁶ cells/mL in T175 flasks and cultured in 25 mL of culture medium consisting in RPMI 1640 medium (Gibco) supplemented with 10% fetal bovine serum (Gibco). Cells were passaged twice per week and used for CETSA assays before reaching confluency. One day prior to the CETSA cells were seeded in 384-well white assay plates (Corning) at 15000 cells per well. Briefly, cells were washed with PBS (Life Technologies Europe BV) and dissociated by adding 2 mL TrypLE™ Select (Life Technologies Europe BV). After 5 min at 37° C., cells were resuspended in culture medium, counted using a ViCell XR cell counter (ViCell) and seeded in the 384-well plates.

### Compound Incubation and CETSA Procedure:

Compounds were pre-diluted in PBS to a final concentration of 10 M and 1% DMSO and serially diluted in PBS 1% DMSO. For compound-cell incubation, medium in the 384 well plates was replaced with 30 μL of serially diluted compounds. After incubation at 37° C. for 1 hour the liquid was removed and plates were heated to 55° C. for 5 minutes and then transferred to ice for 5 minutes. After the cooling step, cells were lysed by adding 15 μL 1× alphaLISA-buffer (Perkin Elmer) containing anti-SMOX monoclonal antibody #2 and anti-SMOX Fab-fragment #33 mixture at a concentration of 5 and 0.5 nM respectively. After one hour at RT, 5 μL anti-6×His alphaLISA acceptor beads (50 mg/L, AL128C, Perkin Elmer) were added to the wells followed by an additional incubation of one hour at RT. Then 5 μL anti-rabbit IgG alpha donor beads (200 mg/L was added to the wells. After one hour incubation at RT the alpha signal was read using an alpha compatible reader. MDL75257 (M2949, Sigma) was used as positive control for compound-SMOX interaction. Compound-SMOX interaction is detected through the comparison of the high alpha signal of the negative control with the much lower signal of the wells that contain SMOX binding compounds.

### Biological Data

| | | PAOx IC50 | SMOx % recovery |
|---|---|---|---|
| Example | SMOx_IC50 (mM) | (mM) | CETSA |
| Example 1 | 0.046 | 0.397 | 12% |
| Example 2 | 0.045 | 0.942 | 26% |
| Example 3 | 4.355 | 4.543 | |
| Example 4 | 0.599 | 8.718 | |
| Example 5 | 0.367 | 0.914 | |
| Example 6 | 0.230 | 4.949 | |
| Example 7 | 0.712 | 1.327 | |
| Example 8 | 0.393 | 10.686 | |
| Example 9 | 0.195 | 1.116 | |
| Example 10 | 0.617 | 1.378 | |
| Example 11 | 0.567 | 10.337 | |
| Example 12 | 0.231 | 0.233 | 0% |
| Example 13 | 0.604 | 5.259 | |
| Example 14 | 0.401 | 2.438 | |
| Example 15 | 0.192 | 6.318 | 4% |
| Example 16 | 0.412 | 4.355 | |
| Example 17 | 0.606 | 10.385 | |
| Example 18 | 0.161 | 0.680 | 0% |
| Example 19 | 0.158 | 0.307 | 2% |
| Example 20 | 0.065 | 0.306 | 10% |
| Example 21 | 0.117 | 8.606 | 2% |
| Example 22 | 0.691 | 0.535 | |
| Example 23 | 0.049 | 0.960 | 52% |
| Example 24 | 0.907 | 1.938 | |
| Example 25 | 0.253 | 0.035 | |
| Example 26 | 0.324 | 0.022 | |
| Example 27 | 0.015 | 0.049 | 43% |
| Example 28 | 0.156 | 0.085 | |
| Example 29 | 0.480 | 1.756 | |
| Example 30 | 0.297 | 0.129 | |
| Example 31 | 0.900 | 2.102 | |
| Example 32 | 0.850 | 6.217 | |
| Example 33 | 0.314 | >31.2 | |
| Example 34 | 0.262 | 0.011 | |
| Example 35 | 0.523 | 0.362 | |
| Example 36 | 0.360 | 0.995 | |
| Example 37 | 0.878 | 12.595 | |
| Example 38 | 4.579 | 1.144 | |
| Example 39 | 2.746 | 1.422 | |
| Example 40 | >31.2 | >31.2 | |
| Example 41 | >31.2 | >31.2 | |
| Example 42 | 2.212 | 6.781 | |
| Example 43 | 0.010 | 0.739 | 74% |
| Example 44 | 7.340 | >31.2 | |
| Example 45 | 1.818 | 11.620 | |
| Example 46 | 0.033 | 5.414 | |
| Example 47 | 0.125 | 20.621 | |
| Example 48 | 0.698 | 7.588 | |
| Example 49 | 0.108 | 9.057 | |
| Example 50 | 0.110 | 3.132 | |
| Example 51 | 0.821 | 7.711 | |
| Example 52 | 0.215 | 1.922 | |
| Example 53 | 0.273 | 13.329 | |
| Example 54 | 1.489 | 30.332 | |
| Example 55 | 0.593 | 6.534 | |
| Example 56 | 0.874 | 3.731 | |
| Example 57 | 0.176 | 4.438 | 1% |
| Example 58 | 0.024 | 6.852 | 29% |
| Example 59 | 2.879 | 5.713 | |
| Example 60 | >31.2 | 24.831 | |
| Example 61 | 0.486 | 3.930 | |
| Example 62 | 0.049 | 6.039 | 87% |
| Example 63 | 0.241 | 2.326 | 6% |
| Example 64 | 0.779 | 1.455 | |
| Example 65 | 0.595 | 2.449 | |
| Example 66 | 0.395 | 2.502 | |
| Example 67 | 0.177 | 3.601 | |
| Example 68 | 0.147 | 5.882 | 39% |
| Example 69 | 15.715 | 24.451 | |
| Example 69 | 0.890 | 2.365 | |
| Example 70 | 0.930 | 3.363 | |
| Example 71 | 0.988 | 5.364 | |
| Example 72 | 1.007 | 7.135 | |
| Example 73 | 1.029 | 4.214 | |
| EXample 74 | 17.709 | 9.827 | |
| Example 75 | 0.737 | 26.662 | |
| Example 76 | 0.725 | 5.375 | |
| Example 77 | 0.519 | 0.544 | |
| Example 78 | 0.036 | 1.409 | 0% |
| Example 79 | 0.242 | 0.914 | |
| Example 80 | 0.285 | 0.289 | |
| Example 81 | 0.122 | 0.123 | 0% |
| Example 82 | 0.256 | 0.291 | |
| Example 83 | 0.681 | 0.818 | |
| Example 84 | 0.330 | 1.308 | |
| Example 85 | 0.812 | 2.372 | |
| Example 86 | 0.024 | 2.189 | 40% |
| Example 87 | 0.303 | 0.387 | |
| Example 88 | 0.102 | 1.566 | 0% |
| Example 89 | 0.159 | 0.879 | 0% |
| Example 90 | 0.078 | 1.609 | 3% |
| Example 91 | 0.043 | 2.046 | 111% |
| Example 92 | 0.239 | 0.520 | 20% |
| Example 93 | 0.996 | 0.643 | |
| Example 94 | no data | no data | |
| Example 95 | >31.2 | 2.068 | |
| Example 96 | 1.252 | 6.910 | |
| Example 97 | 4.574 | 3.607 | |
| Example 98 | 4.591 | >31.2 | |
| Example 99 | 7.714 | 7.076 | |
| Example | 4.007 | >31.2 | |
| 100 | | | |
| Example | 0.072 | 0.181 | |
| 101 | | | |
| Example | 1.552 | 10.046 | |
| 102 | | | |
| Example | 3.120 | 0.547 | |
| 103 | | | |
| Example | 1.289 | 1.399 | |
| 104 | | | |
| Example | 0.114 | 0.666 | |
| 105 | | | |
| Example | 0.164 | 0.485 | |
| 106 | | | |
| Example | 0.022 | 1.271 | 24% |
| 107 | | | |
| Example | 0.305 | 0.840 | |
| 108 | | | |
| Example | 0.956 | 0.470 | |
| 109 | | | |
| Example | 1.751 | 3.070 | |
| 110 | | | |
| Example | 0.058 | 2.920 | 52% |
| 111 | | | |
| Example | 1.631 | 1.945 | |
| 112 | | | |
| Example | 1.201 | 2.292 | |
| 113 | | | |
| Example | 0.776 | 8.464 | |
| 114 | | | |
| Example | 0.282 | 0.213 | |
| 115 | | | |
| Example | 0.296 | 0.119 | |
| 116 | | | |
| Example | 0.142 | 0.197 | |
| 117 | | | |
| Example | 0.230 | 0.103 | |
| 118 | | | |
| Example | 0.801 | 2.399 | |
| 119 | | | |
| Example | 0.582 | 1.220 | |
| 120 | | | |
| Example | 0.416 | 0.641 | |
| 121 | | | |
| Example | 20.649 | >31.2 | |
| 122 | | | |
| Example | 9.775 | 4.043 | |
| 123 | | | |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All documents cited herein are incorporated by reference.

We claim:

1. A method of inhibiting spermine oxidase in a cell comprising contacting said cell with a compound of Formula I 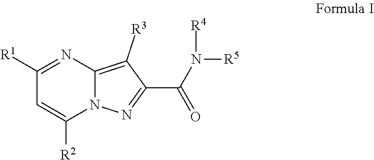
wherein:
R¹ is phenyl, 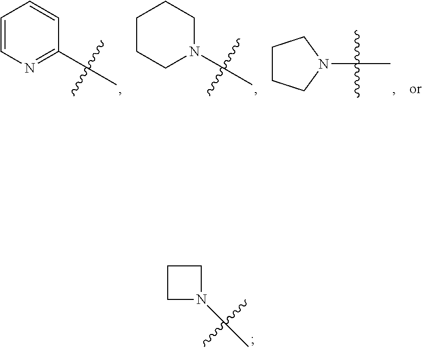 [SMILES: *c1ccccn1 ; *N1CCCCC1 ; *N1CCCC1 ; *N1CCC1]
wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl, 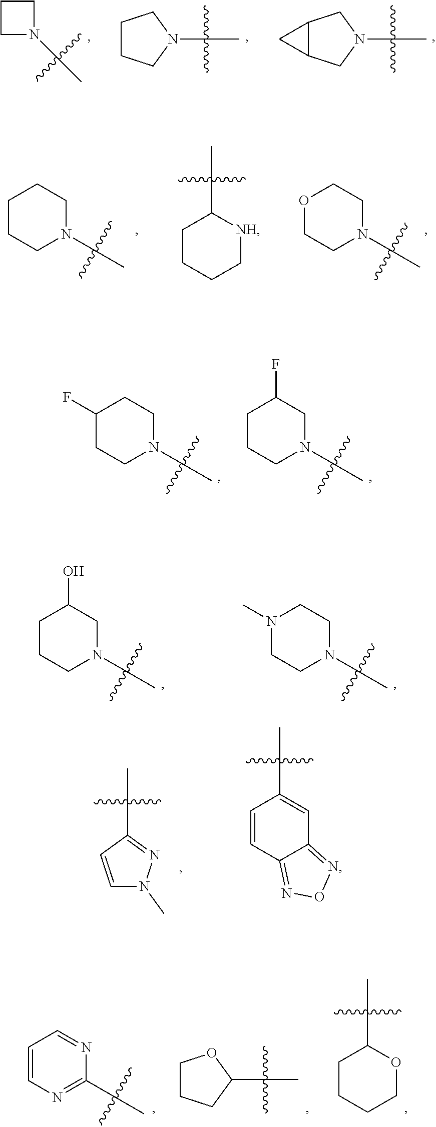
—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H —I, —Cl, —F, —CN, or —CH₃;
R⁴ is —H, or —CH₃;
R⁵ is —C₍₂₋₃₎alkyl-R^{a}, cyclobutyl, 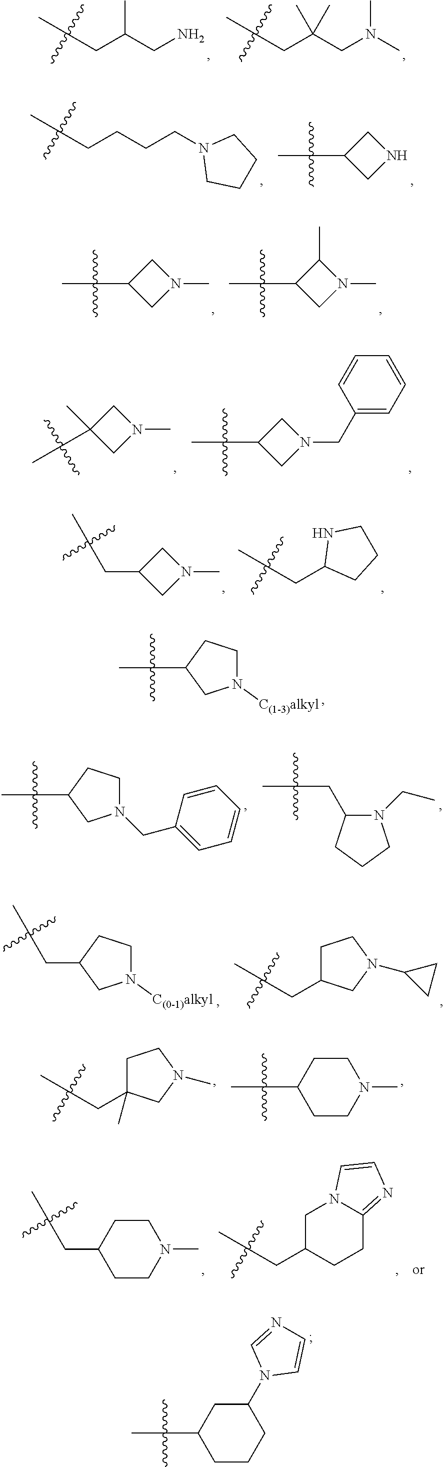
R^{a} is phenyl, imidazolyl, pyridinyl, azetidinyl, morpholinyl, —NH₂, —NHCH₃, —N(CH₃)₂, —NHpyridinyl, or —N(CH₃)pyridinyl; wherein said imidazolyl is optionally substituted with —CH₃;
and pharmaceutically acceptable salts thereof.

2. The method of claim 1, wherein R¹ is phenyl.

3. The method of claim 1, wherein R² is phenyl.

4. The method of claim 1, wherein
R³ is H
R⁴ is H

5. The method of claim 1, wherein the compound is selected from the group consisting of: 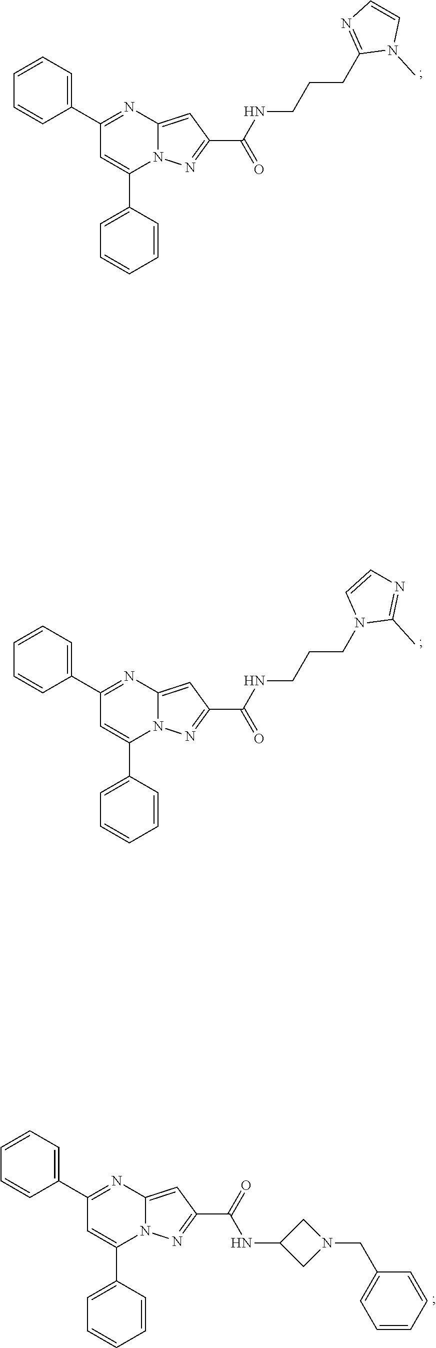 [SMILES: Cn1ccnc1CCCNC(=O)c1cc2nc(-c3ccccc3)cc(-c3ccccc3)n2n1 ; Cc1nccn1CCCNC(=O)c1cc2nc(-c3ccccc3)cc(-c3ccccc3)n2n1 ; O=C(NC1CN(Cc2ccccc2)C1)c1cc2nc(-c3ccccc3)cc(-c3ccccc3)n2n1] 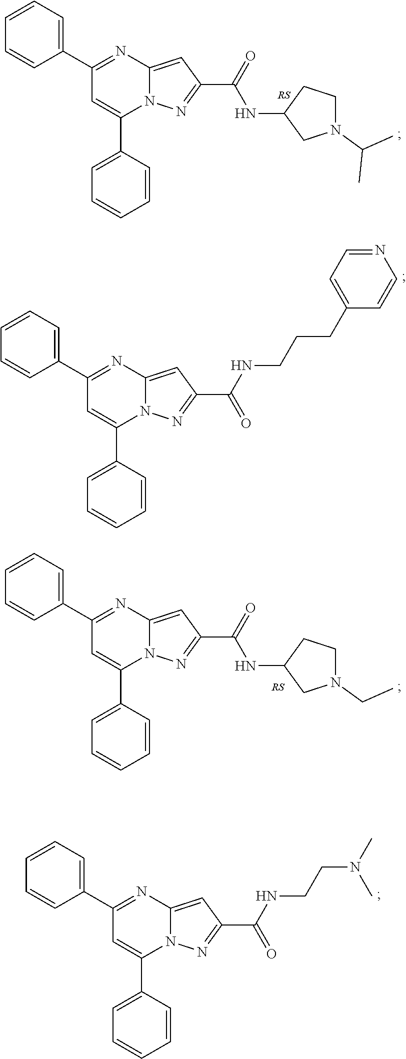 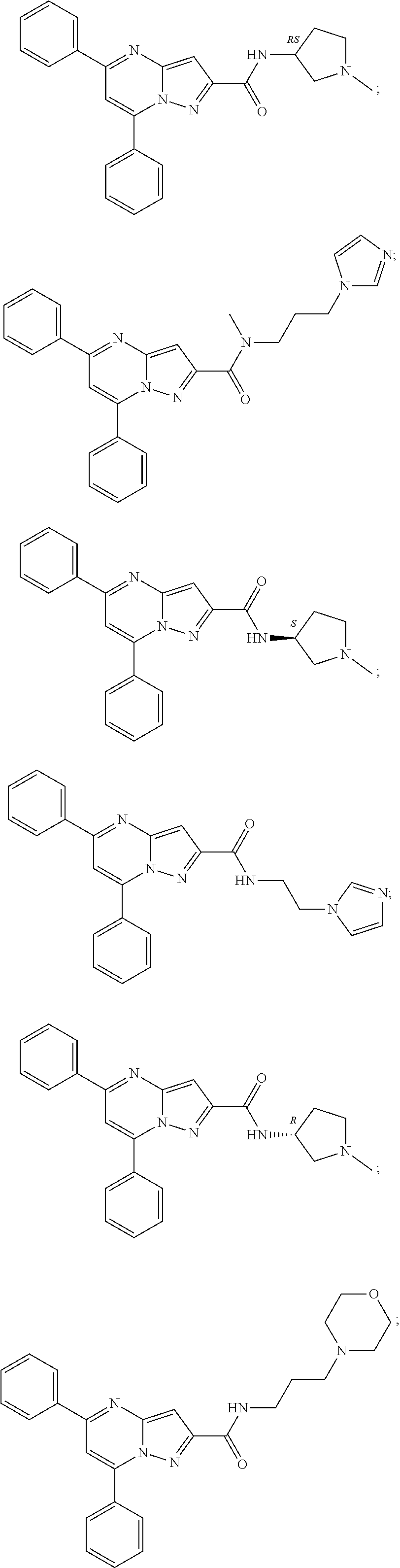 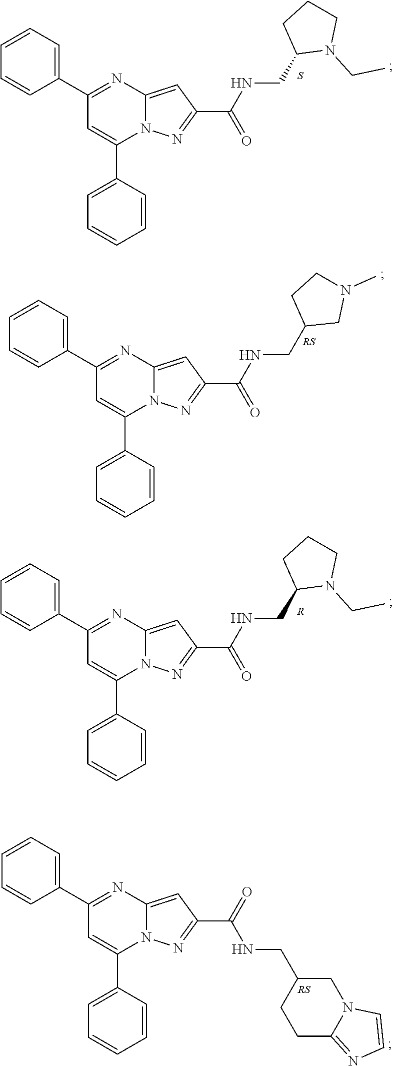 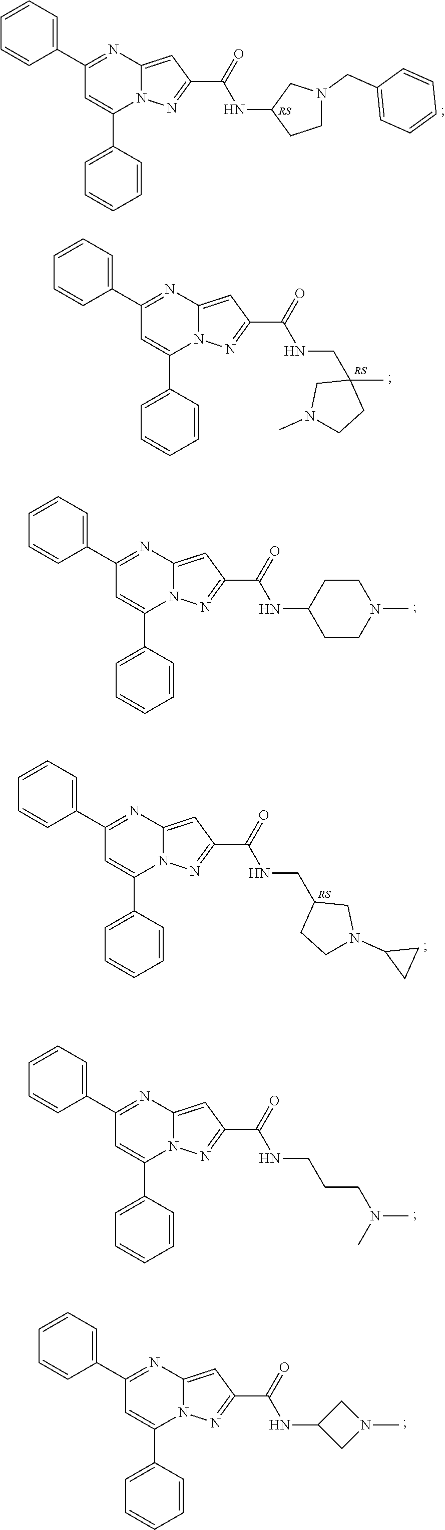 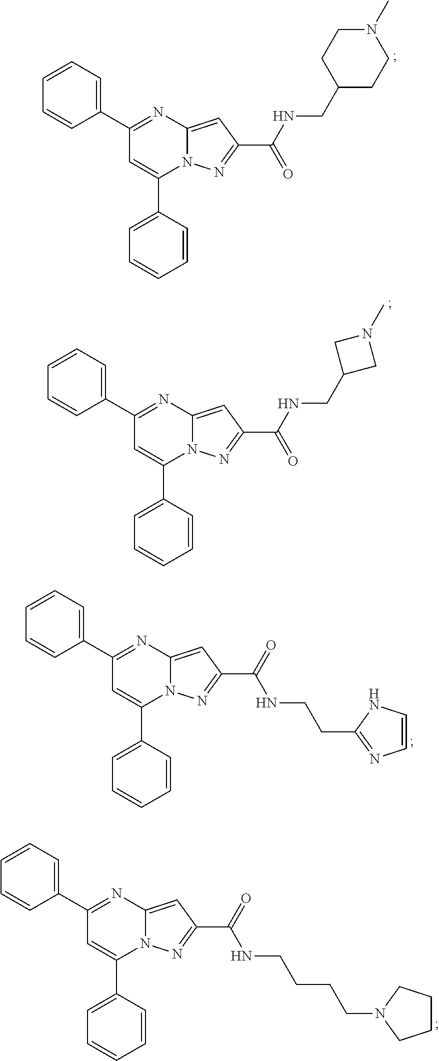 [SMILES: CN1CCC(CNC(=O)c2cc3nc(-c4ccccc4)cc(-c4ccccc4)n3n2)CC1 ; CN1CC(CNC(=O)c2cc3nc(-c4ccccc4)cc(-c4ccccc4)n3n2)C1 ; O=C(NCCc1ncc[nH]1)c1cc2nc(-c3ccccc3)cc(-c3ccccc3)n2n1 ; O=C(NCCCCN1CCCC1)c1cc2nc(-c3ccccc3)cc(-c3ccccc3)n2n1] 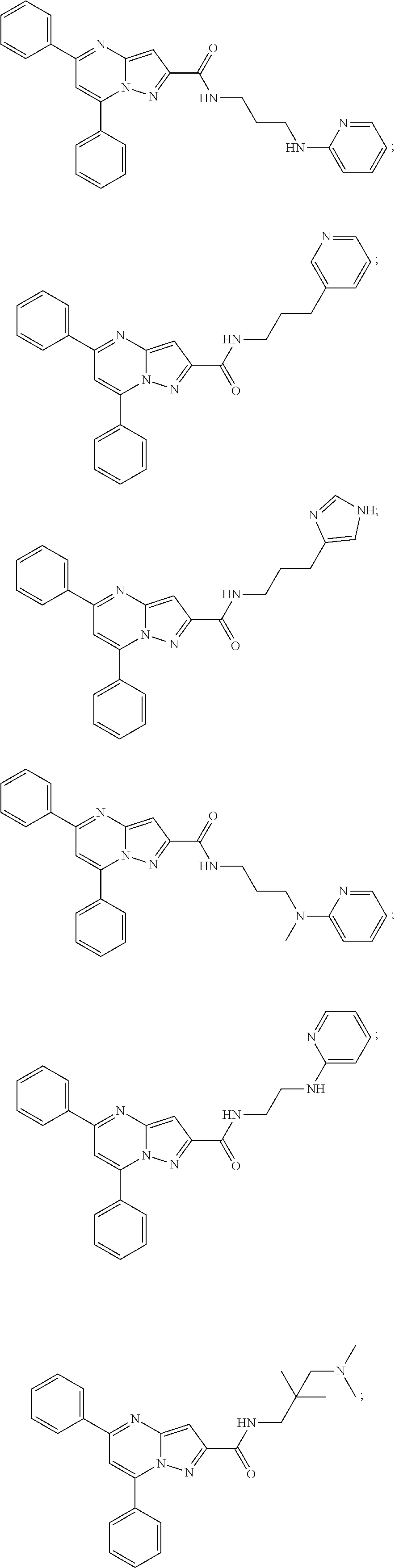 [SMILES: O=C(NCCCNc1ccccn1)c1cc2nc(-c3ccccc3)cc(-c3ccccc3)n2n1 ; O=C(NCCCc1cccnc1)c1cc2nc(-c3ccccc3)cc(-c3ccccc3)n2n1 ; O=C(NCCCc1c[nH]cn1)c1cc2nc(-c3ccccc3)cc(-c3ccccc3)n2n1 ; CN(CCCNC(=O)c1cc2nc(-c3ccccc3)cc(-c3ccccc3)n2n1)c1ccccn1 ; O=C(NCCNc1ccccn1)c1cc2nc(-c3ccccc3)cc(-c3ccccc3)n2n1 ; CN(C)CC(C)(C)CNC(=O)c1cc2nc(-c3ccccc3)cc(-c3ccccc3)n2n1] 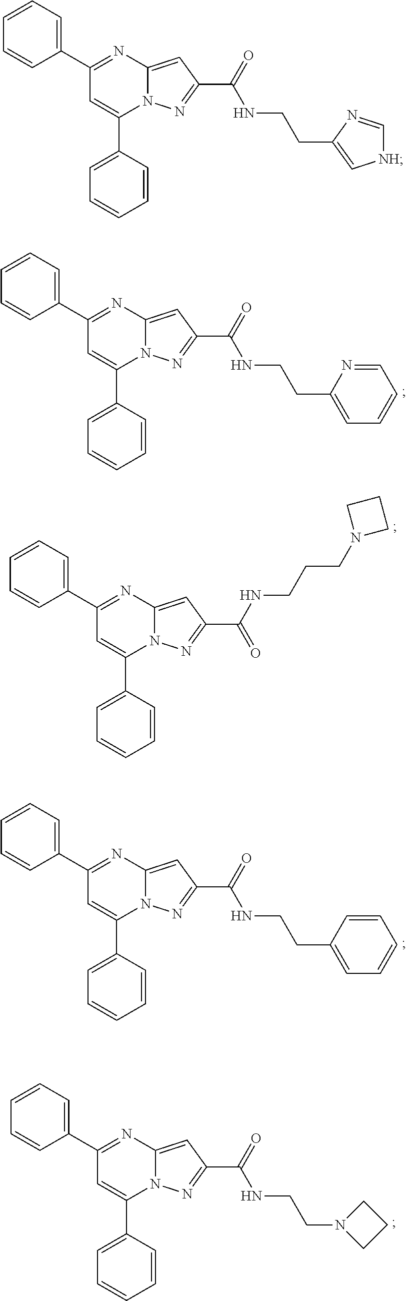 [SMILES: O=C(NCCc1c[nH]cn1)c1cc2nc(-c3ccccc3)cc(-c3ccccc3)n2n1 ; O=C(NCCc1ccccn1)c1cc2nc(-c3ccccc3)cc(-c3ccccc3)n2n1 ; O=C(NCCCN1CCC1)c1cc2nc(-c3ccccc3)cc(-c3ccccc3)n2n1 ; O=C(NCCc1ccccc1)c1cc2nc(-c3ccccc3)cc(-c3ccccc3)n2n1 ; O=C(NCCN1CCC1)c1cc2nc(-c3ccccc3)cc(-c3ccccc3)n2n1] 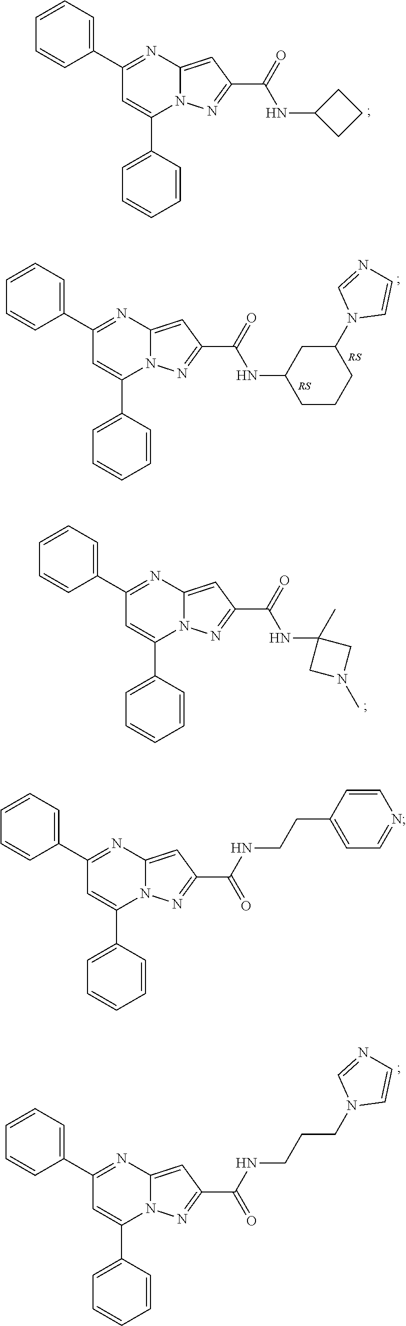 [SMILES: O=C(NC1CCC1)c1cc2nc(-c3ccccc3)cc(-c3ccccc3)n2n1 ; O=C(NC1CCCC(n2ccnc2)C1)c1cc2nc(-c3ccccc3)cc(-c3ccccc3)n2n1 ; CN1CC(C)(NC(=O)c2cc3nc(-c4ccccc4)cc(-c4ccccc4)n3n2)C1 ; O=C(NCCc1ccncc1)c1cc2nc(-c3ccccc3)cc(-c3ccccc3)n2n1 ; O=C(NCCCn1ccnc1)c1cc2nc(-c3ccccc3)cc(-c3ccccc3)n2n1] 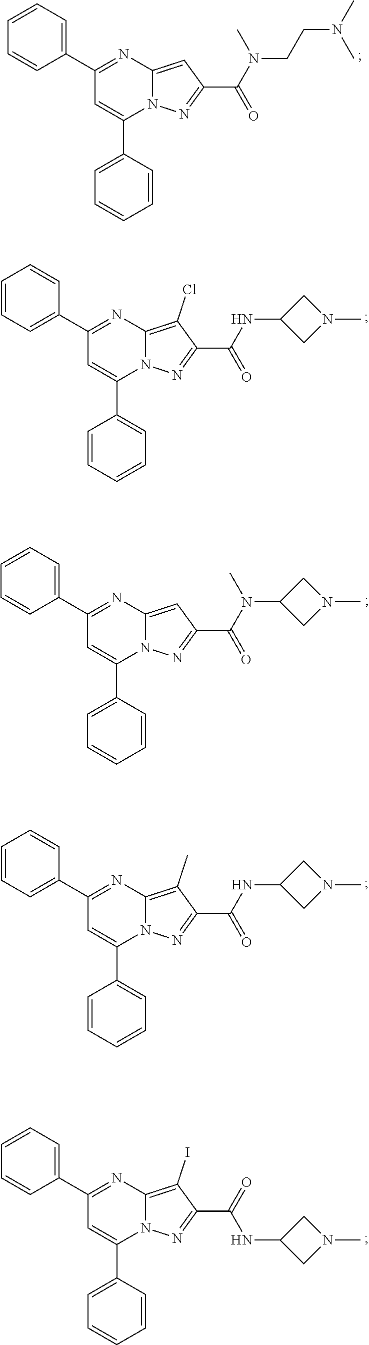 [SMILES: CN(C)CCN(C)C(=O)c1cc2nc(-c3ccccc3)cc(-c3ccccc3)n2n1 ; CN1CC(NC(=O)c2nn3c(-c4ccccc4)cc(-c4ccccc4)nc3c2Cl)C1 ; CN1CC(N(C)C(=O)c2cc3nc(-c4ccccc4)cc(-c4ccccc4)n3n2)C1 ; Cc1c(C(=O)NC2CN(C)C2)nn2c(-c3ccccc3)cc(-c3ccccc3)nc12 ; CN1CC(NC(=O)c2nn3c(-c4ccccc4)cc(-c4ccccc4)nc3c2I)C1] 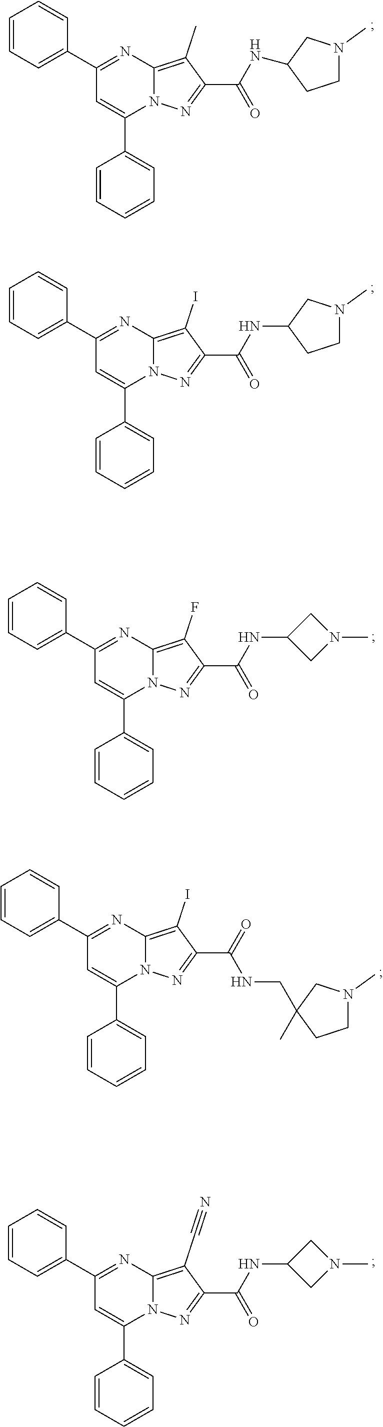 [SMILES: Cc1c(C(=O)NC2CCN(C)C2)nn2c(-c3ccccc3)cc(-c3ccccc3)nc12 ; CN1CCC(NC(=O)c2nn3c(-c4ccccc4)cc(-c4ccccc4)nc3c2I)C1 ; CN1CC(NC(=O)c2nn3c(-c4ccccc4)cc(-c4ccccc4)nc3c2F)C1 ; CN1CCC(C)(CNC(=O)c2nn3c(-c4ccccc4)cc(-c4ccccc4)nc3c2I)C1 ; CN1CC(NC(=O)c2nn3c(-c4ccccc4)cc(-c4ccccc4)nc3c2C#N)C1] 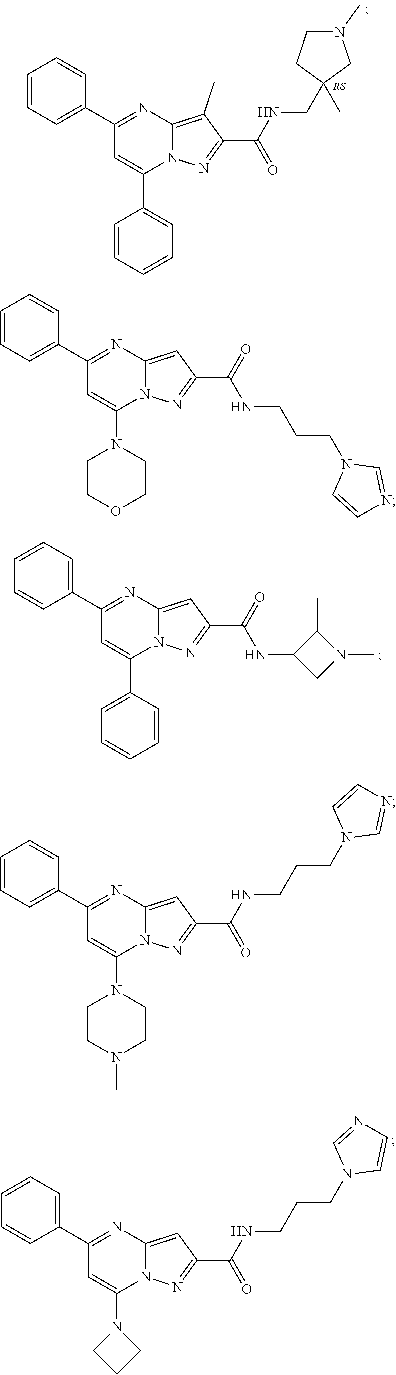 [SMILES: Cc1c(C(=O)NCC2(C)CCN(C)C2)nn2c(-c3ccccc3)cc(-c3ccccc3)nc12 ; O=C(NCCCn1ccnc1)c1cc2nc(-c3ccccc3)cc(N3CCOCC3)n2n1 ; CC1C(NC(=O)c2cc3nc(-c4ccccc4)cc(-c4ccccc4)n3n2)CN1C ; CN1CCN(c2cc(-c3ccccc3)nc3cc(C(=O)NCCCn4ccnc4)nn23)CC1 ; O=C(NCCCn1ccnc1)c1cc2nc(-c3ccccc3)cc(N3CCC3)n2n1] 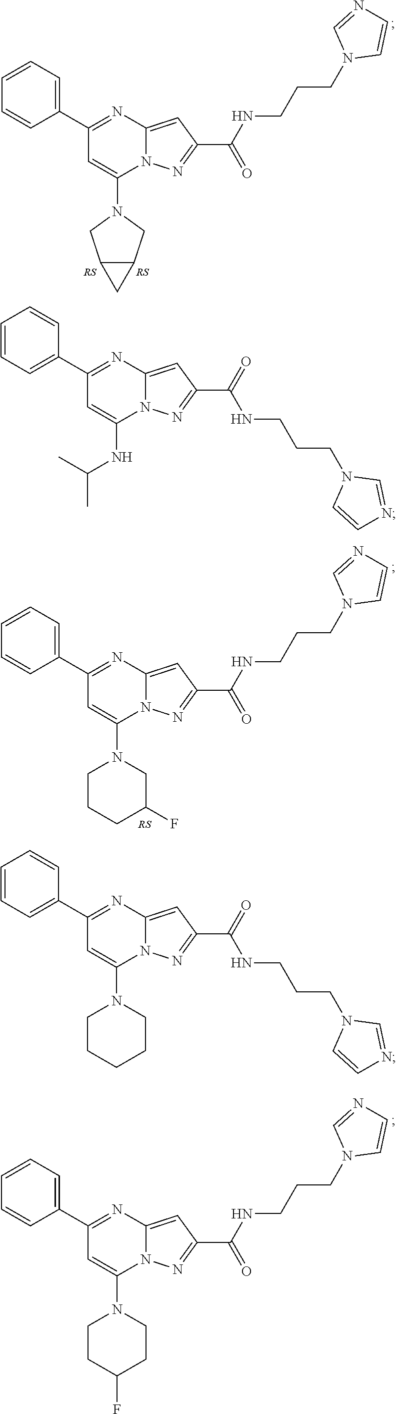 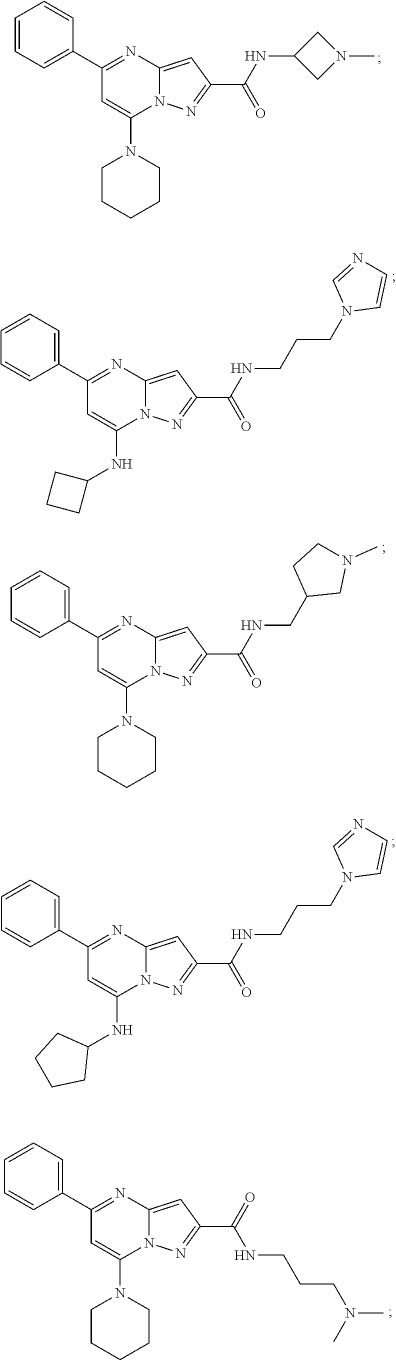 [SMILES: CN1CC(NC(=O)c2cc3nc(-c4ccccc4)cc(N4CCCCC4)n3n2)C1 ; O=C(NCCCn1ccnc1)c1cc2nc(-c3ccccc3)cc(NC3CCC3)n2n1 ; CN1CCC(CNC(=O)c2cc3nc(-c4ccccc4)cc(N4CCCCC4)n3n2)C1 ; O=C(NCCCn1ccnc1)c1cc2nc(-c3ccccc3)cc(NC3CCCC3)n2n1 ; CN(C)CCCNC(=O)c1cc2nc(-c3ccccc3)cc(N3CCCCC3)n2n1] 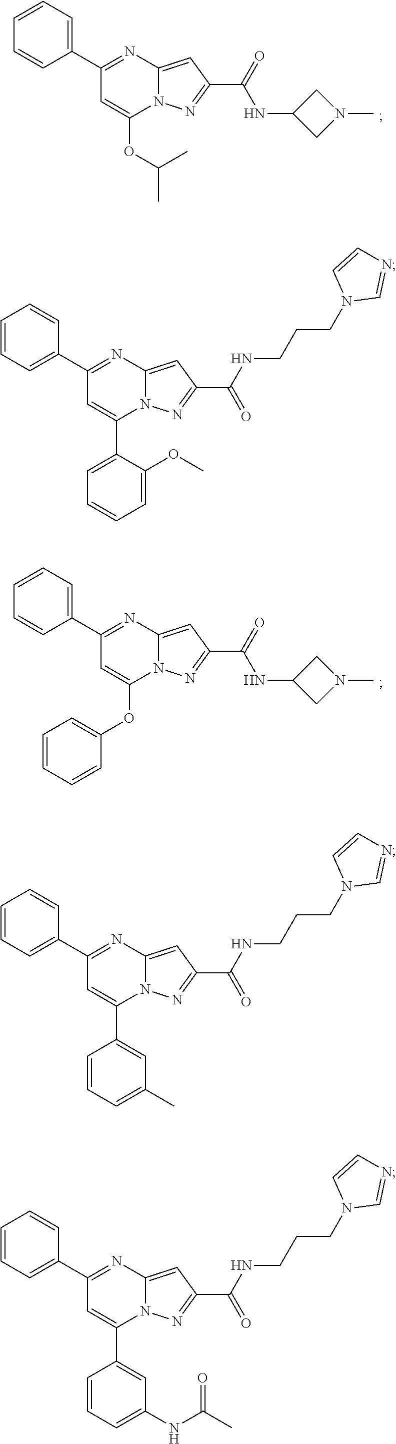 [SMILES: CC(C)Oc1cc(-c2ccccc2)nc2cc(C(=O)NC3CN(C)C3)nn12 ; COc1ccccc1-c1cc(-c2ccccc2)nc2cc(C(=O)NCCCn3ccnc3)nn12 ; CN1CC(NC(=O)c2cc3nc(-c4ccccc4)cc(Oc4ccccc4)n3n2)C1 ; Cc1cccc(-c2cc(-c3ccccc3)nc3cc(C(=O)NCCCn4ccnc4)nn23)c1 ; CC(=O)Nc1cccc(-c2cc(-c3ccccc3)nc3cc(C(=O)NCCCn4ccnc4)nn23)c1] 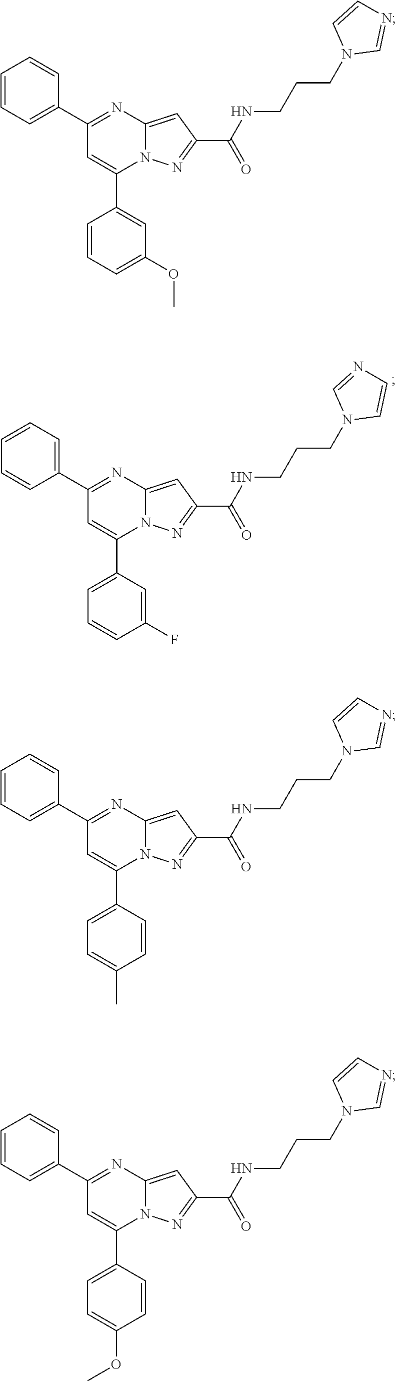 [SMILES: COc1cccc(-c2cc(-c3ccccc3)nc3cc(C(=O)NCCCn4ccnc4)nn23)c1 ; O=C(NCCCn1ccnc1)c1cc2nc(-c3ccccc3)cc(-c3cccc(F)c3)n2n1 ; Cc1ccc(-c2cc(-c3ccccc3)nc3cc(C(=O)NCCCn4ccnc4)nn23)cc1 ; COc1ccc(-c2cc(-c3ccccc3)nc3cc(C(=O)NCCCn4ccnc4)nn23)cc1] 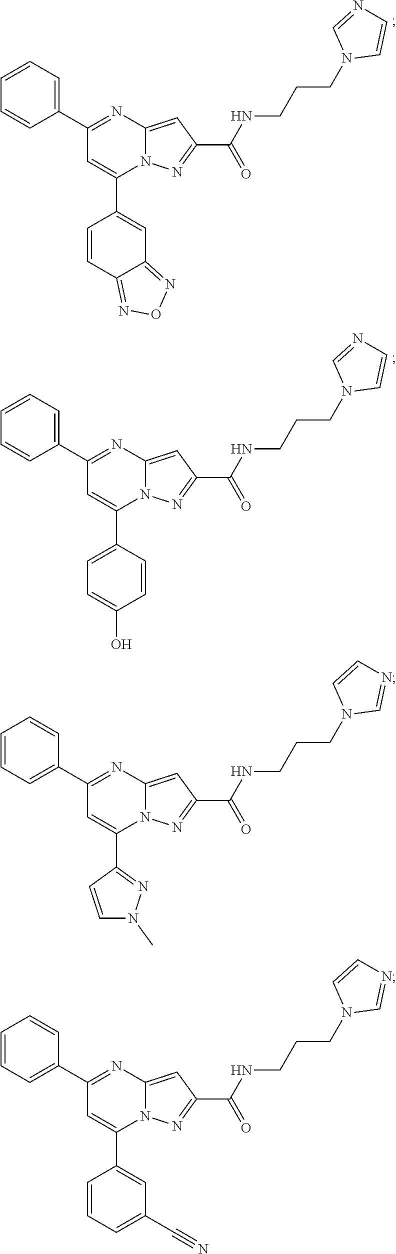 [SMILES: O=C(NCCCn1ccnc1)c1cc2nc(-c3ccccc3)cc(-c3ccc4nonc4c3)n2n1 ; O=C(NCCCn1ccnc1)c1cc2nc(-c3ccccc3)cc(-c3ccc(O)cc3)n2n1 ; Cn1ccc(-c2cc(-c3ccccc3)nc3cc(C(=O)NCCCn4ccnc4)nn23)n1 ; N#Cc1cccc(-c2cc(-c3ccccc3)nc3cc(C(=O)NCCCn4ccnc4)nn23)c1] 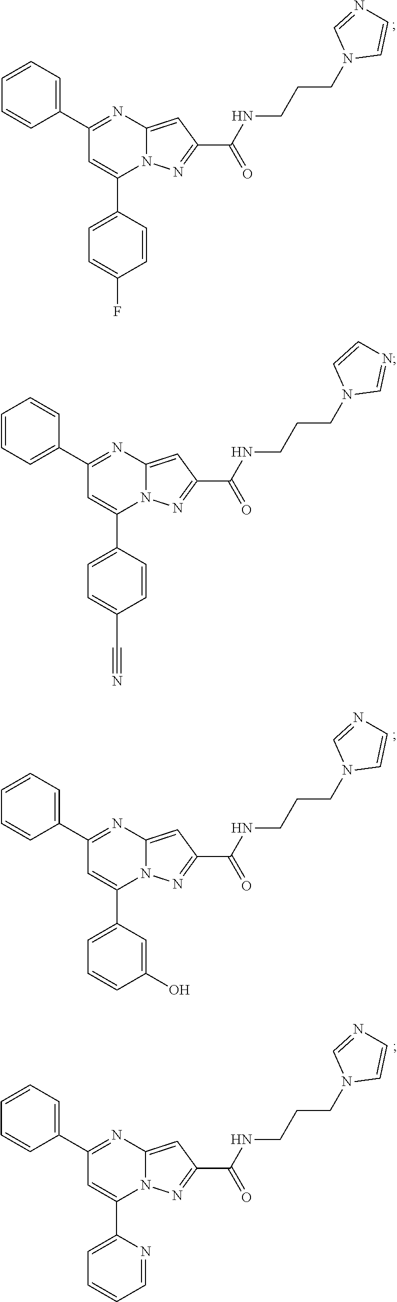 [SMILES: O=C(NCCCn1ccnc1)c1cc2nc(-c3ccccc3)cc(-c3ccc(F)cc3)n2n1 ; N#Cc1ccc(-c2cc(-c3ccccc3)nc3cc(C(=O)NCCCn4ccnc4)nn23)cc1 ; O=C(NCCCn1ccnc1)c1cc2nc(-c3ccccc3)cc(-c3cccc(O)c3)n2n1 ; O=C(NCCCn1ccnc1)c1cc2nc(-c3ccccc3)cc(-c3ccccn3)n2n1] 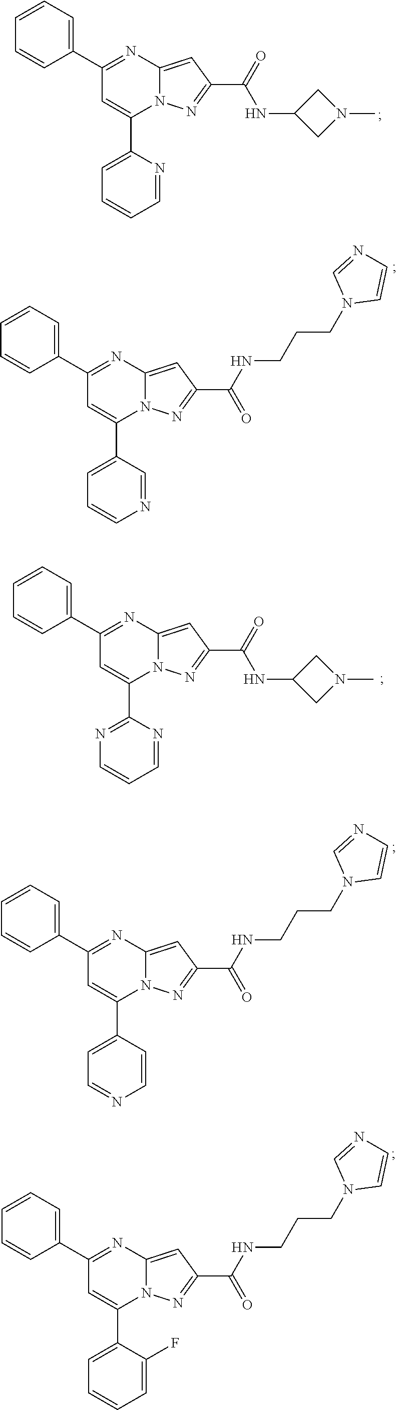 [SMILES: CN1CC(NC(=O)c2cc3nc(-c4ccccc4)cc(-c4ccccn4)n3n2)C1 ; O=C(NCCCn1ccnc1)c1cc2nc(-c3ccccc3)cc(-c3cccnc3)n2n1 ; CN1CC(NC(=O)c2cc3nc(-c4ccccc4)cc(-c4ncccn4)n3n2)C1 ; O=C(NCCCn1ccnc1)c1cc2nc(-c3ccccc3)cc(-c3ccncc3)n2n1 ; O=C(NCCCn1ccnc1)c1cc2nc(-c3ccccc3)cc(-c3ccccc3F)n2n1] 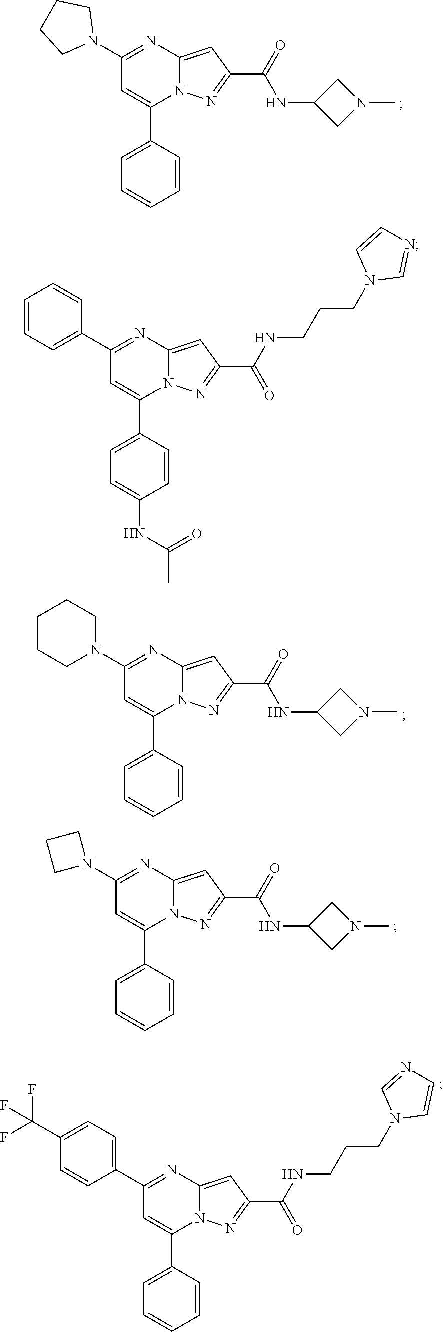 [SMILES: CN1CC(NC(=O)c2cc3nc(N4CCCC4)cc(-c4ccccc4)n3n2)C1 ; CC(=O)Nc1ccc(-c2cc(-c3ccccc3)nc3cc(C(=O)NCCCn4ccnc4)nn23)cc1 ; CN1CC(NC(=O)c2cc3nc(N4CCCCC4)cc(-c4ccccc4)n3n2)C1 ; CN1CC(NC(=O)c2cc3nc(N4CCC4)cc(-c4ccccc4)n3n2)C1 ; O=C(NCCCn1ccnc1)c1cc2nc(-c3ccc(C(F)(F)F)cc3)cc(-c3ccccc3)n2n1] 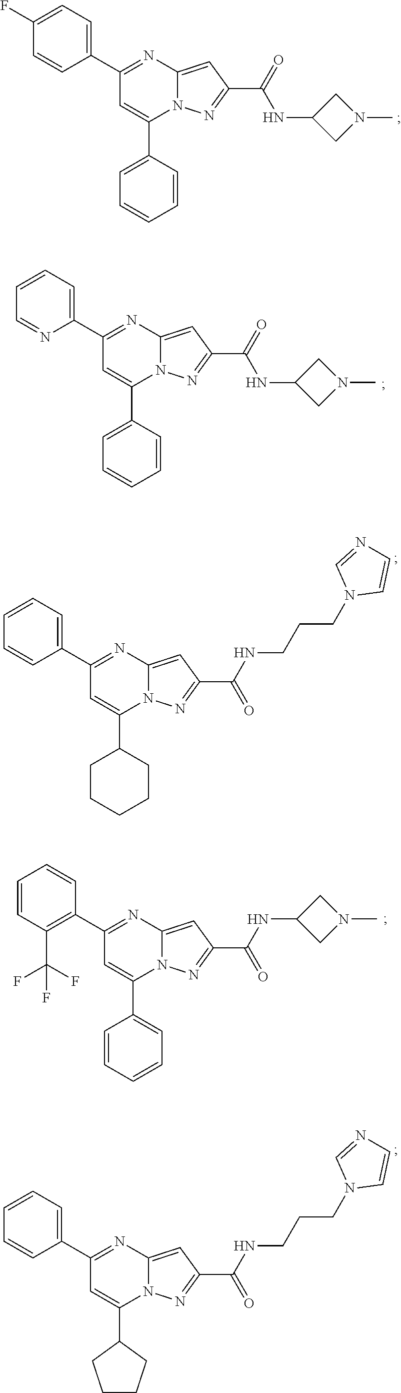 [SMILES: CN1CC(NC(=O)c2cc3nc(-c4ccc(F)cc4)cc(-c4ccccc4)n3n2)C1 ; CN1CC(NC(=O)c2cc3nc(-c4ccccn4)cc(-c4ccccc4)n3n2)C1 ; O=C(NCCCn1ccnc1)c1cc2nc(-c3ccccc3)cc(C3CCCCC3)n2n1 ; CN1CC(NC(=O)c2cc3nc(-c4ccccc4C(F)(F)F)cc(-c4ccccc4)n3n2)C1 ; O=C(NCCCn1ccnc1)c1cc2nc(-c3ccccc3)cc(C3CCCC3)n2n1] 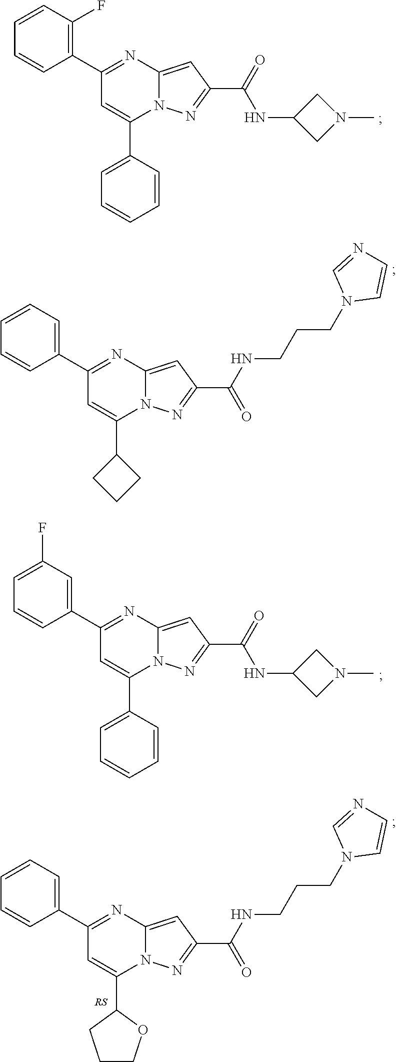 [SMILES: CN1CC(NC(=O)c2cc3nc(-c4ccccc4F)cc(-c4ccccc4)n3n2)C1 ; O=C(NCCCn1ccnc1)c1cc2nc(-c3ccccc3)cc(C3CCC3)n2n1 ; CN1CC(NC(=O)c2cc3nc(-c4cccc(F)c4)cc(-c4ccccc4)n3n2)C1 ; O=C(NCCCn1ccnc1)c1cc2nc(-c3ccccc3)cc(C3CCCO3)n2n1] 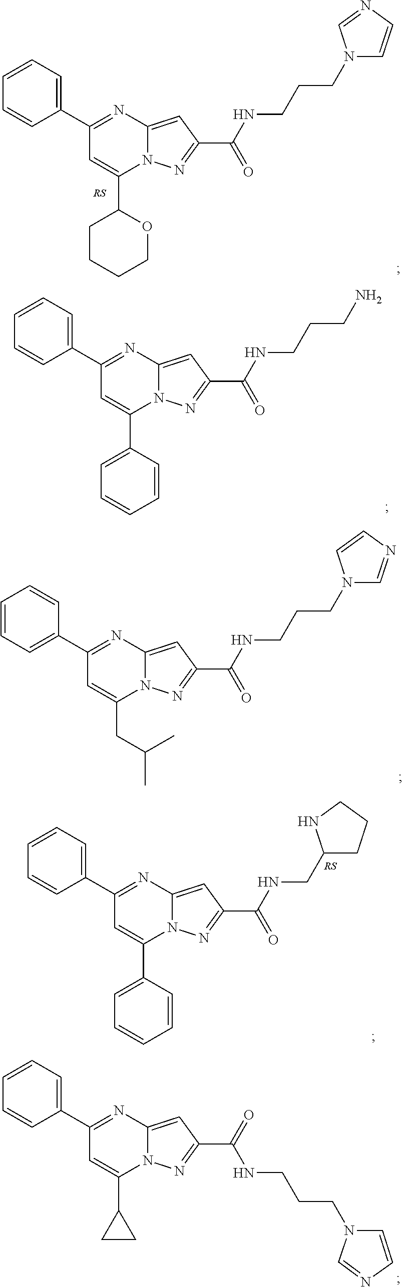 [SMILES: O=C(NCCCn1ccnc1)c1cc2nc(-c3ccccc3)cc(C3CCCCO3)n2n1 ; NCCCNC(=O)c1cc2nc(-c3ccccc3)cc(-c3ccccc3)n2n1 ; CC(C)Cc1cc(-c2ccccc2)nc2cc(C(=O)NCCCn3ccnc3)nn12 ; O=C(NCC1CCCN1)c1cc2nc(-c3ccccc3)cc(-c3ccccc3)n2n1 ; O=C(NCCCn1ccnc1)c1cc2nc(-c3ccccc3)cc(C3CC3)n2n1] 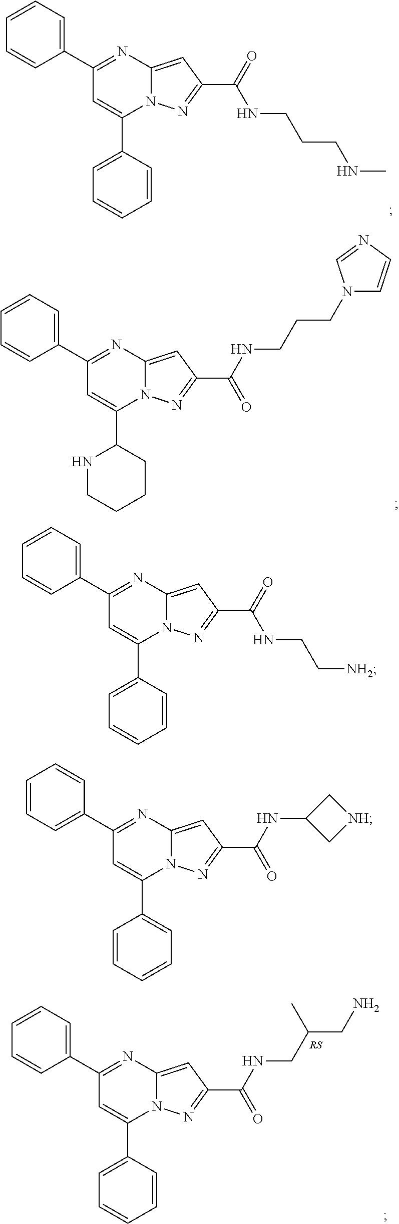 [SMILES: CNCCCNC(=O)c1cc2nc(-c3ccccc3)cc(-c3ccccc3)n2n1 ; O=C(NCCCn1ccnc1)c1cc2nc(-c3ccccc3)cc(C3CCCCN3)n2n1 ; NCCNC(=O)c1cc2nc(-c3ccccc3)cc(-c3ccccc3)n2n1 ; O=C(NC1CNC1)c1cc2nc(-c3ccccc3)cc(-c3ccccc3)n2n1 ; CC(CN)CNC(=O)c1cc2nc(-c3ccccc3)cc(-c3ccccc3)n2n1] 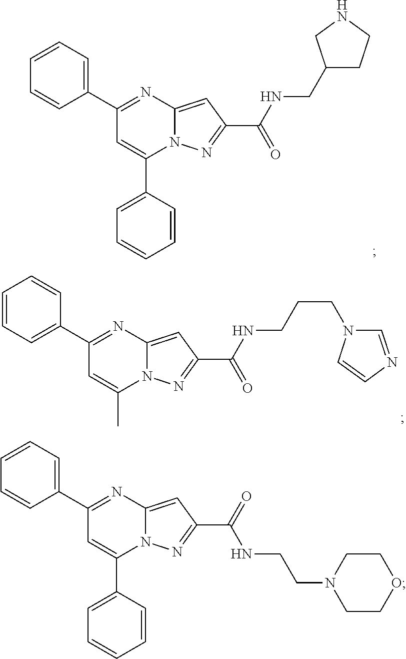 [SMILES: O=C(NCC1CCNC1)c1cc2nc(-c3ccccc3)cc(-c3ccccc3)n2n1 ; Cc1cc(-c2ccccc2)nc2cc(C(=O)NCCCn3ccnc3)nn12 ; O=C(NCCN1CCOCC1)c1cc2nc(-c3ccccc3)cc(-c3ccccc3)n2n1]
and pharmaceutically acceptable salts thereof.

6. The method of claim 5 wherein the compound is 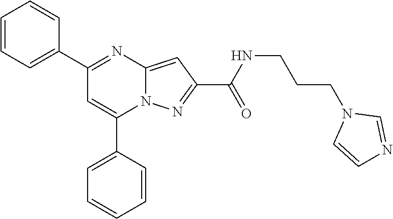 [SMILES: O=C(NCCCn1ccnc1)c1cc2nc(-c3ccccc3)cc(-c3ccccc3)n2n1]
and pharmaceutically acceptable salts thereof.

7. A compound of Formula I 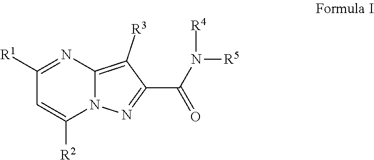
wherein:
R¹ is phenyl, 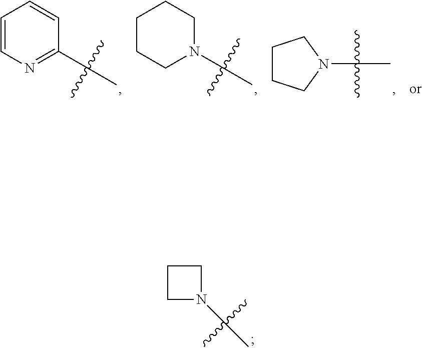 [SMILES: *c1ccccn1 ; *N1CCCCC1 ; *N1CCCC1 ; *N1CCC1]
wherein said phenyl is optionally substituted with —F, or —CF₃;
R² is phenyl, pyridinyl, —CH₃, —CH₂CH(CH₃)₂, —C₍₃₋₆₎cycloalkyl, —NHC₍₄₋₅₎cycloalkyl, 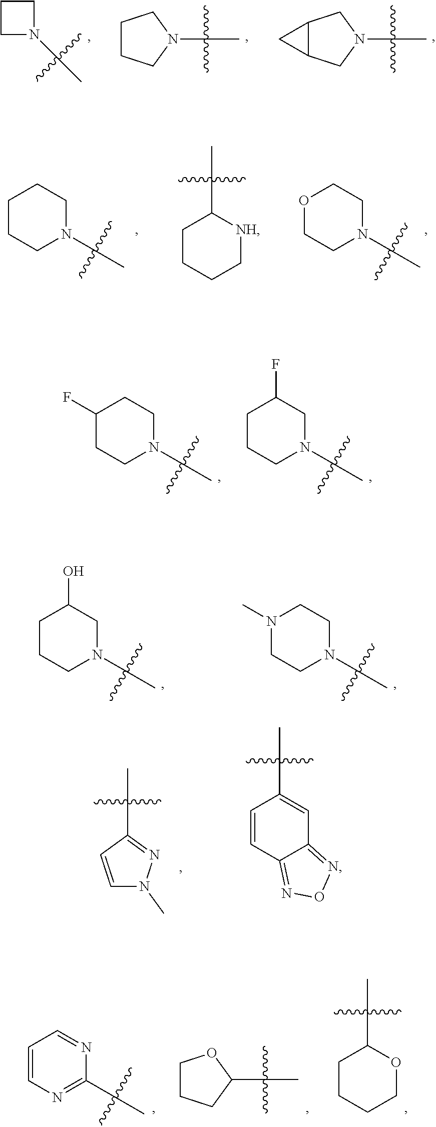
—OCH(CH₃)₂, —NHCH(CH₃)₂, or —OPh; wherein said phenyl is optionally substituted with —NHC(O)CH₃, —CN, —CH₃, —F, —OH, or —OCH₃;
R³ is —H —I, —Cl, —F, —CN, or —CH₃;
R⁴ is —H, or —CH₃;
R⁵ is —C₍₂₋₃₎alkyl-R^{a}, cyclobutyl, 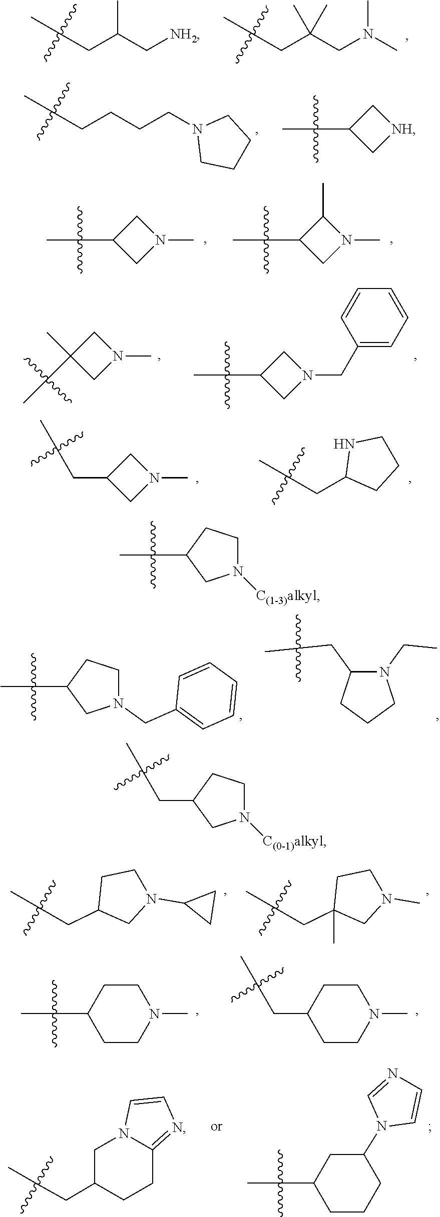
R^{a} is pyridinyl, azetidinyl, —NH₂, —NHCH₃, —N(CH₃)₂, —NHpyridinyl, or —N(CH₃)pyridinyl;
and pharmaceutically acceptable salts thereof.

8. The compound of claim 7, wherein R¹ is phenyl.

9. The compound of claim 7, wherein R² is phenyl.

10. The compound of claim 7, wherein
R³ is H
R⁴ is H

11. The compound of claim 7 selected from the group consisting of: 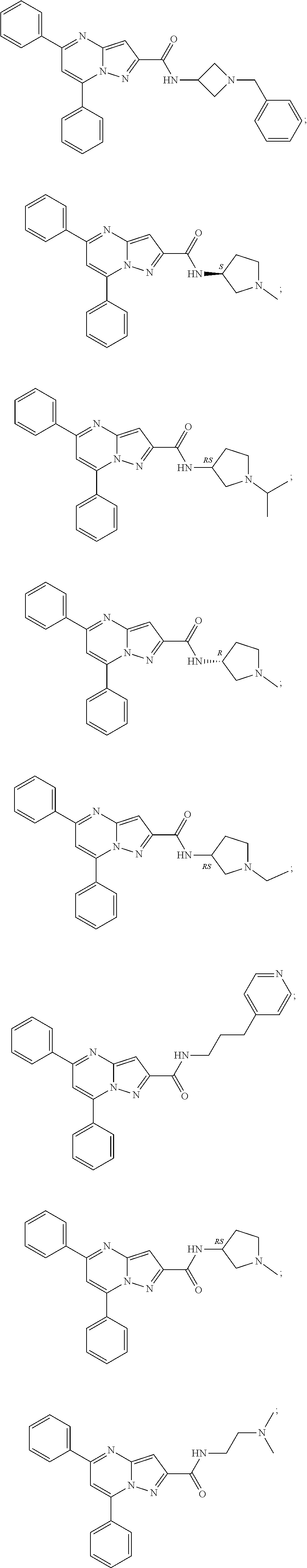 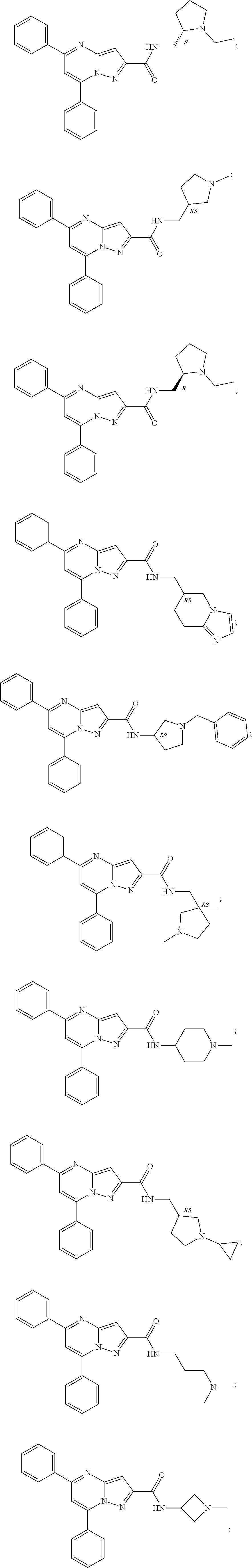 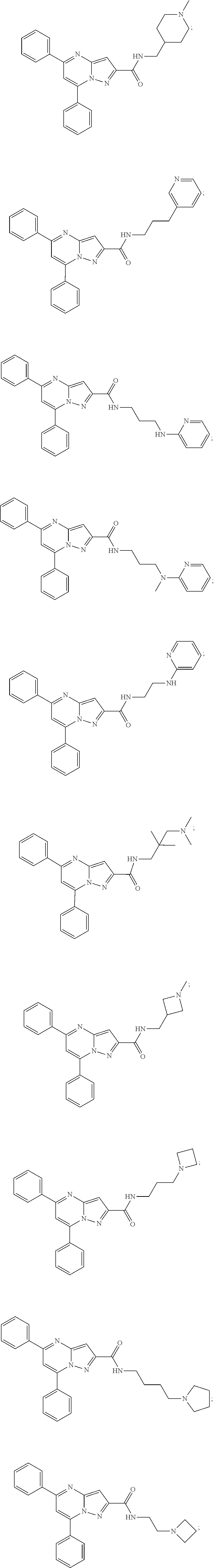 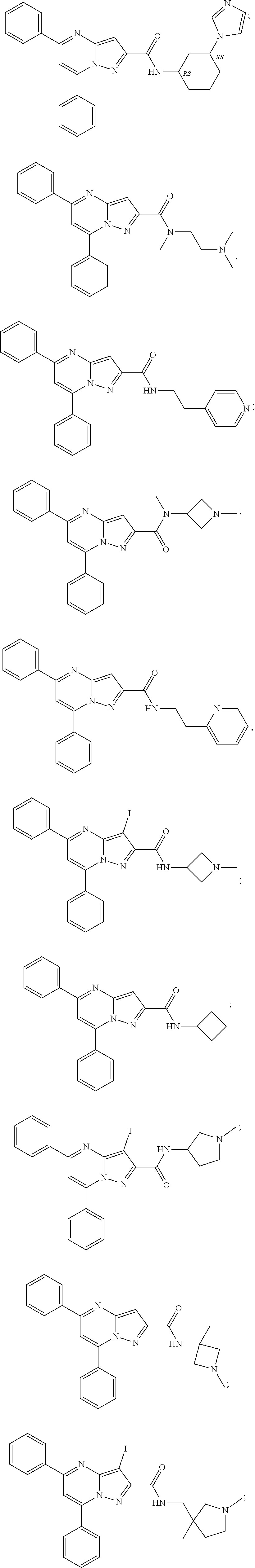 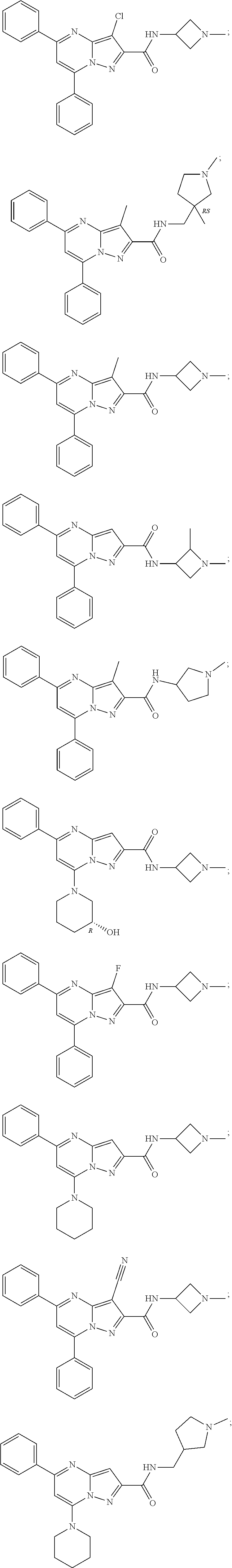 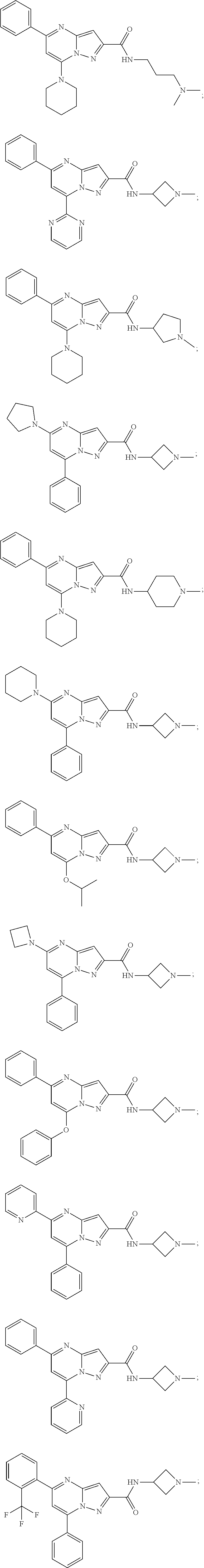 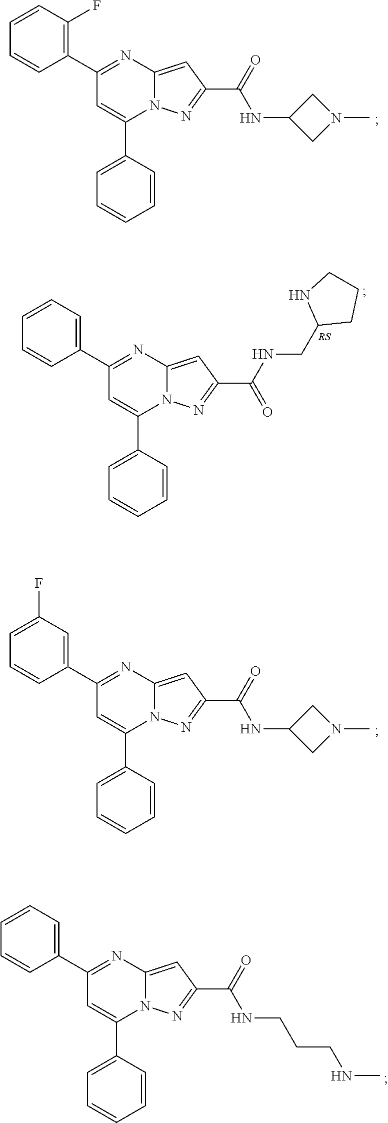 [SMILES: CN1CC(NC(=O)c2cc3nc(-c4ccccc4F)cc(-c4ccccc4)n3n2)C1 ; O=C(NCC1CCCN1)c1cc2nc(-c3ccccc3)cc(-c3ccccc3)n2n1 ; CN1CC(NC(=O)c2cc3nc(-c4cccc(F)c4)cc(-c4ccccc4)n3n2)C1 ; CNCCCNC(=O)c1cc2nc(-c3ccccc3)cc(-c3ccccc3)n2n1] 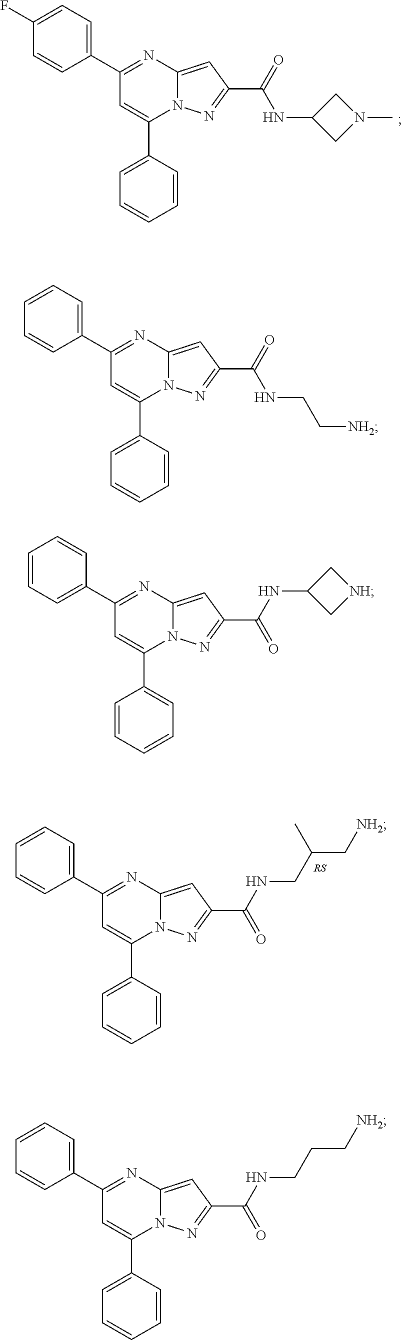 [SMILES: CN1CC(NC(=O)c2cc3nc(-c4ccc(F)cc4)cc(-c4ccccc4)n3n2)C1 ; NCCNC(=O)c1cc2nc(-c3ccccc3)cc(-c3ccccc3)n2n1 ; O=C(NC1CNC1)c1cc2nc(-c3ccccc3)cc(-c3ccccc3)n2n1 ; CC(CN)CNC(=O)c1cc2nc(-c3ccccc3)cc(-c3ccccc3)n2n1 ; NCCCNC(=O)c1cc2nc(-c3ccccc3)cc(-c3ccccc3)n2n1] 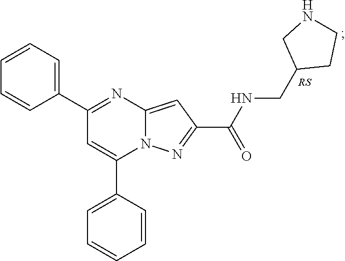 [SMILES: O=C(NCC1CCNC1)c1cc2nc(-c3ccccc3)cc(-c3ccccc3)n2n1]
and pharmaceutically acceptable salts thereof.

12. A process for making a pharmaceutical composition comprising mixing a compound of claim 7 and a pharmaceutically acceptable carrier.

13. A method for treating or ameliorating a SMOX mediated syndrome, disorder or disease comprising administering to a subject in need thereof an effective amount of a compound of claim 7.

14. The method of claim 13, wherein the disease is selected from the group consisting of: colorectal cancer and vision loss.

15. The method of claim 14, wherein the disease is colorectal cancer.

16. The method of claim 14, wherein the disease is vision loss.

17. A method of treating or ameliorating a syndrome, disorder or disease, in a subject in need thereof comprising administering to the subject an effective amount of a compound of claim 7 or composition or medicament thereof in a combination therapy with one or more anti-inflammatory agents, or immunosuppressive agents, wherein said syndrome, disorder or disease is selected from the group consisting of: colorectal cancer and vision loss.

18. The method of claim 17, wherein the disease is colorectal cancer.
